SEQUENCE LISTING (347 sequences)

SEQ ID NO: 1 - protein, 5 aa, synthetic construct
ELSMQ

SEQ ID NO: 2 - protein, 5 aa, synthetic construct
DYFIH

SEQ ID NO: 3 - protein, 5 aa, synthetic construct
SYAIS

SEQ ID NO: 4 - protein, 5 aa, synthetic construct
ESSMH

SEQ ID NO: 5 - protein, 5 aa, synthetic construct
ELSMH

SEQ ID NO: 6 - protein, 7 aa, synthetic construct
TSGMGVG

SEQ ID NO: 7 - protein, 17 aa, synthetic construct
GFDPDDGETMYAEKFQG

SEQ ID NO: 8 - protein, 17 aa, synthetic construct
GFDPDDGETMYAERFQG

SEQ ID NO: 9 - protein, 17 aa, synthetic construct
GFDPDGGETMYAEKFQG

SEQ ID NO: 10 - protein, 17 aa, synthetic construct
GFDPDDGETMYAEKLQG

SEQ ID NO: 11 - protein, 17 aa, synthetic construct
LVDPEDGETIYAEKFQG

SEQ ID NO: 12 - protein, 17 aa, synthetic construct
LVDPEDGETIYAERFQG

SEQ ID NO: 13 - protein, 17 aa, synthetic construct
LVDPEDGETIYAEKFRG

SEQ ID NO: 14 - protein, 17 aa, synthetic construct
LVDPEDGETIYAQKFQG

SEQ ID NO: 15 - protein, 17 aa, synthetic construct
GIIPIFGTANYAQKFQG

SEQ ID NO: 16 - protein, 17 aa, synthetic construct
GFDPEDGETIYAQKFQG

SEQ ID NO: 17 - protein, 16 aa, synthetic construct
HIWWDDDKYYNPYLKN

SEQ ID NO: 18 - protein, 9 aa, synthetic construct
SPGYDFFDY

SEQ ID NO: 19 - protein, 13 aa, synthetic construct
PGGILTDPDAFDI

SEQ ID NO: 20 - protein, 6 aa, synthetic construct
EESYGP

SEQ ID NO: 21 - protein, 13 aa, synthetic construct
EEWSGDGDDAFDI

SEQ ID NO: 22 - protein, 18 aa, synthetic construct
SPLRGSGWYWHYYYGMDV

SEQ ID NO: 23 - protein, 9 aa, synthetic construct
TIGDDYFDY

SEQ ID NO: 24 - protein, 10 aa, synthetic construct
DIQGLAEFDY

SEQ ID NO: 25 - protein, 9 aa, synthetic construct
SAYGSTYGY

SEQ ID NO: 26 - protein, 11 aa, synthetic construct
GGNYIGSKSVH

SEQ ID NO: 27 - protein, 11 aa, synthetic construct
GGDNIGSKSVH

SEQ ID NO: 28 - protein, 11 aa, synthetic construct
GGNNIGSKSVH

SEQ ID NO: 29 - protein, 11 aa, synthetic construct
VGTNFGSKNVH

SEQ ID NO: 30 - protein, 14 aa, synthetic construct
TGTSSDVGGYNYVS

SEQ ID NO: 31 - protein, 11 aa, synthetic construct
GGNNIGSEGVH

SEQ ID NO: 32 - protein, 11 aa, synthetic construct
GGNNIGSKSVN

SEQ ID NO: 33 - protein, 11 aa, synthetic construct
GGNNIGSKSVF

SEQ ID NO: 34 - protein, 11 aa, synthetic construct
GGYNIGSKFVH

SEQ ID NO: 35 - protein, 11 aa, synthetic construct
GGNNTGSKSVH

SEQ ID NO: 36 - protein, 11 aa, synthetic construct
GGSDIGDEIVH

SEQ ID NO: 37 - protein, 11 aa, synthetic construct
GGSNIGDIRVH

SEQ ID NO: 38 - protein, 11 aa, synthetic construct
GGNNIGSKGVH

SEQ ID NO: 39 - protein, 11 aa, synthetic construct
GGNNIGSKNVH

SEQ ID NO: 40 - protein, 11 aa, synthetic construct
GGNNIGIKSVH

SEQ ID NO: 41 - protein, 11 aa, synthetic construct
GGNNIGRKAVH

SEQ ID NO: 42 - protein, 14 aa, synthetic construct
AGTGSDVGGYNHVS

SEQ ID NO: 43 - protein, 14 aa, synthetic construct
AGTGSDVGGYNYVS

SEQ ID NO: 44 - protein, 11 aa, synthetic construct
QASQDISNYLN

SEQ ID NO: 45 - protein, 11 aa, synthetic construct
QASQDISNYLD

SEQ ID NO: 46 - protein, 13 aa, synthetic construct
SGSSSNIGSYSVS

SEQ ID NO: 47 - protein, 11 aa, synthetic construct
GGNNIRAKHVH

SEQ ID NO: 48 - protein, 11 aa, synthetic construct
GGNNIRSKSVH

SEQ ID NO: 49 - protein, 11 aa, synthetic construct
GGNNIGRKSVH

SEQ ID NO: 50 - protein, 11 aa, synthetic construct
GGNKIGTKSVH

SEQ ID NO: 51 - protein, 14 aa, synthetic construct
TGTNTDVGAYIDVS

SEQ ID NO: 52 - protein, 15 aa, synthetic construct
KASQSVDYDGDSFMN

SEQ ID NO: 53 - protein, 7 aa, synthetic construct
DDGDRPS

SEQ ID NO: 54 - protein, 7 aa, synthetic construct
DDSDRPS

SEQ ID NO: 55 - protein, 7 aa, synthetic construct
EVSNRPS

SEQ ID NO: 56 - protein, 7 aa, synthetic construct
DDRDRPS

SEQ ID NO: 57 - protein, 7 aa, synthetic construct
DVSKRPS

SEQ ID NO: 58 - protein, 7 aa, synthetic construct
DDSDRPL

SEQ ID NO: 59 - protein, 7 aa, synthetic construct
DVNKRPS

SEQ ID NO: 60 - protein, 7 aa, synthetic construct
DVSNRPS

SEQ ID NO: 61 - protein, 7 aa, synthetic construct
DASNLET

SEQ ID NO: 62 - protein, 7 aa, synthetic construct
DNNKRPS

SEQ ID NO: 63 - protein, 7 aa, synthetic construct
DDNKRPS

SEQ ID NO: 64 - protein, 7 aa, synthetic construct
QDSKRPS

SEQ ID NO: 65 - protein, 7 aa, synthetic construct
RDSNRPS

SEQ ID NO: 66 - protein, 7 aa, synthetic construct
QDRKRPS

SEQ ID NO: 67 - protein, 7 aa, synthetic construct
DVSKWPS

SEQ ID NO: 68 - protein, 7 aa, synthetic construct
TASILES

SEQ ID NO: 69 - protein, 11 aa, synthetic construct
QVWDSISDHVV

SEQ ID NO: 70 - protein, 11 aa, synthetic construct
QVWDSSIDHLI

SEQ ID NO: 71 - protein, 11 aa, synthetic construct
QVWDSSNDLLI

SEQ ID NO: 72 - protein, 11 aa, synthetic construct
QVWDSSSDHLI

SEQ ID NO: 73 - protein, 11 aa, synthetic construct
QVWDSSSDHLV

SEQ ID NO: 74 - protein, 11 aa, synthetic construct
QVWDADSDQLI

SEQ ID NO: 75 - protein, 11 aa, synthetic construct
QVWDSSSDLLI

SEQ ID NO: 76 - protein, 11 aa, synthetic construct
SSYIPSSTRWV

SEQ ID NO: 77 - protein, 11 aa, synthetic construct
SSYTSSSTPYV

SEQ ID NO: 78 - protein, 10 aa, synthetic construct
SSYTSSSTYV

SEQ ID NO: 79 - protein, 11 aa, synthetic construct
SSYTSSSTFYV

SEQ ID NO: 80 - protein, 9 aa, synthetic construct
QQLNSYPLT

SEQ ID NO: 81 - protein, 11 aa, synthetic construct
GTWDSSLSAWV

SEQ ID NO: 82 - protein, 9 aa, synthetic construct
QAWDSSTVV

SEQ ID NO: 83 - protein, 9 aa, synthetic construct
QAWDSNTVV

SEQ ID NO: 84 - protein, 9 aa, synthetic construct
QVWDSSTVV

SEQ ID NO: 85 - protein, 9 aa, synthetic construct
QVWDSSTAV

SEQ ID NO: 86 - protein, 9 aa, synthetic construct
QAWDSSTAV

SEQ ID NO: 87 - protein, 9 aa, synthetic construct
QVWDSSTEV

SEQ ID NO: 88 - protein, 9 aa, synthetic construct
QAWGSSTVV

SEQ ID NO: 89 - protein, 9 aa, synthetic construct
QQYDNLPLT

SEQ ID NO: 90 - protein, 10 aa, synthetic construct
CSYAGNHSFS

SEQ ID NO: 91 - protein, 9 aa, synthetic construct
QQSNEDPWT

SEQ ID NO: 92 - protein, 17 aa, synthetic construct
GFDPDAGETMYAEKFQG

SEQ ID NO: 93 - protein, 17 aa, synthetic construct
GFDPDSGETMYAEKFQG

SEQ ID NO: 94 - protein, 17 aa, synthetic construct
GFDPDEGETMYAEKFQG

SEQ ID NO: 95 - protein, 17 aa, synthetic construct
GFDPDDAETMYAEKFQG

SEQ ID NO: 96 - skipped

SEQ ID NO: 97 - protein, 17 aa, synthetic construct
  VARIANT 1: note = L or G
  VARIANT 2: note = V, F or I
  VARIANT 3: note = D or I
  VARIANT 5: note = E, D or I
  VARIANT 6: note = D, G, F, A, S or E
  VARIANT 7: note = G or A
  VARIANT 8: note = E or T
  VARIANT 9: note = T or A
  VARIANT 10: note = I, M or N
  VARIANT 14: note = K or R
  VARIANT 15: note = F or L
  VARIANT 16: note = R or Q
XXXPXXXXXXYAZXXXG

SEQ ID NO: 98 - protein, 20 aa, synthetic construct
  VARIANT 1: note = P, E or absent
  VARIANT 2: note = G, E or absent
  VARIANT 3: note = G, W, S or absent
  VARIANT 4: note = I, D, S, P or absent
  VARIANT 5: note = S, L, I, T, G or absent
  VARIANT 6: note = P, T, Q, I, D, R or absent
  VARIANT 7: note = G, D or absent
  VARIANT 8: note = E, Y, P, L, D or S
  VARIANT 9: note = E, D, A or G
  VARIANT 10: note = S, F, A, E, Y or W
  VARIANT 11: note = Y or F
  VARIANT 12: note = G, D or W
  VARIANT 13: note = P, Y, I or H
  VARIANT 14..20: note = These residues may be absent
XXXXXXXXXXXXXYYYGMDV

SEQ ID NO: 99 - protein, 16 aa, synthetic construct
  VARIANT 1: note = T, A or absent
  VARIANT 2: note = This residue may be absent
  VARIANT 3: note = Q, G, V, T or S
  VARIANT 4: note = A, G, S or N
  VARIANT 5: note = S, N, D, T or Y
  VARIANT 6: note = Q, Y, N, D, S or K
  VARIANT 7: note = D, I, F, V, S or T
  VARIANT 8..9: note = These residues may be absent
  VARIANT 10: note = I, G or R
  VARIANT 11: note = S, G, A, D, I, R or T
  VARIANT 12: note = This residue may be absent
  VARIANT 13: note = N, K, I or E
  VARIANT 14: note = Y, S, N, D, H, F, R or G
  VARIANT 15: note = L or V
  VARIANT 16: note = N, H, S, D or F
XGXXXXXNIXXYXXXX

SEQ ID NO: 100-101 - skipped (2 entries)

SEQ ID NO: 102 - protein, 17 aa, synthetic construct
  VARIANT 7: note = D or G
  VARIANT 14: note = K or R
  VARIANT 15: note = F or L
GFDPDDXETMYAEXXQG

SEQ ID NO: 103 - protein, 11 aa, synthetic construct
  VARIANT 3: note = N, D or S
  VARIANT 4: note = Y, N or D
  VARIANT 5: note = I or T
  VARIANT 7: note = S, D, I, R or T
  VARIANT 8: note = K, E or I
  VARIANT 9: note = S, I, R, G, N or A
  VARIANT 11: note = H, F or N
GGXXXGXXXVX

SEQ ID NO: 104 - protein, 7 aa, synthetic construct
  VARIANT 3: note = G, S or R
  VARIANT 7: note = S or L
DDXDRPX

SEQ ID NO: 105 - protein, 11 aa, synthetic construct
  VARIANT 5: note = S or A
  VARIANT 6: note = I or S
  VARIANT 7: note = S, I or N
  VARIANT 9: note = H, L or Q
  VARIANT 10: note = V or L
  VARIANT 11: note = V or I
QVWDXXXDXXX

SEQ ID NO: 106 - protein, 7 aa, synthetic construct
  VARIANT 1: note = E or D
  VARIANT 4: note = N or K
XVSXRPS

SEQ ID NO: 107 - protein, 17 aa, synthetic construct
  VARIANT 14: note = K or R
LVDPEDGETIYAEXFQG

SEQ ID NO: 108 - protein, 14 aa, synthetic construct
  VARIANT 1: note = T or A
  VARIANT 4: note = S or G
  VARIANT 12: note = Y or H
XGTXSDVGGYNXVS

SEQ ID NO: 109 - protein, 7 aa, synthetic construct
  VARIANT 1: note = D or E
  VARIANT 3: note = N or S
  VARIANT 4: note = K or N
XVXXRPS

SEQ ID NO: 110 - protein, 11 aa, synthetic construct
  VARIANT 4: note = I or T
  VARIANT 5: note = P or S
  VARIANT 9: note = R, P, F or absent
  VARIANT 10: note = W or Y
SSYXXSSTXXV

SEQ ID NO: 111 - protein, 17 aa, synthetic construct
  VARIANT 16: note = R or Q
LVDPEDGETIYAEKFXG

SEQ ID NO: 112 - protein, 11 aa, synthetic construct
  VARIANT 11: note = N or D
QASQDISNYLX

SEQ ID NO: 113 - protein, 5 aa, synthetic construct
  VARIANT 2: note = S or L
EXSMH

SEQ ID NO: 114 - protein, 7 aa, synthetic construct
  VARIANT 2: note = N or D
DXNKRPS

SEQ ID NO: 115 - protein, 11 aa, synthetic construct
  VARIANT 4: note = N or K
  VARIANT 6: note = R or G
  VARIANT 7: note = A, S, R or T
  VARIANT 9: note = H or S
GGNXIXXKXVH

SEQ ID NO: 116 - protein, 7 aa, synthetic construct
  VARIANT 1: note = Q or R
  VARIANT 3: note = S or R
  VARIANT 4: note = N or K
XDXXRPS

SEQ ID NO: 117 - protein, 9 aa, synthetic construct
  VARIANT 2: note = A or V
  VARIANT 4: note = D or G
  VARIANT 6: note = S or N
  VARIANT 8: note = V, A or E
QXWXSXTXV

SEQ ID NO: 118 - protein, 11 aa, synthetic construct
GGNNIGTKSVH

SEQ ID NO: 119 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 447 aa, synthetic construct
EVQLVQSGAEVKKPGASVKVSCKVSGYTLTELSMQWVRQAPGKGLEWMGGFDPDDGETMYAERFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 447 aa, synthetic construct
QVQLVQSGAEVKKPGSSVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 122 - protein, 447 aa, synthetic construct
EVRLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 123 - protein, 447 aa, synthetic construct
EVQLVQSGAEVKKPGASVKVSCKVSGYTLTELSMQWVRQAPGKGLEWMGGFDPDGGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 124 - protein, 447 aa, synthetic construct
QVQLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 125 - protein, 447 aa, synthetic construct
EVQLVQSGAEVKKPGASVKVSCKVSGYTLTELSMQWVRQAPGKGLEWMGGFDPDDGETMYAEKFQGRLAVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 447 aa, synthetic construct
EVQLVQSGAEVKKPGASVKVSCKVSGYTLTELSMQWVRQAPGKGLEWMGGFDPDDGETMYAEKFQGRLTVTEDTPTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 127 - protein, 447 aa, synthetic construct
EVQLVQSGAEVKKPGASVKVSCEVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 128 - protein, 447 aa, synthetic construct
EVQLVQSGAEVKKPGASVKVSCKVSGYTLTELSMQWVRQAPGKGLEWMGGFDPDDGETMYAEKLQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 451 aa, synthetic construct
QVQLVQSGAEVKKPGATVKISCKVSGYSFTDYFIHWVQQAPGKGLAWVGLVDPEDGETIYAEKFQGRVTITADTSTDTAYMELSSLRSEDTAVYYCATPGGILTDPDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 130 - protein, 451 aa, synthetic construct
QVQLVQSGAEVKKPGATVKISCKVSGYSFTDYFIHWVQQAPGKGLAWVGLVDPEDGETIYAERFQGRVTITADTSTDTAYMELSSLRSEDTAVYYCATPGGILTDPDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 131 - protein, 444 aa, synthetic construct
EVQPVQSGAEVKKPGSSVKVSCKASGGTFSSYAISWVRQAPGQGLEWMGLVDPEDGETIYAEKFRGRVTITADTSTDTAYMELSSLRSEDTAVYYCATEESYGPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 444 aa, synthetic construct
EVQLVQSGAEVKKPGSSVKVSCKASGGTFSSYAISWVRQAPGQGLEWMGLVDPEDGETIYAEKFQGRVTITADTSTDTAYMELSSLRSEDTAVYYCATEESYGPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 133 - protein, 451 aa, synthetic construct
EVQLVQSGAEVKKPGASVKVSCKVSGYTLTESSMHWVRQAPGKGLEWMGLVDPEDGETIYAQKFQGRVTMTEDTSTDTAYMELSSLRSEDTAVYYCAREEWSGDGDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 134 - protein, 451 aa, synthetic construct
EVQLVQSGAEVKKPGASVKVSCKVSGYTLTELSMHWVRQAPGKGLEWMGLVDPEDGETIYAQKFQGRVTMTEDTSTDTAYMELSSLRSEDTAVYYCAREEWSGDGDDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 456 aa, synthetic construct
EVQLVQSGAEVKKPGASVKVSCKASGGTFSSYAISWVRQAPGQGLEWMGGIIPIFGTANYAQKFQGRVTITADESTSTAYMELSSLRSEDTAVYYCASSPLRGSGWYWHYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 136 - protein, 456 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCASSPLRGSGWYWHYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 456 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCASSPLRGSGWYWHYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 138 - protein, 456 aa, synthetic construct
QMQLVQSGAEVKKPGSSVKVSCKASGGTFSSYAISWVRQAPGQGLEWMGGIIPIFGTANYAQKFQGRVTITADESTSTAYMELSSLRSEDTAVYYCASSPLRGSGWYWHYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 139 - protein, 447 aa, synthetic construct
EVQLVQSGAEVKKPGASVKVSCKVSGYTLTELSMHWVRQAPGKGLEWMGLVDPEDGETIYAEKFQGRVTITADTSTDTAYMELSSPRSEDTAVYYCATTIGDDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 140 - protein, 448 aa, synthetic construct
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDIQGLAEFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 141 - protein, 448 aa, synthetic construct
QVTLKESGPGILKPSQTLSLTCSFSGFSLSTSGMGVGWIRQPSGKGLEWLAHIWWDDDKYYNPYLKNQVTISKDTSRNQVFLKITSVDTADTATYYCARSAYGSTYGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 142 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 143 - protein, 447 aa, synthetic construct
EVQLVQSGAEVKKPGASVKVSCKVSGYTLTELSMQWVRQAPGKGLEWMGGFDPDAGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 144 - protein, 447 aa, synthetic construct
EVQLVQSGAEVKKPGASVKVSCKVSGYTLTELSMQWVRQAPGKGLEWMGGFDPDSGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 145 - protein, 447 aa, synthetic construct
EVQLVQSGAEVKKPGASVKVSCKVSGYTLTELSMQWVRQAPGKGLEWMGGFDPDEGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 146 - protein, 447 aa, synthetic construct
EVQLVQSGAEVKKPGASVKVSCKVSGYTLTELSMQWVRQAPGKGLEWMGGFDPDDAETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 147 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELSSLRSEDTAVYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 148 - protein, 447 aa, synthetic construct
EVQLVQSGAEVKKPGASVKVSCKVSGYTLTELSMQWVRQAPGKGLEWMGGFDPDAGETMYAEKFQGRLTVTEDTSTDTVYMELSSLRSEDTAVYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 149 - protein, 447 aa, synthetic construct
EVQLVQSGAEVKKPGASVKVSCKVSGYTLTELSMQWVRQAPGKGLEWMGGFDPDSGETMYAEKFQGRLTVTEDTSTDTVYMELSSLRSEDTAVYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 150 - protein, 447 aa, synthetic construct
EVQLVQSGAEVKKPGASVKVSCKVSGYTLTELSMQWVRQAPGKGLEWMGGFDPDEGETMYAEKFQGRLTVTEDTSTDTVYMELSSLRSEDTAVYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 151 - protein, 447 aa, synthetic construct
EVQLVQSGAEVKKPGASVKVSCKVSGYTLTELSMQWVRQAPGKGLEWMGGFDPDDAETMYAEKFQGRLTVTEDTSTDTVYMELSSLRSEDTAVYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 152 - protein, 447 aa, synthetic construct
QVQLVQSGAEVKKPGASVKVSCKVSGYTLTELSMQWVRQAPGKGLEWMGGFDPDDGETMYAEKFQGRVTMTEDTSTDTAYMELSSLRSEDTAVYYCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 153 - protein, 447 aa, synthetic construct
QVQLVQSGAEVKKPGASVKVSCKVSGYTLTELSMQWVRQAPGKGLEWMGGFDPDAGETMYAEKFQGRVTMTEDTSTDTAYMELSSLRSEDTAVYYCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 154 - protein, 447 aa, synthetic construct
QVQLVQSGAEVKKPGASVKVSCKVSGYTLTELSMQWVRQAPGKGLEWMGGFDPDSGETMYAEKFQGRVTMTEDTSTDTAYMELSSLRSEDTAVYYCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 155 - protein, 447 aa, synthetic construct
QVQLVQSGAEVKKPGASVKVSCKVSGYTLTELSMQWVRQAPGKGLEWMGGFDPDEGETMYAEKFQGRVTMTEDTSTDTAYMELSSLRSEDTAVYYCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - protein, 447 aa, synthetic construct
QVQLVQSGAEVKKPGASVKVSCKVSGYTLTELSMQWVRQAPGKGLEWMGGFDPDDAETMYAEKFQGRVTMTEDTSTDTAYMELSSLRSEDTAVYYCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 157 - protein, 447 aa, synthetic construct
QVQLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 158 - protein, 447 aa, synthetic construct
EVQLVQSGAEVKKPGASVKVSCKVSGYTLTELSMQWVRQAPGKGLEWMGGFDPDDGETMYAEKFQGRVTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 159 - protein, 447 aa, synthetic construct
EVQLVQSGAEVKKPGASVKVSCKVSGYTLTELSMQWVRQAPGKGLEWMGGFDPDDGETMYAEKFQGRLTMTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTAYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 161 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELSSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLRSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 163 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTAVYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 164 - protein, 447 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTALYYCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 165 - protein, 118 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSS

SEQ ID NO: 166 - protein, 118 aa, synthetic construct
EVQLVQSGAEVKKPGASVKVSCKVSGYTLTELSMQWVRQAPGKGLEWMGGFDPDDGETMYAERFQGRLTVTEDTSTDTVYMELRSLTSEDTALYFCATSPGYDFFDYWGQGTLVTVSS

SEQ ID NO: 167 - protein, 118 aa, synthetic construct
QVQLVQSGAEVKKPGSSVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSS

SEQ ID NO: 168 - protein, 118 aa, synthetic construct
EVRLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSS

SEQ ID NO: 169 - protein, 118 aa, synthetic construct
EVQLVQSGAEVKKPGASVKVSCKVSGYTLTELSMQWVRQAPGKGLEWMGGFDPDGGETMYAEKFQGRLTVTEDTSTDTVYMELRSLTSEDTALYFCATSPGYDFFDYWGQGTLVTVSS

SEQ ID NO: 170 - protein, 118 aa, synthetic construct
QVQLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSS

SEQ ID NO: 171 - protein, 118 aa, synthetic construct
EVQLVQSGAEVKKPGASVKVSCKVSGYTLTELSMQWVRQAPGKGLEWMGGFDPDDGETMYAEKFQGRLAVTEDTSTDTVYMELRSLTSEDTALYFCATSPGYDFFDYWGQGTLVTVSS

SEQ ID NO: 172 - protein, 118 aa, synthetic construct
EVQLVQSGAEVKKPGASVKVSCKVSGYTLTELSMQWVRQAPGKGLEWMGGFDPDDGETMYAEKFQGRLTVTEDTPTDTVYMELRSLTSEDTALYFCATSPGYDFFDYWGQGTLVTVSS

SEQ ID NO: 173 - protein, 118 aa, synthetic construct
EVQLVQSGAEVKKPGASVKVSCEVSGYTLTELSMQWVRQAPGKGLEWMGGFDPDDGETMYAEKFQGRLTVTEDTSTDTVYMELRSLTSEDTALYFCATSPGYDFFDYWGQGTLVTVSS

SEQ ID NO: 174 - protein, 118 aa, synthetic construct
EVQLVQSGAEVKKPGASVKVSCKVSGYTLTELSMQWVRQAPGKGLEWMGGFDPDDGETMYAEKLQGRLTVTEDTSTDTVYMELRSLTSEDTALYFCATSPGYDFFDYWGQGTLVTVSS

SEQ ID NO: 175 - protein, 122 aa, synthetic construct
QVQLVQSGAEVKKPGATVKISCKVSGYSFTDYFIHWVQQAPGKGLAWVGLVDPEDGETIYAEKFQGRVTITADTSTDTAYMELSSLRSEDTAVYYCATPGGILTDPDAFDIWGQGTMVTVSS

SEQ ID NO: 176 - protein, 122 aa, synthetic construct
QVQLVQSGAEVKKPGATVKISCKVSGYSFTDYFIHWVQQAPGKGLAWVGLVDPEDGETIYAERFQGRVTITADTSTDTAYMELSSLRSEDTAVYYCATPGGILTDPDAFDIWGQGTMVTVSS

SEQ ID NO: 177 - protein, 115 aa, synthetic construct
EVQPVQSGAEVKKPGSSVKVSCKASGGTFSSYAISWVRQAPGQGLEWMGLVDPEDGETIYAEKFRGRVTITADTSTDTAYMELSSLRSEDTAVYYCATEESYGPWGQGTLVTVSS

SEQ ID NO: 178 - protein, 115 aa, synthetic construct
EVQLVQSGAEVKKPGSSVKVSCKASGGTFSSYAISWVRQAPGQGLEWMGLVDPEDGETIYAEKFQGRVTITADTSTDTAYMELSSLRSEDTAVYYCATEESYGPWGQGTLVTVSS

SEQ ID NO: 179 - protein, 122 aa, synthetic construct
EVQLVQSGAEVKKPGASVKVSCKVSGYTLTESSMHWVRQAPGKGLEWMGLVDPEDGETIYAQKFQGRVTMTEDTSTDTAYMELSSLRSEDTAVYYCAREEWSGDGDDAFDIWGQGTMVTVSS

SEQ ID NO: 180 - protein, 122 aa, synthetic construct
EVQLVQSGAEVKKPGASVKVSCKVSGYTLTELSMHWVRQAPGKGLEWMGLVDPEDGETIYAQKFQGRVTMTEDTSTDTAYMELSSLRSEDTAVYYCAREEWSGDGDDAFDIWGQGTMVTVSS

SEQ ID NO: 181 - protein, 127 aa, synthetic construct
EVQLVQSGAEVKKPGASVKVSCKASGGTFSSYAISWVRQAPGQGLEWMGGIIPIFGTANYAQKFQGRVTITADESTSTAYMELSSLRSEDTAVYYCASSPLRGSGWYWHYYYGMDVWGQGTTVTVSS

SEQ ID NO: 182 - protein, 127 aa, synthetic construct
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCASSPLRGSGWYWHYYYGMDVWGQGTTVTVSS

SEQ ID NO: 183 - protein, 127 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCASSPLRGSGWYWHYYYGMDVWGQGTTVTVSS

SEQ ID NO: 184 - protein, 127 aa, synthetic construct
QMQLVQSGAEVKKPGSSVKVSCKASGGTFSSYAISWVRQAPGQGLEWMGGIIPIFGTANYAQKFQGRVTITADESTSTAYMELSSLRSEDTAVYYCASSPLRGSGWYWHYYYGMDVWGQGTTVTVSS

SEQ ID NO: 185 - protein, 118 aa, synthetic construct
EVQLVQSGAEVKKPGASVKVSCKVSGYTLTELSMHWVRQAPGKGLEWMGLVDPEDGETIYAEKFQGRVTITADTSTDTAYMELSSPRSEDTAVYYCATTIGDDYFDYWGQGTLVTVSS

SEQ ID NO: 186 - protein, 119 aa, synthetic construct
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCATDIQGLAEFDYWGQGTLVTVSS

SEQ ID NO: 187 - protein, 119 aa, synthetic construct
QVTLKESGPGILKPSQTLSLTCSFSGFSLSTSGMGVGWIRQPSGKGLEWLAHIWWDDDKYYNPYLKNQVTISKDTSRNQVFLKITSVDTADTATYYCARSAYGSTYGYWGQGTTLTVSS

SEQ ID NO: 188 - protein, 118 aa, synthetic construct
EVQLVQSGAEVKKPGASVKVSCKVSGYTLTELSMQWVRQAPGKGLEWMGGFDPDAGETMYAEKFQGRLTVTEDTSTDTVYMELRSLTSEDTALYFCATSPGYDFFDYWGQGTLVTVSS

SEQ ID NO: 189 - protein, 118 aa, synthetic construct
EVQLVQSGAEVKKPGASVKVSCKVSGYTLTELSMQWVRQAPGKGLEWMGGFDPDSGETMYAEKFQGRLTVTEDTSTDTVYMELRSLTSEDTALYFCATSPGYDFFDYWGQGTLVTVSS

SEQ ID NO: 190 - protein, 118 aa, synthetic construct
EVQLVQSGAEVKKPGASVKVSCKVSGYTLTELSMQWVRQAPGKGLEWMGGFDPDEGETMYAEKFQGRLTVTEDTSTDTVYMELRSLTSEDTALYFCATSPGYDFFDYWGQGTLVTVSS

SEQ ID NO: 191 - protein, 118 aa, synthetic construct
EVQLVQSGAEVKKPGASVKVSCKVSGYTLTELSMQWVRQAPGKGLEWMGGFDPDDAETMYAEKFQGRLTVTEDTSTDTVYMELRSLTSEDTALYFCATSPGYDFFDYWGQGTLVTVSS

SEQ ID NO: 192 - protein, 118 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELSSLRSEDTAVYFCATSPGYDFFDYWGQGTLVTVSS

SEQ ID NO: 193 - protein, 118 aa, synthetic construct
EVQLVQSGAEVKKPGASVKVSCKVSGYTLTELSMQWVRQAPGKGLEWMGGFDPDAGETMYAEKFQGRLTVTEDTSTDTVYMELSSLRSEDTAVYFCATSPGYDFFDYWGQGTLVTVSS

SEQ ID NO: 194 - protein, 118 aa, synthetic construct
EVQLVQSGAEVKKPGASVKVSCKVSGYTLTELSMQWVRQAPGKGLEWMGGFDPDSGETMYAEKFQGRLTVTEDTSTDTVYMELSSLRSEDTAVYFCATSPGYDFFDYWGQGTLVTVSS

SEQ ID NO: 195 - protein, 118 aa, synthetic construct
EVQLVQSGAEVKKPGASVKVSCKVSGYTLTELSMQWVRQAPGKGLEWMGGFDPDEGETMYAEKFQGRLTVTEDTSTDTVYMELSSLRSEDTAVYFCATSPGYDFFDYWGQGTLVTVSS

SEQ ID NO: 196 - protein, 118 aa, synthetic construct
EVQLVQSGAEVKKPGASVKVSCKVSGYTLTELSMQWVRQAPGKGLEWMGGFDPDDAETMYAEKFQGRLTVTEDTSTDTVYMELSSLRSEDTAVYFCATSPGYDFFDYWGQGTLVTVSS

SEQ ID NO: 197 - protein, 118 aa, synthetic construct
QVQLVQSGAEVKKPGASVKVSCKVSGYTLTELSMQWVRQAPGKGLEWMGGFDPDDGETMYAEKFQGRVTMTEDTSTDTAYMELSSLRSEDTAVYYCATSPGYDFFDYWGQGTLVTVSS

SEQ ID NO: 198 - protein, 118 aa, synthetic construct
QVQLVQSGAEVKKPGASVKVSCKVSGYTLTELSMQWVRQAPGKGLEWMGGFDPDAGETMYAEKFQGRVTMTEDTSTDTAYMELSSLRSEDTAVYYCATSPGYDFFDYWGQGTLVTVSS

SEQ ID NO: 199 - protein, 118 aa, synthetic construct
QVQLVQSGAEVKKPGASVKVSCKVSGYTLTELSMQWVRQAPGKGLEWMGGFDPDSGETMYAEKFQGRVTMTEDTSTDTAYMELSSLRSEDTAVYYCATSPGYDFFDYWGQGTLVTVSS

SEQ ID NO: 200 - protein, 118 aa, synthetic construct
QVQLVQSGAEVKKPGASVKVSCKVSGYTLTELSMQWVRQAPGKGLEWMGGFDPDEGETMYAEKFQGRVTMTEDTSTDTAYMELSSLRSEDTAVYYCATSPGYDFFDYWGQGTLVTVSS

SEQ ID NO: 201 - protein, 118 aa, synthetic construct
QVQLVQSGAEVKKPGASVKVSCKVSGYTLTELSMQWVRQAPGKGLEWMGGFDPDDAETMYAEKFQGRVTMTEDTSTDTAYMELSSLRSEDTAVYYCATSPGYDFFDYWGQGTLVTVSS

SEQ ID NO: 202 - protein, 118 aa, synthetic construct
EVQLVQSGAEVKKPGASVKVSCKVSGYTLTELSMQWVRQAPGKGLEWMGGFDPDDGETMYAEKFQGRVTVTEDTSTDTVYMELRSLTSEDTALYFCATSPGYDFFDYWGQGTLVTVSS

SEQ ID NO: 203 - protein, 118 aa, synthetic construct
EVQLVQSGAEVKKPGASVKVSCKVSGYTLTELSMQWVRQAPGKGLEWMGGFDPDDGETMYAEKFQGRLTMTEDTSTDTVYMELRSLTSEDTALYFCATSPGYDFFDYWGQGTLVTVSS

SEQ ID NO: 204 - protein, 118 aa, synthetic construct
EVQLVQSGAEVKKPGASVKVSCKVSGYTLTELSMQWVRQAPGKGLEWMGGFDPDDGETMYAEKFQGRLTVTEDTSTDTAYMELRSLTSEDTALYFCATSPGYDFFDYWGQGTLVTVSS

SEQ ID NO: 205 - protein, 118 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELSSLTSEDTALYFCATSPGYDFFDYWGQGTLVTVSS

SEQ ID NO: 206 - protein, 118 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLRSEDTALYFCATSPGYDFFDYWGQGTLVTVSS

SEQ ID NO: 207 - protein, 118 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTAVYFCATSPGYDFFDYWGQGTLVTVSS

SEQ ID NO: 208 - protein, 118 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTALYYCATSPGYDFFDYWGQGTLVTVSS

SEQ ID NO: 209 - protein, 214 aa, synthetic construct
SYVLTQPPSVSVAPGKTARITCGGNYIGSKSVHWYQQKPGQAPVLVVYDDGDRPSGIPARFSGSNSGNTATLTISRVEAGDEADYYCQVWDSISDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 210 - protein, 214 aa, synthetic construct
SYELTQPPSVSVAPGKTARITCGGDNIGSKSVHWYQQKPGQAPVLVVYDDSDRPSGIPERFSGSNSGNTATLTISRVEAGDEADYYCQVWDSSIDHLIFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 211 - protein, 214 aa, synthetic construct
SYVLTQPPSVSVAPGQTARITCGGNNIGSKSVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 212 - protein, 214 aa, synthetic construct
SYVLTQPPSVSVAPGQTASITCVGTNFGSKNVHWYQQRPGQAPVLVVYDDSDRPSGIPERFSGSNSGNTATLTISRVEAGDEADYYCQVWDSSSDHLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 213 - protein, 217 aa, synthetic construct
QSALTQPASVSGSPGQSITISCTGTSSDVGGYNYVSWYQQHPGKAPKLMIYEVSNRPSGVSNRFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 214 - protein, 214 aa, synthetic construct
SYVLTQPPSVSVAPGQTASITCVGTNFGSKNVHWYQQRPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 215 - protein, 214 aa, synthetic construct
SYELTQPPSVSVAPGQTARITCGGNNIGSKSVHWYQQKPGQAPVLVVYDDSDRPSGIPERFSGSNSGRTATLTISRVEAGDEADYHCQVWDSSSDHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 216 - protein, 214 aa, synthetic construct
SYVLTQPPSVSVAPGQTARIPCGGNNIGSKSVHWYQQKPGQAPVLVVYDDRDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 217 - protein, 214 aa, synthetic construct
SYELTQPPSVSVAPGQTARIPCGGNNIGSEGVHWYQQKPGQAPVLVVYDDSDRPSGIPERFSGSNSGNTATLTISRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 218 - protein, 217 aa, synthetic construct
QSALTQPRSVSGSPGQSVTISCTGTSSDVGGYNYVSWYQQHPGKAPKLMIYDVSKRPSGVPDRFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 219 - protein, 214 aa, synthetic construct
SYVLTQPPSVSVAPGQTARVSCGGNNIGSKSVNWYRQKPGQAPVLVIYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 220 - protein, 214 aa, synthetic construct
SYVLTQPPSVSVAPGKTARITCGGNNIGSKSVFWYQQKPGQAPVLVVYDDSDRPSGIPERFSGSNSGNTATLTISGTQAMDEADYYCQVWDSSSDHLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 221 - protein, 214 aa, synthetic construct
SYVLTQPPSVSVAPGKTARITCGGYNIGSKFVHWYRQRPGQAPVLVVYDDRDRPSGIPERFSGSNSGNTATLTISRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 222 - protein, 214 aa, synthetic construct
SYELTQPPSVSVAPGKTARITCGGDNIGSKSVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 223 - protein, 214 aa, synthetic construct
SYVLTQPPSVSVAPGKTARITCGGNNTGSKSVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 224 - protein, 214 aa, synthetic construct
SYVLTQPPSVSVAPGQTARITCGGSDIGDEIVHWYQQKPGQAPVLVVYDDSDRPSGIPERFSGSNSGNTATLTISRAQAGDEADYYCQVWDSSSDHLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 225 - protein, 214 aa, synthetic construct
SYVLTQPPSVSVAPGKTARITCGGNNIGSKSVHWYQQKPGQAPVLVVYDDSDRPLGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 226 - protein, 214 aa, synthetic construct
SYVLTQPLSVSVALGQTARITCGGSNIGDIRVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 227 - protein, 214 aa, synthetic construct
SYVLTQPPSVSVAPGKTARITCGGNNIGSKGVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 228 - protein, 214 aa, synthetic construct
SYVLTQPPSVSVAPGQTARVSCGGNNIGSKSVNWYRQKPGQAPVLVIYDDSDRPSGIPERFSGSNSGNTATLTISRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 229 - protein, 214 aa, synthetic construct
SYVLTQPPSVSVAPGKTARITCGGNNIGSKNVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 230 - protein, 214 aa, synthetic construct
SYVLTQPPSVSVAPGQTARITCGGNNIGIKSVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 231 - protein, 214 aa, synthetic construct
SYVLTQPPSVSVAPGKTARITCGGNNIGSKSVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 232 - protein, 214 aa, synthetic construct
SYELTQPPSVAVAPGQTARITCGGNNIGRKAVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 233 - protein, 214 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSIDHLIFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 234 - protein, 214 aa, synthetic construct
SYVLTQPPSVSVAPGQTARITCGGNNIGIKSVHWYQQKPGQAPVLVVYDDRDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 235 - protein, 214 aa, synthetic construct
SYELTQPPSVSVAPGQTARITCGGNNIGSKSVHWYQQKPGQAPVLVVYDDSDRPSGIPERFSGSNSGNTATLTISRVEAGDEADYYCQVWDADSDQLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 236 - protein, 214 aa, synthetic construct
SYVLTQPPSVSVAPGKTARITCGGNNIGSKSVHWYQQKPGQAPVLVVYDDSDRPSGIPERFSGSNSGNTATLTISRVEAGDEADYYCQVWDSSSDLLIFGGGAKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 237 - protein, 214 aa, synthetic construct
SYVLTQPPSVSVAPGKTATITCGGNNIGTKSVHWYQQKPGQAPVLVVYDDSDRPSGIPERFSGSNSGNTATLTINRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 238 - protein, 217 aa, synthetic construct
QSALTQPASVSGSPGQSVTISCTGTSSDVGGYNYVSWYQHHPGKAPKLMIYDVNKRPSGVPDRFSGSKSGNTASLTISGLQAEDEADYYCSSYIPSSTRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 239 - protein, 217 aa, synthetic construct
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTPYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 240 - protein, 217 aa, synthetic construct
QSALTQPRSVSGSPGQSVTISCAGTGSDVGGYNHVSWYQHHPGKAPKLMIYDVNKRPSGVPDRFSGSKSGNTASLTISGLQAEDEADYYCSSYIPSSTRWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 241 - protein, 216 aa, synthetic construct
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 242 - protein, 217 aa, synthetic construct
QSALTQPRSVSGSPGQSVTISCAGTGSDVGGYNYVSWYQHHPGKAPKLMIYDVNKRPSGVPDRFSGSKSGNTASLTISGLQAEDEADYYCSSYTSSSTFYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 243 - protein, 217 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTFYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 244 - protein, 214 aa, synthetic construct
DIQLTQSPSSLSASVGDRVTITCQASQDISNYLNWYQQKPGKAPKLLIYDASNLETGVPSRFSGSGSGTEFTLTISSLQPEDFATYYCQQLNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 245 - protein, 214 aa, synthetic construct
DIQLTQSPSSLSASVGDRVTITCQASQDISNYLDWYQQKPGKAPKLLIYDASNLETGVPSRFSGSGSGTEFTLTISSLQPEDFATYYCQQLNSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 216 aa, synthetic construct
QSVLTQPPSVSAAPGQKVTISCSGSSSNIGSYSVSWYQQLPGTAPKLLIYDNNKRPSGIPDRFSGSNSGTSATLGITGLQTGDEADYYCGTWDSSLSAWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 247 - protein, 216 aa, synthetic construct
QSVLTQPPSVSAAPGQKVTISCSGSSSNIGSYSVSWYQQLPGTAPKLLIYDDNKRPSGIPDRFSGSNSGTSATLSITGLQTGDEADYYCGTWDSSLSAWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 248 - protein, 212 aa, synthetic construct
SYELTQPPSVSVAPGKTARVTCGGNNIRAKHVHWYQQKPGQSPVLVIYQDSKRPSGIPERFSGSNSGNTATLTISGTQAMDEADYYCQAWDSSTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 249 - protein, 212 aa, synthetic construct
SYVLTQPPSVSAAPGKTARISCGGNNIRSKSVHWYQQKAGQAPVLVIYQDSKRPSGIPERFSGSNSGNTATLTISGTQAMDEADYYCQAWDSNTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 250 - protein, 212 aa, synthetic construct
SYVLTQPLSVSVAPGQTARITCGGNNIGRKSVHWYQQKPGQAPVLVIYRDSNRPSGIPERFSGSNSGNTATLTISRAQAGDEADYYCQVWDSSTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 251 - protein, 212 aa, synthetic construct
SYELTQPPSVSVAPGKTARITCGGNNIRSKSVHWYQQKPGQAPVLVVYQDSKRPSGIPERFSGSNSGNTATLTISGTQAMDEADYYCQVWDSSTAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 252 - protein, 212 aa, synthetic construct
SYELTQPPSVSVAPGKTARITCGGNKIGTKSVHWYQQKPGQAPVLVIYRDSNRPSGIPERFSGSNSGNTATLTISRAQAGDEADYYCQVWDSSTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 253 - protein, 212 aa, synthetic construct
SYVLTQPPSVSVAPGKTARITCGGNNIGSKSVHWYQQKPGQAPVLVIYQDRKRPSGIPERFSGSNSGNTATLTISGTQAMDEADYYCQAWDSSTAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 254 - protein, 212 aa, synthetic construct
SYELTQPPSVSVAPGKTARITCGGNNIRAKHVHWYQQKPGQSPVLVIYQDSKRPSGIPERFSGSNSGSTATLTISRVEAGDEADYYCQVWDSSTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 255 - protein, 212 aa, synthetic construct
SYVLTQPPSVSVAPGKTARITCGGNNIGTKSVHWYQQKPGQSPVLVIYQDRKRPSGIPERFSGSNSGNTATLTISGTQAMDEADYYCQVWDSSTEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 256 - protein, 212 aa, synthetic construct
SYELTQPPSVSVAPGKTARITCGGNKIGTKSVHWYQQKPGQAPVLVIYQDSKRPSGIPERFSGSNSGNTATLTISGTQAMDEADYYCQAWGSSTVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 257 - protein, 214 aa, synthetic construct
DIQLTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - protein, 216 aa, synthetic construct
QSALTQPRSVSGSPGQSVTISCTGTNTDVGAYIDVSWYQQHPGKAPKLIIYDVSKWPSGVPDRFSGSKSGNTASLTISGLQAEDEADYYCCSYAGNHSFSFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 259 - protein, 218 aa, synthetic construct
DIVLTQSPASLAVSLGQRATISCKASQSVDYDGDSFMNWYQQKPGQPPKLLIYTASILESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 260 - protein, 217 aa, synthetic construct
SYVLTQPPSVSVAPGKTARITCTGTSSDVGGYNYVSWYQQKPGQAPVLVVYEVSNRPSGIPERFSGSNSGNTATLTISRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 261 - protein, 214 aa, synthetic construct
SYVLTQPPSVSVAPGKTARITCGGNNIGSKSVHWYQQKPGQAPVLVVYDDSDRPSGIPERFSGSNSGNTATLTISRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 262 - protein, 214 aa, synthetic construct
SYVLTQPPSVSVAPGKTARITCGGDNIGSKSVHWYQQKPGQAPVLVVYDDSDRPSGIPERFSGSNSGNTATLTISRVEAGDEADYYCQVWDSSNDLLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 263 - protein, 214 aa, synthetic construct
SYVLTQPPSVSVAPGQTARITCGGSDIGDEIVHWYQQKPGQAPVLVVYDDSDRPSGIPERFSGSNSGNTATLTISRVEAGDEADYYCQVWDSSSDHLIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 264 - protein, 108 aa, synthetic construct
SYVLTQPPSVSVAPGKTARITCGGNYIGSKSVHWYQQKPGQAPVLVVYDDGDRPSGIPARFSGSNSGNTATLTISRVEAGDEADYYCQVWDSISDHVVFGGGTKLTVL

SEQ ID NO: 265 - protein, 108 aa, synthetic construct
SYELTQPPSVSVAPGKTARITCGGDNIGSKSVHWYQQKPGQAPVLVVYDDSDRPSGIPERFSGSNSGNTATLTISRVEAGDEADYYCQVWDSSIDHLIFGGGTKVTVL

SEQ ID NO: 266 - protein, 108 aa, synthetic construct
SYVLTQPPSVSVAPGQTARITCGGNNIGSKSVHWYQQKPGQAPVLVVYDDSDRPSGIPERFSGSNSGNTATLTINRVEAGDEADYYCQVWDSSNDLLIFGGGTKLTVL

SEQ ID NO: 267 - protein, 108 aa, synthetic construct
SYVLTQPPSVSVAPGQTASITCVGTNFGSKNVHWYQQRPGQAPVLVVYDDSDRPSGIPERFSGSNSGNTATLTISRVEAGDEADYYCQVWDSSSDHLIFGGGTKLTVL

SEQ ID NO: 268 - protein, 111 aa, synthetic construct
QSALTQPASVSGSPGQSITISCTGTSSDVGGYNYVSWYQQHPGKAPKLMIYEVSNRPSGVSNRFSGSNSGNTATLTINRVEAGDEADYYCQVWDSSNDLLIFGGGTKLTVL

SEQ ID NO: 269 - protein, 108 aa, synthetic construct
SYVLTQPPSVSVAPGQTASITCVGTNFGSKNVHWYQQRPGQAPVLVVYDDSDRPSGIPERFSGSNSGNTATLTINRVEAGDEADYYCQVWDSSNDLLIFGGGTKLTVL

SEQ ID NO: 270 - protein, 108 aa, synthetic construct
SYELTQPPSVSVAPGQTARITCGGNNIGSKSVHWYQQKPGQAPVLVVYDDSDRPSGIPERFSGSNSGRTATLTISRVEAGDEADYHCQVWDSSSDHLVFGGGTKLTVL

SEQ ID NO: 271 - protein, 108 aa, synthetic construct
SYVLTQPPSVSVAPGQTARIPCGGNNIGSKSVHWYQQKPGQAPVLVVYDDRDRPSGIPERFSGSNSGNTATLTINRVEAGDEADYYCQVWDSSNDLLIFGGGTKLTVL

SEQ ID NO: 272 - protein, 108 aa, synthetic construct
SYELTQPPSVSVAPGQTARIPCGGNNIGSEGVHWYQQKPGQAPVLVVYDDSDRPSGIPERFSGSNSGNTATLTISRVEAGDEADYYCQVWDSSNDLLIFGGGTKLTVL

SEQ ID NO: 273 - protein, 111 aa, synthetic construct
QSALTQPRSVSGSPGQSVTISCTGTSSDVGGYNYVSWYQQHPGKAPKLMIYDVSKRPSGVPDRFSGSNSGNTATLTINRVEAGDEADYYCQVWDSSNDLLIFGGGTKLTVL

SEQ ID NO: 274 - protein, 108 aa, synthetic construct
SYVLTQPPSVSVAPGQTARVSCGGNNIGSKSVNWYRQKPGQAPVLVIYDDSDRPSGIPERFSGSNSGNTATLTINRVEAGDEADYYCQVWDSSNDLLIFGGGTKLTVL

SEQ ID NO: 275 - protein, 108 aa, synthetic construct
SYVLTQPPSVSVAPGKTARITCGGNNIGSKSVFWYQQKPGQAPVLVVYDDSDRPSGIPERFSGSNSGNTATLTISGTQAMDEADYYCQVWDSSSDHLVFGGGTKLTVL

SEQ ID NO: 276 - protein, 108 aa, synthetic construct
SYVLTQPPSVSVAPGKTARITCGGYNIGSKFVHWYRQRPGQAPVLVVYDDRDRPSGIPERFSGSNSGNTATLTISRVEAGDEADYYCQVWDSSNDLLIFGGGTKLTVL

SEQ ID NO: 277 - protein, 108 aa, synthetic construct
SYELTQPPSVSVAPGKTARITCGGDNIGSKSVHWYQQKPGQAPVLVVYDDSDRPSGIPERFSGSNSGNTATLTINRVEAGDEADYYCQVWDSSNDLLIFGGGTKLTVL

SEQ ID NO: 278 - protein, 108 aa, synthetic construct
SYVLTQPPSVSVAPGKTARITCGGNNTGSKSVHWYQQKPGQAPVLVVYDDSDRPSGIPERFSGSNSGNTATLTINRVEAGDEADYYCQVWDSSNDLLIFGGGTKLTVL

SEQ ID NO: 279 - protein, 108 aa, synthetic construct
SYVLTQPPSVSVAPGQTARITCGGSDIGDEIVHWYQQKPGQAPVLVVYDDSDRPSGIPERFSGSNSGNTATLTISRAQAGDEADYYCQVWDSSSDHLIFGGGTKLTVL

SEQ ID NO: 280 - protein, 108 aa, synthetic construct
SYVLTQPPSVSVAPGKTARITCGGNNIGSKSVHWYQQKPGQAPVLVVYDDSDRPLGIPERFSGSNSGNTATLTINRVEAGDEADYYCQVWDSSNDLLIFGGGTKLTVL

SEQ ID NO: 281 - protein, 108 aa, synthetic construct
SYVLTQPLSVSVALGQTARITCGGSNIGDIRVHWYQQKPGQAPVLVVYDDSDRPSGIPERFSGSNSGNTATLTINRVEAGDEADYYCQVWDSSNDLLIFGGGTKLTVL

SEQ ID NO: 282 - protein, 108 aa, synthetic construct
SYVLTQPPSVSVAPGKTARITCGGNNIGSKGVHWYQQKPGQAPVLVVYDDSDRPSGIPERFSGSNSGNTATLTINRVEAGDEADYYCQVWDSSNDLLIFGGGTKLTVL

SEQ ID NO: 283 - protein, 108 aa, synthetic construct
SYVLTQPPSVSVAPGQTARVSCGGNNIGSKSVNWYRQKPGQAPVLVIYDDSDRPSGIPERFSGSNSGNTATLTISRVEAGDEADYYCQVWDSSNDLLIFGGGTKLTVL

SEQ ID NO: 284 - protein, 108 aa, synthetic construct
SYVLTQPPSVSVAPGKTARITCGGNNIGSKNVHWYQQKPGQAPVLVVYDDSDRPSGIPERFSGSNSGNTATLTINRVEAGDEADYYCQVWDSSNDLLIFGGGTKLTVL

SEQ ID NO: 285 - protein, 108 aa, synthetic construct
SYVLTQPPSVSVAPGQTARITCGGNNIGIKSVHWYQQKPGQAPVLVVYDDSDRPSGIPERFSGSNSGNTATLTINRVEAGDEADYYCQVWDSSNDLLIFGGGTKLTVL

SEQ ID NO: 286 - protein, 108 aa, synthetic construct
SYVLTQPPSVSVAPGKTARITCGGNNIGSKSVHWYQQKPGQAPVLVVYDDSDRPSGIPERFSGSNSGNTATLTINRVEAGDEADYYCQVWDSSNDLLIFGGGTKLTVL

SEQ ID NO: 287 - protein, 108 aa, synthetic construct
SYELTQPPSVAVAPGQTARITCGGNNIGRKAVHWYQQKPGQAPVLVVYDDSDRPSGIPERFSGSNSGNTATLTINRVEAGDEADYYCQVWDSSNDLLIFGGGTKLTVL

SEQ ID NO: 288 - protein, 108 aa, synthetic construct
SYVLTQPPSVSVAPGQTARITCGGNNIGSKSVHWYQQKPGQAPVLVVYDDSDRPSGIPERFSGSNSGNTATLTISRVEAGDEADYYCQVWDSSIDHLIFGGGTKVTVL

SEQ ID NO: 289 - protein, 108 aa, synthetic construct
SYVLTQPPSVSVAPGQTARITCGGNNIGIKSVHWYQQKPGQAPVLVVYDDRDRPSGIPERFSGSNSGNTATLTINRVEAGDEADYYCQVWDSSNDLLIFGGGTKLTVL

SEQ ID NO: 290 - protein, 108 aa, synthetic construct
SYELTQPPSVSVAPGQTARITCGGNNIGSKSVHWYQQKPGQAPVLVVYDDSDRPSGIPERFSGSNSGNTATLTISRVEAGDEADYYCQVWDADSDQLIFGGGTKLTVL

SEQ ID NO: 291 - protein, 108 aa, synthetic construct
SYVLTQPPSVSVAPGKTARITCGGNNIGSKSVHWYQQKPGQAPVLVVYDDSDRPSGIPERFSGSNSGNTATLTISRVEAGDEADYYCQVWDSSSDLLIFGGGAKLTVL

SEQ ID NO: 292 - protein, 108 aa, synthetic construct
SYVLTQPPSVSVAPGKTATITCGGNNIGTKSVHWYQQKPGQAPVLVVYDDSDRPSGIPERFSGSNSGNTATLTINRVEAGDEADYYCQVWDSSNDLLIFGGGTKLTVL

SEQ ID NO: 293 - protein, 111 aa, synthetic construct
QSALTQPASVSGSPGQSVTISCTGTSSDVGGYNYVSWYQHHPGKAPKLMIYDVNKRPSGVPDRFSGSKSGNTASLTISGLQAEDEADYYCSSYIPSSTRWVFGGGTKLTVL

SEQ ID NO: 294 - protein, 111 aa, synthetic construct
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTPYVFGTGTKVTVL

SEQ ID NO: 295 - protein, 111 aa, synthetic construct
QSALTQPRSVSGSPGQSVTISCAGTGSDVGGYNHVSWYQHHPGKAPKLMIYDVNKRPSGVPDRFSGSKSGNTASLTISGLQAEDEADYYCSSYIPSSTRWVFGGGTKLTVL

SEQ ID NO: 296 - protein, 110 aa, synthetic construct
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTYVFGTGTKVTVL

SEQ ID NO: 297 - protein, 111 aa, synthetic construct
QSALTQPRSVSGSPGQSVTISCAGTGSDVGGYNYVSWYQHHPGKAPKLMIYDVNKRPSGVPDRFSGSKSGNTASLTISGLQAEDEADYYCSSYTSSSTFYVFGTGTKVTVL

SEQ ID NO: 298 - protein, 111 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTFYVFGTGTKVTVL

SEQ ID NO: 299 - protein, 107 aa, synthetic construct
DIQLTQSPSSLSASVGDRVTITCQASQDISNYLNWYQQKPGKAPKLLIYDASNLETGVPSRFSGSGSGTEFTLTISSLQPEDFATYYCQQLNSYPLTFGGGTKLEIK

SEQ ID NO: 300 - protein, 107 aa, synthetic construct
DIQLTQSPSSLSASVGDRVTITCQASQDISNYLDWYQQKPGKAPKLLIYDASNLETGVPSRFSGSGSGTEFTLTISSLQPEDFATYYCQQLNSYPLTFGGGTKLEIK

SEQ ID NO: 301 - protein, 110 aa, synthetic construct
QSVLTQPPSVSAAPGQKVTISCSGSSSNIGSYSVSWYQQLPGTAPKLLIYDNNKRPSGIPDRFSGSNSGTSATLGITGLQTGDEADYYCGTWDSSLSAWVFGGGTKLTVL

SEQ ID NO: 302 - protein, 110 aa, synthetic construct
QSVLTQPPSVSAAPGQKVTISCSGSSSNIGSYSVSWYQQLPGTAPKLLIYDDNKRPSGIPDRFSGSNSGTSATLSITGLQTGDEADYYCGTWDSSLSAWVFGGGTKLTVL

SEQ ID NO: 303 - protein, 106 aa, synthetic construct
SYELTQPPSVSVAPGKTARVTCGGNNIRAKHVHWYQQKPGQSPVLVIYQDSKRPSGIPERFSGSNSGNTATLTISGTQAMDEADYYCQAWDSSTVVFGGGTKLTVL

SEQ ID NO: 304 - protein, 106 aa, synthetic construct
SYVLTQPPSVSAAPGKTARISCGGNNIRSKSVHWYQQKAGQAPVLVIYQDSKRPSGIPERFSGSNSGNTATLTISGTQAMDEADYYCQAWDSNTVVFGGGTKLTVL

SEQ ID NO: 305 - protein, 106 aa, synthetic construct
SYVLTQPLSVSVAPGQTARITCGGNNIGRKSVHWYQQKPGQAPVLVIYRDSNRPSGIPERFSGSNSGNTATLTISRAQAGDEADYYCQVWDSSTVVFGGGTKLTVL

SEQ ID NO: 306 - protein, 106 aa, synthetic construct
SYELTQPPSVSVAPGKTARITCGGNNIRSKSVHWYQQKPGQAPVLVVYQDSKRPSGIPERFSGSNSGNTATLTISGTQAMDEADYYCQVWDSSTAVFGGGTKLTVL

SEQ ID NO: 307 - protein, 106 aa, synthetic construct
SYELTQPPSVSVAPGKTARITCGGNKIGTKSVHWYQQKPGQAPVLVIYRDSNRPSGIPERFSGSNSGNTATLTISRAQAGDEADYYCQVWDSSTVVFGGGTKLTVL

SEQ ID NO: 308 - protein, 106 aa, synthetic construct
SYVLTQPPSVSVAPGKTARITCGGNNIGSKSVHWYQQKPGQAPVLVIYQDRKRPSGIPERFSGSNSGNTATLTISGTQAMDEADYYCQAWDSSTAVFGGGTKLTVL

SEQ ID NO: 309 - protein, 106 aa, synthetic construct
SYELTQPPSVSVAPGKTARITCGGNNIRAKHVHWYQQKPGQSPVLVIYQDSKRPSGIPERFSGSNSGSTATLTISRVEAGDEADYYCQVWDSSTVVFGGGTKLTVL

SEQ ID NO: 310 - protein, 106 aa, synthetic construct
SYVLTQPPSVSVAPGKTARITCGGNNIGTKSVHWYQQKPGQSPVLVIYQDRKRPSGIPERFSGSNSGNTATLTISGTQAMDEADYYCQVWDSSTEVFGGGTKLTVL

SEQ ID NO: 311 - protein, 106 aa, synthetic construct
SYELTQPPSVSVAPGKTARITCGGNKIGTKSVHWYQQKPGQAPVLVIYQDSKRPSGIPERFSGSNSGNTATLTISGTQAMDEADYYCQAWGSSTVVFGGGTKLTVL

SEQ ID NO: 312 - protein, 107 aa, synthetic construct
DIQLTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKLEIK

SEQ ID NO: 313 - protein, 110 aa, synthetic construct
QSALTQPRSVSGSPGQSVTISCTGTNTDVGAYIDVSWYQQHPGKAPKLIIYDVSKWPSGVPDRFSGSKSGNTASLTISGLQAEDEADYYCCSYAGNHSFSFGGGTKVTVL

SEQ ID NO: 314 - protein, 111 aa, synthetic construct
DIVLTQSPASLAVSLGQRATISCKASQSVDYDGDSFMNWYQQKPGQPPKLLIYTASILESGIPARFSGSGSGTDFTLNIHPVEEEDAATYYCQQSNEDPWTFGGGTKLEIK

SEQ ID NO: 315 - protein, 111 aa, synthetic construct
SYVLTQPPSVSVAPGKTARITCTGTSSDVGGYNYVSWYQQKPGQAPVLVVYEVSNRPSGIPERFSGSNSGNTATLTISRVEAGDEADYYCQVWDSSNDLLIFGGGTKLTVL

SEQ ID NO: 316 - protein, 108 aa, synthetic construct
SYVLTQPPSVSVAPGKTARITCGGNNIGSKSVHWYQQKPGQAPVLVVYDDSDRPSGIPERFSGSNSGNTATLTISRVEAGDEADYYCQVWDSSNDLLIFGGGTKLTVL

SEQ ID NO: 317 - protein, 108 aa, synthetic construct
SYVLTQPPSVSVAPGKTARITCGGDNIGSKSVHWYQQKPGQAPVLVVYDDSDRPSGIPERFSGSNSGNTATLTISRVEAGDEADYYCQVWDSSNDLLIFGGGTKLTVL

SEQ ID NO: 318 - protein, 108 aa, synthetic construct
SYVLTQPPSVSVAPGQTARITCGGSDIGDEIVHWYQQKPGQAPVLVVYDDSDRPSGIPERFSGSNSGNTATLTISRVEAGDEADYYCQVWDSSSDHLIFGGGTKLTVL

SEQ ID NO: 319 - protein, 417 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDKEDDEDKDEDEEDEEDKEEDEEEDVPGQAKDEL

SEQ ID NO: 320 - protein, 411 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRTRRMMRTKMRMRRMRRTRRKMRRKMSPARPRTSCREACLQGWTEA

SEQ ID NO: 321 - protein, 430 aa, Homo sapiens
MLLSVPLLLGLLGLAVAEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDNCRRMMRTKMRMRRMRRTRRKMRRKMSPARPRTSCREACLQGWTEA

SEQ ID NO: 322 - protein, 36 aa, Homo sapiens
RMRRMRRTRRKMRRKMSPARPRTSCREACLQGWTEA

SEQ ID NO: 323 - DNA, 1905 bp, Homo sapiens
  source 1..1905: mol_type = genomic DNA
atgccctcctgggccctcttcatggtcacctcctgcctcctcctggcccctcaaaacctggcccaagtcagcagccaagatgtctccttgctggcatcagactcagagcccctgaagtgtttctcccgaacatttgaggacctcacttgcttctgggatgaggaagaggcagcgcccagtgggacataccagctgctgtatgcctacccgcgggagaagccccgtgcttgccccctgagttcccagagcatgccccactttggaacccgatacgtgtgccagtttccagaccaggaggaagtgcgtctcttctttccgctgcacctctgggtgaagaatgtgttcctaaaccagactcggactcagcgagtcctctttgtggacagtgtaggcctgccggctccccccagtatcatcaaggccatgggtgggagccagccaggggaacttcagatcagctgggaggagccagctccagaaatcagtgatttcctgaggtacgaactccgctatggccccagagatcccaagaactccactggtcccacggtcatacagctgattgccacagaaacctgctgccctgctctgcagaggcctcactcagcctctgctctggaccagtctccatgtgctcagcccacaatgccctggcaagatggaccaaagcagacctccccaagtagagaagcttcagctctgacagcagagggtggaagctgcctcatctcaggactccagcctggcaactcctactggctgcagctgcgcagcgaacctgatgggatctccctcggtggctcctggggatcctggtccctccctgtgactgtggacctgcctggagatgcagtggcacttggactgcaatgctttaccttggacctgaagaatgttacctgtcaatggcagcaacaggaccatgctagctcccaaggcttcttctaccacagcagggcacggtgctgccccagagacaggtaccccatctgggagaactgcgaagaggaagagaaaacaaatccaggactacagaccccacagttctctcgctgccacttcaagtcacgaaatgacagcattattcacatccttgtggaggtgaccacagccccgggtactgttcacagctacctgggctcccctttctggatccaccaggctgtgcgcctccccaccccaaacttgcactggagggagatctccagtgggcatctggaattggagtggcagcacccatcgtcctgggcagcccaagagacctgttatcaactccgatacacaggagaaggccatcaggactggaaggtgctggagccgcctctcggggcccgaggagggaccctggagctgcgcccgcgatctcgctaccgtttacagctgcgcgccaggctcaacggccccacctaccaaggtccctggagctcgtggtcggacccaactagggtggagaccgccaccgagaccgcctggatctccttggtgaccgctctgcatctagtgctgggcctcagcgccgtcctgggcctgctgctgctgaggtggcagtttcctgcacactacaggagactgaggcatgccctgtggccctcacttccagacctgcaccgggtcctaggccagtaccttagggacactgcagccctgagcccgcccaaggccacagtctcagatacctgtgaagaagtggaacccagcctccttgaaatcctccccaagtcctcagagaggactcctttgcccctgtgttcctcccaggcccagatggactaccgaagattgcagccttcttgcctggggaccatgcccctgtctgtgtgcccacccatggctgagtcagggtcctgctgtaccacccacattgccaaccattcctacctaccactaagctattggcagcagcct

SEQ ID NO: 324 - protein, 635 aa, Homo sapiens
MPSWALFMVTSCLLLAPQNLAQVSSQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQP

SEQ ID NO: 325 - DNA, 1233 bp, Homo sapiens
  source 1..1233: mol_type = genomic DNA
atgctgctatccgtgccgctgctgctcggcctcctcggcctggccgtcgccgagcctgccgtctacttcaaggagcagtttctggacggagacgggtggacttcccgctggatcgaatccaaacacaagtcagattttggcaaattcgttctcagttccggcaagttctacggtgacgaggagaaagataaaggtttgcagacaagccaggatgcacgcttttatgctctgtcggccagtttcgagcctttcagcaacaaaggccagacgctggtggtgcagttcacggtgaaacatgagcagaacatcgactgtgggggcggctatgtgaagctgtttcctaatagtttggaccagacagacatgcacggagactcagaatacaacatcatgtttggtcccgacatctgtggccctggcaccaagaaggttcatgtcatcttcaactacaagggcaagaacgtgctgatcaacaaggacatccgttgcaaggatgatgagtttacacacctgtacacactgattgtgcggccagacaacacctatgaggtgaagattgacaacagccaggtggagtccggctccttggaagacgattgggacttcctgccacccaagaagataaaggatcctgatgcttcaaaaccggaagactgggatgagcgggccaagatcgatgatcccacagactccaagcctgaggactgggacaagcccgagcatatccctgaccctgatgctaagaagcccgaggactgggatgaagagatggacggagagtgggaacccccagtgattcagaaccctgagtacaagggtgagtggaagccccggcagatcgacaacccagattacaagggcacttggatccacccagaaattgacaaccccgagtattctcccgatcccagtatctatgcctatgataactttggcgtgctgggcctggacctctggcaggtcaagtctggcaccatctttgacaacttcctcatcaccaacgatgaggcatacgctgaggagtttggcaacgagacgtggggcgtaacaaaggcagcagagaaacaaatgaaggacaaacaggacgaggagcagaggacaaggaggatgatgaggacaaagatgaggatgaggaggatgaggaggacaaggaggaagatgaggaggaagatgtccccggccaggccaaggacgagctgtagagaggcctgcctccagggctggactgaggcc

SEQ ID NO: 326 - protein, 674 aa, synthetic construct
MDYKDDDDKGGVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYKENLYFQGNKGSIEGREPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGQETWGVTKAAEKQMKDKQDEEQRTRRMMRTKMRMRRMRRTRRKMRRKMSPARPRTSCREACLQGWTEALEVLFQGPGSGAKDEL

SEQ ID NO: 327 - protein, 226 aa, synthetic construct
EVQLVQSGAEVKKPGASVKVSCKVSGYTLTELSMQWVRQAPGKGLEWMGGFDPDDGETMYAEKFQGRLTVTEDTSTDTVYMELRSLTSEDTALYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHT

SEQ ID NO: 328 - protein, 21 aa, Homo sapiens
DEEQRTRRMMRTKMRMRRMRR

SEQ ID NO: 329 - protein, 10 aa, synthetic construct
GYTLTELSMQ

SEQ ID NO: 330 - protein, 226 aa, synthetic construct
EVQLVQSGAEVKKPGASVKVSCKVSGYTLTELSMQWVRQAPGKGLEWMGGFDPDDAETMYAEKFQGRLTVTEDTSTDTVYMELSSLRSEDTAVYFCATSPGYDFFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHT

SEQ ID NO: 331 - protein, 821 aa, synthetic construct
HHHHHHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTRSSSLEVLFQGPGLNDIFEAQKIEWHEENLYFQGEPAVYFKEQFLDGDGWTSRWIESKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRLKEEEEDKKRKEEEEAEDNCTRRMMRTKMRMRRMRRTRRKMRRKMSPARPRTSCREACLQGWTEA

SEQ ID NO: 332 - protein, 55 aa, synthetic construct
MKDKQDEEQRTRRMMRTKMRMRRMRRTRRKMRRKMSPARPRTSSREASLQGWTEA

SEQ ID NO: 333 - protein, 31 aa, synthetic construct
MKDKQDEEQRTRRMMRTKMRMRRMRRTRRKM

SEQ ID NO: 334 - protein, 800 aa, synthetic construct
HHHHHHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSLEVLFQGPGLNDIFEAQKIEWHEENLYFQGEPAVYFKEQFLDGDGWTSRWIEKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRTRRMMRTKMRMRRMRRTRRKMRRKMSPARPRTSCREACLQGWTEA

SEQ ID NO: 335 - protein, 800 aa, synthetic construct
HHHHHHMKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRSSSLEVLFQGPGLNDIFEAQKIEWHEENLYFQGEPAVYFKEQFLDGDGWTSRWIEKHKSDFGKFVLSSGKFYGDEEKDKGLQTSQDARFYALSASFEPFSNKGQTLVVQFTVKHEQNIDCGGGYVKLFPNSLDQTDMHGDSEYNIMFGPDICGPGTKKVHVIFNYKGKNVLINKDIRCKDDEFTHLYTLIVRPDNTYEVKIDNSQVESGSLEDDWDFLPPKKIKDPDASKPEDWDERAKIDDPTDSKPEDWDKPEHIPDPDAKKPEDWDEEMDGEWEPPVIQNPEYKGEWKPRQIDNPDYKGTWIHPEIDNPEYSPDPSIYAYDNFGVLGLDLWQVKSGTIFDNFLITNDEAYAEEFGNETWGVTKAAEKQMKDKQDEEQRTRRMMRTKMRMRRMRRTRRKMRRKMSPARPRTSCREACLQGWTEA

SEQ ID NO: 336 - protein, 664 aa, synthetic construct
MPSWALFMVTSCLLLAPQNLAQVSSQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQPVSQGSSGGGGSGGGGSSGVTGYRLFEEIL

SEQ ID NO: 337 - protein, 811 aa, synthetic construct
MPSWALFMVTSCLLLAPQNLAQVSSQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWISLVTALHLVLGLSAVLGLLLLRWQFPAHYRRLRHALWPSLPDLHRVLGQYLRDTAALSPPKATVSDTCEEVEPSLLEILPKSSERTPLPLCSSQAQMDYRRLQPSCLGTMPLSVCPPMAESGSCCTTHIANHSYLPLSYWQQPVSQGSSGGGGSGGGGSSGVFTLEDFVGDWEQTAAYNLDQVLEQGGVSSLLQNLAVSVTPIQRIVRSGENALKIDIHVIIPYEGLSADQMAQIEEVFKVVYPVDDHHFKVILPYGTLVIDGVTPNMLNYFGRPYEGIAVFDGKKITVTGTLWNGNKIIDERLITPDGSMLFRVTINS

SEQ ID NO: 338 - protein, 473 aa, synthetic construct
SQDVSLLASDSEPLKCFSRTFEDLTCFWDEEEAAPSGTYQLLYAYPREKPRACPLSSQSMPHFGTRYVCQFPDQEEVRLFFPLHLWVKNVFLNQTRTQRVLFVDSVGLPAPPSIIKAMGGSQPGELQISWEEPAPEISDFLRYELRYGPRDPKNSTGPTVIQLIATETCCPALQRPHSASALDQSPCAQPTMPWQDGPKQTSPSREASALTAEGGSCLISGLQPGNSYWLQLRSEPDGISLGGSWGSWSLPVTVDLPGDAVALGLQCFTLDLKNVTCQWQQQDHASSQGFFYHSRARCCPRDRYPIWENCEEEEKTNPGLQTPQFSRCHFKSRNDSIIHILVEVTTAPGTVHSYLGSPFWIHQAVRLPTPNLHWREISSGHLELEWQHPSSWAAQETCYQLRYTGEGHQDWKVLEPPLGARGGTLELRPRSRYRLQLRARLNGPTYQGPWSSWSDPTRVETATETAWHHHHHH

SEQ ID NO: 339 - protein, 21 aa, synthetic construct
  MOD_RES 1: note = N-term acetylated aspartic acid
  MOD_RES 21: note = C-term amidated arginine
DEEQRTRRMMRTKMRMRRMRR

SEQ ID NO: 340 - protein, 31 aa, synthetic construct
  MOD_RES 1: note = N-term hydroxylated aspartic acid
  MOD_RES 31: note = C-term hydroxylated serine
DEEQRTRRMMRTKMRMRRMRRTRRKMRRKMS

SEQ ID NO: 341 - protein, 448 aa, synthetic construct
QVQLQQSGAELVKPGSSVKISCKASGYTFTRNFIHWIKQQPGNGLEWIGWIFPGDGDTEYNQKFNGKATLTADKSSSTAYMQLSSLTSEDSAVYFCARGNYNYEYFDYWGQGVMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 342 - protein, 214 aa, synthetic construct
DIQMTQSPASLSASLGETVSIECLASEDIYSYLAWYQQKPGKSPQLLIFAANRLQDGVPSRFSGSGSGTQFSLKISGMQPEDEGDYFCLQGSKFPYTFGPGTKLELNRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 343 - protein, 442 aa, synthetic construct
QVQLVQSGAEVKKPGASVKVSCKASGYSFTGYYIHWVRQAPGQGLEWIGYISAYNGASSYNQKFKGRATFTVDTSISTAYMELSRLRSDDTAVYYCASSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 344 - protein, 219 aa, synthetic construct
DVVMTQSPLSLPVTLGQPASISCKSSQSLLDSDGKTYLNWLQQRPGQSPKRLIYLVSKLDSGVPDRFSGSGSGTDFTLKISRVEAEDVGVYHCWQGTHFPYTFGQGTKLIEKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 345 - protein, 439 aa, synthetic construct
EVQLKQSGPELVKTGASVKISCKASGYSFTGYYIHWVKQSHGKSLEWIGYISCYNGASSYNQKFKGKATFTVDTSSSTAYMQFNSLTSGDSAVYYCASSMDYWGQGTSVTVSSASTKGPSVFPLAPSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPNNYKTTPPVLDSDGSFFLYSKLTVDKSRWQQGNVFSCSVMHEALHNHYTQKSLSLSPG

SEQ ID NO: 346 - protein, 218 aa, synthetic construct
DVVMTQTPLTLSVTIGQPASISCKSSQSLLDSDGKTYLNWLLQRPGQSPKRLIYLVSKLDSGVPDRFTGSGSGTDFTLKISRVEAEDLGVYHCWQGTHFPYTFGGGTKLEIKRTVAAPSVFIFPPSDQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 347 - protein, 4 aa, Homo sapiens
KDEL

(19) United States
(12) Patent Application Publication
(10) Pub. No.: US 2023/0272055 A1
(43) Pub. Date: Aug. 31, 2023

(54) ANTI-MUTANT CALRETICULIN (CALR) ANTIBODIES AND USES THEREOF

(71) Applicant: Incyte Corporation, Wilmington, DE (US)

(72) Inventors: Patrick Mayes, Devon, PA (US); Horacio G. Nastri, West Chester, PA (US); Rebecca A. Buonpane, Wilmington, DE (US); Edimara S. Reis, Philadelphia, PA (US); Shaun M. Stewart, Chadds Ford, PA (US); Jing Zhou, Boxborough, MA (US); Marc C. Deller, Wilmington, DE (US); Hsiang-Ting Lei, Hockessin, DE (US); Leslie Brooke Epling McQuirter, Wilmington, DE (US); Yonghong Zhao, Trooper, PA (US); Hamza Celik, Wilmington, DE (US); Brittney Melissa Wass, Wilmington, DE (US)

(21) Appl. No.: 18/062,924

(22) Filed: Dec. 7, 2022

Related U.S. Application Data
(60) Provisional application No. 63/287,394, filed on Dec. 8, 2021.
(60) Provisional application No. 63/288,479, filed on Dec. 10, 2021.
(60) Provisional application No. 63/421,052, filed on Oct. 31, 2022.

(51) Int. Cl. C07K 16/18; A61P 35/00; A61K 31/519; A61K 39/395; G01N 33/574

(52) U.S. Cl. CPC C07K 16/18; A61P 35/00; A61K 31/519; A61K 39/3955; G01N 33/57496; C07K 2317/76; C07K 2317/92; C07K 2317/73; A61K 2039/505

(57) ABSTRACT

Anti-mutant calreticulin (mutCALR) antibodies are disclosed. Also disclosed are related nucleic acids, vectors, cells, kits, and pharmaceutical compositions. Methods of treating or diagnosing myeloproliferative neoplasms with the anti-mutCALR antibodies are also disclosed.

### CLAIM OF PRIORITY

This application claims the benefit of U.S. Provisional Patent Application No. 63/287,394, filed on Dec. 8, 2021, U.S. Provisional Patent Application No. 63/288,479, filed on Dec. 10, 2021, and U.S. Provisional Patent Application No. 63/421,052, filed on Oct. 31, 2022, each of which is incorporated by reference herein in its entirety.

### SEQUENCE LISTING

This application contains a Sequence Listing that has been submitted electronically as an XML file named 20443-0736001_SL.xml. The XML file, created on Dec. 7, 2022, is 388,039 bytes in size. The material in the XML file is hereby incorporated by reference in its entirety.

### BACKGROUND

Calreticulin (CALR) is a highly conserved chaperone protein that resides primarily in the endoplasmic reticulum and is involved in a variety of cellular processes including protein folding, calcium homeostasis, cell adhesion, and integrin signaling. CALR is also found in the nucleus, suggesting that it may have a role in transcription regulation. Mutations in the gene for CALR have been identified in patients with myeloproliferative neoplasms.

### SUMMARY

The present disclosure is based, at least in part, on the development of antibodies having high binding affinity and specificity to mutant CALR (“mutCALR”).

Accordingly, aspects of the present disclosure provide an antibody that binds to human mutant calreticulin (CALR), wherein the antibody comprises a heavy chain variable region (VH) comprising a VH CDR1, a VH CDR2, and a VH CDR3, wherein: the VH CDR1 comprises the amino acid sequence X₁X₂X₃X₄X₅, wherein X₁ is S, E, or D; wherein X₂ is Y, L, or S; wherein X₃ is A, S, or F; wherein X₄ is I or M; and wherein X₅ is S, Q, or H; the VH CDR2 comprises the amino acid sequence X₆X₇X₈PX₉X₁₀X₁₁X₁₂X₁₃X₁₄YAX₁₅X₁₆X₁₇X₁₈G (SEQ ID NO:97), wherein X₆ is L or G; wherein X₇ is V, F, or I; wherein X₈ is D or I; wherein X₉ is E, D, or I; wherein X₁₀ is D, G, F, A, S, or E; wherein X₁₁ is G or A; wherein X₁₂ is E or T; wherein X₁₃ is T or A; wherein X₁₄ is I, M, or N; wherein X₁₅ is E or Q; wherein X₁₆ is K or R; wherein X₁₇ is F or L; and wherein X₁₈ is R or Q; the VH CDR3 comprises the amino acid sequence X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇X₃₈ (SEQ ID NO:98), wherein X₁₉ is P, E, or absent; wherein X₂₀ is G, E, or absent; wherein X₂₁ is G, W, S, or absent; wherein X₂₂ is I, D, S, P, or absent; wherein X₂₃ is S, L, I, T, G, or absent; wherein X₂₄ is P, T, Q, I, D, R, or absent; wherein X₂₅ is G, D, or absent; wherein X₂₆ is E, Y, P, L, D, or S; wherein X₂₇ is E, D, A, or G; wherein X₂₈ is S, F, A, E, Y, or W; wherein X₂₉ is Y or F; wherein X₃₀ is G, D, or W; wherein X₃₁ is P, Y, 1, or H; wherein X₃₂ is Y or absent; wherein X₃₃ is Y or absent; wherein X₃₄ is Y or absent; wherein X₃₅ is G or absent; wherein X₃₆ is M or absent; wherein X₃₇ is D or absent; wherein X₃₈ is V or absent; wherein the antibody comprises a light chain variable region (VL) comprising a VL CDR1, a VL CDR2, and a VL CDR3, wherein: the VL CDR1 comprises the amino acid sequence X₃₉X₄₀X₄₁X₄₂X₄₃X₄₄X₄₅X₄₆X₄₇X₄₈X₄₉X₅₀X₅₁X₅₂X₅₃X₅₄ (SEQ ID NO:99), wherein X₃₉ is T, A, or absent; wherein X₄₀ is G or absent; wherein X₄₁ is Q, G, V, T, or S; wherein X₄₂ is A, G, S, or N; wherein X₄₃ is S, N, D, T, or Y; wherein X₄₄ is Q, Y, N, D, S, or K; wherein X₄₅ is D, I, F, V, S, or T; wherein X₄₆ is N or absent; wherein X₄₇ is I or absent; wherein X₄₈ is I, G, or R; wherein X₄₉ is S, G, A, D, I, R, or T; wherein X₅₀ is Y or absent; wherein X₅₁ is N, K, I, or E; wherein X₅₂ is Y, S, N, D, H, F, R, or G; wherein X₅₃ is L or V; and wherein X₅₄ is N, H, S, D, or F; the VL CDR2 comprises the amino acid sequence X₅₅X₅₆X₅₇X₅₈X₅₉X₆₀X₆₁, wherein X₅₅ is T, D, E, Q, or R; wherein X₅₆ is A, D, V, or N; wherein X₅₇ is S, G, N, or R; wherein X₅₈ is I, N, D, or K; wherein X₅₉ is L, R, or W; wherein X₆₀ is E or P; and wherein X₆₁ is S, T, or L; the VL CDR3 comprises the amino acid sequence X₆₂X₆₃X₆₄X₆₅X₆₆X₆₇X₆₈X₆₉X₇₀X₇₁X₇₂, wherein X₆₂ is Q, S, C, or G; wherein X₆₃ is Q, V, S, T, or A; wherein X₆₄ is Q, L, W, or Y; wherein X₆₅ is Q, N, D, I, T, A, or G; wherein X₆₆ is S, P, G, N, or A; wherein X₆₇ is N, Y, I, S, N, L, or D; wherein X₆₈ is E, P, S, I, N, H, L, or T; wherein X₆₉ is D, T, S, or absent; wherein X₇₀ is P, H, L, R, F, A, Q, or absent; wherein X₇₁ is W, L, V, Y, S, A, or E; and wherein X₇₂ is T, V, or I.

In some embodiments, the VH CDR1 comprises the amino acid sequence of any one of SEQ ID NOs:1-6; the VH CDR2 comprises the amino acid sequence of any one of SEQ ID NOs:7-17 and 92-95; the VH CDR3 comprises the amino acid sequence of any one of SEQ ID NOs:18-25; the VL CDR1 comprises the amino acid sequence of any one of SEQ ID NOs:26-52 or 118; the VL CDR2 comprises the amino acid sequence of any one of SEQ ID NOs:53-68; and the VL CDR3 comprises the amino acid sequence of any one of SEQ ID NOs:69-91.

In some embodiments, the VH CDR1, the VH CDR2, and the VH CDR3 each correspond to the VH CDRs set forth in Tables 1-2 for a single VH clone, and wherein the VL CDR1, the VL CDR2, and the VL CDR3 each correspond to the VL CDRs set forth in Tables 1-2 for a single VL clone.

In some embodiments, the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:26; the VL CDR2 comprises the amino acid sequence of SEQ ID NO:53; and the VL CDR3 comprises the amino acid sequence of SEQ ID NO:69;
the VH CDR1 comprises the amino acid sequence of SEQ ID NO: 1 the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:27; the VL CDR2 comprises the amino acid sequence of SEQ ID NO:54; and the VL CDR3 comprises the amino acid sequence of SEQ ID NO:70;
the VH CDR1 comprises the amino acid sequence of SEQ ID NO:1; the VH CDR2 comprises the amino acid sequence of SEQ ID NO:8; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:29; the VL CDR2 comprises the amino acid sequence of SEQ ID NO:54; and the VL CDR3 comprises the amino acid sequence of SEQ ID NO:72;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:71;
the VH CDR1 comprises the amino acid sequence of SEQ ID NO: 1 the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:29; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO: 1 the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:73;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:56; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:31; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:30; the VL CDR2 comprises the amino acid sequence of SEQ ID NO:57; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:9; the VH CDR3 comprises the amino acid sequence of SEQ ID NO:18; the VL CDR1 comprises the amino acid sequence of SEQ ID NO:32; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO: 1 the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:33; the VL CDR2 comprises the amino acid sequence of SEQ ID NO:54; and the VL CDR3 comprises the amino acid sequence of SEQ ID NO:73;
the VH CDR1 comprises the amino acid sequence of SEQ ID NO: 1 the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:34; the VL CDR2 comprises the amino acid sequence of SEQ ID NO:56; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:27; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:35; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:36; the VL CDR2 comprises the amino acid sequence of SEQ ID NO:54; and the VL CDR3 comprises the amino acid sequence of SEQ ID NO:72;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:58; and the VL CDR3 comprises the amino acid sequence of SEQ ID NO:71;
the VH CDR1 comprises the amino acid sequence of SEQ ID NO: 1 the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:37; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO: 1 the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:38; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:32; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:39; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:40; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:10; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO: 1 the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:41; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO: 1 the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:70;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:40; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:74;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:75;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO: 118; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:2; the VH CDR2 comprises the amino acid sequence of SEQ ID NO:11; the VH CDR3 comprises the amino acid sequence of SEQ ID NO:19; the VL CDR1 comprises the amino acid sequence of SEQ ID NO:30; the VL CDR2 comprises the amino acid sequence of SEQ ID NO:59; and the VL CDR3 comprises the amino acid sequence of SEQ ID NO:76;
the VH CDR1 comprises the amino acid sequence of SEQ ID NO:2; the VH CDR2 comprises the amino acid sequence of SEQ ID NO:11; the VH CDR3 comprises the amino acid sequence of SEQ ID NO:19; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:77;
the VH CDR1 comprises the amino acid sequence of SEQ ID NO:2; the VH CDR2 comprises the amino acid sequence of SEQ ID NO:11; the VH CDR3 comprises the amino acid sequence of SEQ ID NO:19; the VL CDR1 comprises the amino acid sequence of SEQ ID NO:42; the VL CDR2 comprises the amino acid sequence of SEQ ID NO:59; and the VL CDR3 comprises the amino acid sequence of SEQ ID NO:76;
the VH CDR1 comprises the amino acid sequence of SEQ ID NO:2; the VH CDR2 comprises the amino acid sequence of SEQ ID NO:11; the VH CDR3 comprises the amino acid sequence of SEQ ID NO:19; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:78;
the VH CDR1 comprises the amino acid sequence of SEQ ID NO:2; the VH CDR2 comprises the amino acid sequence of SEQ ID NO:12; the VH CDR3 comprises the amino acid sequence of SEQ ID NO:19; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:77;
the VH CDR1 comprises the amino acid sequence of SEQ ID NO:2; the VH CDR2 comprises the amino acid sequence of SEQ ID NO:11; the VH CDR3 comprises the amino acid sequence of SEQ ID NO:19; the VL CDR1 comprises the amino acid sequence of SEQ ID NO:43; the VL CDR2 comprises the amino acid sequence of SEQ ID NO:59; and the VL CDR3 comprises the amino acid sequence of SEQ ID NO:79;
the VH CDR1 comprises the amino acid sequence of SEQ ID NO:2; the VH CDR2 comprises the amino acid sequence of SEQ ID NO:11; the VH CDR3 comprises the amino acid sequence of SEQ ID NO:19; the VL CDR1 comprises the amino acid sequence of SEQ ID NO:30; the VL CDR2 comprises the amino acid sequence of SEQ ID NO:60; and the VL CDR3 comprises the amino acid sequence of SEQ ID NO:79;
the VH CDR1 comprises the amino acid sequence of SEQ ID NO:3; the VH CDR2 comprises the amino acid sequence of SEQ ID NO:13; the VH CDR3 comprises the amino acid sequence of SEQ ID NO:20; the VL CDR1 comprises the amino acid sequence of SEQ ID NO:44; the VL CDR2 comprises the amino acid sequence of SEQ ID NO:61; and the VL CDR3 comprises the amino acid sequence of SEQ ID NO:80;
the VH CDR1 comprises the amino acid sequence of SEQ ID NO:3; the VH CDR2 comprises the amino acid sequence of SEQ ID NO:11; the VH CDR3 comprises the amino acid sequence of SEQ ID NO:20; the VL CDR1 comprises the amino acid sequence of SEQ ID NO:45; the VL CDR2 comprises the amino acid sequence of SEQ ID NO:61; and the VL CDR3 comprises the amino acid sequence of SEQ ID NO:80;
the VH CDR1 comprises the amino acid sequence of SEQ ID NO:4; the VH CDR2 comprises the amino acid sequence of SEQ ID NO:14; the VH CDR3 comprises the amino acid sequence of SEQ ID NO:21; the VL CDR1 comprises the amino acid sequence of SEQ ID NO:46; the VL CDR2 comprises the amino acid sequence of SEQ ID NO:62; and the VL CDR3 comprises the amino acid sequence of SEQ ID NO:81;
the VH CDR1 comprises the amino acid sequence of SEQ ID NO:5; the VH CDR2 comprises the amino acid sequence of SEQ ID NO:14; the VH CDR3 comprises the amino acid sequence of SEQ ID NO:21; the VL CDR1 comprises the amino acid sequence of SEQ ID NO:46; the VL CDR2 comprises the amino acid sequence of SEQ ID NO:62; and the VL CDR3 comprises the amino acid sequence of SEQ ID NO:81;
the VH CDR1 comprises the amino acid sequence of SEQ ID NO:5; the VH CDR2 comprises the amino acid sequence of SEQ ID NO:14; the VH CDR3 comprises the amino acid sequence of SEQ ID NO:21; the VL CDR1 comprises the amino acid sequence of SEQ ID NO:46; the VL CDR2 comprises the amino acid sequence of SEQ ID NO:63; and the VL CDR3 comprises the amino acid sequence of SEQ ID NO:81;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:47; the VL CDR2 comprises the amino acid sequence of SEQ ID NO:64; and the VL CDR3 comprises the amino acid sequence of SEQ ID NO:82;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:48; the VL CDR2 comprises the amino acid sequence of SEQ ID NO:64; and the VL CDR3 comprises the amino acid sequence of SEQ ID NO:83;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:49; the VL CDR2 comprises the amino acid sequence of SEQ ID NO:65; and the VL CDR3 comprises the amino acid sequence of SEQ ID NO:84;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:48; the VL CDR2 comprises the amino acid sequence of SEQ ID NO:64; and the VL CDR3 comprises the amino acid sequence of SEQ ID NO:85;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:50; the VL CDR2 comprises the amino acid sequence of SEQ ID NO:65; and the VL CDR3 comprises the amino acid sequence of SEQ ID NO:84;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:28; the VL CDR2 comprises the amino acid sequence of SEQ ID NO:66; and the VL CDR3 comprises the amino acid sequence of SEQ ID NO:86;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:47; the VL CDR2 comprises the amino acid sequence of SEQ ID NO:64; and the VL CDR3 comprises the amino acid sequence of SEQ ID NO:84;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:118; the VL CDR2 comprises the amino acid sequence of SEQ ID NO:66; and the VL CDR3 comprises the amino acid sequence of SEQ ID NO:87;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:50; the VL CDR2 comprises the amino acid sequence of SEQ ID NO:64; and the VL CDR3 comprises the amino acid sequence of SEQ ID NO:88;
the VH CDR1 comprises the amino acid sequence of SEQ ID NO:5; the VH CDR2 comprises the amino acid sequence of SEQ ID NO:11; the VH CDR3 comprises the amino acid sequence of SEQ ID NO:23; the VL CDR1 comprises the amino acid sequence of SEQ ID NO:44; the VL CDR2 comprises the amino acid sequence of SEQ ID NO:61; and the VL CDR3 comprises the amino acid sequence of SEQ ID NO:89;
the VH CDR1 comprises the amino acid sequence of SEQ ID NO:5; the VH CDR2 comprises the amino acid sequence of SEQ ID NO:16; the VH CDR3 comprises the amino acid sequence of SEQ ID NO:24; the VL CDR1 comprises the amino acid sequence of SEQ ID NO:51; the VL CDR2 comprises the amino acid sequence of SEQ ID NO:67; and the VL CDR3 comprises the amino acid sequence of SEQ ID NO:90;
the VH CDR1 comprises the amino acid sequence of SEQ ID NO:6; the VH CDR2 comprises the amino acid sequence of SEQ ID NO:17; the VH CDR3 comprises the amino acid sequence of SEQ ID NO:25; the VL CDR1 comprises the amino acid sequence of SEQ ID NO:52; the VL CDR2 comprises the amino acid sequence of SEQ ID NO:68; and the VL CDR3 comprises the amino acid sequence of SEQ ID NO:91;
the VH CDR1 comprises the amino acid sequence of SEQ ID NO: 1 the VH CDR2 comprises the amino acid sequence of SEQ ID NO:92; the VH CDR3 comprises the amino acid sequence of SEQ ID NO:18; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:71;
the VH CDR1 comprises the amino acid sequence of SEQ ID NO: 1 the VH CDR2 comprises the amino acid sequence of SEQ ID NO:93; the VH CDR3 comprises the amino acid sequence of SEQ ID NO:18; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:94; the VH CDR3 comprises the amino acid sequence of SEQ ID NO:18; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:71;
the VH CDR1 comprises the amino acid sequence of SEQ ID NO: 1; the VH CDR2 comprises the amino acid sequence of SEQ ID NO:95; the VH CDR3 comprises the amino acid sequence of SEQ ID NO:18; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:92; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:93; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO: 1 the VH CDR2 comprises the amino acid sequence of SEQ ID NO:94; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:95; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:92; the VH CDR3 comprises the amino acid sequence of SEQ ID NO:18; the VL CDR1 comprises the amino acid sequence of SEQ ID NO:27; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO: 1; the VH CDR2 comprises the amino acid sequence of SEQ ID NO:93; the VH CDR3 comprises the amino acid sequence of SEQ ID NO:18; the VL CDR1 comprises the amino acid sequence of SEQ ID NO:27; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:94; the VH CDR3 comprises the amino acid sequence of SEQ ID NO:18; the VL CDR1 comprises the amino acid sequence of SEQ ID NO:27; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:95; the VH CDR3 comprises the amino acid sequence of SEQ ID NO:18; the VL CDR1 comprises the amino acid sequence of SEQ ID NO:27; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO: 1 the VH CDR2 comprises the amino acid sequence of SEQ ID NO:92; the VH CDR3 comprises the amino acid sequence of SEQ ID NO:18; the VL CDR1 comprises the amino acid sequence of SEQ ID NO:36; the VL CDR2 comprises the amino acid sequence of SEQ ID NO:54; and the VL CDR3 comprises the amino acid sequence of SEQ ID NO:72;
the VH CDR1 comprises the amino acid sequence of SEQ ID NO:1; the VH CDR2 comprises the amino acid sequence of SEQ ID NO:93; the VH CDR3 comprises the amino acid sequence of SEQ ID NO:18; the VL CDR1 comprises the amino acid sequence of SEQ ID NO:36; the VL CDR2 comprises the amino acid sequence of SEQ ID NO:54; and the VL CDR3 comprises the amino acid sequence of SEQ ID NO:72;
the VH CDR1 comprises the amino acid sequence of SEQ ID NO:1; the VH CDR2 comprises the amino acid sequence of SEQ ID NO:94; the VH CDR3 comprises the amino acid sequence of SEQ ID NO:18; the VL CDR1 comprises the amino acid sequence of SEQ ID NO:36; the VL CDR2 comprises the amino acid sequence of SEQ ID NO:54; and the VL CDR3 comprises the amino acid sequence of SEQ ID NO:72;
the VH CDR1 comprises the amino acid sequence of SEQ ID NO:1; the VH CDR2 comprises the amino acid sequence of SEQ ID NO:95; the VH CDR3 comprises the amino acid sequence of SEQ ID NO:18; the VL CDR1 comprises the amino acid sequence of SEQ ID NO:36; the VL CDR2 comprises the amino acid sequence of SEQ ID NO:54; and the VL CDR3 comprises the amino acid sequence of SEQ ID NO:72; or
the VH CDR1 comprises the amino acid sequence of SEQ ID NO:329; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:71.

In some embodiments, the VH is at least 80% identical to the amino acid sequence of any one of SEQ ID NOs:165-208; and the VL is at least 80% identical to the amino acid sequence of any one of SEQ ID NOs:264-318.

In some embodiments, the VH comprises the amino acid sequence of any one of SEQ ID NOs:165-208; and the VL comprises the amino acid sequence of any one of SEQ ID NOs:264-318.

In some embodiments, the VH comprises the amino acid sequence of SEQ ID NO:165 and the VL comprises the amino acid sequence of SEQ ID NO:264;
the VH comprises the amino acid sequence of SEQ ID NO:165 and the VL comprises the amino acid sequence of SEQ ID NO:265;
the VH comprises the amino acid sequence of SEQ ID NO:166 and the VL comprises the amino acid sequence of SEQ ID NO:266;
the VH comprises the amino acid sequence of SEQ ID NO: 165 and the VL comprises the amino acid sequence of SEQ ID NO:266;
the VH comprises the amino acid sequence of SEQ ID NO:165 and the VL comprises the amino acid sequence of SEQ ID NO:267;
the VH comprises the amino acid sequence of SEQ ID NO:165 and the VL comprises the amino acid sequence of SEQ ID NO:268;
the VH comprises the amino acid sequence of SEQ ID NO:165 and the VL comprises the amino acid sequence of SEQ ID NO:269;
the VH comprises the amino acid sequence of SEQ ID NO:167 and the VL comprises the amino acid sequence of SEQ ID NO:270;
the VH comprises the amino acid sequence of SEQ ID NO:165 and the VL comprises the amino acid sequence of SEQ ID NO:271;
the VH comprises the amino acid sequence of SEQ ID NO:165 and the VL comprises the amino acid sequence of SEQ ID NO:272;
the VH comprises the amino acid sequence of SEQ ID NO: 168 and the VL comprises the amino acid sequence of SEQ ID NO:273;
the VH comprises the amino acid sequence of SEQ ID NO:169 and the VL comprises the amino acid sequence of SEQ ID NO:274;
the VH comprises the amino acid sequence of SEQ ID NO:170 and the VL comprises the amino acid sequence of SEQ ID NO:275;
the VH comprises the amino acid sequence of SEQ ID NO:171 and the VL comprises the amino acid sequence of SEQ ID NO:276;
the VH comprises the amino acid sequence of SEQ ID NO:165 and the VL comprises the amino acid sequence of SEQ ID NO:277;
the VH comprises the amino acid sequence of SEQ ID NO:165 and the VL comprises the amino acid sequence of SEQ ID NO:278;
the VH comprises the amino acid sequence of SEQ ID NO:165 and the VL comprises the amino acid sequence of SEQ ID NO:279;
the VH comprises the amino acid sequence of SEQ ID NO:165 and the VL comprises the amino acid sequence of SEQ ID NO:280;
the VH comprises the amino acid sequence of SEQ ID NO: 172 and the VL comprises the amino acid sequence of SEQ ID NO:281;
the VH comprises the amino acid sequence of SEQ ID NO:165 and the VL comprises the amino acid sequence of SEQ ID NO:282;
the VH comprises the amino acid sequence of SEQ ID NO:173 and the VL comprises the amino acid sequence of SEQ ID NO:283;
the VH comprises the amino acid sequence of SEQ ID NO:165 and the VL comprises the amino acid sequence of SEQ ID NO:284;
the VH comprises the amino acid sequence of SEQ ID NO:165 and the VL comprises the amino acid sequence of SEQ ID NO:285;
the VH comprises the amino acid sequence of SEQ ID NO:174 and the VL comprises the amino acid sequence of SEQ ID NO:286;
the VH comprises the amino acid sequence of SEQ ID NO:165 and the VL comprises the amino acid sequence of SEQ ID NO:287;
the VH comprises the amino acid sequence of SEQ ID NO: 165 and the VL comprises the amino acid sequence of SEQ ID NO:288;
the VH comprises the amino acid sequence of SEQ ID NO:165 and the VL comprises the amino acid sequence of SEQ ID NO:289;
the VH comprises the amino acid sequence of SEQ ID NO:165 and the VL comprises the amino acid sequence of SEQ ID NO:290;
the VH comprises the amino acid sequence of SEQ ID NO:165 and the VL comprises the amino acid sequence of SEQ ID NO:291;
the VH comprises the amino acid sequence of SEQ ID NO:165 and the VL comprises the amino acid sequence of SEQ ID NO:292;
the VH comprises the amino acid sequence of SEQ ID NO:175 and the VL comprises the amino acid sequence of SEQ ID NO:293;
the VH comprises the amino acid sequence of SEQ ID NO:175 and the VL comprises the amino acid sequence of SEQ ID NO:294;
the VH comprises the amino acid sequence of SEQ ID NO:175 and the VL comprises the amino acid sequence of SEQ ID NO:295;
the VH comprises the amino acid sequence of SEQ ID NO: 175 and the VL comprises the amino acid sequence of SEQ ID NO:296;
the VH comprises the amino acid sequence of SEQ ID NO:176 and the VL comprises the amino acid sequence of SEQ ID NO:294;
the VH comprises the amino acid sequence of SEQ ID NO:175 and the VL comprises the amino acid sequence of SEQ ID NO:297;
the VH comprises the amino acid sequence of SEQ ID NO:175 and the VL comprises the amino acid sequence of SEQ ID NO:298;
the VH comprises the amino acid sequence of SEQ ID NO:177 and the VL comprises the amino acid sequence of SEQ ID NO:299;
the VH comprises the amino acid sequence of SEQ ID NO:178 and the VL comprises the amino acid sequence of SEQ ID NO:300;
the VH comprises the amino acid sequence of SEQ ID NO:179 and the VL comprises the amino acid sequence of SEQ ID NO:301;
the VH comprises the amino acid sequence of SEQ ID NO: 180 and the VL comprises the amino acid sequence of SEQ ID NO:301;
the VH comprises the amino acid sequence of SEQ ID NO:180 and the VL comprises the amino acid sequence of SEQ ID NO:302;
the VH comprises the amino acid sequence of SEQ ID NO:181 and the VL comprises the amino acid sequence of SEQ ID NO:303;
the VH comprises the amino acid sequence of SEQ ID NO:182 and the VL comprises the amino acid sequence of SEQ ID NO:304;
the VH comprises the amino acid sequence of SEQ ID NO:182 and the VL comprises the amino acid sequence of SEQ ID NO:305;
the VH comprises the amino acid sequence of SEQ ID NO:183 and the VL comprises the amino acid sequence of SEQ ID NO:306;
the VH comprises the amino acid sequence of SEQ ID NO:182 and the VL comprises the amino acid sequence of SEQ ID NO:307;
the VH comprises the amino acid sequence of SEQ ID NO:182 and the VL comprises the amino acid sequence of SEQ ID NO:308;
the VH comprises the amino acid sequence of SEQ ID NO: 182 and the VL comprises the amino acid sequence of SEQ ID NO:309;
the VH comprises the amino acid sequence of SEQ ID NO:184 and the VL comprises the amino acid sequence of SEQ ID NO:310;
the VH comprises the amino acid sequence of SEQ ID NO:182 and the VL comprises the amino acid sequence of SEQ ID NO:311;
the VH comprises the amino acid sequence of SEQ ID NO:185 and the VL comprises the amino acid sequence of SEQ ID NO:312;
the VH comprises the amino acid sequence of SEQ ID NO:186 and the VL comprises the amino acid sequence of SEQ ID NO:313;
the VH comprises the amino acid sequence of SEQ ID NO:187 and the VL comprises the amino acid sequence of SEQ ID NO:314;
the VH comprises the amino acid sequence of SEQ ID NO:188 and the VL comprises the amino acid sequence of SEQ ID NO:268;
the VH comprises the amino acid sequence of SEQ ID NO: 189 and the VL comprises the amino acid sequence of SEQ ID NO:268;
the VH comprises the amino acid sequence of SEQ ID NO:190 and the VL comprises the amino acid sequence of SEQ ID NO:268;
the VH comprises the amino acid sequence of SEQ ID NO:191 and the VL comprises the amino acid sequence of SEQ ID NO:268;
the VH comprises the amino acid sequence of SEQ ID NO:192 and the VL comprises the amino acid sequence of SEQ ID NO:268;
the VH comprises the amino acid sequence of SEQ ID NO:193 and the VL comprises the amino acid sequence of SEQ ID NO:268;
the VH comprises the amino acid sequence of SEQ ID NO:194 and the VL comprises the amino acid sequence of SEQ ID NO:268;
the VH comprises the amino acid sequence of SEQ ID NO:195 and the VL comprises the amino acid sequence of SEQ ID NO:268;
the VH comprises the amino acid sequence of SEQ ID NO:196 and the VL comprises the amino acid sequence of SEQ ID NO:268;
the VH comprises the amino acid sequence of SEQ ID NO: 165 and the VL comprises the amino acid sequence of SEQ ID NO:315;
the VH comprises the amino acid sequence of SEQ ID NO:188 and the VL comprises the amino acid sequence of SEQ ID NO:315;
the VH comprises the amino acid sequence of SEQ ID NO:189 and the VL comprises the amino acid sequence of SEQ ID NO:315;
the VH comprises the amino acid sequence of SEQ ID NO:190 and the VL comprises the amino acid sequence of SEQ ID NO:315;
the VH comprises the amino acid sequence of SEQ ID NO:191 and the VL comprises the amino acid sequence of SEQ ID NO:315;
the VH comprises the amino acid sequence of SEQ ID NO:192 and the VL comprises the amino acid sequence of SEQ ID NO:315;
the VH comprises the amino acid sequence of SEQ ID NO:193 and the VL comprises the amino acid sequence of SEQ ID NO:315;
the VH comprises the amino acid sequence of SEQ ID NO: 194 and the VL comprises the amino acid sequence of SEQ ID NO:315;
the VH comprises the amino acid sequence of SEQ ID NO:195 and the VL comprises the amino acid sequence of SEQ ID NO:315;
the VH comprises the amino acid sequence of SEQ ID NO:196 and the VL comprises the amino acid sequence of SEQ ID NO:315;
the VH comprises the amino acid sequence of SEQ ID NO:165 and the VL comprises the amino acid sequence of SEQ ID NO:316;
the VH comprises the amino acid sequence of SEQ ID NO:188 and the VL comprises the amino acid sequence of SEQ ID NO:316;
the VH comprises the amino acid sequence of SEQ ID NO:189 and the VL comprises the amino acid sequence of SEQ ID NO:316;
the VH comprises the amino acid sequence of SEQ ID NO:190 and the VL comprises the amino acid sequence of SEQ ID NO:316;
the VH comprises the amino acid sequence of SEQ ID NO:191 and the VL comprises the amino acid sequence of SEQ ID NO:316;
the VH comprises the amino acid sequence of SEQ ID NO: 192 and the VL comprises the amino acid sequence of SEQ ID NO:316;
the VH comprises the amino acid sequence of SEQ ID NO:193 and the VL comprises the amino acid sequence of SEQ ID NO:316;
the VH comprises the amino acid sequence of SEQ ID NO:194 and the VL comprises the amino acid sequence of SEQ ID NO:316;
the VH comprises the amino acid sequence of SEQ ID NO:195 and the VL comprises the amino acid sequence of SEQ ID NO:316;
the VH comprises the amino acid sequence of SEQ ID NO:196 and the VL comprises the amino acid sequence of SEQ ID NO:316;
the VH comprises the amino acid sequence of SEQ ID NO:197 and the VL comprises the amino acid sequence of SEQ ID NO:268;
the VH comprises the amino acid sequence of SEQ ID NO:198 and the VL comprises the amino acid sequence of SEQ ID NO:268;
the VH comprises the amino acid sequence of SEQ ID NO: 199 and the VL comprises the amino acid sequence of SEQ ID NO:268;
the VH comprises the amino acid sequence of SEQ ID NO:200 and the VL comprises the amino acid sequence of SEQ ID NO:268;
the VH comprises the amino acid sequence of SEQ ID NO:201 and the VL comprises the amino acid sequence of SEQ ID NO:268;
the VH comprises the amino acid sequence of SEQ ID NO:197 and the VL comprises the amino acid sequence of SEQ ID NO:315;
the VH comprises the amino acid sequence of SEQ ID NO:198 and the VL comprises the amino acid sequence of SEQ ID NO:315;
the VH comprises the amino acid sequence of SEQ ID NO:199 and the VL comprises the amino acid sequence of SEQ ID NO:315;
the VH comprises the amino acid sequence of SEQ ID NO:200 and the VL comprises the amino acid sequence of SEQ ID NO:315;
the VH comprises the amino acid sequence of SEQ ID NO:201 and the VL comprises the amino acid sequence of SEQ ID NO:315;
the VH comprises the amino acid sequence of SEQ ID NO: 197 and the VL comprises the amino acid sequence of SEQ ID NO:316;
the VH comprises the amino acid sequence of SEQ ID NO:198 and the VL comprises the amino acid sequence of SEQ ID NO:316;
the VH comprises the amino acid sequence of SEQ ID NO:199 and the VL comprises the amino acid sequence of SEQ ID NO:316;
the VH comprises the amino acid sequence of SEQ ID NO:200 and the VL comprises the amino acid sequence of SEQ ID NO:316;
the VH comprises the amino acid sequence of SEQ ID NO:201 and the VL comprises the amino acid sequence of SEQ ID NO:316;
the VH comprises the amino acid sequence of SEQ ID NO:170 and the VL comprises the amino acid sequence of SEQ ID NO:268;
the VH comprises the amino acid sequence of SEQ ID NO:202 and the VL comprises the amino acid sequence of SEQ ID NO:268;
the VH comprises the amino acid sequence of SEQ ID NO:203 and the VL comprises the amino acid sequence of SEQ ID NO:268;
the VH comprises the amino acid sequence of SEQ ID NO:204 and the VL comprises the amino acid sequence of SEQ ID NO:268;
the VH comprises the amino acid sequence of SEQ ID NO:205 and the VL comprises the amino acid sequence of SEQ ID NO:268;
the VH comprises the amino acid sequence of SEQ ID NO:206 and the VL comprises the amino acid sequence of SEQ ID NO:268;
the VH comprises the amino acid sequence of SEQ ID NO:207 and the VL comprises the amino acid sequence of SEQ ID NO:268;
the VH comprises the amino acid sequence of SEQ ID NO:208 and the VL comprises the amino acid sequence of SEQ ID NO:268;
the VH comprises the amino acid sequence of SEQ ID NO:170 and the VL comprises the amino acid sequence of SEQ ID NO:315;
the VH comprises the amino acid sequence of SEQ ID NO:202 and the VL comprises the amino acid sequence of SEQ ID NO:315;
the VH comprises the amino acid sequence of SEQ ID NO:203 and the VL comprises the amino acid sequence of SEQ ID NO:315;
the VH comprises the amino acid sequence of SEQ ID NO:204 and the VL comprises the amino acid sequence of SEQ ID NO:315;
the VH comprises the amino acid sequence of SEQ ID NO:205 and the VL comprises the amino acid sequence of SEQ ID NO:315;
the VH comprises the amino acid sequence of SEQ ID NO:206 and the VL comprises the amino acid sequence of SEQ ID NO:315;
the VH comprises the amino acid sequence of SEQ ID NO:207 and the VL comprises the amino acid sequence of SEQ ID NO:315;
the VH comprises the amino acid sequence of SEQ ID NO:208 and the VL comprises the amino acid sequence of SEQ ID NO:315;
the VH comprises the amino acid sequence of SEQ ID NO:170 and the VL comprises the amino acid sequence of SEQ ID NO:316;
the VH comprises the amino acid sequence of SEQ ID NO:202 and the VL comprises the amino acid sequence of SEQ ID NO:316;
the VH comprises the amino acid sequence of SEQ ID NO:203 and the VL comprises the amino acid sequence of SEQ ID NO:316;
the VH comprises the amino acid sequence of SEQ ID NO:204 and the VL comprises the amino acid sequence of SEQ ID NO:316;
the VH comprises the amino acid sequence of SEQ ID NO:205 and the VL comprises the amino acid sequence of SEQ ID NO:316;
the VH comprises the amino acid sequence of SEQ ID NO:206 and the VL comprises the amino acid sequence of SEQ ID NO:316;
the VH comprises the amino acid sequence of SEQ ID NO:207 and the VL comprises the amino acid sequence of SEQ ID NO:316;
the VH comprises the amino acid sequence of SEQ ID NO:208 and the VL comprises the amino acid sequence of SEQ ID NO:316;
the VH comprises the amino acid sequence of SEQ ID NO:188 and the VL comprises the amino acid sequence of SEQ ID NO:277;
the VH comprises the amino acid sequence of SEQ ID NO: 189 and the VL comprises the amino acid sequence of SEQ ID NO:277;
the VH comprises the amino acid sequence of SEQ ID NO:190 and the VL comprises the amino acid sequence of SEQ ID NO:277;
the VH comprises the amino acid sequence of SEQ ID NO:191 and the VL comprises the amino acid sequence of SEQ ID NO:277;
the VH comprises the amino acid sequence of SEQ ID NO:192 and the VL comprises the amino acid sequence of SEQ ID NO:277;
the VH comprises the amino acid sequence of SEQ ID NO:193 and the VL comprises the amino acid sequence of SEQ ID NO:277;
the VH comprises the amino acid sequence of SEQ ID NO:194 and the VL comprises the amino acid sequence of SEQ ID NO:277;
the VH comprises the amino acid sequence of SEQ ID NO:195 and the VL comprises the amino acid sequence of SEQ ID NO:277;
the VH comprises the amino acid sequence of SEQ ID NO: 196 and the VL comprises the amino acid sequence of SEQ ID NO:277;
the VH comprises the amino acid sequence of SEQ ID NO:165 and the VL comprises the amino acid sequence of SEQ ID NO:317;
the VH comprises the amino acid sequence of SEQ ID NO:188 and the VL comprises the amino acid sequence of SEQ ID NO:317;
the VH comprises the amino acid sequence of SEQ ID NO:189 and the VL comprises the amino acid sequence of SEQ ID NO:317;
the VH comprises the amino acid sequence of SEQ ID NO:190 and the VL comprises the amino acid sequence of SEQ ID NO:317;
the VH comprises the amino acid sequence of SEQ ID NO:191 and the VL comprises the amino acid sequence of SEQ ID NO:317;
the VH comprises the amino acid sequence of SEQ ID NO:192 and the VL comprises the amino acid sequence of SEQ ID NO:317;
the VH comprises the amino acid sequence of SEQ ID NO:193 and the VL comprises the amino acid sequence of SEQ ID NO:317;
the VH comprises the amino acid sequence of SEQ ID NO: 194 and the VL comprises the amino acid sequence of SEQ ID NO:317;
the VH comprises the amino acid sequence of SEQ ID NO:195 and the VL comprises the amino acid sequence of SEQ ID NO:317;
the VH comprises the amino acid sequence of SEQ ID NO:196 and the VL comprises the amino acid sequence of SEQ ID NO:317;
the VH comprises the amino acid sequence of SEQ ID NO:197 and the VL comprises the amino acid sequence of SEQ ID NO:277;
the VH comprises the amino acid sequence of SEQ ID NO:198 and the VL comprises the amino acid sequence of SEQ ID NO:277;
the VH comprises the amino acid sequence of SEQ ID NO:199 and the VL comprises the amino acid sequence of SEQ ID NO:277;
the VH comprises the amino acid sequence of SEQ ID NO:200 and the VL comprises the amino acid sequence of SEQ ID NO:277;
the VH comprises the amino acid sequence of SEQ ID NO:201 and the VL comprises the amino acid sequence of SEQ ID NO:277;
the VH comprises the amino acid sequence of SEQ ID NO:197 and the VL comprises the amino acid sequence of SEQ ID NO:317;
the VH comprises the amino acid sequence of SEQ ID NO:198 and the VL comprises the amino acid sequence of SEQ ID NO:317;
the VH comprises the amino acid sequence of SEQ ID NO:199 and the VL comprises the amino acid sequence of SEQ ID NO:317;
the VH comprises the amino acid sequence of SEQ ID NO:200 and the VL comprises the amino acid sequence of SEQ ID NO:317;
the VH comprises the amino acid sequence of SEQ ID NO:201 and the VL comprises the amino acid sequence of SEQ ID NO:317;
the VH comprises the amino acid sequence of SEQ ID NO:170 and the VL comprises the amino acid sequence of SEQ ID NO:277;
the VH comprises the amino acid sequence of SEQ ID NO:202 and the VL comprises the amino acid sequence of SEQ ID NO:277;
the VH comprises the amino acid sequence of SEQ ID NO:203 and the VL comprises the amino acid sequence of SEQ ID NO:277;
the VH comprises the amino acid sequence of SEQ ID NO:204 and the VL comprises the amino acid sequence of SEQ ID NO:277;
the VH comprises the amino acid sequence of SEQ ID NO:205 and the VL comprises the amino acid sequence of SEQ ID NO:277;
the VH comprises the amino acid sequence of SEQ ID NO:206 and the VL comprises the amino acid sequence of SEQ ID NO:277;
the VH comprises the amino acid sequence of SEQ ID NO:207 and the VL comprises the amino acid sequence of SEQ ID NO:277;
the VH comprises the amino acid sequence of SEQ ID NO:208 and the VL comprises the amino acid sequence of SEQ ID NO:277;
the VH comprises the amino acid sequence of SEQ ID NO:170 and the VL comprises the amino acid sequence of SEQ ID NO:317;
the VH comprises the amino acid sequence of SEQ ID NO:202 and the VL comprises the amino acid sequence of SEQ ID NO:317;
the VH comprises the amino acid sequence of SEQ ID NO:203 and the VL comprises the amino acid sequence of SEQ ID NO:317;
the VH comprises the amino acid sequence of SEQ ID NO:204 and the VL comprises the amino acid sequence of SEQ ID NO:317;
the VH comprises the amino acid sequence of SEQ ID NO:205 and the VL comprises the amino acid sequence of SEQ ID NO:317;
the VH comprises the amino acid sequence of SEQ ID NO:206 and the VL comprises the amino acid sequence of SEQ ID NO:317;
the VH comprises the amino acid sequence of SEQ ID NO:207 and the VL comprises the amino acid sequence of SEQ ID NO:317;
the VH comprises the amino acid sequence of SEQ ID NO:208 and the VL comprises the amino acid sequence of SEQ ID NO:317;
the VH comprises the amino acid sequence of SEQ ID NO:188 and the VL comprises the amino acid sequence of SEQ ID NO:279;
the VH comprises the amino acid sequence of SEQ ID NO: 189 and the VL comprises the amino acid sequence of SEQ ID NO:279;
the VH comprises the amino acid sequence of SEQ ID NO:190 and the VL comprises the amino acid sequence of SEQ ID NO:279;
the VH comprises the amino acid sequence of SEQ ID NO:191 and the VL comprises the amino acid sequence of SEQ ID NO:279;
the VH comprises the amino acid sequence of SEQ ID NO:192 and the VL comprises the amino acid sequence of SEQ ID NO:279;
the VH comprises the amino acid sequence of SEQ ID NO:193 and the VL comprises the amino acid sequence of SEQ ID NO:279;
the VH comprises the amino acid sequence of SEQ ID NO:194 and the VL comprises the amino acid sequence of SEQ ID NO:279;
the VH comprises the amino acid sequence of SEQ ID NO:195 and the VL comprises the amino acid sequence of SEQ ID NO:279;
the VH comprises the amino acid sequence of SEQ ID NO: 196 and the VL comprises the amino acid sequence of SEQ ID NO:279;
the VH comprises the amino acid sequence of SEQ ID NO:165 and the VL comprises the amino acid sequence of SEQ ID NO:318;
the VH comprises the amino acid sequence of SEQ ID NO:188 and the VL comprises the amino acid sequence of SEQ ID NO:318;
the VH comprises the amino acid sequence of SEQ ID NO:189 and the VL comprises the amino acid sequence of SEQ ID NO:318;
the VH comprises the amino acid sequence of SEQ ID NO:190 and the VL comprises the amino acid sequence of SEQ ID NO:318;
the VH comprises the amino acid sequence of SEQ ID NO:191 and the VL comprises the amino acid sequence of SEQ ID NO:318;
the VH comprises the amino acid sequence of SEQ ID NO:192 and the VL comprises the amino acid sequence of SEQ ID NO:318;
the VH comprises the amino acid sequence of SEQ ID NO:193 and the VL comprises the amino acid sequence of SEQ ID NO:318;
the VH comprises the amino acid sequence of SEQ ID NO: 194 and the VL comprises the amino acid sequence of SEQ ID NO:318;
the VH comprises the amino acid sequence of SEQ ID NO:195 and the VL comprises the amino acid sequence of SEQ ID NO:318;
the VH comprises the amino acid sequence of SEQ ID NO:196 and the VL comprises the amino acid sequence of SEQ ID NO:318;
the VH comprises the amino acid sequence of SEQ ID NO:197 and the VL comprises the amino acid sequence of SEQ ID NO:279;
the VH comprises the amino acid sequence of SEQ ID NO:198 and the VL comprises the amino acid sequence of SEQ ID NO:279;
the VH comprises the amino acid sequence of SEQ ID NO:199 and the VL comprises the amino acid sequence of SEQ ID NO:279;
the VH comprises the amino acid sequence of SEQ ID NO:200 and the VL comprises the amino acid sequence of SEQ ID NO:279;
the VH comprises the amino acid sequence of SEQ ID NO:201 and the VL comprises the amino acid sequence of SEQ ID NO:279;
the VH comprises the amino acid sequence of SEQ ID NO:197 and the VL comprises the amino acid sequence of SEQ ID NO:318;
the VH comprises the amino acid sequence of SEQ ID NO:198 and the VL comprises the amino acid sequence of SEQ ID NO:318;
the VH comprises the amino acid sequence of SEQ ID NO:199 and the VL comprises the amino acid sequence of SEQ ID NO:318;
the VH comprises the amino acid sequence of SEQ ID NO:200 and the VL comprises the amino acid sequence of SEQ ID NO:318;
the VH comprises the amino acid sequence of SEQ ID NO:201 and the VL comprises the amino acid sequence of SEQ ID NO:318;
the VH comprises the amino acid sequence of SEQ ID NO:170 and the VL comprises the amino acid sequence of SEQ ID NO:279;
the VH comprises the amino acid sequence of SEQ ID NO:202 and the VL comprises the amino acid sequence of SEQ ID NO:279;
the VH comprises the amino acid sequence of SEQ ID NO:203 and the VL comprises the amino acid sequence of SEQ ID NO:279;
the VH comprises the amino acid sequence of SEQ ID NO:204 and the VL comprises the amino acid sequence of SEQ ID NO:279;
the VH comprises the amino acid sequence of SEQ ID NO:205 and the VL comprises the amino acid sequence of SEQ ID NO:279;
the VH comprises the amino acid sequence of SEQ ID NO:206 and the VL comprises the amino acid sequence of SEQ ID NO:279;
the VH comprises the amino acid sequence of SEQ ID NO:207 and the VL comprises the amino acid sequence of SEQ ID NO:279;
the VH comprises the amino acid sequence of SEQ ID NO:208 and the VL comprises the amino acid sequence of SEQ ID NO:279;
the VH comprises the amino acid sequence of SEQ ID NO:170 and the VL comprises the amino acid sequence of SEQ ID NO:318;
the VH comprises the amino acid sequence of SEQ ID NO:202 and the VL comprises the amino acid sequence of SEQ ID NO:318;
the VH comprises the amino acid sequence of SEQ ID NO:203 and the VL comprises the amino acid sequence of SEQ ID NO:318;
the VH comprises the amino acid sequence of SEQ ID NO:204 and the VL comprises the amino acid sequence of SEQ ID NO:318;
the VH comprises the amino acid sequence of SEQ ID NO:205 and the VL comprises the amino acid sequence of SEQ ID NO:318;
the VH comprises the amino acid sequence of SEQ ID NO:206 and the VL comprises the amino acid sequence of SEQ ID NO:318;
the VH comprises the amino acid sequence of SEQ ID NO:207 and the VL comprises the amino acid sequence of SEQ ID NO:318; or the VH comprises the amino acid sequence of SEQ ID NO:208 and the VL comprises the amino acid sequence of SEQ ID NO:318.

In some embodiments, the VH comprises the amino acid sequence of SEQ ID NO: 165 and the VL comprises the amino acid sequence of SEQ ID NO:268.

In some embodiments, the VH comprises the amino acid sequence of SEQ ID NO:165 and the VL comprises the amino acid sequence of SEQ ID NO:277.

In some embodiments, the VH comprises the amino acid sequence of SEQ ID NO:165 and the VL comprises the amino acid sequence of SEQ ID NO:279.

In some embodiments, the VH comprises the amino acid sequence of SEQ ID NO:165 and the VL comprises the amino acid sequence of SEQ ID NO:268.

In some embodiments, the VH comprises the amino acid sequence of SEQ ID NO:165 and the VL comprises the amino acid sequence of SEQ ID NO:315.

In some embodiments, the VH comprises the amino acid sequence of SEQ ID NO: 190 and the VL comprises the amino acid sequence of SEQ ID NO:315.

In some embodiments, the VH comprises the amino acid sequence of SEQ ID NO:196 and the VL comprises the amino acid sequence of SEQ ID NO:315.

In some embodiments, the VH comprises the amino acid sequence of SEQ ID NO 191 and the VL comprises the amino acid sequence of SEQ ID NO:277.

In some embodiments, the VH comprises the amino acid sequence of SEQ ID NO:165 and the VL comprises the amino acid sequence of SEQ ID NO:317.

In some embodiments, the VH comprises the amino acid sequence of SEQ ID NO:191 and the VL comprises the amino acid sequence of SEQ ID NO:318.

In some embodiments, the VH comprises the amino acid sequence of SEQ ID NO:195 and the VL comprises the amino acid sequence of SEQ ID NO:318.

In some embodiments, the antibody comprises:
a heavy chain comprising the amino acid sequence of any one of SEQ ID NOs:119-135 and 137-164; and
a light chain comprising the amino acid sequence of any one of SEQ ID NOs:209-249 and 251-263.

In some embodiments, the antibody comprises:
a heavy chain and a light chain each corresponding to the heavy chain and the light chain set forth in Tables 4-5 for a single clone selected from the group consisting of clones 1-53 and 55-215.

In some embodiments, the heavy chain comprises the amino acid sequence of SEQ ID NO: 119 and the light chain comprises the amino acid sequence of SEQ ID NO:213.

In some embodiments, the heavy chain comprises the amino acid sequence of SEQ ID NO:119 and the light chain comprises the amino acid sequence of SEQ ID NO:222.

In some embodiments, the heavy chain comprises the amino acid sequence of SEQ ID NO: 119 and the light chain comprises the amino acid sequence of SEQ ID NO:224.

In some embodiments, the heavy chain comprises the amino acid sequence of SEQ ID NO:142 and the light chain comprises the amino acid sequence of SEQ ID NO:213.

In some embodiments, the heavy chain comprises the amino acid sequence of SEQ ID NO:142 and the light chain comprises the amino acid sequence of SEQ ID NO:260.

In some embodiments, the heavy chain comprises the amino acid sequence of SEQ ID NO:145 and the light chain comprises the amino acid sequence of SEQ ID NO:260.

In some embodiments, the heavy chain comprises the amino acid sequence of SEQ ID NO:151 and the light chain comprises the amino acid sequence of SEQ ID NO:260.

In some embodiments, the heavy chain comprises the amino acid sequence of SEQ ID NO:146 and the light chain comprises the amino acid sequence of SEQ ID NO:222.

In some embodiments, the heavy chain comprises the amino acid sequence of SEQ ID NO:142 and the light chain comprises the amino acid sequence of SEQ ID NO:262.

In some embodiments, the heavy chain comprises the amino acid sequence of SEQ ID NO:146 and the light chain comprises the amino acid sequence of SEQ ID NO:263.

In some embodiments, the heavy chain comprises the amino acid sequence of SEQ ID NO:150 and the light chain comprises the amino acid sequence of SEQ ID NO:263.

In some embodiments, the antibody comprises a heavy chain variable region (VH) comprising a VH CDR1; a VH CDR2; and a VH CDR3; wherein: the VH CDR1 comprises the amino acid sequence ELSMQ (SEQ ID NO:1); the VH CDR2 comprises the amino acid sequence GFDPDDX₁₀₁ETMYAEX₁₀₂X₁₀₃QG (SEQ ID NO:102; Group 1 clones); wherein X₁₀₁ is D or G; wherein X₁₀₂ is K or R; and wherein X₁₀₃ is F or L; the VH CDR3 is SPGYDFFDY (SEQ ID NO:18); wherein the antibody comprises a light chain variable region (VL) comprising a VL CDR1, a VL CDR2, and a VL CDR3, wherein: the VL CDR1 comprises the amino acid sequence GGX₁₀₄X₁₀₅X₁₀₆GX₁₀₇X₁₀₈X₁₀₉VX₁₁₀ (SEQ ID NO:103; Group 1 clones), wherein X₁₀₄ is N, D, or S; wherein X₁₀₅ is Y, N, or D; wherein X₁₀₆ is I or T; wherein X₁₀₇ is S, D, I, R, or T; wherein X₁₀₈ is K, E, or I; wherein X₁₀₉ is S, I, R, G, N, or A; and wherein X₁₁₀ is H, F, or N; the VL CDR2 comprises the amino acid sequence DDX₁₁₁DRPX₁₁₂ (SEQ ID NO:104; Group 1 clones), wherein X₁₁₁ is G, S, or R; and wherein X₁₁₂ is S or L; and the VL CDR3 comprises the amino acid sequence QVWDX₁₁₃X₁₁₄X₁₁₅DX₁₁₆X₁₁₇X₁₁₈ (SEQ ID NO:105; Group 1 clones), wherein X₁₁₃ is S or A; wherein X₁₁₄ is I or S; wherein X₁₁₅ is S, I, or N; wherein X₁₁₆ is H, L, or Q; wherein X₁₁₇ is V or L; and wherein X₁₁₈ is V or I.

In some embodiments, the antibody comprises a heavy chain variable region (VH) comprising a VH CDR1; a VH CDR2; and a VH CDR3; wherein: the VH CDR1 comprises the amino acid sequence GYTLTELSMQ (SEQ ID NO:329); the VH CDR2 comprises the amino acid sequence GFDPDDX₁₀₁ETMYAEX₁₀₂X₁₀₃QG (SEQ ID NO:102; Group 1 clones); wherein X₁₀₁ is D or G; wherein X₁₀₂ is K or R; and wherein X₁₀₃ is F or L; the VH CDR3 is SPGYDFFDY (SEQ ID NO:18); wherein the antibody comprises a light chain variable region (VL) comprising a VL CDR1, a VL CDR2, and a VL CDR3, wherein: the VL CDR1 comprises the amino acid sequence GGX₁₀₄X₁₀₅X₁₀₆GX₁₀₇X₁₀₈X₁₀₉VX₁₁₀ (SEQ ID NO:103; Group 1 clones), wherein X₁₀₄ is N, D, or S; wherein X₁₀₅ is Y, N, or D; wherein X₁₀₆ is I or T; wherein X₁₀₇ is S, D, I, R, or T; wherein X₁₀₈ is K, E, or I; wherein X₁₀₉ is S, I, R, G, N, or A; and wherein X₁₁₀ is H, F, or N; the VL CDR2 comprises the amino acid sequence DDX₁₁₁DRPX₁₁₂ (SEQ ID NO:104; Group 1 clones), wherein X₁₁₁ is G, S, or R; and wherein X₁₁₂ is S or L; and the VL CDR3 comprises the amino acid sequence QVWDX₁₁₃X₁₁₄X₁₁₅DX₁₁₆X₁₁₇X₁₁₈ (SEQ ID NO:105; Group 1 clones), wherein X₁₁₃ is S or A; wherein X₁₁₄ is I or S; wherein X₁₁₅ is S, I, or N; wherein X₁₁₆ is H, L, or Q; wherein X₁₁₇ is V or L; and wherein X₁₁₈ is V or I.

In some embodiments, the antibody comprises a heavy chain variable region (VH) comprising a VH CDR1; a VH CDR2; and a VH CDR3; wherein: the VH CDR1 comprises the amino acid sequence ELSMQ (SEQ ID NO:1); the VH CDR2 comprises the amino acid sequence GFDPDDX₁₀₁ETMYAEX₁₀₂X₁₀₃QG (SEQ ID NO:102; Group 1 clones); wherein X₁₀₁ is D or G; wherein X₁₀₂ is K or R; and wherein X₁₀₃ is F or L; the VH CDR3 is SPGYDFFDY (SEQ ID NO: 18); wherein the antibody comprises a light chain variable region (VL) comprising a VL CDR1, a VL CDR2, and a VL CDR3, wherein: the VL CDR1 comprises the amino acid sequence TGTSSDVGGYNYVS (SEQ ID NO:30); the VL CDR2 comprises the amino acid sequence X₁₁₉VSX₁₂₀RPS (SEQ ID NO:106; Group 1 clones); wherein X₁₁₉ is E or D; and wherein X₁₂₀ is N or K; and the VL CDR3 comprises the amino acid sequence QVWDSSNDLLI (SEQ ID NO:71).

In some embodiments, the antibody comprises a heavy chain variable region (VH) comprising a VH CDR1; a VH CDR2; and a VH CDR3; wherein: the VH CDR1 comprises the amino acid sequence GYTLTELSMQ (SEQ ID NO:329); the VH CDR2 comprises the amino acid sequence GFDPDDX₁₀₁ETMYAEX₁₀₂X₁₀₃QG (SEQ ID NO:102; Group 1 clones); wherein X₁₁₀ is D or G; wherein X₁₀₂ is K or R; and wherein X₁₀₃ is F or L; the VH CDR3 is SPGYDFFDY (SEQ ID NO:18); wherein the antibody comprises a light chain variable region (VL) comprising a VL CDR1, a VL CDR2, and a VL CDR3, wherein: the VL CDR1 comprises the amino acid sequence TGTSSDVGGYNYVS (SEQ ID NO:30); the VL CDR2 comprises the amino acid sequence X₁₁₉VSX₁₂₀RPS (SEQ ID NO:106; Group 1 clones); wherein X₁₁₉ is E or D; and wherein X₁₂₀ is N or K; and the VL CDR3 comprises the amino acid sequence QVWDSSNDLLI (SEQ ID NO:71).

In some embodiments, the antibody comprises a heavy chain variable region (VH) comprising a VH CDR1; a VH CDR2; and a VH CDR3; wherein: the VH CDR1 comprises the amino acid sequence DYFIH (SEQ ID NO:2); the VH CDR2 comprises the amino acid sequence LVDPEDGETIYAEX₁₂₁FQG (SEQ ID NO:107; Group 2 clones), wherein X₁₂₁ is K or R; the VH CDR3 comprises the amino acid sequence PGGILTDPDAFDI (SEQ ID NO:19); wherein the antibody comprises a light chain variable region (VL) comprising a VL CDR1, a VL CDR2, and a VL CDR3, wherein: the VL CDR1 comprises the amino acid sequence X₁₂₂GTX₁₂₃SDVGGYNX₁₂₄VS (SEQ ID NO:108; Group 2 clones), wherein X₁₂₂ is T or A; wherein X₁₂₃ is S or G; and wherein X₁₂₄ is Y or H; the VL CDR2 comprises the amino acid sequence X₁₂₅VX₁₂₆X₁₂₇RPS (SEQ ID NO:109; Group 2 clones), wherein X₁₂₅ is D or E; wherein X₁₂₆ is N or S; and wherein X₁₂₇ is K or N; and the VL CDR3 comprises the amino acid sequence SSYX₁₂₈X₁₂₉SSTX₁₃₀X₁₃₁V (SEQ ID NO:110; Group 2 clones), wherein X₁₂₈ is I or T; wherein X₁₂₉ is P or S; wherein X₁₃₀ is R, P, F, or absent; and wherein X₁₃₁ is W or Y.

In some embodiments, the antibody comprises a heavy chain variable region (VH) comprising a VH CDR1; a VH CDR2; and a VH CDR3; wherein: the VH CDR1 comprises the amino acid sequence SYAIS (SEQ ID NO:3); the VH CDR2 comprises the amino acid sequence LVDPEDGETIYAEKFX₁₃₂G (SEQ ID NO:111; Group 3 clones), wherein X₁₃₂ is R or Q; the VH CDR3 comprises the amino acid sequence EESYGP (SEQ ID NO:20); wherein the antibody comprises a light chain variable region (VL) comprising a VL CDR1, a VL CDR2, and a VL CDR3, wherein: the VL CDR1 comprises the amino acid sequence QASQDISNYLX₁₃₃ (SEQ ID NO:112; Group 3 clones), X₁₃₃ is N or D; the VL CDR2 comprises the amino acid sequence DASNLET (SEQ ID NO:61); and the VL CDR3 comprises the amino acid sequence QQLNSYPLT (SEQ ID NO:80).

In some embodiments, the antibody comprises a heavy chain variable region (VH) comprising a VH CDR1; a VH CDR2; and a VH CDR3; wherein: the VH CDR1 comprises the amino acid sequence EX₁₃₄SMH (SEQ ID NO:113; Group 4 clones), wherein X₁₃₄ is S or L; the VH CDR2 comprises the amino acid sequence LVDPEDGETIYAQKFQG (SEQ ID NO:14); the VH CDR3 comprises the amino acid sequence EEWSGDGDDAFDI (SEQ ID NO:21); wherein the antibody comprises a light chain variable region (VL) comprising a VL CDR1, a VL CDR2, and a VL CDR3, wherein: the VL CDR1 comprises the amino acid sequence SGSSSNIGSYSVS (SEQ ID NO:46); the VL CDR2 comprises the amino acid sequence DX₁₃₅NKRPS (SEQ ID NO:114; Group 4 clones), wherein X₁₃₅ is N or D; and the VL CDR3 comprises the amino acid sequence GTWDSSLSAWV (SEQ ID NO:81).

In some embodiments, the antibody comprises a heavy chain variable region (VH) comprising a VH CDR1; a VH CDR2; and a VH CDR3; wherein: the VH CDR1 comprises the amino acid sequence SYAIS (SEQ ID NO:3); the VH CDR2 comprises the amino acid sequence GIIPIFGTANYAQKFQG (SEQ ID NO: 15); the VH CDR3 comprises the amino acid sequence SPLRGSGWYWHYYYGMDV (SEQ ID NO:22); wherein the antibody comprises a light chain variable region (VL) comprising a VL CDR1, a VL CDR2, and a VL CDR3, wherein: the VL CDR1 comprises the amino acid sequence GGNX₁₃₆IX₁₃₇X₁₃₈KX₁₃₉VH (SEQ ID NO:115; Group 6 clones), wherein X₁₃₆ is N or K; wherein X₁₃₇ is R or G; wherein X₁₃₈ is A, S, R, or T; and wherein X₁₃₉ is H or S; the VL CDR2 comprises the amino acid sequence X₁₄₀DX₁₄₁X₁₄₂RPS (SEQ ID NO:116; Group 6 clones), wherein X₁₄₀ is Q or R; wherein X₁₄₁ is S or R; and wherein X₁₄₂ is N or K; and the VL CDR3 comprises the amino acid sequence QX₁₄₃WX₁₄₄SX₁₄₅TX₁₄₆V (SEQ ID NO:117; Group 6 clones), wherein X₁₄₃ is A or V; wherein X₁₄₄ is D or G; wherein X₁₄₅ is S or N; and wherein X₁₄₆ is V, A, or E.

In some embodiments, the human mutant CALR is human Type 1 mutant CALR comprising the amino acid sequence of SEQ ID NO:320. In some embodiments, the human mutant CALR is human Type 2 mutant CALR comprising the amino acid sequence of SEQ ID NO:321.

In some embodiments, the anti-mutCALR antibody inhibits one or more signaling pathways downstream of thrombopoietin receptor (MPL) in a cell expressing human mutant CALR; inhibits oncogenic cell proliferation in a cell expressing human mutant CALR; and/or inhibits dimerization of MPL in a cell expressing human mutant CALR.

In some embodiments, the anti-mutCALR antibody inhibits one or more signaling pathways downstream of MPL in both a first cell expressing human Type 1 mutant CALR and in a second cell expressing human Type 2 mutant CALR; inhibits oncogenic cell proliferation in both a first cell expressing human Type 1 mutant CALR and in a second cell expressing human Type 2 mutant CALR; and/or inhibits dimerization of MPL in both a first cell expressing human Type 1 mutant CALR and in a second cell expressing human Type 2 mutant CALR.

In some embodiments, the one or more signaling pathways downstream of MPL are selected from the group consisting of Janus tyrosine kinase (JAK) and signal transducers and activators of transcription (STAT) signaling, mitogen-activated protein kinase (MEK) and extracellular signal-regulated kinase (ERK) signaling, serine/threonine kinase (AKT) signaling, and mammalian target of rapamycin (mTOR) signaling.

In some embodiments, the anti-mutCALR antibody has modulated Fc effector function. In some embodiments, the modulated Fc effector function is increased Fc effector function or reduced Fc effector function. In some embodiments, the anti-mutCALR antibody has reduced Fc effector function. In some embodiments, the Fc effector function is antibody-dependent cell-mediated cytotoxicity (ADCC), complement dependent cytotoxicity (CDC), or antibody dependent cellular phagocytosis (ADCP). In some embodiments, the anti-mutCALR antibody having reduced Fc effector function has increased binding affinity to human mutant CALR as compared to an antibody without reduced Fc effector function. In some embodiments, the Fc effector function is ADCC.

In some embodiments, the anti-mutCALR antibody is a human or humanized antibody. In some embodiments, the anti-mutCALR antibody is a full-length antibody. In some embodiments, the anti-mutCALR antibody is an IgG1, IgG2, IgG3 or IgG4 antibody. In some embodiments, the anti-mutCALR antibody is an IgG1 antibody. In some embodiments, the anti-mutCALR antibody is a bispecific antibody, a biparatopic antibody, a single chain antibody (scFv), an Fab fragment, an F(ab′)₂ fragment, an Fab′ fragment, an Fsc fragment, an Fv fragment, an scFv, an sc(Fv)₂, or a diabody.

In some embodiments, the anti-mutCALR antibody is a biparatopic antibody comprising two heavy chain-light chain pairs or one heavy chain-light chain pair. In some embodiments, the biparatopic antibody is a full-length antibody. In some embodiments, the biparatopic antibody comprises one heavy chain-light chain pair.

In some embodiments, the anti-mutCALR antibody is conjugated to a toxic substance. In some embodiments, the toxic substance is a radioisotope or a cytotoxic agent.

Aspects of the present disclosure provide a nucleic acid or a set of nucleic acids, which collectively encodes any one of the anti-mutCALR antibodies described herein.

Aspects of the present disclosure provide an expression vector or a set of expression vectors comprising the nucleic acid or the set of nucleic acids encoding any one of the anti-mutCALR antibodies described herein operably linked to a promoter.

Aspects of the present disclosure provide an isolated cell comprising the nucleic acid or the set of nucleic acids encoding any one of the anti-mutCALR antibodies or the expression vector or the set of expression vectors comprising the nucleic acid or the set of nucleic acids encoding any one of the anti-mutCALR antibodies described herein operably linked to a promoter.

Aspects of the present disclosure provide a method of making the anti-mutCALR antibody, comprising culturing the cell described herein and isolating the antibody.

Aspects of the present disclosure provide a pharmaceutical composition comprising the anti-mutCALR antibody, the nucleic acid or the set of nucleic acids, the expression vector or the set of expression vectors, or the isolated cell, and a pharmaceutically acceptable carrier.

Aspects of the present disclosure provide a method of treating a myeloproliferative neoplasm in a human subject in need thereof, the method comprising administering to the human subject an effective amount of an anti-mutCALR antibody described herein or the pharmaceutical composition thereof. Other aspects of the present disclosure provide an anti-mutCALR antibody described herein for use in the treatment of a myeloproliferative neoplasm, or the use of an anti-mutCALR antibody described herein for the manufacture of a medicament for the treatment of a myeloproliferative neoplasm.

Aspects of the present disclosure provide a method of detecting a CALR exon 9 mutation in a biological sample, the method comprising obtaining a biological sample from a human subject who has or is suspected of having a myeloproliferative neoplasm and contacting the sample with an anti-mutCALR antibody described herein such that the anti-mutCALR antibody binds to a mutCALR protein if the mutCALR protein is present in the biological sample.

Another aspect of the present disclosure provides a method of diagnosing a human subject with a myeloproliferative neoplasm, the method comprising obtaining a biological sample from a human subject who has or is suspected of having a myeloproliferative neoplasm and contacting the sample with an anti-mutCALR antibody described herein such that the anti-mutCALR antibody binds to a mutCALR protein if the mutCALR protein is present in the biological sample.

In some embodiments, the myeloproliferative neoplasm is selected from the group consisting of chronic myelogenous leukemia, polycythemia vera, primary myelofibrosis, essential thrombocythemia, chronic neutrophilic leukemia, acute myelogenus leukemia, and chronic eosinophilic leukemia, chronic myelomonocytic leukemia, myeloproliferative neoplasm and myelodysplastic syndrome, including myelodysplastic syndrome with refractory anaemia with ring sideroblasts, myelodysplastic syndrome with refractory anemia, and myelodysplastic syndrome with refractory anemia with excess blasts.

In some embodiments, methods described herein further comprise administering to the human subject an additional therapy selected from the group consisting of a Janus tyrosine kinase (JAK) inhibitor, a phosphoinositide 3-kinase (PI3K) inhibitor, a standard of care therapy, or a combination thereof. In some embodiments, the JAK inhibitor is ruxolitinib and itaticinib. In some embodiments, the PI3K inhibitor is parsaclisib. In some embodiments, the standard of care therapy is selected from the group consisting of IFN-alpha, hydroxyurea, thalidomide, lenalidomide, an androgen, an erythropoietin-stimulating agent, a chemotherapeutic agent, or a combination thereof.

In some embodiments, the administration of the antibody or the pharmaceutical composition thereof in combination with the JAK inhibitor produces a synergistic effect. In some embodiments, the JAK inhibitor is ruxolitinib.

Aspects of the present disclosure provide a kit comprising the anti-mutCALR antibody, the nucleic acid or the set of nucleic acids, the expression vector or the set of expression vectors, or the isolated cell, and instructions for use in treating a myeloproliferative neoplasm in a human subject in need thereof, optionally with instructions for use in combination with an additional therapy.

### BRIEF DESCRIPTION OF THE DRAWINGS

FIG. 1 includes a graph showing that anti-mutCALR antibodies inhibit STAT5 activation in Ba/F3 cells expressing MPL/mutCALR.

FIG. 2 includes graphs showing that anti-mutCALR antibodies inhibit mutCALR induced cell proliferation in the Ba/F3 cells stably transfected with MPL/mutCALR.

FIG. 3 includes graphs showing that anti-mutCALR antibodies are efficient in inhibiting oncogenic cell proliferation in Ba/F3 cells stably transfected with either MPU/mutCALR Type 1 (top) or MPL/mutCALR Type 2 (bottom).

FIGS. 4A-4G include graphs showing that anti-mutCALR antibodies inhibit the dimerization of the MPL protein.

FIG. 5 includes a schematic depiction of the dosing schedule of anti-mutCALR antibodies in mice injected with tumor cells expressing MPL/mutCALR.

FIG. 6 includes a graph of mouse survival after treatment with anti-mutCALR antibodies.

FIG. 7 includes a graph of spleen weight in mice after treatment with anti-mutCALR antibodies.

FIG. 8 includes a graph of number of platelets in blood after treatment of mice with anti-mutCALR antibodies.

FIG. 9 includes a graph of number of tumor cells in blood after treatment of mice with anti-mutCALR antibodies.

FIG. 10 includes graphs showing the ability of an anti-mutCALR antibody to potentiate the therapeutic response of ruxolitinib in the inhibition of oncogenic cell proliferation triggered by mutCALR Type 1 (top) or Type 2 (bottom).

FIG. 11 includes a graph showing that anti-mutCALR antibodies compete with MPL for the binding to mutCALR.

FIGS. 12A-12C include structural data of the Fab-mutCALR-peptide complex.

FIGS. 13A-13C include structural data of the Fab fragment binding to the first mutCALR peptide in the asymmetric unit of the crystal structure.

FIGS. 14A-14C include structural data of the Fab fragment binding to the second mutCALR peptide in the asymmetric unit of the crystal structure.

FIG. 15 includes an image of the sequence of mutant CALR peptide (SEQ ID NO:328) showing the CalR1 conformation binding residues (top) and CalR2 conformation binding residues (bottom), with the residues having close contact with Fab1 (<4.5 Å) shaded in grey. Note that the CalR1 and CalR2 conformation residues represent two opposite faces of the mutant CALR C-terminal helix, with more residues at the N-terminal that are covered in the CalR1 binding conformation than are covered in the CalR2 binding conformation. Close contact of Fab1 was assessed in MOE (Molecular Operating Environment).

FIGS. 16A-16C include structural data of anti-mutCALR antibody Fab fragment bound to 31-mer mutant CalR peptide.

FIGS. 17A-17C include structural data from an antigen-antibody interaction analysis.

FIGS. 18A-18C include data showing that anti-mutCALR antibody treatment restored normal blood counts, spleen volume, and bone marrow environment in CALR^{DEL/DEL} engineered mice.

FIGS. 19A-19D include data showing that anti-mutCALR antibodies inhibit mutCALR-derived oncogenic functions in CD34⁺ cells isolated from MPN patients carrying the CALR mutation.

FIG. 20. includes data showing cell-cycle profiles of BaF3 cells with indicated genotypes in the presence of different concentrations of an anti-mutCALR antibody.

FIG. 21A includes a graph showing data that AB1-AB4 and clone 4 bind to mutCALR.

FIG. 21B includes a graph showing data that clone 4 inhibits cell proliferation.

### DETAILED DESCRIPTION

Provided herein are anti-mutCALR antibodies and related nucleic acids, expression vectors, cells, kits, and pharmaceutical compositions. The anti-mutCALR antibodies described herein are useful in the treatment or prevention or diagnosis of myeloproliferative neoplasms (e.g., chronic myelogenous leukemia, polycythemia vera, primary myelofibrosis, essential thrombocythemia, chronic neutrophilic leukemia, acute myelogenus leukemia, chronic eosinophilic leukemia, chronic myelomonocytic leukemia, myeloproliferative neoplasm and myelodysplastic syndrome, including myelodysplastic syndrome with refractory anaemia with ring sideroblasts, myelodysplastic syndrome with refractory anemia, and myelodysplastic syndrome with refractory anemia with excess blasts.).

### Mutant CALR and Anti-mutCALR Antibodies

CALR is a highly conserved chaperone protein that resides primarily in the endoplasmic reticulum and is involved in a variety of cellular processes including protein folding, calcium homeostasis, cell adhesion, and integrin signaling. Mutations in the CALR gene have been identified in patients with myeloproliferative neoplasms. The two most frequent CALR mutations are a 52 base pair (bp) deletion and a 5 bp insertion, which are referred to as Type 1 and Type 2 mutations, respectively. Type 1 and Type 2 mutations cause a +1 frameshift within exon 9 that generates a novel, positively-charged C-terminal amino acid sequence that lacks the KDEL domain (SEQ ID NO:347) of the WT protein, thereby enabling the mutCALR to escape the ER and activate the thrombopoietin receptor (MPL) and induce constitutive activation of Janus kinase 2 (JAK2) signaling. The amino acid sequences of human WT CALR, Type 1 and Type 2 mutCALR, and the novel C-terminal sequence are shown below. The frameshift amino acid residues in Type 1 and Type 2 mutCALR are shown in bold and the novel C-terminal sequence is marked by underlining.

Human WT CALR (GenBank Accession No. NP_004334.1)
(SEQ ID NO: 319)
MLLSVPLLLGLLGLAVAEPAVYFKEQFLDGDGWTSRWIESKHKSDFGKFV
LSSGKFYGDEEKDKGLQTSQDARFYALSASFEPFSNKGQTLVVQFTVKHE
QNIDCGGGYVKLFPNSLDQTDMHGDSEYNIMFGPDICGPGTKKVHVIFNY
KGKNVLINKDIRCKDDEFTHLYTLIVRPDNTYEVKIDNSQVESGSLEDDW
DFLPPKKIKDPDASKPEDWDERAKIDDPTDSKPEDWDKPEHIPDPDAKKP
EDWDEEMDGEWEPPVIQNPEYKGEWKPRQIDNPDYKGTWIHPEIDNPEYS
PDPSIYAYDNFGVLGLDLWQVKSGTIFDNFLITNDEAYAEEFGNETWGVT
KAAEKQMKDKQDEEQRLKEEEEDKKRKEEEEAEDKEDDEDKDEDEEDEED
KEEDEEEDVPGQAKDEL
Human Type 1 mutCALR
(SEQ ID NO: 320)
MLLSVPLLLGLLGLAVAEPAVYFKEQFLDGDGWTSRWIESKHKSDFGKFV
LSSGKFYGDEEKDKGLQTSQDARFYALSASFEPFSNKGQTLVVQFTVKHE
QNIDCGGGYVKLFPNSLDQTDMHGDSEYNIMFGPDICGPGTKKVHVIFNY
KGKNVLINKDIRCKDDEFTHLYTLIVRPDNTYEVKIDNSQVESGSLEDDW
DFLPPKKIKDPDASKPEDWDERAKIDDPTDSKPEDWDKPEHIPDPDAKKP
EDWDEEMDGEWEPPVIQNPEYKGEWKPRQIDNPDYKGTWIHPEIDNPEYS
PDPSIYAYDNFGVLGLDLWQVKSGTIFDNFLITNDEAYAEEFGNETWGVT
KAAEKQMKDKQDEEQRTRRMMRTKMRMRRMRRTRRKMRRKMSPARPRTSC
REACLQGWTEA
Human Type 2 mutCALR
(SEQ ID NO: 321)
MLLSVPLLLGLLGLAVAEPAVYFKEQFLDGDGWTSRWIESKHKSDFGKFV
LSSGKFYGDEEKDKGLQTSQDARFYALSASFEPFSNKGQTLVVQFTVKHE
QNIDCGGGYVKLFPNSLDQTDMHGDSEYNIMFGPDICGPGTKKVHVIFNY
KGKNVLINKDIRCKDDEFTHLYTLIVRPDNTYEVKIDNSQVESGSLEDDW
DFLPPKKIKDPDASKPEDWDERAKIDDPTDSKPEDWDKPEHIPDPDAKKP
EDWDEEMDGEWEPPVIQNPEYKGEWKPROIDNPDYKGTWIHPEIDNPEYS
PDPSIYAYDNFGVLGLDLWQVKSGTIFDNFLITNDEAYAEEFGNETWGVT
KAAEKQMKDKQDEEQRLKEEEEDKKRKEEEEAEDNCRRMMRTKMRMRRMR
RTRRKMRRKMSPARPRTSCREACLQGWTEA
mutCALR C-terminal consensus mutant sequence
(SEQ ID NO: 322)
RMRRMRRTRRKMRRKMSPARPRTSCREACLQGWTEA

This disclosure provides anti-mutCALR antibodies that are useful in treating or diagnosing myeloproliferative neoplasms. Note that in the present disclosure, unless stated otherwise, amino acid positions assigned to CDRs and frameworks in a variable region of the anti-mutCALR antibodies are specified according to Kabat; see EA Kabat, Sequences of Proteins of Immunological Interest, U.S. Dept. of Health and Human Services, Public Health Service, National Institutes of Health, 1991, (OCoLC)1138727707.

In some embodiments, the anti-mutCALR antibody is an anti-mutCALR antibody that comprises one, two, three, four, five, and/or six CDRs of any one of the antibodies described herein. In some embodiments, an anti-mutCALR antibody comprises (i) one, two, and/or three heavy chain CDRs of any one of the clones presented in Tables 1-2, and/or (ii) one, two, and/or three light chain CDRs from any one of the clones presented in Tables 1-2.

In some embodiments, an anti-mutCALR antibody comprises (i) three heavy chain CDRs from any one of the clones presented in Tables 4-5, and (ii) three light chain CDRs from any one of the clones presented in Tables 4-5.

In some embodiments, an anti-mutCALR antibody comprises a heavy chain CDR1, CDR2, and CDR3 and/or a light chain variable region CDR1, CDR2, and CDR3 from an antibody described herein. In some embodiments, an anti-mutCALR antibody comprises a heavy chain CDR1, CDR2, and CDR3 and a light chain CDR1, CDR2, and CDR3 from an antibody described herein. In some embodiments, an anti-mutCALR antibody comprises a mouse version, mouse variant, human version, human variant, humanized version, humanized variant, or affinity matured variant of an antibody described herein.

In some embodiments, an anti-mutCALR antibody comprises a heavy chain CDR1, CDR2, and CDR3 and/or a light chain variable region CDR1, CDR2, and CDR3 from any clone disclosed herein, a humanized version thereof, or variants thereof (including affinity matured variants). In some embodiments, an anti-mutCALR antibody comprises a heavy chain CDR1, a heavy chain variable region CDR2, and a heavy chain variable region CDR3 from any clone disclosed herein. In other embodiments, an anti-mutCALR antibody comprises a light chain variable region CDR1, a light chain variable region CDR2, and a light chain variable region CDR3 from any clone disclosed herein. In certain embodiments, an anti-mutCALR antibody comprises a heavy chain CDR1, a heavy chain variable region CDR2, a heavy chain variable region CDR3, a light chain variable region CDR1, a light chain variable region CDR2, and a light chain variable region CDR3 from antibody any clone disclosed herein. In some embodiments, an anti-mutCALR antibody is a mouse version of any clone disclosed herein. In some embodiments, an anti-mutCALR antibody is a mouse variant of any clone disclosed herein. In some embodiments, an anti-mutCALR antibody is a human version of any clone disclosed herein. In some embodiments, an anti-mutCALR antibody is a human variant of any clone disclosed herein. In some embodiments, an anti-mutCALR antibody is a humanized version of any clone disclosed herein. In some embodiments, an anti-mutCALR antibody is a variant of any clone disclosed herein. In some embodiments, an anti-mutCALR antibody is an affinity matured variant of any clone disclosed herein.

In some embodiments, the anti-mutCALR antibody is a variant of an anti-mutCALR antibody described herein which comprises one to thirty conservative amino acid substitution(s), e.g., one to twenty-five, one to twenty, one to fifteen, one to ten, one to five, or one to three conservative amino acid substitution(s). In some embodiments, the conservative amino acid substitution(s) is in a CDR of the antibody. In some embodiments, the conservative amino acid substitution(s) is not in a CDR of the antibody. In some embodiments, the conservative amino acid substitution(s) is in a framework region of the antibody.

In some embodiments, a CDR comprises one amino acid substitution. In some embodiments, a CDR comprises two amino acid substitutions. In some embodiments, a CDR comprises three amino acid substitutions. In some embodiments, a CDR comprises four amino acid substitutions. In some embodiments, the one or more amino acid substitutions are conservative substitutions. In some embodiments, the CDR is a heavy chain CDR1. In some embodiments, the CDR is a heavy chain variable region CDR2. In some embodiments, the CDR is a heavy chain variable region CDR3. In some embodiments, the CDR is a light chain variable region CDR1. In some embodiments, the CDR is a light chain variable region CDR2. In some embodiments, the CDR is a light chain variable region CDR3. In some embodiments, the one or more substitutions are made as part of a humanization process. In some embodiments, the one or more substitutions are made as part of a germline humanization process. In some embodiments, the one or more substitutions are made as part of an affinity maturation process. In some embodiments, the one or more substitutions are made as part of an optimization process.

In some embodiments, an anti-mutCALR antibody comprises a heavy chain variable region CDR1, a heavy chain variable region CDR2, and a heavy chain variable region CDR3, each of which correspond to the heavy chain variable region CDRs set forth in Tables 1-2 for a single clone, and a light chain variable region CDR1, a light chain variable region VL CDR2, and a light chain variable region VL CDR3, each of which correspond to the VL CDRs set forth in Tables 1-2 for a single clone.

In some embodiments, an anti-mutCALR antibody comprises a heavy chain variable region CDR1, a heavy chain variable region CDR2, a heavy chain variable region CDR3, a light chain variable region CDR1, a light chain variable region CDR2, and a light chain variable region CDR3, each of which correspond to the VH and VL CDRs set forth in Tables 1-2 for a single clone.

In some embodiments, an anti-mutCALR antibody comprises a heavy chain variable region (VH) comprising a VH CDR1, a VH CDR2, and a VH CDR3, wherein the VH CDR1 comprises the amino acid sequence X₁X₂X₃X₄X₅, wherein X₁ is S, E, or D; wherein X₂ is Y, L, or S; wherein X₃ is A, S, or F; wherein X₄ is I or M; and wherein X₅ is S, Q, or H; the VH CDR2 comprises the amino acid sequence X₆X₇X₈PX₉X₁₀X₁₁X₁₂X₁₃X₁₄YAX₁₅X₁₆X₁₇X₁₈G (SEQ ID NO:97), wherein X₆ is L or G; wherein X₇ is V, F, or I; wherein X₈ is D or I; wherein X₉ is E, D, or I; wherein X₁₀ is D, G, F, A, S, or E; wherein X₁₁ is G or A; wherein X₁₂ is E or T; wherein X₁₃ is T or A; wherein X₁₄ is I, M, or N; wherein X₁₅ is E or Q; wherein X₁₆ is K or R; wherein X₁₇ is F or L; and wherein X₁₈ is R or Q; the VH CDR3 comprises the amino acid sequence X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇X₃₈ (SEQ ID NO:98), wherein X₁₉ is P, E, or absent; wherein X₂₀ is G, E, or absent; wherein X₂₁ is G, W, S, or absent; wherein X₂₂ is I, D, S, P, or absent; wherein X₂₃ is S, L, I, T, G, or absent; wherein X₂₄ is P, T, Q, I, D, R, or absent; wherein X₂₅ is G, D, or absent; wherein X₂₆ is E, Y, P, L, D, or S; wherein X₂₇ is E, D, A, or G; wherein X₂₈ is S, F, A, E, Y, or W; wherein X₂₉ is Y or F; wherein X₃₀ is G, D, or W; wherein X₃₁ is P, Y, I, or H; wherein X₃₂ is Y or absent; wherein X₃₃ is Y or absent; wherein X₃₄ is Y or absent; wherein X₃₅ is G or absent; wherein X₃₆ is M or absent; wherein X₃₇ is D or absent; wherein X₃₈ is V or absent; wherein the antibody comprises a light chain variable region (VL) comprising a VL CDR1, a VL CDR2, and a VL CDR3, wherein the VL CDR1 comprises the amino acid sequence X₃₉X₄₀X₄₁X₄₂X₄₃X₄₄X₄₅X₄₆X₄₇X₄₈X₄₉X₅₀X₅₁X₅₂X₅₃X₅₄ (SEQ ID NO:99), wherein X₃₉ is T, A, or absent; wherein X₄₀ is G or absent; wherein X₄₁ is Q, G, V, T, or S; wherein X₄₂ is A, G, S, or N; wherein X₄₃ is S, N, D, T, or Y; wherein X₄₄ is Q, Y, N, D, S, or K; wherein X₄₅ is D, I, F, V, S, or T; wherein X₄₆ is N or absent; wherein X₄₇ is I or absent; wherein X₄₈ is I, G, or R; wherein X₄₉ is S, G, A, D, I, R, or T; wherein X₅₀ is Y or absent; wherein X₅₁ is N, K, I, or E; wherein X₅₂ is Y, S, N, D, H, F, R, or G; wherein X₅₃ is L or V; and wherein X₅₄ is N, H, S, D, or F; the VL CDR2 comprises the amino acid sequence X₅₅X₅₆X₅₇X₅₈X₅₉X₆₀X₆₁, wherein X₅₅ is T, D, E, Q, or R; wherein X₅₆ is A, D, V, or N; wherein X₅₇ is S, G, N, or R; wherein X₅₈ is I, N, D, or K; wherein X₅₉ is L, R, or W; wherein X₆₀ is E or P; and wherein X₆₁ is S, T, or L; the VL CDR3 comprises the amino acid sequence X₆₂X₆₃X₆₄X₆₅X₆₆X₆₇X₆₈X₆₉X₇₀X₇₁X₇₂, wherein X₆₂ is Q, S, C, or G; wherein X₆₃ is Q, V, S, T, or A; wherein X₆₄ is Q, L, W, or Y; wherein X₆₅ is Q, N, D, I, T, A, or G; wherein X₆₆ is S, P, G, N, or A; wherein X₆₇ is N, Y, I, S, N, L, or D; wherein X₆₈ is E, P, S, I, N, H, L, or T; wherein X₆₉ is D, T, S, or absent; wherein X₇₀ is P, H, L, R, F, A, Q, or absent; wherein X₇₁ is W, L, V, Y, S, A, or E; and wherein X₇₂ is T, V, or I.

In some embodiments, an anti-mutCALR antibody comprises a heavy chain variable region (VH) comprising a VH CDR1; a VH CDR2; and a VH CDR3; wherein the VH CDR1 comprises the amino acid sequence ELSMQ (SEQ ID NO:1); the VH CDR2 comprises the amino acid sequence GFDPDDX₁₀₁ETMYAEX₁₀₂X₁₀₃QG (SEQ ID NO:102; Group 1 clones); wherein X₁₀₁ is D or G; wherein X₁₀₂ is K or R; and wherein X₁₀₃ is F or L; the VH CDR3 is SPGYDFFDY (SEQ ID NO:18); wherein the antibody comprises a light chain variable region (VL) comprising a VL CDR1, a VL CDR2, and a VL CDR3, wherein the VL CDR1 comprises the amino acid sequence GGX₁₀₄X₁₀₅X₁₀₆GX₁₀₇X₁₀₈X₁₀₉VX₁₁₀ (SEQ ID NO:103; Group 1 clones), wherein X₁₀₄ is N, D, or S; wherein X₁₀₅ is Y, N, or D; wherein X₁₀₆ is I or T; wherein X₁₀₇ is S, D, I, R, or T; wherein X₁₀₈ is K, E, or I; wherein X₁₀₉ is S, I, R, G, N, or A; and wherein X₁₁₀ is H, F, or N; the VL CDR2 comprises the amino acid sequence DDX₁₁₁DRPX₁₁₂ (SEQ ID NO:104; Group 1 clones), wherein X₁₁₁ is G, S, or R; and wherein X₁₁₂ is S or L; and the VL CDR3 comprises the amino acid sequence QVWDX₁₁₃X₁₁₄X₁₁₅DX₁₁₆X₁₁₇X₁₁₈ (SEQ ID NO:105; Group 1 clones), wherein X₁₁₃ is S or A; wherein X₁₁₄ is I or S; wherein X₁₁₅ is S, I, or N; wherein X₁₁₆ is H, L, or Q; wherein X₁₁₇ is V or L; and wherein X₁₁₈ is V or I.

In some embodiments, an anti-mutCALR antibody comprises a heavy chain variable region (VH) comprising a VH CDR1; a VH CDR2; and a VH CDR3; wherein the VH CDR1 comprises the amino acid sequence ELSMQ (SEQ ID NO:1); the VH CDR2 comprises the amino acid sequence GFDPDDX₁₀₁ETMYAEX₁₀₂X₁₀₃QG (SEQ ID NO:102; Group 1 clones); wherein X₁₀₁ is D or G; wherein X₁₀₂ is K or R; and wherein X₁₀₃ is F or L; the VH CDR3 is SPGYDFFDY (SEQ ID NO:18); wherein the antibody comprises a light chain variable region (VL) comprising a VL CDR1, a VL CDR2, and a VL CDR3, wherein the VL CDR1 comprises the amino acid sequence TGTSSDVGGYNYVS (SEQ ID NO:30); the VL CDR2 comprises the amino acid sequence X₁₁₉VSX₁₂₀RPS (SEQ ID NO:106; Group 1 clones); wherein X₁₁₉ is E or D; and wherein X₁₂₀ is N or K; and the VL CDR3 comprises the amino acid sequence QVWDSSNDLLI (SEQ ID NO:71).

In some embodiments, the antibody comprises a heavy chain variable region (VH) comprising a VH CDR1; a VH CDR2; and a VH CDR3; wherein: the VH CDR1 comprises the amino acid sequence GYTLTELSMQ (SEQ ID NO:329); the VH CDR2 comprises the amino acid sequence GFDPDDX₁₀₁ETMYAEX₁₀₂X₁₀₃QG (SEQ ID NO:102; Group 1 clones); wherein X₁₀₁ is D or G; wherein X₁₀₂ is K or R; and wherein X₁₀₃ is F or L; the VH CDR3 is SPGYDFFDY (SEQ ID NO:18); wherein the antibody comprises a light chain variable region (VL) comprising a VL CDR1, a VL CDR2, and a VL CDR3, wherein: the VL CDR1 comprises the amino acid sequence TGTSSDVGGYNYVS (SEQ ID NO:30); the VL CDR2 comprises the amino acid sequence X₁₁₉VSX₁₂₀RPS (SEQ ID NO:106; Group 1 clones); wherein X₁₁₉ is E or D; and wherein X₁₂₀ is N or K; and the VL CDR3 comprises the amino acid sequence QVWDSSNDLLI (SEQ ID NO:71).

In some embodiments, an anti-mutCALR antibody comprises a heavy chain variable region (VH) comprising a VH CDR1; a VH CDR2; and a VH CDR3; wherein the VH CDR1 comprises the amino acid sequence DYFIH (SEQ ID NO:2); the VH CDR2 comprises the amino acid sequence LVDPEDGETIYAEX₁₂₁FQG (SEQ ID NO:107; Group 2 clones), wherein X₁₂₁ is K or R; the VH CDR3 comprises the amino acid sequence PGGILTDPDAFDI (SEQ ID NO: 19); wherein the antibody comprises a light chain variable region (VL) comprising a VL CDR1, a VL CDR2, and a VL CDR3, wherein the VL CDR1 comprises the amino acid sequence X₁₂₂GTX₁₂₃SDVGGYNX₁₂₄VS (SEQ ID NO:108; Group 2 clones), wherein X₁₂₂ is T or A; wherein X₁₂₃ is S or G; and wherein X₁₂₄ is Y or H; the VL CDR2 comprises the amino acid sequence X₁₂₅VX₁₂₆X₁₂₇RPS (SEQ ID NO:109; Group 2 clones), wherein X₁₂₅ is D or E; wherein X₁₂₆ is N or S; and wherein X₁₂₇ is K or N; and the VL CDR3 comprises the amino acid sequence SSYX₁₂₈X₁₂₉SSTX₁₃₀X₁₃₁V (SEQ ID NO:110; Group 2 clones), wherein X₁₂₈ is I or T; wherein X₁₂₉ is P or S; wherein X₁₃₀ is R, P, F, or absent; and wherein X₁₃₁ is W or Y.

In some embodiments, an anti-mutCALR antibody comprises a heavy chain variable region (VH) comprising a VH CDR1; a VH CDR2; and a VH CDR3; wherein the VH CDR1 comprises the amino acid sequence SYAIS (SEQ ID NO:3); the VH CDR2 comprises the amino acid sequence LVDPEDGETIYAEKFX₁₃₂G (SEQ ID NO: 111; Group 3 clones), wherein X₁₃₂ is R or Q; the VH CDR3 comprises the amino acid sequence EESYGP (SEQ ID NO:20); wherein the antibody comprises a light chain variable region (VL) comprising a VL CDR1, a VL CDR2, and a VL CDR3, wherein the VL CDR1 comprises the amino acid sequence QASQDISNYLX₁₃₃ (SEQ ID NO:112; Group 3 clones), X₁₃₃ is N or D; the VL CDR2 comprises the amino acid sequence DASNLET (SEQ ID NO:61); and the VL CDR3 comprises the amino acid sequence QQLNSYPLT (SEQ ID NO:80).

In some embodiments, an anti-mutCALR antibody comprises a heavy chain variable region (VH) comprising a VH CDR1; a VH CDR2; and a VH CDR3; wherein the VH CDR1 comprises the amino acid sequence EX₁₃₄SMH (SEQ ID NO:113; Group 4 clones), wherein X₁₃₄ is S or L; the VH CDR2 comprises the amino acid sequence LVDPEDGETIYAQKFQG (SEQ ID NO:14); the VH CDR3 comprises the amino acid sequence EEWSGDGDDAFDI (SEQ ID NO:21); wherein the antibody comprises a light chain variable region (VL) comprising a VL CDR1, a VL CDR2, and a VL CDR3, wherein the VL CDR1 comprises the amino acid sequence SGSSSNIGSYSVS (SEQ ID NO:46); the VL CDR2 comprises the amino acid sequence DX₁₃₅NKRPS (SEQ ID NO:114; Group 4 clones), wherein X₁₃₅ is N or D; and the VL CDR3 comprises the amino acid sequence GTWDSSLSAWV (SEQ ID NO:81).

In some embodiments, an anti-mutCALR antibody comprises a heavy chain variable region (VH) comprising a VH CDR1; a VH CDR2; and a VH CDR3; wherein: the VH CDR1 comprises the amino acid sequence SYAIS (SEQ ID NO:3); the VH CDR2 comprises the amino acid sequence GIIPIFGTANYAQKFQG (SEQ ID NO:15); the VH CDR3 comprises the amino acid sequence SPLRGSGWYWHYYYGMDV (SEQ ID NO:22); wherein the antibody comprises a light chain variable region (VL) comprising a VL CDR1, a VL CDR2, and a VL CDR3, wherein the VL CDR1 comprises the amino acid sequence GGNX₁₃₆IX₁₃₇X₁₃₈KX₁₃₉VH (SEQ ID NO: 115; Group 6 clones), wherein X₁₃₆ is N or K; wherein X₁₃₇ is R or G; wherein X₁₃₈ is A, S, R, or T; and wherein X₁₃₉ is H or S; the VL CDR2 comprises the amino acid sequence X₁₄₀DX₁₄₁X₁₄₂RPS (SEQ ID NO:116; Group 6 clones), wherein X₁₄₀ is Q or R; wherein X₁₄₁ is S or R; and wherein X₁₄₂ is N or K; and the VL CDR3 comprises the amino acid sequence QX₁₄₃WX₁₄₄SX₁₄₅TX₁₄₆V (SEQ ID NO:117; Group 6 clones), wherein X₁₄₃ is A or V; wherein X₁₄₄ is D or G; wherein X₁₄₅ is S or N; and wherein X₁₄₆ is V, A, or E.

In some embodiments, an anti-mutCALR antibody comprises a heavy chain variable region with the C-terminal lysine removed. In some embodiments, an anti-mutCALR antibody comprises a heavy chain variable region comprising an amino acid sequence that has the three VH CDRs of any anti-mutCALR clone disclosed herein and which has at least 75%, 80%, 85%, 90%, 95%, 96%, 97%, 98%, 99%, or 100% identity to any one of the VH sequences set forth in Tables 4-5 and a light chain variable region comprising an amino acid sequence that has the three VL CDRs of any anti-mutCALR clone disclosed herein and which has at least 75%, 80%, 85%, 90%, 95%, 96%, 97%, 98%, 99%, or 100% identity to any one of the VL sequences set forth in Tables 4-5.

In some embodiments, an anti-mutCALR antibody comprises a heavy chain variable region comprising any one of the VH sequences set forth in Tables 4-5. In some embodiments, an anti-mutCALR antibody comprises a light chain variable region comprising any one of the VL sequences set forth in Tables 4-5. In some embodiments, an anti-mutCALR antibody comprises a heavy chain variable region comprising any one of the VH sequences set forth in Tables 4-5 and a light chain variable region comprising any one of the VL sequences set forth in Tables 4-5.

In some embodiments, an anti-mutCALR antibody comprises a VH comprising the amino acid sequence of SEQ ID NO:165 and a VL comprising the amino acid sequence of SEQ ID NO:264.

In some embodiments, an anti-mutCALR antibody comprises a VH comprising the amino acid sequence of SEQ ID NO:165 and a VL comprising the amino acid sequence of SEQ ID NO:265.

In some embodiments, an anti-mutCALR antibody comprises a VH comprising the amino acid sequence of SEQ ID NO:166 and a VL comprising the amino acid sequence of SEQ ID NO:266.

In some embodiments, an anti-mutCALR antibody comprises a VH comprising the amino acid sequence of SEQ ID NO:165 and a VL comprising the amino acid sequence of SEQ ID NO:266.

In some embodiments, an anti-mutCALR antibody comprises a VH comprising the amino acid sequence of SEQ ID NO:165 and a VL comprising the amino acid sequence of SEQ ID NO:267.

In some embodiments, an anti-mutCALR antibody comprises a VH comprising the amino acid sequence of SEQ ID NO:165 and a VL comprising the amino acid sequence of SEQ ID NO:268.

In some embodiments, an anti-mutCALR antibody comprises a VH comprising the amino acid sequence of SEQ ID NO:165 and a VL comprising the amino acid sequence of SEQ ID NO:269.

In some embodiments, an anti-mutCALR antibody comprises a VH comprising the amino acid sequence of SEQ ID NO:167 and a VL comprising the amino acid sequence of SEQ ID NO:270.

In some embodiments, an anti-mutCALR antibody comprises a VH comprising the amino acid sequence of SEQ ID NO:165 and a VL comprising the amino acid sequence of SEQ ID NO:271.

In some embodiments, an anti-mutCALR antibody comprises a VH comprising the amino acid sequence of SEQ ID NO:165 and a VL comprising the amino acid sequence of SEQ ID NO:272.

In some embodiments, an anti-mutCALR antibody comprises a VH comprising the amino acid sequence of SEQ ID NO:168 and a VL comprising the amino acid sequence of SEQ ID NO:273.

In some embodiments, an anti-mutCALR antibody comprises a VH comprising the amino acid sequence of SEQ ID NO:169 and a VL comprising the amino acid sequence of SEQ ID NO:274.

In some embodiments, an anti-mutCALR antibody comprises a VH comprising the amino acid sequence of SEQ ID NO:170 and a VL comprising the amino acid sequence of SEQ ID NO:275.

In some embodiments, an anti-mutCALR antibody comprises a VH comprising the amino acid sequence of SEQ ID NO:171 and a VL comprising the amino acid sequence of SEQ ID NO:276.

In some embodiments, an anti-mutCALR antibody comprises a VH comprising the amino acid sequence of SEQ ID NO:165 and a VL comprising the amino acid sequence of SEQ ID NO:277.

In some embodiments, an anti-mutCALR antibody comprises a VH comprising the amino acid sequence of SEQ ID NO:165 and a VL comprising the amino acid sequence of SEQ ID NO:278.

In some embodiments, an anti-mutCALR antibody comprises a VH comprising the amino acid sequence of SEQ ID NO:165 and a VL comprising the amino acid sequence of SEQ ID NO:279.

In some embodiments, an anti-mutCALR antibody comprises a VH comprising the amino acid sequence of SEQ ID NO:165 and a VL comprising the amino acid sequence of SEQ ID NO:280.

In some embodiments, an anti-mutCALR antibody comprises a VH comprising the amino acid sequence of SEQ ID NO:172 and a VL comprising the amino acid sequence of SEQ ID NO:281.

In some embodiments, an anti-mutCALR antibody comprises a VH comprising the amino acid sequence of SEQ ID NO:165 and a VL comprising the amino acid sequence of SEQ ID NO:282.

In some embodiments, an anti-mutCALR antibody comprises a VH comprising the amino acid sequence of SEQ ID NO:173 and a VL comprising the amino acid sequence of SEQ ID NO:283.

In some embodiments, an anti-mutCALR antibody comprises a VH comprising the amino acid sequence of SEQ ID NO:165 and a VL comprising the amino acid sequence of SEQ ID NO:284.

In some embodiments, an anti-mutCALR antibody comprises a VH comprising the amino acid sequence of SEQ ID NO:165 and a VL comprising the amino acid sequence of SEQ ID NO:285.

In some embodiments, an anti-mutCALR antibody comprises a VH comprising the amino acid sequence of SEQ ID NO:174 and a VL comprising the amino acid sequence of SEQ ID NO:286.

In some embodiments, an anti-mutCALR antibody comprises a VH comprising the amino acid sequence of SEQ ID NO:165 and a VL comprising the amino acid sequence of SEQ ID NO:287.

In some embodiments, an anti-mutCALR antibody comprises a VH comprising the amino acid sequence of SEQ ID NO:165 and a VL comprising the amino acid sequence of SEQ ID NO:288.

In some embodiments, an anti-mutCALR antibody comprises a VH comprising the amino acid sequence of SEQ ID NO:165 and a VL comprising the amino acid sequence of SEQ ID NO:289.

In some embodiments, an anti-mutCALR antibody comprises a VH comprising the amino acid sequence of SEQ ID NO:165 and a VL comprising the amino acid sequence of SEQ ID NO:290.

In some embodiments, an anti-mutCALR antibody comprises a VH comprising the amino acid sequence of SEQ ID NO:165 and a VL comprising the amino acid sequence of SEQ ID NO:291.

In some embodiments, an anti-mutCALR antibody comprises a VH comprising the amino acid sequence of SEQ ID NO:165 and a VL comprising the amino acid sequence of SEQ ID NO:292.

In some embodiments, an anti-mutCALR antibody comprises a VH comprising the amino acid sequence of SEQ ID NO:175 and a VL comprising the amino acid sequence of SEQ ID NO:293.

In some embodiments, an anti-mutCALR antibody comprises a VH comprising the amino acid sequence of SEQ ID NO:175 and a VL comprising the amino acid sequence of SEQ ID NO:294.

In some embodiments, an anti-mutCALR antibody comprises a VH comprising the amino acid sequence of SEQ ID NO:175 and a VL comprising the amino acid sequence of SEQ ID NO:295.

In some embodiments, an anti-mutCALR antibody comprises a VH comprising the amino acid sequence of SEQ ID NO:175 and a VL comprising the amino acid sequence of SEQ ID NO:296.

In some embodiments, an anti-mutCALR antibody comprises a VH comprising the amino acid sequence of SEQ ID NO:176 and a VL comprising the amino acid sequence of SEQ ID NO:294.

In some embodiments, an anti-mutCALR antibody comprises a VH comprising the amino acid sequence of SEQ ID NO:175 and a VL comprising the amino acid sequence of SEQ ID NO:297.

In some embodiments, an anti-mutCALR antibody comprises a VH comprising the amino acid sequence of SEQ ID NO:175 and a VL comprising the amino acid sequence of SEQ ID NO:298.

In some embodiments, an anti-mutCALR antibody comprises a VH comprising the amino acid sequence of SEQ ID NO:177 and a VL comprising the amino acid sequence of SEQ ID NO:299.

In some embodiments, an anti-mutCALR antibody comprises a VH comprising the amino acid sequence of SEQ ID NO:178 and a VL comprising the amino acid sequence of SEQ ID NO:300.

In some embodiments, an anti-mutCALR antibody comprises a VH comprising the amino acid sequence of SEQ ID NO:179 and a VL comprising the amino acid sequence of SEQ ID NO:301.

In some embodiments, an anti-mutCALR antibody comprises a VH comprising the amino acid sequence of SEQ ID NO:180 and a VL comprising the amino acid sequence of SEQ ID NO:301.

In some embodiments, an anti-mutCALR antibody comprises a VH comprising the amino acid sequence of SEQ ID NO:180 and a VL comprising the amino acid sequence of SEQ ID NO:302.

In some embodiments, an anti-mutCALR antibody comprises a VH comprising the amino acid sequence of SEQ ID NO:181 and a VL comprising the amino acid sequence of SEQ ID NO:303.

In some embodiments, an anti-mutCALR antibody comprises a VH comprising the amino acid sequence of SEQ ID NO:182 and a VL comprising the amino acid sequence of SEQ ID NO:304.

In some embodiments, an anti-mutCALR antibody comprises a VH comprising the amino acid sequence of SEQ ID NO:182 and a VL comprising the amino acid sequence of SEQ ID NO:305.

In some embodiments, an anti-mutCALR antibody comprises a VH comprising the amino acid sequence of SEQ ID NO:183 and a VL comprising the amino acid sequence of SEQ ID NO:306.

In some embodiments, an anti-mutCALR antibody comprises a VH comprising the amino acid sequence of SEQ ID NO:182 and a VL comprising the amino acid sequence of SEQ ID NO:307.

In some embodiments, an anti-mutCALR antibody comprises a VH comprising the amino acid sequence of SEQ ID NO:182 and a VL comprising the amino acid sequence of SEQ ID NO:308.

In some embodiments, an anti-mutCALR antibody comprises a VH comprising the amino acid sequence of SEQ ID NO:182 and a VL comprising the amino acid sequence of SEQ ID NO:309.

In some embodiments, an anti-mutCALR antibody comprises a VH comprising the amino acid sequence of SEQ ID NO:184 and a VL comprising the amino acid sequence of SEQ ID NO:310.

In some embodiments, an anti-mutCALR antibody comprises a VH comprising the amino acid sequence of SEQ ID NO:182 and a VL comprising the amino acid sequence of SEQ ID NO:311.

In some embodiments, an anti-mutCALR antibody comprises a VH comprising the amino acid sequence of SEQ ID NO:185 and a VL comprising the amino acid sequence of SEQ ID NO:312.

In some embodiments, an anti-mutCALR antibody comprises a VH comprising the amino acid sequence of SEQ ID NO:186 and a VL comprising the amino acid sequence of SEQ ID NO:313.

In some embodiments, an anti-mutCALR antibody comprises a VH comprising the amino acid sequence of SEQ ID NO:187 and a VL comprising the amino acid sequence of SEQ ID NO:314.

In some embodiments, an anti-mutCALR antibody comprises a VH comprising the amino acid sequence of SEQ ID NO:188 and a VL comprising the amino acid sequence of SEQ ID NO:268.

In some embodiments, an anti-mutCALR antibody comprises a VH comprising the amino acid sequence of SEQ ID NO:189 and a VL comprising the amino acid sequence of SEQ ID NO:268.

In some embodiments, an anti-mutCALR antibody comprises a VH comprising the amino acid sequence of SEQ ID NO:190 and a VL comprising the amino acid sequence of SEQ ID NO:268.

In some embodiments, an anti-mutCALR antibody comprises a VH comprising the amino acid sequence of SEQ ID NO:191 and a VL comprising the amino acid sequence of SEQ ID NO:268.

In some embodiments, an anti-mutCALR antibody comprises a VH comprising the amino acid sequence of SEQ ID NO:192 and a VL comprising the amino acid sequence of SEQ ID NO:268.

In some embodiments, an anti-mutCALR antibody comprises a VH comprising the amino acid sequence of SEQ ID NO:193 and a VL comprising the amino acid sequence of SEQ ID NO:268.

In some embodiments, an anti-mutCALR antibody comprises a VH comprising the amino acid sequence of SEQ ID NO:194 and a VL comprising the amino acid sequence of SEQ ID NO:268.

In some embodiments, an anti-mutCALR antibody comprises a VH comprising the amino acid sequence of SEQ ID NO:195 and a VL comprising the amino acid sequence of SEQ ID NO:268.

In some embodiments, an anti-mutCALR antibody comprises a VH comprising the amino acid sequence of SEQ ID NO:196 and a VL comprising the amino acid sequence of SEQ ID NO:268.

In some embodiments, an anti-mutCALR antibody comprises a VH comprising the amino acid sequence of SEQ ID NO:165 and a VL comprising the amino acid sequence of SEQ ID NO:315.

In some embodiments, an anti-mutCALR antibody comprises a VH comprising the amino acid sequence of SEQ ID NO:188 and a VL comprising the amino acid sequence of SEQ ID NO:315.

In some embodiments, an anti-mutCALR antibody comprises a VH comprising the amino acid sequence of SEQ ID NO:189 and a VL comprising the amino acid sequence of SEQ ID NO:315.

In some embodiments, an anti-mutCALR antibody comprises a VH comprising the amino acid sequence of SEQ ID NO:190 and a VL comprising the amino acid sequence of SEQ ID NO:315.

In some embodiments, an anti-mutCALR antibody comprises a VH comprising the amino acid sequence of SEQ ID NO:191 and a VL comprising the amino acid sequence of SEQ ID NO:315.

In some embodiments, an anti-mutCALR antibody comprises a VH comprising the amino acid sequence of SEQ ID NO:192 and a VL comprising the amino acid sequence of SEQ ID NO:315.

In some embodiments, an anti-mutCALR antibody comprises a VH comprising the amino acid sequence of SEQ ID NO:193 and a VL comprising the amino acid sequence of SEQ ID NO:315.

In some embodiments, an anti-mutCALR antibody comprises a VH comprising the amino acid sequence of SEQ ID NO:194 and a VL comprising the amino acid sequence of SEQ ID NO:315.

In some embodiments, an anti-mutCALR antibody comprises a VH comprising the amino acid sequence of SEQ ID NO:195 and a VL comprising the amino acid sequence of SEQ ID NO:315.

In some embodiments, an anti-mutCALR antibody comprises a VH comprising the amino acid sequence of SEQ ID NO:196 and a VL comprising the amino acid sequence of SEQ ID NO:315.

In some embodiments, an anti-mutCALR antibody comprises a VH comprising the amino acid sequence of SEQ ID NO:165 and a VL comprising the amino acid sequence of SEQ ID NO:316.

In some embodiments, an anti-mutCALR antibody comprises a VH comprising the amino acid sequence of SEQ ID NO:188 and a VL comprising the amino acid sequence of SEQ ID NO:316.

In some embodiments, an anti-mutCALR antibody comprises a VH comprising the amino acid sequence of SEQ ID NO:189 and a VL comprising the amino acid sequence of SEQ ID NO:316.

In some embodiments, an anti-mutCALR antibody comprises a VH comprising the amino acid sequence of SEQ ID NO:190 and a VL comprising the amino acid sequence of SEQ ID NO:316.

In some embodiments, an anti-mutCALR antibody comprises a VH comprising the amino acid sequence of SEQ ID NO:191 and a VL comprising the amino acid sequence of SEQ ID NO:316.

In some embodiments, an anti-mutCALR antibody comprises a VH comprising the amino acid sequence of SEQ ID NO:192 and a VL comprising the amino acid sequence of SEQ ID NO:316.

In some embodiments, an anti-mutCALR antibody comprises a VH comprising the amino acid sequence of SEQ ID NO:193 and a VL comprising the amino acid sequence of SEQ ID NO:316.

In some embodiments, an anti-mutCALR antibody comprises a VH comprising the amino acid sequence of SEQ ID NO:194 and a VL comprising the amino acid sequence of SEQ ID NO:316.

In some embodiments, an anti-mutCALR antibody comprises a VH comprising the amino acid sequence of SEQ ID NO:195 and a VL comprising the amino acid sequence of SEQ ID NO:316.

In some embodiments, an anti-mutCALR antibody comprises a VH comprising the amino acid sequence of SEQ ID NO:196 and a VL comprising the amino acid sequence of SEQ ID NO:316.

In some embodiments, an anti-mutCALR antibody comprises a VH comprising the amino acid sequence of SEQ ID NO:197 and a VL comprising the amino acid sequence of SEQ ID NO:268.

In some embodiments, an anti-mutCALR antibody comprises a VH comprising the amino acid sequence of SEQ ID NO:198 and a VL comprising the amino acid sequence of SEQ ID NO:268.

In some embodiments, an anti-mutCALR antibody comprises a VH comprising the amino acid sequence of SEQ ID NO:199 and a VL comprising the amino acid sequence of SEQ ID NO:268.

In some embodiments, an anti-mutCALR antibody comprises a VH comprising the amino acid sequence of SEQ ID NO:200 and a VL comprising the amino acid sequence of SEQ ID NO:268.

In some embodiments, an anti-mutCALR antibody comprises a VH comprising the amino acid sequence of SEQ ID NO:201 and a VL comprising the amino acid sequence of SEQ ID NO:268.

In some embodiments, an anti-mutCALR antibody comprises a VH comprising the amino acid sequence of SEQ ID NO:197 and a VL comprising the amino acid sequence of SEQ ID NO:315.

In some embodiments, an anti-mutCALR antibody comprises a VH comprising the amino acid sequence of SEQ ID NO:198 and a VL comprising the amino acid sequence of SEQ ID NO:315.

In some embodiments, an anti-mutCALR antibody comprises a VH comprising the amino acid sequence of SEQ ID NO:199 and a VL comprising the amino acid sequence of SEQ ID NO:315.

In some embodiments, an anti-mutCALR antibody comprises a VH comprising the amino acid sequence of SEQ ID NO:200 and a VL comprising the amino acid sequence of SEQ ID NO:315.

In some embodiments, an anti-mutCALR antibody comprises a VH comprising the amino acid sequence of SEQ ID NO:201 and a VL comprising the amino acid sequence of SEQ ID NO:315.

In some embodiments, an anti-mutCALR antibody comprises a VH comprising the amino acid sequence of SEQ ID NO:197 and a VL comprising the amino acid sequence of SEQ ID NO:316.

In some embodiments, an anti-mutCALR antibody comprises a VH comprising the amino acid sequence of SEQ ID NO:198 and a VL comprising the amino acid sequence of SEQ ID NO:316.

In some embodiments, an anti-mutCALR antibody comprises a VH comprising the amino acid sequence of SEQ ID NO:199 and a VL comprising the amino acid sequence of SEQ ID NO:316.

In some embodiments, an anti-mutCALR antibody comprises a VH comprising the amino acid sequence of SEQ ID NO:200 and a VL comprising the amino acid sequence of SEQ ID NO:316.

In some embodiments, an anti-mutCALR antibody comprises a VH comprising the amino acid sequence of SEQ ID NO:201 and a VL comprising the amino acid sequence of SEQ ID NO:316.

In some embodiments, an anti-mutCALR antibody comprises a VH comprising the amino acid sequence of SEQ ID NO:170 and a VL comprising the amino acid sequence of SEQ ID NO:268.

In some embodiments, an anti-mutCALR antibody comprises a VH comprising the amino acid sequence of SEQ ID NO:202 and a VL comprising the amino acid sequence of SEQ ID NO:268.

In some embodiments, an anti-mutCALR antibody comprises a VH comprising the amino acid sequence of SEQ ID NO:203 and a VL comprising the amino acid sequence of SEQ ID NO:268.

In some embodiments, an anti-mutCALR antibody comprises a VH comprising the amino acid sequence of SEQ ID NO:204 and a VL comprising the amino acid sequence of SEQ ID NO:268.

In some embodiments, an anti-mutCALR antibody comprises a VH comprising the amino acid sequence of SEQ ID NO:205 and a VL comprising the amino acid sequence of SEQ ID NO:268.

In some embodiments, an anti-mutCALR antibody comprises a VH comprising the amino acid sequence of SEQ ID NO:206 and a VL comprising the amino acid sequence of SEQ ID NO:268.

In some embodiments, an anti-mutCALR antibody comprises a VH comprising the amino acid sequence of SEQ ID NO:207 and a VL comprising the amino acid sequence of SEQ ID NO:268.

In some embodiments, an anti-mutCALR antibody comprises a VH comprising the amino acid sequence of SEQ ID NO:208 and a VL comprising the amino acid sequence of SEQ ID NO:268.

In some embodiments, an anti-mutCALR antibody comprises a VH comprising the amino acid sequence of SEQ ID NO:170 and a VL comprising the amino acid sequence of SEQ ID NO:315.

In some embodiments, an anti-mutCALR antibody comprises a VH comprising the amino acid sequence of SEQ ID NO:202 and a VL comprising the amino acid sequence of SEQ ID NO:315.

In some embodiments, an anti-mutCALR antibody comprises a VH comprising the amino acid sequence of SEQ ID NO:203 and a VL comprising the amino acid sequence of SEQ ID NO:315.

In some embodiments, an anti-mutCALR antibody comprises a VH comprising the amino acid sequence of SEQ ID NO:204 and a VL comprising the amino acid sequence of SEQ ID NO:315.

In some embodiments, an anti-mutCALR antibody comprises a VH comprising the amino acid sequence of SEQ ID NO:205 and a VL comprising the amino acid sequence of SEQ ID NO:315.

In some embodiments, an anti-mutCALR antibody comprises a VH comprising the amino acid sequence of SEQ ID NO:206 and a VL comprising the amino acid sequence of SEQ ID NO:315.

In some embodiments, an anti-mutCALR antibody comprises a VH comprising the amino acid sequence of SEQ ID NO:207 and a VL comprising the amino acid sequence of SEQ ID NO:315.

In some embodiments, an anti-mutCALR antibody comprises a VH comprising the amino acid sequence of SEQ ID NO:208 and a VL comprising the amino acid sequence of SEQ ID NO:315.

In some embodiments, an anti-mutCALR antibody comprises a VH comprising the amino acid sequence of SEQ ID NO:170 and a VL comprising the amino acid sequence of SEQ ID NO:316.

In some embodiments, an anti-mutCALR antibody comprises a VH comprising the amino acid sequence of SEQ ID NO:202 and a VL comprising the amino acid sequence of SEQ ID NO:316.

In some embodiments, an anti-mutCALR antibody comprises a VH comprising the amino acid sequence of SEQ ID NO:203 and a VL comprising the amino acid sequence of SEQ ID NO:316.

In some embodiments, an anti-mutCALR antibody comprises a VH comprising the amino acid sequence of SEQ ID NO:204 and a VL comprising the amino acid sequence of SEQ ID NO:316.

In some embodiments, an anti-mutCALR antibody comprises a VH comprising the amino acid sequence of SEQ ID NO:205 and a VL comprising the amino acid sequence of SEQ ID NO:316.

In some embodiments, an anti-mutCALR antibody comprises a VH comprising the amino acid sequence of SEQ ID NO:206 and a VL comprising the amino acid sequence of SEQ ID NO:316.

In some embodiments, an anti-mutCALR antibody comprises a VH comprising the amino acid sequence of SEQ ID NO:207 and a VL comprising the amino acid sequence of SEQ ID NO:316.

In some embodiments, an anti-mutCALR antibody comprises a VH comprising the amino acid sequence of SEQ ID NO:208 and a VL comprising the amino acid sequence of SEQ ID NO:316.

In some embodiments, an anti-mutCALR antibody comprises a VH comprising the amino acid sequence of SEQ ID NO:188 and a VL comprising the amino acid sequence of SEQ ID NO:277.

In some embodiments, an anti-mutCALR antibody comprises a VH comprising the amino acid sequence of SEQ ID NO:189 and a VL comprising the amino acid sequence of SEQ ID NO:277.

In some embodiments, an anti-mutCALR antibody comprises a VH comprising the amino acid sequence of SEQ ID NO:190 and a VL comprising the amino acid sequence of SEQ ID NO:277.

In some embodiments, an anti-mutCALR antibody comprises a VH comprising the amino acid sequence of SEQ ID NO:191 and a VL comprising the amino acid sequence of SEQ ID NO:277.

In some embodiments, an anti-mutCALR antibody comprises a VH comprising the amino acid sequence of SEQ ID NO:192 and a VL comprising the amino acid sequence of SEQ ID NO:277.

In some embodiments, an anti-mutCALR antibody comprises a VH comprising the amino acid sequence of SEQ ID NO:193 and a VL comprising the amino acid sequence of SEQ ID NO:277.

In some embodiments, an anti-mutCALR antibody comprises a VH comprising the amino acid sequence of SEQ ID NO:194 and a VL comprising the amino acid sequence of SEQ ID NO:277.

In some embodiments, an anti-mutCALR antibody comprises a VH comprising the amino acid sequence of SEQ ID NO:195 and a VL comprising the amino acid sequence of SEQ ID NO:277.

In some embodiments, an anti-mutCALR antibody comprises a VH comprising the amino acid sequence of SEQ ID NO:196 and a VL comprising the amino acid sequence of SEQ ID NO:277.

In some embodiments, an anti-mutCALR antibody comprises a VH comprising the amino acid sequence of SEQ ID NO:165 and a VL comprising the amino acid sequence of SEQ ID NO:317.

In some embodiments, an anti-mutCALR antibody comprises a VH comprising the amino acid sequence of SEQ ID NO:188 and a VL comprising the amino acid sequence of SEQ ID NO:317.

In some embodiments, an anti-mutCALR antibody comprises a VH comprising the amino acid sequence of SEQ ID NO:189 and a VL comprising the amino acid sequence of SEQ ID NO:317.

In some embodiments, an anti-mutCALR antibody comprises a VH comprising the amino acid sequence of SEQ ID NO:190 and a VL comprising the amino acid sequence of SEQ ID NO:317.

In some embodiments, an anti-mutCALR antibody comprises a VH comprising the amino acid sequence of SEQ ID NO:191 and a VL comprising the amino acid sequence of SEQ ID NO:317.

In some embodiments, an anti-mutCALR antibody comprises a VH comprising the amino acid sequence of SEQ ID NO:192 and a VL comprising the amino acid sequence of SEQ ID NO:317.

In some embodiments, an anti-mutCALR antibody comprises a VH comprising the amino acid sequence of SEQ ID NO:193 and a VL comprising the amino acid sequence of SEQ ID NO:317.

In some embodiments, an anti-mutCALR antibody comprises a VH comprising the amino acid sequence of SEQ ID NO:194 and a VL comprising the amino acid sequence of SEQ ID NO:317.

In some embodiments, an anti-mutCALR antibody comprises a VH comprising the amino acid sequence of SEQ ID NO:195 and a VL comprising the amino acid sequence of SEQ ID NO:317.

In some embodiments, an anti-mutCALR antibody comprises a VH comprising the amino acid sequence of SEQ ID NO:196 and a VL comprising the amino acid sequence of SEQ ID NO:317.

In some embodiments, an anti-mutCALR antibody comprises a VH comprising the amino acid sequence of SEQ ID NO:197 and a VL comprising the amino acid sequence of SEQ ID NO:277.

In some embodiments, an anti-mutCALR antibody comprises a VH comprising the amino acid sequence of SEQ ID NO:198 and a VL comprising the amino acid sequence of SEQ ID NO:277.

In some embodiments, an anti-mutCALR antibody comprises a VH comprising the amino acid sequence of SEQ ID NO:199 and a VL comprising the amino acid sequence of SEQ ID NO:277.

In some embodiments, an anti-mutCALR antibody comprises a VH comprising the amino acid sequence of SEQ ID NO:200 and a VL comprising the amino acid sequence of SEQ ID NO:277.

In some embodiments, an anti-mutCALR antibody comprises a VH comprising the amino acid sequence of SEQ ID NO:201 and a VL comprising the amino acid sequence of SEQ ID NO:277.

In some embodiments, an anti-mutCALR antibody comprises a VH comprising the amino acid sequence of SEQ ID NO:197 and a VL comprising the amino acid sequence of SEQ ID NO:317.

In some embodiments, an anti-mutCALR antibody comprises a VH comprising the amino acid sequence of SEQ ID NO:198 and a VL comprising the amino acid sequence of SEQ ID NO:317.

In some embodiments, an anti-mutCALR antibody comprises a VH comprising the amino acid sequence of SEQ ID NO:199 and a VL comprising the amino acid sequence of SEQ ID NO:317.

In some embodiments, an anti-mutCALR antibody comprises a VH comprising the amino acid sequence of SEQ ID NO:200 and a VL comprising the amino acid sequence of SEQ ID NO:317.

In some embodiments, an anti-mutCALR antibody comprises a VH comprising the amino acid sequence of SEQ ID NO:201 and a VL comprising the amino acid sequence of SEQ ID NO:317.

In some embodiments, an anti-mutCALR antibody comprises a VH comprising the amino acid sequence of SEQ ID NO:170 and a VL comprising the amino acid sequence of SEQ ID NO:277.

In some embodiments, an anti-mutCALR antibody comprises a VH comprising the amino acid sequence of SEQ ID NO:202 and a VL comprising the amino acid sequence of SEQ ID NO:277.

In some embodiments, an anti-mutCALR antibody comprises a VH comprising the amino acid sequence of SEQ ID NO:203 and a VL comprising the amino acid sequence of SEQ ID NO:277.

In some embodiments, an anti-mutCALR antibody comprises a VH comprising the amino acid sequence of SEQ ID NO:204 and a VL comprising the amino acid sequence of SEQ ID NO:277.

In some embodiments, an anti-mutCALR antibody comprises a VH comprising the amino acid sequence of SEQ ID NO:205 and a VL comprising the amino acid sequence of SEQ ID NO:277.

In some embodiments, an anti-mutCALR antibody comprises a VH comprising the amino acid sequence of SEQ ID NO:206 and a VL comprising the amino acid sequence of SEQ ID NO:277.

In some embodiments, an anti-mutCALR antibody comprises a VH comprising the amino acid sequence of SEQ ID NO:207 and a VL comprising the amino acid sequence of SEQ ID NO:277.

In some embodiments, an anti-mutCALR antibody comprises a VH comprising the amino acid sequence of SEQ ID NO:208 and a VL comprising the amino acid sequence of SEQ ID NO:277.

In some embodiments, an anti-mutCALR antibody comprises a VH comprising the amino acid sequence of SEQ ID NO:170 and a VL comprising the amino acid sequence of SEQ ID NO:317.

In some embodiments, an anti-mutCALR antibody comprises a VH comprising the amino acid sequence of SEQ ID NO:202 and a VL comprising the amino acid sequence of SEQ ID NO:317.

In some embodiments, an anti-mutCALR antibody comprises a VH comprising the amino acid sequence of SEQ ID NO:203 and a VL comprising the amino acid sequence of SEQ ID NO:317.

In some embodiments, an anti-mutCALR antibody comprises a VH comprising the amino acid sequence of SEQ ID NO:204 and a VL comprising the amino acid sequence of SEQ ID NO:317.

In some embodiments, an anti-mutCALR antibody comprises a VH comprising the amino acid sequence of SEQ ID NO:205 and a VL comprising the amino acid sequence of SEQ ID NO:317.

In some embodiments, an anti-mutCALR antibody comprises a VH comprising the amino acid sequence of SEQ ID NO:206 and a VL comprising the amino acid sequence of SEQ ID NO:317.

In some embodiments, an anti-mutCALR antibody comprises a VH comprising the amino acid sequence of SEQ ID NO:207 and a VL comprising the amino acid sequence of SEQ ID NO:317.

In some embodiments, an anti-mutCALR antibody comprises a VH comprising the amino acid sequence of SEQ ID NO:208 and a VL comprising the amino acid sequence of SEQ ID NO:317.

In some embodiments, an anti-mutCALR antibody comprises a VH comprising the amino acid sequence of SEQ ID NO:188 and a VL comprising the amino acid sequence of SEQ ID NO:279.

In some embodiments, an anti-mutCALR antibody comprises a VH comprising the amino acid sequence of SEQ ID NO:189 and a VL comprising the amino acid sequence of SEQ ID NO:279.

In some embodiments, an anti-mutCALR antibody comprises a VH comprising the amino acid sequence of SEQ ID NO:190 and a VL comprising the amino acid sequence of SEQ ID NO:279.

In some embodiments, an anti-mutCALR antibody comprises a VH comprising the amino acid sequence of SEQ ID NO:191 and a VL comprising the amino acid sequence of SEQ ID NO:279.

In some embodiments, an anti-mutCALR antibody comprises a VH comprising the amino acid sequence of SEQ ID NO:192 and a VL comprising the amino acid sequence of SEQ ID NO:279.

In some embodiments, an anti-mutCALR antibody comprises a VH comprising the amino acid sequence of SEQ ID NO:193 and a VL comprising the amino acid sequence of SEQ ID NO:279.

In some embodiments, an anti-mutCALR antibody comprises a VH comprising the amino acid sequence of SEQ ID NO:194 and a VL comprising the amino acid sequence of SEQ ID NO:279.

In some embodiments, an anti-mutCALR antibody comprises a VH comprising the amino acid sequence of SEQ ID NO:195 and a VL comprising the amino acid sequence of SEQ ID NO:279.

In some embodiments, an anti-mutCALR antibody comprises a VH comprising the amino acid sequence of SEQ ID NO:196 and a VL comprising the amino acid sequence of SEQ ID NO:279.

In some embodiments, an anti-mutCALR antibody comprises a VH comprising the amino acid sequence of SEQ ID NO:165 and a VL comprising the amino acid sequence of SEQ ID NO:318.

In some embodiments, an anti-mutCALR antibody comprises a VH comprising the amino acid sequence of SEQ ID NO:188 and a VL comprising the amino acid sequence of SEQ ID NO:318.

In some embodiments, an anti-mutCALR antibody comprises a VH comprising the amino acid sequence of SEQ ID NO:189 and a VL comprising the amino acid sequence of SEQ ID NO:318.

In some embodiments, an anti-mutCALR antibody comprises a VH comprising the amino acid sequence of SEQ ID NO:190 and a VL comprising the amino acid sequence of SEQ ID NO:318.

In some embodiments, an anti-mutCALR antibody comprises a VH comprising the amino acid sequence of SEQ ID NO:191 and a VL comprising the amino acid sequence of SEQ ID NO:318.

In some embodiments, an anti-mutCALR antibody comprises a VH comprising the amino acid sequence of SEQ ID NO:192 and a VL comprising the amino acid sequence of SEQ ID NO:318.

In some embodiments, an anti-mutCALR antibody comprises a VH comprising the amino acid sequence of SEQ ID NO:193 and a VL comprising the amino acid sequence of SEQ ID NO:318.

In some embodiments, an anti-mutCALR antibody comprises a VH comprising the amino acid sequence of SEQ ID NO:194 and a VL comprising the amino acid sequence of SEQ ID NO:318.

In some embodiments, an anti-mutCALR antibody comprises a VH comprising the amino acid sequence of SEQ ID NO:195 and a VL comprising the amino acid sequence of SEQ ID NO:318.

In some embodiments, an anti-mutCALR antibody comprises a VH comprising the amino acid sequence of SEQ ID NO:196 and a VL comprising the amino acid sequence of SEQ ID NO:318.

In some embodiments, an anti-mutCALR antibody comprises a VH comprising the amino acid sequence of SEQ ID NO:197 and a VL comprising the amino acid sequence of SEQ ID NO:279.

In some embodiments, an anti-mutCALR antibody comprises a VH comprising the amino acid sequence of SEQ ID NO:198 and a VL comprising the amino acid sequence of SEQ ID NO:279.

In some embodiments, an anti-mutCALR antibody comprises a VH comprising the amino acid sequence of SEQ ID NO:199 and a VL comprising the amino acid sequence of SEQ ID NO:279.

In some embodiments, an anti-mutCALR antibody comprises a VH comprising the amino acid sequence of SEQ ID NO:200 and a VL comprising the amino acid sequence of SEQ ID NO:279.

In some embodiments, an anti-mutCALR antibody comprises a VH comprising the amino acid sequence of SEQ ID NO:201 and a VL comprising the amino acid sequence of SEQ ID NO:279.

In some embodiments, an anti-mutCALR antibody comprises a VH comprising the amino acid sequence of SEQ ID NO:197 and a VL comprising the amino acid sequence of SEQ ID NO:318.

In some embodiments, an anti-mutCALR antibody comprises a VH comprising the amino acid sequence of SEQ ID NO:198 and a VL comprising the amino acid sequence of SEQ ID NO:318.

In some embodiments, an anti-mutCALR antibody comprises a VH comprising the amino acid sequence of SEQ ID NO:199 and a VL comprising the amino acid sequence of SEQ ID NO:318.

In some embodiments, an anti-mutCALR antibody comprises a VH comprising the amino acid sequence of SEQ ID NO:200 and a VL comprising the amino acid sequence of SEQ ID NO:318.

In some embodiments, an anti-mutCALR antibody comprises a VH comprising the amino acid sequence of SEQ ID NO:201 and a VL comprising the amino acid sequence of SEQ ID NO:318.

In some embodiments, an anti-mutCALR antibody comprises a VH comprising the amino acid sequence of SEQ ID NO:170 and a VL comprising the amino acid sequence of SEQ ID NO:279.

In some embodiments, an anti-mutCALR antibody comprises a VH comprising the amino acid sequence of SEQ ID NO:202 and a VL comprising the amino acid sequence of SEQ ID NO:279.

In some embodiments, an anti-mutCALR antibody comprises a VH comprising the amino acid sequence of SEQ ID NO:203 and a VL comprising the amino acid sequence of SEQ ID NO:279.

In some embodiments, an anti-mutCALR antibody comprises a VH comprising the amino acid sequence of SEQ ID NO:204 and a VL comprising the amino acid sequence of SEQ ID NO:279.

In some embodiments, an anti-mutCALR antibody comprises a VH comprising the amino acid sequence of SEQ ID NO:205 and a VL comprising the amino acid sequence of SEQ ID NO:279.

In some embodiments, an anti-mutCALR antibody comprises a VH comprising the amino acid sequence of SEQ ID NO:206 and a VL comprising the amino acid sequence of SEQ ID NO:279.

In some embodiments, an anti-mutCALR antibody comprises a VH comprising the amino acid sequence of SEQ ID NO:207 and a VL comprising the amino acid sequence of SEQ ID NO:279.

In some embodiments, an anti-mutCALR antibody comprises a VH comprising the amino acid sequence of SEQ ID NO:208 and a VL comprising the amino acid sequence of SEQ ID NO:279.

In some embodiments, an anti-mutCALR antibody comprises a VH comprising the amino acid sequence of SEQ ID NO:170 and a VL comprising the amino acid sequence of SEQ ID NO:318.

In some embodiments, an anti-mutCALR antibody comprises a VH comprising the amino acid sequence of SEQ ID NO:202 and a VL comprising the amino acid sequence of SEQ ID NO:318.

In some embodiments, an anti-mutCALR antibody comprises a VH comprising the amino acid sequence of SEQ ID NO:203 and a VL comprising the amino acid sequence of SEQ ID NO:318.

In some embodiments, an anti-mutCALR antibody comprises a VH comprising the amino acid sequence of SEQ ID NO:204 and a VL comprising the amino acid sequence of SEQ ID NO:318.

In some embodiments, an anti-mutCALR antibody comprises a VH comprising the amino acid sequence of SEQ ID NO:205 and a VL comprising the amino acid sequence of SEQ ID NO:318.

In some embodiments, an anti-mutCALR antibody comprises a VH comprising the amino acid sequence of SEQ ID NO:206 and a VL comprising the amino acid sequence of SEQ ID NO:318.

In some embodiments, an anti-mutCALR antibody comprises a VH comprising the amino acid sequence of SEQ ID NO:207 and a VL comprising the amino acid sequence of SEQ ID NO:318.

In some embodiments, an anti-mutCALR antibody comprises a VH comprising the amino acid sequence of SEQ ID NO:208 and a VL comprising the amino acid sequence of SEQ ID NO:318.

In some embodiments, an anti-mutCALR antibody comprises a modification which modulates (e.g., reduces or increases) the Fc region-mediated effector function, such as complement-dependent cytotoxicity (CDC), antibody-dependent cellular cytotoxicity (ADCC) and antibody-dependent cell phagocytosis (ADCP). Depending on the therapeutic antibody or Fc fusion protein application, it may be desired to either reduce or increase the effector function.

In certain embodiments, an anti-mutCALR antibody has Fc effector function. In certain embodiments, an anti-mutCALR antibody has enhanced Fc effector function. In certain embodiments, an anti-mutCALR antibody exhibits antibody-dependent cell-mediated cytotoxicity (ADCC). An anti-mutCALR antibody can be engineered to enhance the ADCC activity (for review, see Kubota T el al. Cancer Sci. 2009; 100(9):1566-72). For example, ADCC activity of an antibody can be improved when the antibody itself has a low ADCC activity, by slightly modifying the constant region of the antibody (Junttila T T. et al. Cancer Res. 2010; 70(11):4481-9). Changes are sometimes also made to improve storage or production or to remove C-terminal lysines (Kubota T el al. Cancer Sci. 2009; 100(9):1566-72). Another suitable method to improve ADCC activity of an antibody is by enzymatically interfering with the glycosylation pathway resulting in a reduced fucose (von Horsten H H. el al. Glycobiology. 2010; 20(12):1607-18). Alternatively, or additionally, other suitable methods can be used to achieve ADCC enhancement, for instance including glycoengineering (Kyowa Hakko/Biowa, GlycArt (Roche) and Eureka Therapeutics) and mutagenesis, all of which seek to improve Fc binding to low-affinity activating FcγRIIIa, and/or to reduce binding to the low affinity inhibitory FcγRIIb. In certain embodiments, a binding moiety of the present disclosure exhibits enhanced antibody-dependent cell-mediated cytotoxicity (ADCC). In certain embodiments, a binding moiety of the present disclosure is afucosylated.

In certain embodiments, an anti-mutCALR antibody has reduced Fc effector function. In certain embodiments, an anti-mutCALR antibody exhibits reduced or substantially no complement-dependent cytotoxicity (CDC), antibody-dependent cellular cytotoxicity (ADCC) or antibody-dependent cell phagocytosis (ADCP). In certain embodiments, an anti-mutCALR antibody exhibits reduced or substantially no antibody-dependent cell-mediated cytotoxicity (ADCC). An anti-mutCALR antibody can be engineered to reduce effector function, for example ADCC activity, by any suitable method including removal of glycosylation sites in the Fc region. In certain embodiments, an anti-mutCALR antibody that has a reduced Fc effector function (e.g., a reduced ADCC effector function) comprises an N297A mutation on the heavy chain.

In some embodiments, an anti-mutCALR antibody is an IgG1 isotype (e.g., IgG1, IgG2, IgG3 or IgG4). In some embodiments, an anti-mutCALR antibody is an IgG1. In some embodiments, an IgG1, IgG2, IgG3 or IgG4 anti-mutCALR antibody has Fc-effector function. In some embodiments, an IgG1, IgG2, IgG3 or IgG4 anti-mutCALR antibody is Fc-effector function null. In some embodiments, an IgG1 anti-mutCALR antibody has Fc-effector function. In some embodiments, an IgG1 anti-mutCALR antibody is Fc-effector function null.

In some instances, the anti-mutCALR antibody is an antibody fragment. Fragments of the antibodies described herein (e.g., Fab, Fab′, F(ab′)₂, Facb, and Fv) may be prepared by proteolytic digestion of intact antibodies. For example, antibody fragments can be obtained by treating the whole antibody with an enzyme such as papain, pepsin, or plasmin or the FabRICATOR® (IdeS) recombinant enzyme (Genovis AB) that digests IgG antibodies to produce a homogeneous pool of F(ab′)2 and Fc/2 fragments. Papain digestion of whole antibodies produces F(ab)₂ or Fab fragments; pepsin digestion of whole antibodies yields F(ab′)₂ or Fab′; and plasmin digestion of whole antibodies yields Facb fragments.

Alternatively, antibody fragments can be produced recombinantly. For example, nucleic acids encoding the antibody fragments of interest can be constructed, introduced into an expression vector, and expressed in suitable host cells. See, e.g., Co, M. S. et al., J. Immunol., 152:2968-2976 (1994); Better, M. and Horwitz, A. H., Methods in Enzymology, 178:476-496 (1989); Plueckthun, A. and Skerra, A., Methods in Enzymology, 178:476-496 (1989); Lamoyi, E., Methods in Enzymology, 121:652-663 (1989); Rousseaux, J. et al., Methods in Enzymology, (1989) 121:663-669 (1989); and Bird, R. E. et al., TIBTECH, 9:132-137 (1991)). Antibody fragments can be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)₂ fragments (Carter et al., Bio/Technology, 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046.

In some instances, the anti-mutCALR antibody is a minibody. Minibodies of anti-mutCALR antibodies include diabodies, single chain (scFv), and single-chain (Fv)₂ (sc(Fv)₂).

A “diabody” is a bivalent minibody constructed by gene fusion (see, e.g., Holliger, P. et al., Proc. Natl. Acad. Sci. U.S.A, 90:6444-6448 (1993); EP 404,097; WO 93/11161). Diabodies are dimers composed of two polypeptide chains. The VL and VH domain of each polypeptide chain of the diabody are bound by linkers. The number of amino acid residues that constitute a linker can be between 2 to 12 residues (e.g., 3-10 residues or five or about five residues). The linkers of the polypeptides in a diabody are typically too short to allow the VL and VH to bind to each other. Thus, the VL and VH encoded in the same polypeptide chain cannot form a single-chain variable region fragment, but instead form a dimer with a different single-chain variable region fragment. As a result, a diabody has two antigen-binding sites.

An scFv is a single-chain polypeptide antibody obtained by linking the VH and VL with a linker (see, e.g., Huston et al., Proc. Natl. Acad. Sci. U.S.A, 85:5879-5883 (1988); and Plickthun, “The Pharmacology of Monoclonal Antibodies” Vol. 113, Ed Resenburg and Moore, Springer Verlag, New York, pp. 269-315, (1994)). The order of VHs and VLs to be linked is not particularly limited, and they may be arranged in any order. Examples of arrangements include: [VH] linker [VL]; or [VL] linker [VH]. The heavy chain variable domain and light chain variable domain in an scFv may be derived from any anti-mutCALR antibody described herein.

An sc(Fv)₂ is a minibody in which two VHs and two VLs are linked by a linker to form a single chain (Hudson, et al., J. Immunol. Methods, (1999) 231: 177-189 (1999)). An sc(Fv)₂ can be prepared, for example, by connecting scFvs with a linker. The sc(Fv)₂ of the present invention include antibodies preferably in which two VHs and two VLs are arranged in the order of: VH, VL, VH, and VL ([VH] linker [VL] linker [VH] linker [VL]), beginning from the N terminus of a single-chain polypeptide; however the order of the two VHs and two VLs is not limited to the above arrangement, and they may be arranged in any order.

In some instances, the anti-mutCALR antibody is a bispecific antibody. Bispecific antibodies are antibodies that have binding specificities for at least two different epitopes. Exemplary bispecific antibodies may bind to two different epitopes of the mutCALR protein. Other such antibodies may combine a mutCALR binding site with a binding site for another antigen. Bispecific antibodies can be prepared as full length antibodies or low molecular weight forms thereof (e.g., F(ab′)₂ bispecific antibodies, sc(Fv)₂ bispecific antibodies, diabody bispecific antibodies).

Traditional production of full length bispecific antibodies is based on the co-expression of two immunoglobulin heavy chain-light chain pairs, where the two chains have different specificities (Millstein et al., Nature, 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the CH3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites.

In certain embodiments, the bispecific anti-mutCALR antibody is a biparatopic antibody A biparatopic antibody is antibody which recognizes two non-identical epitopes (overlapping or non-overlapping epitopes) on the same target antigen (e.g., the C-terminal of mutCALR domain). A biparatopic anti-mutCALR antibody can comprise two immunoglobulin heavy chain-light chain pairs or one immunoglobulin heavy chain-light chain pair. In some embodiments, a biparatopic anti-mutCALR antibody comprises one immunoglobulin heavy-chain-light chain pair. In some embodiments, a biparatopic anti-mutCALR antibody is a full-length antibody comprising one immunoglobulin heavy-chain-light chain pair.

In some instances, the anti-mutCALR antibody is a multivalent antibody. A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X₁)ₙ-VD₂-(X₂)ₙ-Fc, wherein VD1 is a first variable domain, VD2 is a second variable domain, Fc is a polypeptide chain of an Fc region, X₁ and X₂ represent an amino acid or peptide spacer, and n is 0 or 1.

In some instances, the anti-mutCALR antibody is a conjugated antibody. The antibodies disclosed herein may be conjugated antibodies, which are bound to various molecules including macromolecular substances such as polymers (e.g., polyethylene glycol (PEG), polyethylenimine (PEI) modified with PEG (PEI-PEG), polyglutamic acid (PGA) (N-(2-Hydroxypropyl) methacrylamide (HPMA) copolymers), hyaluronic acid, radioactive materials (e.g., ⁹⁰Y, ¹³¹I), fluorescent substances, luminescent substances, haptens, enzymes, metal chelates, drugs, and toxins (e.g., calcheamicin, Pseudomonas exotoxin A, ricin (e.g., deglycosylated ricin A chain) and auristatins (e.g., auristatin E or auristatin F)).

In one embodiment, to improve the cytotoxic actions of anti-mutCALR antibodies and consequently their therapeutic effectiveness, the antibodies are conjugated with highly toxic substances, including radioisotopes and cytotoxic agents. These conjugates can deliver a toxic load selectively to the target site (i.e., cells expressing the antigen recognized by the antibody) while cells that are not recognized by the antibody are spared. In order to minimize toxicity, conjugates are generally engineered based on molecules with a short serum half-life (thus, the use of murine sequences, and IgG3 or IgG4 isotypes).

In certain embodiments, an anti-mutCALR antibody is modified with a moiety that improves its stabilization and/or retention in circulation, e.g., in blood, serum, or other tissues, e.g., by at least 1.5, 2, 5, 10, or 50 fold. For example, the anti-mutCALR antibody can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used. For example, the anti-mutCALR antibody can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

The above-described conjugated antibodies can be prepared by performing chemical modifications on the antibodies, respectively, or the lower molecular weight forms thereof described herein. Methods for modifying antibodies are well known in the art (see, e.g., U.S. Pat. Nos. 5,057,313 and 5,156,840).

### Methods of Producing Antibodies

Antibodies may be produced in bacterial or eukaryotic cells. Some antibodies, e.g., Fabs, can be produced in bacterial cells, e.g., E. coli cells. Antibodies can also be produced in eukaryotic cells such as transformed cell lines (e.g., CHO, 293E, COS). In addition, antibodies (e.g., scFvs) can be expressed in a yeast cell such as Pichia (see, e.g., Powers et al., J Immunol Methods. 251:123-35 (2001)), Hansenula, or Saccharomyces. To produce the antibody of interest, a polynucleotide encoding the antibody is constructed, introduced into an expression vector, and then expressed in suitable host cells. Standard molecular biology techniques are used to prepare the recombinant expression vector, transfect the host cells, select for transformants, culture the host cells and recover the antibody.

If the antibody is to be expressed in bacterial cells (e.g., E. coli), the expression vector should have characteristics that permit amplification of the vector in the bacterial cells. Additionally, when E. coli such as JM109, DH5α, HB101, or XL1-Blue is used as a host, the vector must have a promoter, for example, a lacZ promoter (Ward et al., 341:544-546 (1989), araB promoter (Better et al., Science, 240:1041-1043 (1988)), or T7 promoter that can allow efficient expression in E. coli. Examples of such vectors include, for example, M13-series vectors, pUC-series vectors, pBR322, pBluescript, pCR-Script, pGEX-5X-1 (Pharmacia), “QIAexpress system” (QIAGEN), pEGFP, and pET (when this expression vector is used, the host is preferably BL21 expressing T7 RNA polymerase). The expression vector may contain a signal sequence for antibody secretion. For production into the periplasm of E. coli, the pelB signal sequence (Lei et al., J. Bacteriol., 169:4379 (1987)) may be used as the signal sequence for antibody secretion. For bacterial expression, calcium chloride methods or electroporation methods may be used to introduce the expression vector into the bacterial cell.

If the antibody is to be expressed in animal cells such as CHO, COS, and NIH3T3 cells, the expression vector includes a promoter necessary for expression in these cells, for example, an SV40 promoter (Mulligan et al., Nature, 277:108 (1979)), MMLV-LTR promoter, EF1α promoter (Mizushima et al., Nucleic Acids Res., 18:5322 (1990)), or CMV promoter. In addition to the nucleic acid sequence encoding the immunoglobulin or domain thereof, the recombinant expression vectors may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced. Examples of vectors with selectable markers include pMAM, pDR2, pBK-RSV, pBK-CMV, pOPRSV, and pOP13.

In one embodiment, antibodies are produced in mammalian cells. Exemplary mammalian host cells for expressing an antibody include Chinese Hamster Ovary (CHO cells) (including dhfr-CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601 621), human embryonic kidney 293 cells (e.g., 293, 293E, 293T), COS cells, NIH3T3 cells, lymphocytic cell lines, e.g., NS0 myeloma cells and SP2 cells, and a cell from a transgenic animal, e.g., a transgenic mammal. For example, the cell is a mammary epithelial cell.

In an exemplary system for antibody expression, a recombinant expression vector encoding both the antibody heavy chain and the antibody light chain of an anti-mutCALR antibody is introduced into dhfr-CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

The antibodies of the present disclosure can be isolated from inside or outside (such as from the medium) of the host cell and purified as substantially pure and homogenous antibodies. Methods for isolation and purification commonly used for antibody purification may be used for the isolation and purification of antibodies, and are not limited to any particular method. Antibodies may be isolated and purified by appropriately selecting and combining, for example, column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, for example, affinity chromatography, ion exchange chromatography, hydrophobic chromatography, gel filtration, reverse-phase chromatography, and adsorption chromatography (Strategies for Protein Purification and Characterization: A Laboratory Course Manual. Ed Daniel R. Marshak et al., Cold Spring Harbor Laboratory Press, 1996). Chromatography can be carried out using liquid phase chromatography such as HPLC and FPLC. Columns used for affinity chromatography include protein A column and protein G column. Examples of columns using protein A column include Hyper D, POROS, and Sepharose FF (GE Healthcare Biosciences). The present disclosure also includes antibodies that are highly purified using these purification methods.

### Polynucleotides, Expression Vectors, and Cells

The disclosure also provides polynucleotides and vectors encoding an anti-mutCALR antibody or portion thereof (e.g., VH, VL, HC, or LC) described herein. The polynucleotides of the disclosure can be in the form of RNA or in the form of DNA. In some instances, the polynucleotide is DNA. In some instances, the polynucleotide is complementary DNA (cDNA). In some instances, the polynucleotide is RNA. In some embodiments, a polynucleotide described herein is isolated. In some embodiments, a polynucleotide described herein is purified.

In some instances, the polynucleotide encodes a VH comprising the VH CDR1, VH CDR2, and VH CDR3 of any antibody described herein (see, e.g., Tables 1-5). In some instances, the polynucleotide encodes a VL comprising the VL CDR1, VL CDR2, and VL CDR3 of any antibody described herein (see, e.g., Tables 1-5). In some instances, the polynucleotide encodes a heavy chain comprising a VH comprising the VH CDR1, VH CDR2, and VH CDR3 of any antibody described herein (see, e.g., Tables 1-5). In some instances, the polynucleotide encodes a light chain comprising a VL comprising the VL CDR1, VL CDR2, and VL CDR3 of any antibody described herein (see, e.g., Tables 1-5). In some instances, the polynucleotide is operably linked to a promoter.

In some instances, the polynucleotide comprises: (i) a first nucleic acid sequence encoding a first polypeptide, wherein the first polypeptide comprises a VH comprising the VH CDR1, VH CDR2, and VH CDR3 of any antibody described herein (see, e.g., Tables 1-5); and (ii) a second nucleic acid sequence encoding a second polypeptide, wherein the second polypeptide comprises a VL comprising the VL CDR1, VL CDR2, and VL CDR3 of any antibody described herein (see, e.g., Tables 1-5). In some instances, the polynucleotide comprises: (i) a first nucleic acid sequence encoding a first polypeptide, wherein the first polypeptide comprises a heavy chain comprising a VH comprising the VH CDR1, VH CDR2, and VH CDR3 of any antibody described herein (see, e.g., Tables 1-5); and (ii) a second nucleic acid sequence encoding a second polypeptide, wherein the second polypeptide comprises a light chain comprising a VL comprising the VL CDR1, VL CDR2, and VL CDR3 of any antibody described herein (see, e.g., Tables 1-5). In some instances, the first nucleic acid is operably linked to a first promoter and the second nucleic acid is operably linked to a second promoter.

In some instances, the polynucleotide encodes a VH described herein (see, e.g., Tables 4-5) or a variant thereof. In some instances, the polynucleotide encodes a polypeptide comprising an amino acid sequence having at least 80%, at least 85%, at least 90%, at least 95%, at least 97%, or 100% identity to the amino acid sequence set forth in any one of SEQ ID NOs:165-208. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:165-208. In some instances, the polynucleotide encodes a polypeptide comprising the amino acid sequence set forth in any one of SEQ ID NOs:165-208. In some instances, the polynucleotide is operably linked to a promoter.

In some instances, the polynucleotide encodes a VL described herein (see, e.g., Tables 4-5) or a variant thereof. In some instances, the polynucleotide encodes a polypeptide comprising an amino acid sequence having at least 80%, at least 85%, at least 90%, at least 95%, at least 97%, or 100% identity to the amino acid sequence set forth in any one of SEQ ID NOs:264-318. In some instances, the polynucleotide encodes a polypeptide comprising an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:264-318. In some instances, the polynucleotide encodes a polypeptide comprising the amino acid sequence set forth in any one of SEQ ID NOs:264-318. In some instances, the polynucleotide is operably linked to a promoter.

In some instances, the polynucleotide comprises: (i) a first nucleic acid encoding a first polypeptide, wherein the first polypeptide comprises a VH described herein (see, e.g., Tables 4-5) or a variant thereof; and (ii) a second nucleic acid encoding a second polypeptide, wherein the second polypeptide comprises a VL described herein (see, e.g., Tables 4-5) or a variant thereof. In some instances, the polynucleotide comprises: (i) a first nucleic acid sequence encoding a first polypeptide, wherein the first polypeptide comprises an amino acid sequence having at least 80%, at least 85%, at least 90%, at least 95%, at least 97%, or 100% identity to the amino acid sequence set forth in any one of SEQ ID NOs:165-208, and (ii) a second nucleic acid sequence encoding a second polypeptide, wherein the second polypeptide comprises an amino acid sequence having at least 80%, at least 85%, at least 90%, at least 95%, at least 97%, or 100% identity to the amino acid sequence set forth in any one of SEQ ID NOs:264-318. In some instances, the polynucleotide comprises: (i) a first nucleic acid sequence encoding a first polypeptide, wherein the first polypeptide comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:165-208; and (ii) a second nucleic acid sequence encoding a second polypeptide, wherein the second polypeptide comprises an amino acid sequence having one or more (e.g., 1, 2, 3, 4, 5, 6, 7, 8, 9, 10) amino acid substitutions, additions, and/or deletions relative to the amino acid sequence set forth in any one of SEQ ID NOs:264-318. In some instances, the first nucleic acid encodes the amino acid sequence set forth in any one of SEQ ID NOs:165-208 and the second nucleic acid encodes the amino acid sequence set forth in any one of SEQ ID NOs:264-318. In some instances, the first nucleic acid is operably linked to a first promoter and the second nucleic acid is operably linked to a second promoter.

Also provided herein are expression vectors encoding the anti-mutCALR antibodies or portions thereof (e.g., VH, VL, HC, and/or LC) described herein. Also provided herein are expression vectors comprising one or more polynucleotides described herein. Various types of expression vectors are known in the art and described herein.

Also provided herein are cells comprising the anti-mutCALR antibodies described herein. Also provided herein are cells comprising one or more polynucleotides described herein. Also provided herein are cells comprising one or more expression vectors described herein. Various types of cells are known in the art and described herein.

### Indications

The anti-mutCALR antibodies of the present disclosure can inhibit the activity of mutCALR, inhibit the activity of one or more signaling pathways downstream of MPL, inhibit oncogenic cell proliferation, inhibit dimerization of MPL, compete with MPL for binding to mutCALR, or a combination thereof.

As used herein, an anti-mutCALR antibody that competes with MPL for binding to mutCALR means that the anti-mutCALR antibody binds to mutCALR with a greater affinity than MPL. In some embodiments, the anti-mutCALR antibody binds to mutCALR with about 10-fold, 50-fold, 100-fold, 500-fold or 1000-fold greater affinity than MPL. In some embodiments, the anti-mutCALR antibody binds to mutCALR with an IC₅₀ of about 10-fold, 50-fold, 100-fold, 500-fold or 1000-fold less than MPL. In some embodiments, the anti-mutCALR antibody binds to mutCALR with an IC₅₀ of between about 0.1 and 1 nM. In some embodiments, the anti-mutCALR antibody binds to mutCALR with an IC₅₀ of about 0.1 nM, 0.2 nM, 0.3 nM, 0.4 nM, 0.5 nM, 0.6 nM, 0.7 nM, 0.8 nM, 0.9 nM or 1 nM.

Accordingly, the antibodies or compositions described herein can be used in methods of inhibiting activity of mutCALR, inhibiting the activity of one or more signaling pathways downstream of MPL, inhibiting oncogenic cell proliferation, inhibiting dimerization of MPL, inhibit the binding of MPL to mutCALR, or a combination thereof in an individual/patient in need of the inhibition by administering an effective amount of an antibody described herein.

Non-limiting examples of signaling pathways downstream of MPL include Janus tyrosine kinase (JAK) and signal transducers and activators of transcription (STAT) signaling, mitogen-activated protein kinase (MEK) and extracellular signal-regulated kinase (ERK) signaling, serine/threonine kinase (AKT) signaling, and mammalian target of rapamycin (mTOR) signaling.

Another aspect of the present disclosure pertains to methods of treating a mutCALR-associated disease or disorder in an individual (e.g., patient) by administering to the individual in need of such treatment a therapeutically effective amount or dose of one or more antibodies of the present disclosure or a pharmaceutical composition thereof. A mutCALR-associated disease or disorder can include any disease, disorder or condition that is directly or indirectly linked to expression or activity of mutCALR.

Another aspect of the present disclosure pertains to methods of treating a myeloproliferative neoplasm in an individual (e.g., patient) by administering to the individual in need of such treatment a therapeutically effective amount or dose of one or more antibodies of the present disclosure or a pharmaceutical composition thereof.

Non-limiting examples of a myeloproliferative neoplasm include chronic myelogenous leukemia, polycythemia vera, primary myelofibrosis, essential thrombocythemia, chronic neutrophilic leukemia, acute myelogenus leukemia, chronic eosinophilic leukemia, chronic myelomonocytic leukemia, myeloproliferative neoplasm and myelodysplastic syndrome, including myelodysplastic syndrome with refractory anaemia with ring sideroblasts, myelodysplastic syndrome with refractory anemia, and myelodysplastic syndrome with refractory anemia with excess blasts.

Anti-mutCALR antibodies disclosed herein can be used to treat, alone or in combination with other therapies, a myeloproliferative neoplasm, or can be used, alone or in combination with other therapies, for the manufacture of a medicament for the treatment of a myeloproliferative neoplasm. Non-limiting examples of other therapies include a JAK inhibitor (e.g., ruxolitinib, itaticinib), a PI3K inhibitor (e.g., parsaclisib), a standard of care therapy (e.g., IFN-alpha, hydroxyurea, thalidomide, lenalidomide, an androgen, an erythropoietin-stimulating agent, a chemotherapeutic agent), or a combination thereof.

Non-limiting examples of JAK inhibitors for use as described herein are provided in U.S. Pat. Nos. 7,335,667; 9,359,358; 8,691,807; 9,181,271; and 9,034,884, each of which is incorporated herein by reference in its entirety.

Non-limiting examples of PI3K inhibitors for use as described herein are provided in U.S. Pat. Nos. 9,108,984; 9,062,055; 8,759,359; and 9,434,746, each of which is incorporated herein by reference in its entirety.

The terms “individual” or “patient” or “subject”, used interchangeably, refer to any animal, including mammals, preferably mice, rats, other rodents, rabbits, dogs, cats, swine, cattle, sheep, horses, or primates, and most preferably humans (i.e., a human subject).

The phrase “therapeutically effective amount” refers to the amount of active antibody or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the antibodies of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Pharmaceutical Compositions

An anti-mutCALR antibody described herein can be formulated as a pharmaceutical composition for administration to a subject, e.g., to treat a disease or disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see, e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7th Ed., Lippincott Williams & Wilkins Publishers (1999) (ISBN: 0683305727); and Kibbe (ed.), Handbook of Pharmaceutical Excipients American Pharmaceutical Association, 3rd ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

The pharmaceutical compositions may be in a variety of forms. These include, for example, liquid, semi-solid and solid dosage forms, such as liquid solutions (e.g., injectable and infusible solutions), dispersions or suspensions, and liposomes. A suitable form can depend on the intended mode of administration and therapeutic application. Typically compositions for the agents described herein are in the form of injectable or infusible solutions.

The composition can be formulated as a solution, microemulsion, dispersion, liposome, or other ordered structure suitable for stable storage at high concentration or as a lyophilized preparation. Sterile injectable solutions can be prepared by incorporating an anti-mutCALR antibody described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. Prolonged absorption of injectable compositions can be brought about by including in the composition an agent that delays absorption, for example, monostearate salts.

The anti-mutCALR antibodies can also be formulated as liposomes prepared by any suitable method known in the art.

Pharmaceutical compositions formulated for subcutaneous administration may be suitable in some circumstances because the subject can self-administer the pharmaceutical composition. Pharmaceutical formulations for subcutaneous administration can further comprise protein formulations comprising arginine-HCl, histidine, and/or polysorbate, which may confer increased potency, improved serum half-life, or enhanced solubility to the anti-mutCALR antibodies.

### Administration

The anti-mutCALR antibody can be administered to a subject, e.g., a subject in need thereof, for example, a human subject, by a variety of methods. For many applications, the route of administration can be intravenous injection or infusion (IV), subcutaneous injection (SC), intraperitoneally (IP), or intramuscular injection.

The route and/or mode of administration of the antibody can also be tailored for the individual case, e.g., by monitoring the subject, e.g., using tomographic imaging, e.g., to visualize a tumor.

The anti-mutCALR antibody can be administered as a fixed dose, or in a mg/kg patient weight dose. The dose can also be chosen to reduce or avoid production of antibodies against the anti-mutCALR antibody. Dosage regimens are adjusted to provide the desired response, e.g., a therapeutic response or a combinatorial therapeutic effect. Generally, doses of the anti-mutCALR antibody (and optionally a second agent) can be used in order to provide a subject with the agent in bioavailable quantities.

Dosage unit form or “fixed dose” or “flat dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion.

The disclosure also provides a kit comprising one or more containers of an anti-mutCALR antibody or a pharmaceutical formulation thereof, optionally with one or more other prophylactic or therapeutic agents useful for the treatment of a disease or disorder, and optionally with instructions for using the anti-mutCALR antibody or a pharmaceutical formulation thereof.

The instructions relating to the use of an anti-mutCALR antibody generally include information as to dosage, dosing schedule, and route of administration for the intended treatment. The containers can be unit doses, bulk packages (e.g., multi-dose packages) or sub-unit doses. Instructions supplied in the kits of the disclosure are typically written instructions on a label or package insert. The label package insert indicates that an anti-mutCALR antibody is used for treating, delaying the onset, and/or alleviating a myeloproliferative neoplasm.

### Detection and Diagnosis

Mutant CALR can be detected in a biological sample of a subject who has a myeloproliferative neoplasm. Thus an aspect of the present disclosure provides a method of detecting a CALR exon 9 mutation in a subject's biological sample, the method comprising obtaining a biological sample from a subject who has or is suspected of having a myeloproliferative neoplasm and contacting the sample with an anti-mutCALR antibody of described herein such that the anti-mutCALR antibody binds to the mutCALR protein if the mutCALR protein is present in the biological sample.

Another aspect of the present disclosure provides a method of diagnosing a subject with a myeloproliferative neoplasm, the method comprising obtaining a biological sample from a subject who has or is suspected of having a myeloproliferative neoplasm and contacting the sample with an anti-mutCALR antibody described herein such that the anti-mutCALR antibody binds to the mutCALR protein if the mutCALR protein is present in the biological sample.

The biological sample can be a blood sample, a bone marrow sample or a serum sample. In some embodiments, the biological sample is fresh or frozen. In some embodiments, the biological sample is fixed, for example in formaldehyde or paraformaldehyde.

Non-limiting examples of a myeloproliferative neoplasm that can be diagnosed with the present method include chronic myelogenous leukemia, polycythemia vera, primary myelofibrosis, essential thrombocythemia, chronic neutrophilic leukemia, acute myelogenus leukemia, chronic eosinophilic leukemia, chronic myelomonocytic leukemia, myeloproliferative neoplasm and myelodysplastic syndrome, including myelodysplastic syndrome with refractory anaemia with ring sideroblasts, myelodysplastic syndrome with refractory anemia, and myelodysplastic syndrome with refractory anemia with excess blasts.

An anti-mutCALR antibody described herein for use in the present detection or diagnostic methods can also comprise (e.g., be conjugated to) a detectable label. Suitable detectable labels include a radioisotope, a nanoparticle, a fluorescent compound, a bioluminescent compound, chemiluminescent compound, a metal chelator, magnetic beads, metallic beads, colloidal particles, fluorescent dyes, electron-dense reagents, enzymes (for example, as used in an ELISA), biotin, digoxigenin, or haptens. Suitable techniques for conjugating diagnostic agents to antibodies are known in the art (see, e.g., Jazayeri etl al., Sensing and Bio-Sensing Research (2016); 9:17-22 and Balasubramanya, Mater. Methods (2018); 8:2670).

An anti-mutCALR antibody described herein bound to mutCALR protein, whether labeled or unlabeled, can be detected by any suitable detection method, including immunologic techniques such as immunohistochemistry (IHC), immunocytochemistry, Western blot, or ELISA immunoassay; gel- or blot-based methods; mass spectrometry, flow cytometry; or fluorescent activated cell sorting (FACS).

The disclosure also provides a kit for the diagnosis of a myeloproliferative neoplasm comprising one or more containers of an anti-mutCALR antibody described herein or a diagnostic formulation thereof, and optionally with instructions for using the anti-mutCALR antibody or a diagnostic formulation thereof to detect a mutCALR exon 9 mutation or diagnose a myeloproliferative neoplasm.

The following are examples of the practice of the invention. They are not to be construed as limiting the scope of the invention in any way.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art can develop equivalent means or reactants without the exercise of inventive capacity and without departing from the scope of the invention.

### Example 1: Generation of Anti-mutCALR Antibodies

To generate anti-human mutCALR monoclonal antibodies, mice were immunized with one plasmid encoding human MPL and a second plasmid encoding human Type 1 mutCalR. The sequences for mutant CalR and MPL were cloned into the pVAC2 expression plasmid (Invivogen). The nucleotide sequences used in the vectors as well as the sequences of the encoded proteins are shown below.

Human MPL nucleotide sequence:
(SEQ ID NO: 323)
ATGCCCTCCTGGGCCCTCTTCATGGTCACCTCCTGCCTCCTCCTGGCCCCTCAAAACCTGGCCC
AAGTCAGCAGCCAAGATGTCTCCTTGCTGGCATCAGACTCAGAGCCCCTGAAGTGTTTCTCCCG
AACATTTGAGGACCTCACTTGCTTCTGGGATGAGGAAGAGGCAGCGCCCAGTGGGACATACCAG
CTGCTGTATGCCTACCCGCGGGAGAAGCCCCGTGCTTGCCCCCTGAGTTCCCAGAGCATGCCCC
ACTTTGGAACCCGATACGTGTGCCAGTTTCCAGACCAGGAGGAAGTGCGTCTCTTCTTTCCGCT
GCACCTCTGGGTGAAGAATGTGTTCCTAAACCAGACTCGGACTCAGCGAGTCCTCTTTGTGGAC
AGTGTAGGCCTGCCGGCTCCCCCCAGTATCATCAAGGCCATGGGTGGGAGCCAGCCAGGGGAAC
TTCAGATCAGCTGGGAGGAGCCAGCTCCAGAAATCAGTGATTTCCTGAGGTACGAACTCCGCTA
TGGCCCCAGAGATCCCAAGAACTCCACTGGTCCCACGGTCATACAGCTGATTGCCACAGAAACC
TGCTGCCCTGCTCTGCAGAGGCCTCACTCAGCCTCTGCTCTGGACCAGTCTCCATGTGCTCAGC
CCACAATGCCCTGGCAAGATGGACCAAAGCAGACCTCCCCAAGTAGAGAAGCTTCAGCTCTGAC
AGCAGAGGGTGGAAGCTGCCTCATCTCAGGACTCCAGCCTGGCAACTCCTACTGGCTGCAGCTG
CGCAGCGAACCTGATGGGATCTCCCTCGGTGGCTCCTGGGGATCCTGGTCCCTCCCTGTGACTG
TGGACCTGCCTGGAGATGCAGTGGCACTTGGACTGCAATGCTTTACCTTGGACCTGAAGAATGT
TACCTGTCAATGGCAGCAACAGGACCATGCTAGCTCCCAAGGCTTCTTCTACCACAGCAGGGCA
CGGTGCTGCCCCAGAGACAGGTACCCCATCTGGGAGAACTGCGAAGAGGAAGAGAAAACAAATC
CAGGACTACAGACCCCACAGTTCTCTCGCTGCCACTTCAAGTCACGAAATGACAGCATTATTCA
CATCCTTGTGGAGGTGACCACAGCCCCGGGTACTGTTCACAGCTACCTGGGCTCCCCTTTCTGG
ATCCACCAGGCTGTGCGCCTCCCCACCCCAAACTTGCACTGGAGGGAGATCTCCAGTGGGCATC
TGGAATTGGAGTGGCAGCACCCATCGTCCTGGGCAGCCCAAGAGACCTGTTATCAACTCCGATA
CACAGGAGAAGGCCATCAGGACTGGAAGGTGCTGGAGCCGCCTCTCGGGGCCCGAGGAGGGACC
CTGGAGCTGCGCCCGCGATCTCGCTACCGTTTACAGCTGCGCGCCAGGCTCAACGGCCCCACCT
ACCAAGGTCCCTGGAGCTCGTGGTCGGACCCAACTAGGGTGGAGACCGCCACCGAGACCGCCTG
GATCTCCTTGGTGACCGCTCTGCATCTAGTGCTGGGCCTCAGCGCCGTCCTGGGCCTGCTGCTG
CTGAGGTGGCAGTTTCCTGCACACTACAGGAGACTGAGGCATGCCCTGTGGCCCTCACTTCCAG
ACCTGCACCGGGTCCTAGGCCAGTACCTTAGGGACACTGCAGCCCTGAGCCCGCCCAAGGCCAC
AGTCTCAGATACCTGTGAAGAAGTGGAACCCAGCCTCCTTGAAATCCTCCCCAAGTCCTCAGAG
AGGACTCCTTTGCCCCTGTGTTCCTCCCAGGCCCAGATGGACTACCGAAGATTGCAGCCTTCTT
GCCTGGGGACCATGCCCCTGTCTGTGTGCCCACCCATGGCTGAGTCAGGGTCCTGCTGTACCAC
CCACATTGCCAACCATTCCTACCTACCACTAAGCTATTGGCAGCAGCCT
Human MPL amino acid sequence:
(SEQ ID NO: 324)
MPSWALFMVTSCLLLAPQNLAQVSSQDVSLLASDSEPLKCFSRTFEDLTCFWDEEEAAPSGTYQ
LLYAYPREKPRACPLSSQSMPHFGTRYVCQFPDQEEVRLFFPLHLWVKNVFLNQTRTQRVLFVD
SVGLPAPPSIIKAMGGSQPGELQISWEEPAPEISDFLRYELRYGPRDPKNSTGPTVIQLIATET
CCPALQRPHSASALDQSPCAQPTMPWQDGPKQTSPSREASALTAEGGSCLISGLQPGNSYWLQL
RSEPDGISLGGSWGSWSLPVTVDLPGDAVALGLQCFTLDLKNVTCQWQQQDHASSQGFFYHSRA
RCCPRDRYPIWENCEEEEKTNPGLQTPQFSRCHFKSRNDSITHILVEVTTAPGTVHSYLGSPFW
IHQAVRLPTPNLHWREISSGHLELEWQHPSSWAAQETCYQLRYTGEGHQDWKVLEPPLGARGGT
LELRPRSRYRLQLRARINGPTYQGPWSSWSDPTRVETATETAWISLVTALHLVLGLSAVLGLLL
LRWQFPAHYRRLRHALWPSLPDLHRVLGQYLRDTAALSPPKATVSDTCEEVEPSLLEILPKSSE
RTPLPLCSSQAQMDYRRLQPSCLGTMPLSVCPPMAESGSCCTTHIANHSYLPLSYWQQP
Human Type 1 mutCALR nucleotide sequence:
(SEQ ID NO: 325)
ATGCTGCTATCCGTGCCGCTGCTGCTCGGCCTCCTCGGCCTGGCCGTCGCCGAGCCTGCCGTCT
ACTTCAAGGAGCAGTTTCTGGACGGAGACGGGTGGACTTCCCGCTGGATCGAATCCAAACACAA
GTCAGATTTTGGCAAATTCGTTCTCAGTTCCGGCAAGTTCTACGGTGACGAGGAGAAAGATAAA
GGTTTGCAGACAAGCCAGGATGCACGCTTTTATGCTCTGTCGGCCAGTTTCGAGCCTTTCAGCA
ACAAAGGCCAGACGCTGGTGGTGCAGTTCACGGTGAAACATGAGCAGAACATCGACTGTGGGGG
CGGCTATGTGAAGCTGTTTCCTAATAGTTTGGACCAGACAGACATGCACGGAGACTCAGAATAC
AACATCATGTTTGGTCCCGACATCTGTGGCCCTGGCACCAAGAAGGTTCATGTCATCTTCAACT
ACAAGGGCAAGAACGTGCTGATCAACAAGGACATCCGTTGCAAGGATGATGAGTTTACACACCT
GTACACACTGATTGTGCGGCCAGACAACACCTATGAGGTGAAGATTGACAACAGCCAGGTGGAG
TCCGGCTCCTTGGAAGACGATTGGGACTTCCTGCCACCCAAGAAGATAAAGGATCCTGATGCTT
CAAAACCGGAAGACTGGGATGAGCGGGCCAAGATCGATGATCCCACAGACTCCAAGCCTGAGGA
CTGGGACAAGCCCGAGCATATCCCTGACCCTGATGCTAAGAAGCCCGAGGACTGGGATGAAGAG
ATGGACGGAGAGTGGGAACCCCCAGTGATTCAGAACCCTGAGTACAAGGGTGAGTGGAAGCCCC
GGCAGATCGACAACCCAGATTACAAGGGCACTTGGATCCACCCAGAAATTGACAACCCCGAGTA
TTCTCCCGATCCCAGTATCTATGCCTATGATAACTTTGGCGTGCTGGGCCTGGACCTCTGGCAG
GTCAAGTCTGGCACCATCTTTGACAACTTCCTCATCACCAACGATGAGGCATACGCTGAGGAGT
TTGGCAACGAGACGTGGGGCGTAACAAAGGCAGCAGAGAAACAAATGAAGGACAAACAGGACGA
GGAGCAGAGGACAAGGAGGATGATGAGGACAAAGATGAGGATGAGGAGGATGAGGAGGACAAGG
AGGAAGATGAGGAGGAAGATGTCCCCGGCCAGGCCAAGGACGAGCTGTAGAGAGGCCTGCCTCC
AGGGCTGGACTGAGGCC
Human Type I mutCALR amino acid sequence:
(SEQ ID NO: 320)
MLLSVPLLLGLLGLAVAEPAVYFKEQFLDGDGWTSRWIESKHKSDFGKFVLSSGKFYGDEEKDK
GLQTSQDARFYALSASFEPFSNKGQTLVVQFTVKHEQNIDCGGGYVKLFPNSLDQTDMHGDSEY
NIMFGPDICGPGTKKVHVIFNYKGKNVLINKDIRCKDDEFTHLYTLIVRPDNTYEVKIDNSQVE
SGSLEDDWDFLPPKKIKDPDASKPEDWDERAKIDDPTDSKPEDWDKPEHIPDPDAKKPEDWDEE
MDGEWEPPVIQNPEYKGEWKPRQIDNPDYKGTWIHPEIDNPEYSPDPSIYAYDNFGVLGLDLWQ
VKSGTIFDNFLITNDEAYAEEFGNETWGVTKAAEKQMKDKQDEEQRTRRMMRTKMRMRRMRRTR
RKMRRKMSPARPRTSCREACLQGWTEA

Plasmablasts were isolated from the mouse spleen and lymph nodes by flow cytometry. Antibody sequences of the plasmablasts were determined using 10× Genomics VH/VL paired B cell sequencing. The murine VH/VL pairs were expressed as chimeras with huIgG1 Fc and tested for binding and functionality. An antibody designated clone 54 was produced by this process.

In addition, multiple selection rounds of single-donor and multi-donor phage display libraries were performed according to the method of Erasmus et al., Communications Biology 4, article no. 350 (2021). Phage libraries were enriched for either 2 or 3 rounds on biotinylated MBP-mutCalR Type 1 fusion protein (Cepter Biopartners) with deselection using MBP (Rockland Immunochemicals) and streptavidin beads (Thermo Fisher) each round (see, e.g., Example 2). scFv cassettes from the library pools showing the strongest specific enrichment for mutCalR were then recombined into a yeast display vector to create yeast display libraries. These were selected over four rounds using decreasing concentrations of MBP-mutCalR with and without blocking with either a scrambled peptide or oligo-lysine peptide (Alamanda Polymers). In the final round of selection, yeast were also selected for binding to a biotinylated mutCalR long peptide (Biot-LC-MKDKQDEEQRTRRMMRTKMRMRRMRRTRRKMRRKMSPARPRTSSREASLQGWTEA; SEQ ID NO:332) or short peptide (Biot-LC-MKDKQDEEQRTRRMMRTKMRMRRMRRTRRKM, SEQ ID NO:333). Unique sequences were obtained from the final sorting output by Sanger sequencing of yeast colonies. For screening, these scFv were reformatted by cloning into a human IgG1 expression vector, then expressed and purified from Expi293F cell (Thermo Fisher, cat. #A14635) supernatants. The MBP-mutCalR Type 1 fusion protein used in this experiment is:

(SEQ ID NO: 334)
HHHHHHMKIEEGKLVIWINGDKGYNGLAEVGKKFEKDTGIKVTVEHPDKL
EEKFPQVAATGDGPDIIFWAHDRFGGYAQSGLLAEITPDKAFQDKLYPFT
WDAVRYNGKLIAYPIAVEALSLIYNKDLLPNPPKTWEEIPALDKELKAKG
KSALMFNLQEPYFTWPLIAADGGYAFKYENGKYDIKDVGVDNAGAKAGLT
FLVDLIKNKHMNADTDYSIAEAAFNKGETAMTINGPWAWSNIDTSKVNYG
VTVLPTFKGQPSKPFVGVLSAGINAASPNKELAKEFLENYLLTDEGLEAV
NKDKPLGAVALKSYEEELAKDPRIAATMENAQKGEIMPNIPQMSAFWYAV
RTAVINAASGRQTVDEALKDAQTNSSSLEVLFQGPGLNDIFEAQKIEWHE
ENLYFQGEPAVYFKEQFLDGDGWTSRWIEKHKSDFGKFVLSSGKFYGDEE
KDKGLQTSQDARFYALSASFEPFSNKGQTLVVQFTVKHEQNIDCGGGYVK
LFPNSLDQTDMHGDSEYNIMFGPDICGPGTKKVHVIFNYKGKNVLINKDI
RCKDDEFTHLYTLIVRPDNTYEVKIDNSQVESGSLEDDWDFLPPKKIKDP
DASKPEDWDERAKIDDPTDSKPEDWDKPEHIPDPDAKKPEDWDEEMDGEW
EPPVIQNPEYKGEWKPRQIDNPDYKGTWIHPEIDNPEYSPDPSIYAYDNF
GVLGLDLWQVKSGTIFDNFLITNDEAYAEEFGNETWGVTKAAEKQMKDKQ
DEEQRTRRMMRTKMRMRRMRRTRRKMRRKMSPARPRTSCREACLQGWTEA

The amino acid sequences of the six CDRs for each of the 54 unique clones are shown in Table 1. The heavy chain, VH, light chain, and VL sequences of each clone are shown in Table 4.

Mutations were introduced into three of the identified clones (clones 6, 15, and 17) to generate 161 unique mutant clones (clones 55-215). The amino acid sequences of the six CDRs for each of the mutant clones are shown in Table 2. Light chain and heavy chain sequences of the parental clones (clones 6, 15, and 17) and mutant clones (clones 55-215) are shown in Table 5. One such mutation to the heavy chain, N297A, resulted in Fc effector function “null” mutants, where the Fc effector function, particularly ADCC, was eliminated or substantially eliminated. As can be seen from the activity data in the Examples, those antibodies with N297A Fc effector null mutations have a generally increased binding affinity for mutCALR than those antibodies without an Fc effector null mutation.

TABLE 1
CDR sequences.

| Clone No. | Group | VH CDR1 | VH CDR2 | VH CDR3 | VL CDR1 | VL CDR2 | VL CDR3 |
|---|---|---|---|---|---|---|---|
| 1 | 1 | ELSMQ | GFDPDDGETMYAEKFQG | SPGYDFFDY | GGNYIGSKSVH | DDGDRPS | QVWDSISDHVV |
| | | (SEQ ID NO: 1) | (SEQ ID NO: 7) | (SEQ ID NO: 18) | (SEQ ID NO: 26) | (SEQ ID NO: 53) | (SEQ ID NO: 69) |
| 2 | 1 | ELSMQ | GFDPDDGETMYAEKFQG | SPGYDFFDY | GGDNIGSKSVH | DDSDRPS | QVWDSSIDHLI |
| | | (SEQ ID NO: 1) | (SEQ ID NO: 7) | (SEQ ID NO: 18) | (SEQ ID NO: 27) | (SEQ ID NO: 54) | (SEQ ID NO: 70) |
| 3 | 1 | ELSMQ | GFDPDDGETMYAERFQG | SPGYDFFDY | GGNNIGSKSVH | DDSDRPS | QVWDSSNDLLI |
| | | (SEQ ID NO: 1) | (SEQ ID NO: 8) | (SEQ ID NO: 18) | (SEQ ID NO: 28) | (SEQ ID NO: 54) | (SEQ ID NO: 71) |
| 4 | 1 | ELSMQ | GFDPDDGETMYAEKFQG | SPGYDFFDY | GGNNIGSKSVH | DDSDRPS | QVWDSSNDLLI |
| | | (SEQ ID NO: 1) | (SEQ ID NO: 7) | (SEQ ID NO: 18) | (SEQ ID NO: 28) | (SEQ ID NO: 54) | (SEQ ID NO: 71) |
| 5 | 1 | ELSMQ | GFDPDDGETMYAEKFQG | SPGYDFFDY | VGTNFGSKNVH | DDSDRPS | QVWDSSSDHLI |
| | | (SEQ ID NO: 1) | (SEQ ID NO: 7) | (SEQ ID NO: 18) | (SEQ ID NO: 29) | (SEQ ID NO: 54) | (SEQ ID NO: 72) |
| 6 | 1 | ELSMQ | GFDPDDGETMYAEKFQG | SPGYDFFDY | TGTSSDVGGYNYVS | EVSNRPS | QVWDSSNDLL |
| | | (SEQ ID NO: 1) | (SEQ ID NO: 7) | (SEQ ID NO: 18) | (SEQ ID NO: 30) | (SEQ ID NO: 55) | (SEQ ID NO: 71) |
| 7 | 1 | ELSMQ | GFDPDDGETMYAEKFQG | SPGYDFFDY | VGTNFGSKNVH | DDSDRPS | QVWDSSNDLLI |
| | | (SEQ ID NO: 1) | (SEQ ID NO: 7) | (SEQ ID NO: 18) | (SEQ ID NO: 29) | (SEQ ID NO: 54) | (SEQ ID NO: 71) |
| 8 | 1 | ELSMQ | GFDPDDGETMYAEKFQG | SPGYDFFDY | GGNNIGSKSVH | DDSDRPS | QVWDSSSDHLV |
| | | (SEQ ID NO: 1) | (SEQ ID NO: 7) | (SEQ ID NO: 18) | (SEQ ID NO: 28) | (SEQ ID NO: 54) | (SEQ ID NO: 73) |
| 9 | 1 | ELSMQ | GFDPDDGETMYAEKFQG | SPGYDFFDY | GGNNIGSKSVH | DDRDRPS | QVWDSSNDLLI |
| | | (SEQ ID NO: 1) | (SEQ ID NO: 7) | (SEQ ID NO: 18) | (SEQ ID NO: 28) | (SEQ ID NO: 56) | (SEQ ID NO: 71) |
| 10 | 1 | ELSMQ | GFDPDDGETMYAEKFQG | SPGYDFFDY | GGNNIGSEGVH | DDSDRPS | QVWDSSNDLLI |
| | | (SEQ ID NO: 1) | (SEQ ID NO: 7) | (SEQ ID NO: 18) | (SEQ ID NO: 31) | (SEQ ID NO: 54) | SEQ ID NO: 71) |
| 11 | 1 | ELSMQ | GFDPDDGETMYAEKFQG | SPGYDFFDY | TGTSSDVGGYNYVS | DVSKRPS | QVWDSSNDLLI |
| | | (SEQ ID NO: 1) | (SEQ ID NO: 7) | (SEQ ID NO: 18) | (SEQ ID NO: 30) | (SEQ ID NO: 57) | (SEQ ID NO: 71) |
| 12 | 1 | ELSMQ | GFDPDGGETMYAEKFQG | SPGYDFFDY | GGNNIGSKSVN | DDSDRPS | QVWDSSNDLLI |
| | | (SEQ ID NO: 1) | (SEQ ID NO: 9) | (SEQ ID NO: 18) | (SEQ ID NO: 32) | (SEQ ID NO: 54) | (SEQ ID NO: 71) |
| 13 | 1 | ELSMQ | GFDPDDGETMYAEKFQG | SPGYDFFDY | GGNNIGSKSVF | DDSDRPS | QVWDSSSDHLV |
| | | (SEQ ID NO: 1) | (SEQ ID NO: 7) | (SEQ ID NO: 18) | (SEQ ID NO: 33) | (SEQ ID NO: 54) | (SEQ ID NO: 73) |
| 14 | 1 | ELSMQ | GFDPDDGETMYAEKFQG | SPGYDFFDY | GGYNIGSKFVH | DDRDRPS | QVWDSSNDLLI |
| | | (SEQ ID NO: 1) | (SEQ ID NO: 7) | (SEQ ID NO: 18) | (SEQ ID NO: 34) | (SEQ ID NO: 56) | (SEQ ID NO: 71) |
| 15 | 1 | ELSMQ | GFDPDDGETMYAEKFQG | SPGYDFFDY | GGDNIGSKSVH | DDSDRPS | QVWDSSNDLLI |
| | | (SEQ ID NO: 1) | (SEQ ID NO: 7) | (SEQ ID NO: 18) | (SEQ ID NO: 27) | (SEQ ID NO: 54) | (SEQ ID NO: 71) |
| 16 | 1 | ELSMQ | GFDPDDGETMYAEKFQG | SPGYDFFDY | GGNNTGSKSVH | DDSDRPS | QVWDSSNDLLI |
| | | (SEQ ID NO: 1) | (SEQ ID NO: 7) | (SEQ ID NO: 18) | (SEQ ID NO: 35) | (SEQ ID NO: 54) | (SEQ ID NO: 71) |
| 17 | 1 | ELSMQ | GFDPDDGETMYAEKFQG | SPGYDFFDY | GGSDIGDEIVH | DDSDRPS | QVWDSSSDHLI |
| | | (SEQ ID NO: 1) | (SEQ ID ID NO: 7) | (SEQ ID NO: 18) | (SEQ ID NO: 36) | (SEQ ID NO: 54) | (SEQ ID NO: 72) |
| 18 | 1 | ELSMQ | GFDPDDGETMYAEKFOG | SPGYDFFDY | GGNNIGSKSVH | DDSDRPL | QVWDSSNDLLI |
| | | (SEQ ID NO: 1) | (SEQ ID NO: 7) | (SEQ ID NO: 18) | (SEQ ID NO: 28) | (SEQ ID NO: 58) | (SEQ ID NO: 71) |
| 19 | 1 | ELSMQ | GFDPDDGETMYAEKFQG | SPGYDFFDY | GGSNIGDIRVH | DDSDRPS | QVWDSSNDLLI |
| | | (SEQ ID NO: 1) | (SEQ ID NO: 7) | (SEQ ID NO: 18) | (SEQ ID NO: 37) | (SEQ ID NO: 54) | (SEQ ID NO: 71) |
| 20 | 1 | ELSMQ | GFDPDDGETMYAEKFQG | SPGYDFFDY | GGNNIGSKGVH | DDSDRPS | QVWDSSNDLLI |
| | | (SEQ ID NO: 1) | (SEQ ID NO: 7) | (SEQ ID NO: 18) | (SEQ ID NO: 38) | (SEQ ID NO: 54) | (SEQ ID NO: 71) |
| 21 | 1 | ELSMQ | GFDPDDGETMYAEKFQG | SPGYDFFDY | GGNNIGSKSVN | DDSDRPS | QVWDSSNDLLI |
| | | (SEQ ID NO: 1) | (SEQ ID NO: 7) | (SEQ ID NO: 18) | (SEQ ID NO: 32) | (SEQ ID NO: 54) | (SEQ ID NO: 71) |
| 22 | 1 | ELSMQ | GFDPDDGETMYAEKFQG | SPGYDFFDY | GGNNIGSKNVH | DDSDRPS | QVWDSSNDLLI |
| | | (SEQ ID NO: 1) | (SEQ ID NO: 7) | (SEQ ID NO: 18) | (SEQ ID NO: 39) | (SEQ ID NO: 54) | (SEQ ID NO: 71) |
| 23 | 1 | ELSMQ | GFDPDDGETMYAEKFQG | SPGYDFFDY | GGNNIGIKSVH | DDSDRPS | QVWDSSNDLLI |
| | | (SEQ ID NO: 1) | (SEQ ID NO: 7) | (SEQ ID NO: 18) | (SEQ ID NO: 40) | (SEQ ID NO: 54) | (SEQ ID NO: 71) |
| 24 | 1 | ELSMQ | GFDPDDGETMYAEKLQG | SPGYDFFDY | GGNNIGSKSVH | DDSDRPS | QVWDSSNDLLI |
| | | (SEQ ID NO: 1) | (SEQ ID NO: 10) | (SEQ ID NO: 18) | (SEQ ID NO: 28) | (SEQ ID NO: 54) | (SEQ ID NO: 71) |
| 25 | 1 | ELSMQ | GFDPDDGETMYAEKFQG | SPGYDFFDY | GGNNIGRKAVH | DDSDRPS | QVWDSSNDLLI |
| | | (SEQ ID NO: 1) | (SEQ ID NO: 7) | (SEQ ID NO: 18) | (SEQ ID NO: 41) | (SEQ ID NO: 54) | (SEQ ID NO: 71) |
| 26 | 1 | ELSMQ | GFDPDDGETMYAEKFQG | SPGYDFFDY | GGNNIGSKSVH | DDSDRPS | QVWDSSIDHLI |
| | | (SEQ ID NO: 1) | (SEQ ID NO: 7) | (SEQ ID NO: 18) | (SEQ ID NO: 28) | (SEQ ID NO: 54) | (SEQ ID NO: 70) |
| 27 | 1 | ELSMQ | GFDPDDGETMYAEKFQG | SPGYDFFDY | GGNNIGIKSVH | DDRDRPS | QVWDSSNDLLI |
| | | (SEQ ID NO: 1) | (SEQ ID NO: 7) | (SEQ ID NO: 18) | (SEQ ID NO: 40) | (SEQ ID NO: 56) | (SEQ ID NO: 71) |
| 28 | 1 | ELSMQ | GFDPDDGETMYAEKFQG | SPGYDFFDY | GGNNIGSKSVH | DDSDRPS | QVWDADSDQLI |
| | | (SEQ ID NO: 1) | (SEQ ID NO: 7) | (SEQ ID NO: 18) | (SEQ ID NO: 28) | (SEQ ID NO: 54) | (SEQ ID NO: 74) |
| 29 | 1 | ELSMQ | GFDPDDGETMYAEKFQG | SPGYDFFDY | GGNNIGSKSVH | DDSDRPS | QVWDSSSDLLI |
| | | (SEQ ID NO: 1) | (SEQ ID NO: 7) | (SEQ ID NO: 18) | (SEQ ID NO: 28) | (SEQ ID NO: 54) | (SEQ ID NO: 75) |
| 30 | 1 | ELSMQ | GFDPDDGETMYAEKFQG | SPGYDFFDY | GGNNIGTKSVH | DDSDRPS | QVWDSSNDLLI |
| | | (SEQ ID NO: 1) | (SEQ ID NO: 7) | (SEQ ID NO: 18) | (SEQ ID NO: 118) | (SEQ ID NO: 54) | (SEQ ID NO: 71) |
| 31 | 2 | DYFIH | LVDPEDGETIYAEKFQG | PGGILTDPDAFDI | TGTSSDVGGYNYVS | DVNKRPS | SSYIPSSTRWV |
| | | SEQ ID NO: 2) | (SEQ ID NO: 11) | (SEQ ID NO: 19) | (SEQ ID NO: 30) | (SEQ ID NO: 59) | (SEQ ID NO: 76) |
| 32 | 2 | DYFIH | LVDPEDGETIYAEKFQG | PGGILTDPDAFDI | TGTSSDVGGYNYVS | EVSNRPS | SSYTSSSTPYV |
| | | (SEQ ID NO: 2) | (SEQ ID NO: 11) | (SEQ ID NO: 19) | (SEQ ID NO: 30) | (SEQ ID NO: 55) | (SEQ ID NO: 77) |
| 33 | 2 | DYFIH | LVDPEDGETIYAEKFQG | PGGILTDPDAFDI | AGTGSDVGGYNHVS | DVNKRPS | SSYIPSSTRWV |
| | | (SEQ ID NO: 2) | (SEQ ID NO: 11) | (SEQ ID NO: 19) | (SEQ ID NO: 42) | (SEQ ID NO: 59) | (SEQ ID NO: 76) |
| 34 | 2 | DYFIH | LVDPEDGETIYAEKFQG | PGGILTDPDAFDI | TGTSSDVGGYNYVS | EVSNRPS | SSYTSSSTYV |
| | | (SEQ ID NO: 2) | (SEQ ID NO: 11) | (SEQ ID NO: 19) | (SEQ ID NO: 30) | (SEQ ID NO: 55) | (SEQ ID NO: 78) |
| 35 | 2 | DYFIH | LVDPEDGETIYAERFQG | PGGILTDPDAFDI | TGTSSDVGGYNYVS | EVSNRPS | SSYTSSSTPYV |
| | | (SEQ ID NO: 2) | (SEQ ID NO: 12) | (SEQ ID NO: 19) | (SEQ ID NO: 30) | (SEQ ID NO: 55) | (SEQ ID NO: 77) |
| 36 | 2 | DYFIH | LVDPEDGETIYAEKFQG | PGGILTDPDAFDI | AGTGSDVGGYNYVS | DVNKRPS | SSYTSSSTFYV |
| | | SEQ ID NO: 2) | (SEQ ID NO: 11) | (SEQ ID NO: 19) | (SEQ ID NO: 43) | ISEQ ID NO: 59) | (SEQ ID NO: 79) |
| 37 | 2 | DYFIH | LVDPEDGETIYAEKFQG | PGGILTDPDAFDI | TGTSSDVGGYNYVS | DVSNRPS | SSYTSSSTFYV |
| | | (SEQ ID NO: 2) | (SEQ ID NO: 11) | (SEQ ID NO: 19) | (SEQ ID NO: 30) | (SEQ ID NO: 60) | (SEQ ID NO: 79) |
| 38 | 3 | SYAIS | LVDPEDGETIYAEKFRG | EESYGP | QASQDISNYLN | DASNLET | QQLNSYPLT |
| | | (SEQ ID NO: 3) | (SEQ ID NO: 13) | (SEQ ID NO: 20) | (SEQ ID NO: 44) | (SEQ ID NO: 61) | (SEQ ID NO: 80) |
| 39 | 3 | SYAIS | LVDPEDGETIYAEKFQG | EESYGP | QASQDISNYLD | DASNLET | QQLNSYPLT (SEQ |
| | | (SEQ ID NO: 3) | (SEQ ID NO: 11) | (SEQ ID NO: 20) | (SEQ ID NO: 45) | (SEQ ID NO: 61) | ID NO: 80) |
| 40 | 4 | ESSMH | LVDPEDGETIYAQKFQG | EEWSGDGDDAFDI | SGSSSNIGSYSVS | DNNKRPS | GTWDSSLSAWV |
| | | (SEQ ID NO: 4) | (SEQ ID NO: 14) | (SEQ ID NO: 21) | (SEQ ID NO: 46) | (SEQ ID NO: 62) | (SEQ ID NO: 81) |
| 41 | 4 | ELSMH | LVDPEDGETIYAQKFQG | EEWSGDGDDAFDI | SGSSSNIGSYSVS | DNNKRPS | GTWDSSLSAWV |
| | | (SEQ ID NO: 5) | (SEQ ID NO: 14) | (SEQ ID NO: 21) | (SEQ ID NO: 46) | (SEQ ID NO: 62) | (SEQ ID NO: 81) |
| 42 | 4 | ELSMH | LVDPEDGETIYAQKFQG | EEWSGDGDDAFDI | SGSSSNIGSYSVS | DDNKRPS | GTWDSSLSAWV |
| | | (SEQ ID NO: 5) | (SEQ ID NO: 14) | (SEQ ID NO: 21) | (SEQ ID NO: 46) | (SEQ ID NO: 63) | (SEQ ID NO: 81) |
| 43 | 6 | SYAIS | GIIPIFGTANYAQKEQG | SPLRGSGWYWHYYYGMDV | GGNNIRAKHVH | QDSKRPS | QAWDSSTVV |
| | | (SEQ ID NO: 3) | (SEQ ID NO: 15) | (SEQ ID NO: 22) | (SEQ ID NO: 47) | (SEQ ID NO: 64) | (SEQ ID NO: 82) |
| 44 | 6 | SYAIS | GIIPIFGTANYAQKFQG | SPLRGSGWYWHYYYGMDV | GGNNIRSKSVH | QDSKRPS | QAWDSNTVV |
| | | (SEQ ID NO: 3) | (SEQ ID NO: 15) | (SEQ ID NO: 22) | (SEQ ID NO: 48) | (SEQ ID NO: 64) | (SEQ ID NO: 83) |
| 45 | 6 | SYAIS | GIIPIFGTANYAQKFQG | SPLRGSGWYWHYYYGMDV | GGNNIGRKSVH | RDSNRPS | QVWDSSTVV |
| | | (SEQ ID NO: 3) | (SEQ ID NO: 15) | (SEQ ID NO: 22) | (SEQ ID NO: 49) | (SEQ ID NO: 65) | (SEQ ID NO: 84) |
| 46 | 6 | SYAIS | GIIPIFGTANYAQKFQG | SPLRGSGWYWHYYYGMDV | GGNNIRSKSVH | QDSKRPS | QVWDSSTAV |
| | | (SEQ ID NO: 3 | (SEQ ID NO: 15) | (SEQ ID NO: 22) | (SEQ ID NO: 48) | (SEQ ID NO: 64) | (SEQ ID NO: 85) |
| 47 | 6 | SYAIS | GIIPIFGTANYAQKFOG | SPLRGSGWYWHYYYGMDV | GGNKIGTKSVH | RDSNRPS | QVWDSSTVV |
| | | (SEQ ID NO: 3) | (SEQ ID NO: 15) | SEQ ID NO: 22) | (SEQ ID NO: 50) | (SEQ ID NO: 65) | (SEQ ID NO: 84) |
| 48 | 6 | SYAIS | GIIPIFGTANYAQKFQG | SPLRGSGWYWHYYYGMDV | GGNNIGSKSVH | QDRKRPS | QAWDSSTAV |
| | | (SEQ ID NO: 3) | (SEQ ID NO: 15) | (SEQ ID NO: 22) | (SEQ ID NO: 28) | SEQ ID NO: 66) | (SEQ ID NO: 86) |
| 49 | 6 | SYAIS | GIIPIFGTANYAQKFQG | SPLRGSGWYWHYYYGMDV | GGNNIRAKHVH | QDSKRPS | QVWDSSTVV |
| | | (SEQ ID NO: 3) | (SEQ ID NO: 15) | (SEQ ID NO: 22) | (SEQ ID NO: 47) | (SEQ ID NO: 64) | (SEQ ID NO: 84) |
| 50 | 6 | SYAIS | GIIPIFGTANYAQKFQG | SPLRGSGWYWHYYYGMDV | GGNNIGTKSVH | QDRKRPS | QVWDSSTEV |
| | | (SEQ ID NO: 3) | (SEQ ID NO: 15) | (SEQ ID NO: 22) | (SEQ ID NO: 118) | (SEQ ID NO: 66 | (SEQ ID NO: 87) |
| 51 | 6 | SYAIS | GIIPIFGTANYAQKFQG | SPLRGSGWYWHYYYGMDV | GGNKIGTKSVH | QDSKRPS | QAWGSSTVV |
| | | (SEQ ID NO: 3) | (SEQ ID NO: 15) | (SEQ ID NO: 22) | (SEQ ID NO: 50) | (SEQ ID NO: 64) | (SEQ ID NO: 88) |
| 52 | 12 | ELSMH | LVDPEDGETIYAEKFQG | TIGDDYFDY | QASQDISNYLN | DASNLET | QQYDNLPLT |
| | | (SEQ ID NO: 5) | (SEQ ID NO: 11) | (SEQ ID NO: 23) | (SEQ ID NO: 44) | (SEQ ID NO: 61) | (SEQ ID NO: 89) |
| 53 | 13 | ELSMH | GFDPEDGETIYAQKFQG | DIQGLAEFDY | TGTNTDVGAYIDVS | DVSKWPS | CSYAGNHSFS |
| | | (SEQ ID NO: 5) | (SEQ ID NO: 16) | (SEQ ID NO: 24) | (SEQ ID NO: 51) | (SEQ ID NO: 67) | (SEQ ID NO: 90) |
| 54 | 14 | TSGMGVG | HIWWDDDKYYNPYLKN | SAYGSTYGY | KASQSVDYDGDSFMN | TASILES | QQSNEDPWT |
| | | (SEQ ID NO: 6) | (SEQ ID NO: 17) | (SEQ ID NO: 25) | (SEQ ID NO: 52) | (SEQ ID NO: 68) | (SEQ ID NO: 91) |

TABLE 2
CDRs of mutant clones.

| | Heavy | Light | | | | | | |
|---|---|---|---|---|---|---|---|---|
| Clone | Chain | Chain | | | | | | |
| No. | Mutations | mutations | VH CDR1 | VH CDR2 | VHCDR3 | VL CDR1 | VL CDR2 | VLCDR3 |
| 6 | Parental | Parental | ELSMQ | GFDPDDGETMYAEKFQG | SPGYDFFDY | TGTSSDVGGYNYVS | EVSNRPS | QVWDSSNDLLI |
| | | | (SEQ ID NO: 1) | (SEQ ID NO: 7) | (SEQ ID NO: 18) | (SEQ ID NO: 30) | (SEQ ID NO: 55) | (SEQ ID NO: 71) |
| 55 | N297A | Parental | ELSMQ | GFDPDDGETMYAEKFQG | SPGYDFFDY | TGTSSDVGGYNYVS | EVSNRPS | QVWDSSNDLLI |
| | | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 56 | D54A | Parental | ELSMQ | GFDPDAGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 92) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 57 | D54S | Parental | ELSMQ | GFDPDSGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 93) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 58 | D54E | Parental | ELSMQ | GFDPDEGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 94) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 59 | G55A | Parental | ELSMQ | GFDPDDAETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 95) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 60 | R82aS | Parental | ELSMQ | GFDPDDGETMYAEKFQG | SPGYDFFDY | TGTSSDVGGYNYVS | EVSNRPS | QVWDSSNDLLI |
| | T83R | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | L89V | (mutations | | | | | | |
| | N297A | in heavy | | | | | | |
| | | chain | | | | | | |
| | | only) | | | | | | |
| 61 | D54A | Parental | ELSMQ | GFDPDAGETMYAEKFQG | SPGYDFFDY | TGTSSDVGGYNYVS | EVSNRPS | QVWDSSNDLLI |
| | R82aS | clone 6 | (SEQ ID NO: 1) | (SEQ ID NO: 92) | (SEQ ID NO: 18) | (SEQ ID NO: 30) | (SEQ ID NO: 55) | (SEQ ID NO: 71) |
| | T83R | (mutations | | | | | | |
| | L89V | in heavy | | | | | | |
| | N297A | chain | | | | | | |
| | | only) | | | | | | |
| 62 | D54S | Parental | ELSMQ | GFDPDSGETMYAEKFQG | SPGYDFFDY | TGTSSDVGGYNYVS | EVSNRPS | QVWDSSNDLLI |
| | R82aS | clone 6 | (SEQ ID NO: 1) | (SEQ ID NO: 93) | (SEQ ID NO: 18) | (SEQ ID NO: 30) | (SEQ ID NO: 55) | (SEQ ID NO: 71) |
| | T83R | (mutations | | | | | | |
| | L89V | in heavy | | | | | | |
| | N297A | chain | | | | | | |
| | | only) | | | | | | |
| 63 | D54E | Parental | ELSMQ | GFDPDEGETMYAEKFQG | SPGYDFFDY | TGTSSDVGGYNYVS | EVSNRPS | QVWDSSNDLLI |
| | R82aS | clone 6 | (SEQ ID NO: 1) | (SEQ ID NO: 94) | (SEQ ID NO: 18) | (SEQ ID NO: 30) | (SEQ ID NO: 55) | (SEQ ID NO: 71) |
| | T83R | (mutations | | | | | | |
| | L89V | in heavy | | | | | | |
| | N297A | chain | | | | | | |
| | | only) | | | | | | |
| 64 | G55A | Parental | ELSMQ | GFDPDDAETMYAEKFQG | SPGYDFFDY | TGTSSDVGGYNYVS | EVSNRPS | QVWDSSNDLLI |
| | R82as | clone 6 | (SEQ ID NO: 1) | (SEQ ID NO: 95) | (SEQ ID NO: 18) | (SEQ ID NO: 30) | (SEQ ID NO: 55) | (SEQ ID NO: 71) |
| | T83R | (mutations | | | | | | |
| | L89V | in heavy | | | | | | |
| | N297A | chain | | | | | | |
| | | only) | | | | | | |
| 65 | N297A | Germline | ELSMQ | GFDPDDGETMYAEKFQG | SPGYDFFDY | TGTSSDVGGYNYVS | EVSNRPS | QVWDSSNDLLI |
| | | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 66 | D54A | Germline | ELSMQ | GFDPDAGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 92) | (SEQ ID NO: 18) | (SEQ ID NO: 30) | (SEQ ID NO: 55) | (SEQ ID NO: 71) |
| | | graft | | | | | | |
| 67 | DS4S | Germline | ELSMQ | GFDPDSGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 93) | (SEQ ID NO: 18) | (SEQ ID NO: 30) | (SEQ ID NO: 55) | (SEQ ID NO: 71) |
| | | graft | | | | | | |
| 68 | D54E | Germline | ELSMQ | GFDPDEGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 94) | (SEQ ID NO: 18) | (SEQ ID NO: 30) | (SEQ ID NO: 55) | (SEQ ID NO: 71) |
| | | graft | | | | | | |
| 69 | G55A | Germline | ELSMQ | GFDPDDAETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 95) | (SEQ ID NO: 18) | (SEQ ID NO: 30) | (SEQ ID NO: 55) | (SEQ ID NO: 71) |
| | | graft | | | | | | |
| 70 | R82aS | Germline | ELSMQ | GFDPDDGETMYAEKFQG | SPGYDFFDY | TGTSSDVGGYNYVS | EVSNRPS | QVWDSSNDLLI |
| | T83R | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | L89V | graft | | | | | | |
| | N297A | | | | | | | |
| 71 | D54A | Germline | ELSMQ | GFDPDAGETMYAEKFQG | SPGYDFFDY | TGTSSDVGGYNYVS | EVSNRPS | QVWDSSNDLLI |
| | R82aS | 3-21 CDR | (SEQ ID NO: 1) | (SEQ ID NO: 92) | (SEQ ID NO: 18) | (SEQ ID NO: 30) | (SEQ ID NO: 55) | (SEQ ID NO: 71) |
| | T83R | graft | | | | | | |
| | L89V | | | | | | | |
| | N297A | | | | | | | |
| 72 | D54S | Germline | ELSMQ | GFDPDSGETMYAEKFQG | SPGYDFFDY | TGTSSDVGGYNYVS | EVSNRPS | QVWDSSNDLLI |
| | R82aS | 3-21 CDR | (SEQ ID NO: 1) | (SEQ ID NO: 93) | (SEQ ID NO: 18) | (SEQ ID NO: 30) | (SEQ ID NO: 55) | (SEQ ID NO: 71) |
| | T83R | graft | | | | | | |
| | L89V | | | | | | | |
| | N297A | | | | | | | |
| 73 | D54E | Germline | ELSMQ | GFDPDEGETMYAEKFQG | SPGYDFFDY | TGTSSDVGGYNYVS | EVSNRPS | QVWDSSNDLLI |
| | R82aS | 3-21 CDR | (SEQ ID NO: 1) | (SEQ ID NO: 94) | (SEQ ID NO: 18) | (SEQ ID NO: 30) | (SEQ ID NO: 55) | (SEQ ID NO: 71) |
| | T83R | graft | | | | | | |
| | L89V | | | | | | | |
| | N297A | | | | | | | |
| 74 | G55A | Germline | ELSMQ | GFDPDDAETMYAEKFQG | SPGYDFFDY | TGTSSDVGGYNYVS | EVSNRPS | QVWDSSNDLLI |
| | R82as | 3-21 CDR | (SEQ ID NO: 1) | (SEQ ID NO: 95) | (SEQ ID NO: 18) | (SEQ ID NO: 30) | (SEQ ID NO: 55) | (SEQ ID NO: 71) |
| | T83R | graft | | | | | | |
| | L89V | | | | | | | |
| | N297A | | | | | | | |
| 75 | N297A | Germline | ELSMQ | GFDPDDGETMYAEKFQG | SPGYDFFDY | GGNNIGSKSVH | DDSDRPS | QVWDSSNDLLI |
| | | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 76 | D54A | Germline | ELSMQ | GFDPDAGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 92) | (SEQ ID NO: 18) | (SEQ ID NO: 28) | (SEQ ID NO: 54) | (SEQ ID NO: 71) |
| | | 6 CDR3 | | | | | | |
| 77 | D54S | Germline | ELSMQ | GFDPDSGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 93) | (SEQ ID NO: 18) | (SEQ ID NO: 28) | (SEQ ID NO: 54) | (SEQ ID NO: 71) |
| | | 6 CDR3 | | | | | | |
| 78 | D54E | Germline | ELSMQ | GFDPDEGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 94) | (SEQ ID NO: 18) | (SEQ ID NO: 28) | (SEQ ID NO: 54) | (SEQ ID NO: 71) |
| | | 6 CDR3 | | | | | | |
| 79 | G55A | Germline | ELSMQ | GFDPDDAETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 95) | (SEQ ID NO: 18) | (SEQ ID NO: 28) | (SEQ ID NO: 54) | (SEQ ID NO: 71) |
| | | 6 CDR3 | | | | | | |
| 80 | R82aS | Germline | ELSMQ | GFDPDDGETMYAEKFQG | SPGYDFFDY | GGNNIGSKSVH | DDSDRPS | QVWDSSNDLLI |
| | T83R | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | L89V | 6 CDR3 | | | | | | |
| | N297A | | | | | | | |
| 81 | D54A | Germline | ELSMQ | GFDPDAGETMYAEKFQG | SPGYDFFDY | GGNNIGSKSVH | DDSDRPS | QVWDSSNDLLI |
| | R82aS | 3-21 clone | (SEQ ID NO: 1) | (SEQ ID NO: 92) | (SEQ ID NO: 18) | (SEQ ID NO: 28) | (SEQ ID NO: 54) | (SEQ ID NO: 71) |
| | T83R | 6 CDR3 | | | | | | |
| | L89V | | | | | | | |
| | N297A | | | | | | | |
| 82 | D54S | Germline | ELSMQ | GFDPDSGETMYAEKFQG | SPGYDFFDY | GGNNIGSKSVH | DDSDRPS | QVWDSSNDLLI |
| | R82aS | 3-21 clone | (SEQ ID NO: 1) | (SEQ ID NO: 93) | (SEQ ID NO: 18) | (SEQ ID NO: 28) | (SEQ ID NO: 54) | (SEQ ID NO: 71) |
| | T83R | 6 CDR3 | | | | | | |
| | L89V | | | | | | | |
| | N297A | | | | | | | |
| 83 | D54E | Germline | ELSMQ | GFDPDEGETMYAEKFQG | SPGYDFFDY | GGNNIGSKSVH | DDSDRPS | QVWDSSNDLLI |
| | R82aS | 3-21 clone | (SEQ ID NO: 1) | (SEQ ID NO: 94) | (SEQ ID NO: 18) | (SEQ ID NO: 28) | (SEQ ID NO: 54) | (SEQ ID NO: 71) |
| | T83R | 6 CDR3 | | | | | | |
| | L89V | | | | | | | |
| | N297A | | | | | | | |
| 84 | G55A | Germline | ELSMQ | GFDPDDAETMYAEKFQG | SPGYDFFDY | GGNNIGSKSVH | DDSDRPS | QVWDSSNDLLI |
| | R82aS | 3-21 clone | (SEQ ID NO: 1) | (SEQ ID NO: 95) | (SEQ ID NO: 18) | (SEQ ID NO: 28) | (SEQ ID NO: 54) | (SEQ ID NO: 71) |
| | T83R | 6 CDR3 | | | | | | |
| | L89V | | | | | | | |
| | N297A | | | | | | | |
| 85 | E1Q | Parental | ELSMQ | GFDPDDGETMYAEKFQG | SPGYDFFDY | TGTSSDVGGYNYVS | EVSNRPS | QVWDSSNDLLI |
| | L67V | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | V69M | (mutations | | | | | | |
| | V78A | in heavy | | | | | | |
| | R82aS | chain | | | | | | |
| | T83R | only) | | | | | | |
| | L89V | | | | | | | |
| | F91Y | | | | | | | |
| | N297A | | | | | | | |
| 86 | D54A | Parental | ELSMQ | GFDPDAGETMYAEKFQG | SPGYDFFDY | TGTSSDVGGYNYVS | EVSNRPS | QVWDSSNDLLI |
| | E10 | clone 6 | (SEQ ID NO: 1) | (SEQ ID NO: 92) | (SEQ ID NO: 18) | (SEQ ID NO: 30) | (SEQ ID NO: 55) | (SEQ ID NO: 71) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 87 | D54S | Parental | ELSMQ | GFDPDSGETMYAEKFQG | SPGYDFFDY | TGTSSDVGGYNYVS | EVSNRPS | QVWDSSNDLLI |
| | E1Q | clone 6 | (SEQ ID NO: 1) | (SEQ ID NO: 93) | (SEQ ID NO: 18) | (SEQ ID NO: 30) | (SEQ ID NO: 55) | (SEQ ID NO: 71) |
| | L67V | (mutations | | | | | | |
| | V69M | in heavy | | | | | | |
| | V78A | chain | | | | | | |
| | R82as | only) | | | | | | |
| | T83R | | | | | | | |
| | L89V | | | | | | | |
| | F91Y | | | | | | | |
| | N297A | | | | | | | |
| 88 | D54E | Parental | ELSMQ | GFDPDEGETMYAEKFQG | SPGYDFFDY | TGTSSDVGGYNYVS | EVSNRPS | QVWDSSNDLLI |
| | E1Q | clone 6 | (SEQ ID NO: 1) | (SEQ ID NO: 94) | (SEQ ID NO: 18) | (SEQ ID NO: 30) | (SEQ ID NO: 55) | (SEQ ID NO: 71) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 89 | G55A | Parental | ELSMQ | GFDPDDAETMYAEKFQG | SPGYDFFDY | TGTSSDVGGYNYVS | EVSNRPS | QVWDSSNDLLI |
| | E1Q | clone 6 | (SEQ ID NO: 1) | (SEQ ID NO: 95) | (SEQ ID NO: 18) | (SEQ ID NO: 30) | (SEQ ID NO: 55) | (SEQ ID NO: 71) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 90 | E10 | Germline | ELSMQ | GFDPDDGETMYAEKFQG | SPGYDFFDY | TGTSSDVGGYNYVS | EVSNRPS | QVWDSSNDLLI |
| | L67V | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | V69M | graft | | | | | | |
| | V78A | | | | | | | |
| | R82aS | | | | | | | |
| | T83R | | | | | | | |
| | L89V | | | | | | | |
| | F91Y | | | | | | | |
| | N297A | | | | | | | |
| 91 | D54A | Germline | ELSMQ | GFDPDAGETMYAEKFQG | SPGYDFFDY | TGTSSDVGGYNYVS | EVSNRPS | QVWDSSNDLLI |
| | E1Q | 3-21 CDR | (SEQ ID NO: 1) | (SEQ ID NO: 92) | (SEQ ID NO: 18) | (SEQ ID NO: 30) | (SEQ ID NO: 55) | (SEQ ID NO: 71) |
| | L67V | graft | | | | | | |
| | V69M | | | | | | | |
| | V78A | | | | | | | |
| | R82aS | | | | | | | |
| | T83R | | | | | | | |
| | L89V | | | | | | | |
| | F91Y | | | | | | | |
| | N297A | | | | | | | |
| 92 | D54S | Germline | ELSMQ | GFDPDSGETMYAEKFQG | SPGYDFFDY | TGTSSDVGGYNYVS | EVSNRPS | QVWDSSNDLLI |
| | E10 | 3-21 CDR | (SEQ ID NO: 1) | (SEQ ID NO: 93) | (SEQ ID NO: 18) | (SEQ ID NO: 30) | (SEQ ID NO: 55) | (SEQ ID NO: 71) |
| | L67V | graft | | | | | | |
| | V69M | | | | | | | |
| | V78A | | | | | | | |
| | R82aS | | | | | | | |
| | T83R | | | | | | | |
| | L89V | | | | | | | |
| | F91Y | | | | | | | |
| | N297A | | | | | | | |
| 93 | D54E | Germline | ELSMQ | GFDPDEGETMYAEKFQG | SPGYDFFDY | TGTSSDVGGYNYVS | EVSNRPS | QVWDSSNDLLI |
| | E1Q_ | 3-21 CDR | (SEQ ID NO: 1) | (SEQ ID NO: 94) | (SEQ ID NO: 18) | (SEQ ID NO: 30) | (SEQ ID NO: 55) | (SEQ ID NO: 71) |
| | L67V_ | graft | | | | | | |
| | V69M | | | | | | | |
| | V78A_ | | | | | | | |
| | R82aS | | | | | | | |
| | T83R | | | | | | | |
| | L89V | | | | | | | |
| | F91Y_ | | | | | | | |
| | N297A | | | | | | | |
| 94 | G55A | Germline | ELSMQ | GFDPDDAETMYAEKFQG | SPGYDFFDY | TGTSSDVGGYNYVS | EVSNRPS | QVWDSSNDLLI |
| | E1Q_ | 3-21 CDR | (SEQ ID NO: 1) | (SEQ ID NO: 95) | (SEQ ID NO: 18) | (SEQ ID NO: 30) | (SEQ ID NO: 55) | (SEQ ID NO: 71) |
| | L67V | graft | | | | | | |
| | V69M | | | | | | | |
| | V78A | | | | | | | |
| | R82aS_ | | | | | | | |
| | T83R | | | | | | | |
| | L89V | | | | | | | |
| | F91Y | | | | | | | |
| | N297A | | | | | | | |
| 95 | E1Q | Germline | ELSMQ | GFDPDDGETMYAEKFQG | SPGYDFFDY | GGNNIGSKSVH | DDSDRPS | QVWDSSNDLLI |
| | L67V_ | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | V69M | 6 CDR3 | | | | | | |
| | V78A | | | | | | | |
| | R82aS | | | | | | | |
| | T83R_ | | | | | | | |
| | L89V | | | | | | | |
| | F91Y | | | | | | | |
| | N297A | | | | | | | |
| 96 | D54A | Germline | ELSMQ | GFDPDAGETMYAEKFQG | SPGYDFFDY | GGNNIGSKSVH | DDSDRPS | QVWDSSNDLLI |
| | E1Q_ | 3-21 clone | (SEQ ID NO: 1) | (SEQ ID NO: 92) | (SEQ ID NO: 18) | (SEQ ID NO: 28) | (SEQ ID NO: 54) | (SEQ ID NO: 71) |
| | L67V | 6 CDR3 | | | | | | |
| | V69M | | | | | | | |
| | V78A | | | | | | | |
| | R82aS_ | | | | | | | |
| | T83R | | | | | | | |
| | L89V | | | | | | | |
| | F91Y | | | | | | | |
| | N297A | | | | | | | |
| 97 | D54S | Germline | ELSMQ | GFDPDSGETMYAEKFQG | SPGYDFFDY | GGNNIGSKSVH | DDSDRPS | QVWDSSNDLLI |
| | E1Q_ | 3-21 clone | (SEQ ID NO: 1) | (SEQ ID NO: 93) | (SEQ ID NO: 18) | (SEQ ID NO: 28) | (SEQ ID NO: 54) | (SEQ ID NO: 71) |
| | L67V | 6 CDR3 | | | | | | |
| | V69M | | | | | | | |
| | V78A | | | | | | | |
| | R82aS_ | | | | | | | |
| | T83R | | | | | | | |
| | L89V | | | | | | | |
| | F91Y | | | | | | | |
| | N297A | | | | | | | |
| 98 | D54E | Germline | ELSMQ | GFDPDEGETMYAEKFQG | SPGYDFFDY | GGNNIGSKSVH | DDSDRPS | QVWDSSNDLLI |
| | E1Q_ | 3-21 clone | (SEQ ID NO: 1) | (SEQ ID NO: 94) | (SEQ ID NO: 18) | (SEQ ID NO: 28) | (SEQ ID NO: 54) | (SEQ ID NO: 71) |
| | L67V | 6 CDR3 | | | | | | |
| | V69M | | | | | | | |
| | V78A | | | | | | | |
| | R82aS | | | | | | | |
| | T83R | | | | | | | |
| | L89V | | | | | | | |
| | F91Y | | | | | | | |
| | N297A | | | | | | | |
| 99 | G55A | Germline | ELSMQ | GFDPDDAETMYAEKFQG | SPGYDFFDY | GGNNIGSKSVH | DDSDRPS | QVWDSSNDLLI |
| | E11_ | 3-21 clone | (SEQ ID NO: 1) | (SEQ ID NO: 95) | (SEQ ID NO: 18) | (SEQ ID NO: 28) | (SEQ ID NO: 54) | (SEQ ID NO: 71) |
| | L67V | 6 CDR3 | | | | | | |
| | V69M | | | | | | | |
| | V78A | | | | | | | |
| | R82aS_ | | | | | | | |
| | T83R | | | | | | | |
| | L89V | | | | | | | |
| | F91Y | | | | | | | |
| | N297A | | | | | | | |
| 100 | E1Q | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 101 | L67V | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 102 | V69M | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 103 | V78A | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 104 | R82aS | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 105 | T83R | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 106 | L89V | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 107 | F91Y | Parental | ELSMQ | GFDPDDGETMYAEKFQG | SPGYDFFDY | TGTSSDVGGYNYVS | EVSNRPS | QVWDSSNDLLI |
| | N297A | clone 6 | (SEQ ID NO: 1) | (SEQ ID NO: 7) | (SEQ ID NO: 18) | (SEQ ID NO: 30) | (SEQ ID NO: 55) | (SEQ ID NO: 71) |
| | | (mutations | | | | | | |
| | | in heavy | | | | | | |
| | | chain | | | | | | |
| | | only) | | | | | | |
| 108 | E1Q | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 109 | L67V | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 110 | V69M | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 111 | V78A | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 112 | R82aS | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 113 | T83R | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 114 | L89V | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 115 | F91Y | Germline | ELSMQ | GFDPDDGETMYAEKFQG | SPGYDFFDY | TGTSSDVGGYNYVS | EVSNRPS | QVWDSSNDLLI |
| | N297A | 3-21 CDR | (SEQ ID NO: 1) | (SEQ ID NO: 7) | (SEQ ID NO: 18) | (SEQ ID NO: 30) | (SEQ ID NO: 55) | (SEQ ID NO: 71) |
| | | graft | | | | | | |
| 116 | E1Q | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 117 | L67V | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 118 | V69M | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 119 | V78A | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 120 | R82aS | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 121 | T83R | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 122 | L89V | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 123 | F91Y | Germline | ELSMQ | GFDPDDGETMYAEKFQG | SPGYDFFDY | GGNNIGSKSVH | DDSDRPS | QVWDSSNDLLI |
| | N297A | 3-21 clone | (SEQ ID NO: 1) | (SEQ ID NO: 7) | (SEQ ID NO: 18) | (SEQ ID NO: 28) | (SEQ ID NO: 54) | (SEQ ID NO: 71) |
| | | 6 CDR3 | | | | | | |
| 15 | Parental | Parental | ELSMQ | GFDPDDGETMYAEKFQG | SPGYDFFDY | GGDNIGSKSVH | DDSDRPS | QVWDSSNDLLI |
| | | | (SEQ ID NO: 1) | (SEQ ID NO: 7) | (SEQ ID NO: 18) | (SEQ ID NO: 27) | (SEQ ID NO: 54) | (SEQ ID NO: 71) |
| 124 | N297A | Parental | ELSMQ | GFDPDDGETMYAEKFQG | SPGYDFFDY | GGDNIGSKSVH | DDSDRPS | QVWDSSNDLLI |
| | | clone 15 | (SEQ ID NO: 1) | (SEQ ID NO: 7) | (SEQ ID NO: 18) | (SEQ ID NO: 27) | (SEQ ID NO: 54) | (SEQ ID NO: 71) |
| | | (mutations | | | | | | |
| | | in heavy | | | | | | |
| | | chain | | | | | | |
| | | only) | | | | | | |
| 125 | D54A | Parental | ELSMQ | GFDPDAGETMYAEKFQG | SPGYDFFDY | GGDNIGSKSVH | DDSDRPS | QVWDSSNDLLI |
| | N297A | clone 15 | (SEQ ID NO: 1) | (SEQ ID NO: 92) | (SEQ ID NO: 18) | (SEQ ID NO: 27) | (SEQ ID NO: 54) | (SEQ ID NO: 71) |
| | | (mutations | | | | | | |
| | | in heavy | | | | | | |
| | | chain | | | | | | |
| | | only) | | | | | | |
| 126 | D54S | Parental | ELSMQ | GFDPDSGETMYAEKFQG | SPGYDFFDY | GGDNIGSKSVH | DDSDRPS | QVWDSSNDLLI |
| | N297A | clone 15 | (SEQ ID NO: 1) | (SEQ ID NO: 93) | (SEQ ID NO: 18) | (SEQ ID NO: 27) | (SEQ ID NO: 54) | (SEQ ID NO: 71) |
| | | (mutations | | | | | | |
| | | in heavy | | | | | | |
| | | chain | | | | | | |
| | | only) | | | | | | |
| 127 | D54E | Parental | ELSMQ | GFDPDEGETMYAEKFQG | SPGYDFFDY | GGDNIGSKSVH | DDSDRPS | QVWDSSNDLLI |
| | N297A | clone 15 | (SEQ ID NO: 1) | (SEQ ID NO: 94) | (SEQ ID NO: 18) | (SEQ ID NO: 27) | (SEQ ID NO: 54) | (SEQ ID NO: 71) |
| | | (mutations | | | | | | |
| | | in heavy | | | | | | |
| | | chain | | | | | | |
| | | only) | | | | | | |
| 128 | G55A | Parental | ELSMQ | GFDPDDAETMYAEKFQG | SPGYDFFDY | GGDNIGSKSVH | DDSDRPS | QVWDSSNDLLI |
| | N297A | clone 15 | (SEQ ID NO: 1) | (SEQ ID NO: 95) | (SEQ ID NO: 18) | (SEQ ID NO: 27) | (SEQ ID NO: 54) | (SEQ ID NO: 71) |
| | | (mutations | | | | | | |
| | | in heavy | | | | | | |
| | | chain | | | | | | |
| | | only) | | | | | | |
| 129 | R82aS | Parental | ELSMQ | GFDPDDGETMYAEKFQG | SPGYDFFDY | GGDNIGSKSVH | DDSDRPS | QVWDSSNDLLI |
| | T83R | clone 15 | (SEQ ID NO: 1) | (SEQ ID NO: 7) | (SEQ ID NO: 18) | (SEQ ID NO: 27) | (SEQ ID NO: 54) | (SEQ ID NO: 71) |
| | L89V | (mutations | | | | | | |
| | N297A | in heavy | | | | | | |
| | | chain | | | | | | |
| | | only) | | | | | | |
| 130 | D54A | Parental | ELSMQ | GFDPDAGETMYAEKFQG | SPGYDFFDY | GGDNIGSKSVH | DDSDRPS | QVWDSSNDLLJ |
| | R82aS | clone 15 | (SEQ ID NO: 1) | (SEQ ID NO: 92) | (SEQ ID NO: 18) | (SEQ ID NO: 27) | (SEQ ID NO: 54) | (SEQ ID NO: 71) |
| | T83R | (mutations | | | | | | |
| | L89V | in heavy | | | | | | |
| | N297A | chain | | | | | | |
| | | only) | | | | | | |
| 131 | D54S | Parental | ELSMQ | GFDPDSGETMYAEKFQG | SPGYDFFDY | GGDNIGSKSVH | DDSDRPS | QVWDSSNDLLI |
| | R82aS | clone 15 | (SEQ ID NO: 1) | (SEQ ID NO: 93) | (SEQ ID NO: 18) | (SEQ ID NO: 27) | (SEQ ID NO: 54) | (SEQ ID NO: 71) |
| | T83R | (mutations | | | | | | |
| | L89V | in heavy | | | | | | |
| | N297A | chain | | | | | | |
| | | only) | | | | | | |
| 132 | D54E | Parental | ELSMQ | GFDPDEGETMYAEKFQG | SPGYDFFDY | GGDNIGSKSVH | DDSDRPS | QVWDSSNDLLI |
| | R82aS | clone 15 | (SEQ ID NO: 1) | (SEQ ID NO: 94) | (SEQ ID NO: 18) | (SEQ ID NO: 27) | (SEQ ID NO: 54) | (SEQ ID NO: 71) |
| | T83R | (mutations | | | | | | |
| | L89V | in heavy | | | | | | |
| | N297A | chain | | | | | | |
| | | only) | | | | | | |
| 133 | G55A | Parental | ELSMQ | GFDPDDAETMYAEKFQG | SPGYDFFDY | GGDNIGSKSVH | DDSDRPS | QVWDSSNDLLI |
| | R82aS | clone 15 | (SEQ I NO: 1) | (SEQ ID NO: 95) | (SEQ ID NO: 18) | (SEQ ID NO: 27) | (SEQ ID NO: 54) | (SEQ ID NO: 71) |
| | T83R | (mutations | | | | | | |
| | L89V | in heavy | | | | | | |
| | N297A | chain | | | | | | |
| | | only) | | | | | | |
| 134 | N297A | Parental | ELSMQ | GFDPDDGETMYAEKFQG | SPGYDFFDY | GGDNIGSKSVH | DDSDRPS | QVWDSSNDLLI |
| | | clone 15 | (SEQ ID NO: 1) | (SEQ ID NO: 7) | (SEQ ID NO: 18) | (SEQ ID NO: 27) | (SEQ ID NO: 54) | (SEQ ID NO: 71) |
| | | mutated | | | | | | |
| | | at: | | | | | | |
| | | E3V | | | | | | |
| | | N76S | | | | | | |
| 135 | D54A | Parental | ELSMQ | GFDPDAGETMYAEKFQG | SPGYDFFDY | GGDNIGSKSVH | DDSDRPS | QVWDSSNDLLI |
| | N297A | clone 15 | (SEQ ID NO: 1) | (SEQ ID NO: 92) | (SEQ ID NO: 18) | (SEQ ID NO: 27) | (SEQ ID NO: 54) | (SEQ ID NO: 71) |
| | | mutated | | | | | | |
| | | at: | | | | | | |
| | | E3V | | | | | | |
| | | N76S | | | | | | |
| 136 | D54S | Parental | ELSMQ | GFDPDSGETMYAEKFQG | SPGYDFFDY | GGDNIGSKSVH | DDSDRPS | QVWDSSNDLLI |
| | N297A | clone 15 | (SEQ ID NO: 1) | (SEQ ID NO: 93) | (SEQ ID NO: 18) | (SEQ ID NO: 27) | (SEQ ID NO: 54) | (SEQ ID NO: 71) |
| | | mutated | | | | | | |
| | | at: | | | | | | |
| | | E3V | | | | | | |
| | | N76S | | | | | | |
| 137 | D54E | Parental | ELSMQ | GFDPDEGETMYAEKFQG | SPGYDFFDY | GGDNIGSKSVH | DDSDRPS | QVWDSSNDLLI |
| | N297A | clone 15 | (SEQ ID NO: 1) | (SEQ ID NO: 94) | (SEQ ID NO: 18) | (SEQ ID NO: 27) | (SEQ ID NO: 54) | (SEQ ID NO: 71) |
| | | mutated | | | | | | |
| | | at: | | | | | | |
| | | E3V | | | | | | |
| | | N76S | | | | | | |
| 138 | G55A | Parental | ELSMQ | GFDPDDAETMYAEKFQG | SPGYDFFDY | GGDNIGSKSVH | DDSDRPS | QVWDSSNDLLI |
| | N297A | clone 15 | (SEQ ID NO: 1) | (SEQ ID NO: 95) | (SEQ ID NO: 18) | (SEQ ID NO: 27) | (SEQ ID NO: 54) | (SEQ ID NO: 71) |
| | | mutated | | | | | | |
| | | at: | | | | | | |
| | | E3V | | | | | | |
| | | N76S | | | | | | |
| 139 | R82aS | Parental | ELSMQ | GFDPDDGETMYAEKFQG | SPGYDFFDY | GGDNIGSKSVH | DDSDRPS | QVWDSSNDLLI |
| | T83R | clone 15 | (SEQ ID NO: 1) | (SEQ ID NO: 7) | (SEQ ID NO: 18) | (SEQ ID NO: 27) | (SEQ ID NO: 54) | (SEQ ID NO: 71) |
| | L89V | mutated | | | | | | |
| | N297A | at: | | | | | | |
| | | E3V | | | | | | |
| | | N76S | | | | | | |
| 140 | D54A | Parental | ELSMQ | GFDPDAGETMYAEKFQG | SPGYDFFDY | GGDNIGSKSVH | DDSDRPS | QVWDSSNDLLI |
| | R82aS | clone 15 | (SEQ ID NO: 1) | (SEQ ID NO: 92) | (SEQ ID NO: 18) | (SEQ ID NO: 27) | (SEQ ID NO: 54) | (SEQ ID NO: 71) |
| | T83R | mutated | | | | | | |
| | L89V | at: | | | | | | |
| | N297A | E3V | | | | | | |
| | | N76S | | | | | | |
| 141 | D54S | Parental | ELSMQ | GFDPDSGETMYAEKFQG | SPGYDFFDY | GGDNIGSKSVH | DDSDRPS | QVWDSSNDLLI |
| | R82aS | clone 15 | (SEQ ID NO: 1) | (SEQ ID NO: 93) | (SEQ ID NO: 18) | (SEQ ID NO: 27) | (SEQ ID NO: 54) | (SEQ ID NO: 71) |
| | T83R | mutated | | | | | | |
| | L89V | at: | | | | | | |
| | N297A | E3V | | | | | | |
| | | N76S | | | | | | |
| 142 | D54E | Parental | ELSMQ | GFDPDEGETMYAEKFQG | SPGYDFFDY | GGDNIGSKSVH | DDSDRPS | QVWDSSNDLLI |
| | R82aS | clone 15 | (SEQ ID NO: 1) | (SEQ ID NO: 94) | (SEQ ID NO: 18) | (SEQ ID NO: 27) | (SEQ ID NO: 54) | (SEQ ID NO: 71) |
| | T83R | mutated | | | | | | |
| | L89V | at: | | | | | | |
| | N297A | E3V | | | | | | |
| | | N76S | | | | | | |
| 143 | G55A | Parental | ELSMQ | GFDPDDAETMYAEKFQG | SPGYDFFDY | GGDNIGSKSVH | DDSDRPS | QVWDSSNDLLI |
| | R82aS | clone 15 | (SEQ ID NO: 1) | (SEQ ID NO: 95) | (SEQ ID NO: 18) | (SEQ ID NO: 27) | (SEQ ID NO: 54) | (SEQ ID NO: 71) |
| | T83R | mutated | | | | | | |
| | L89V | at: | | | | | | |
| | N297A | E3V | | | | | | |
| | | N76S | | | | | | |
| 144 | E1Q | Parental | ELSMQ | GFDPDDGETMYAEKFQG | SPGYDFFDY | GGDNIGSKSVH | DDSDRPS | QVWDSSNDLLI |
| | L67V | clone 15 | (SEQ ID NO: 1) | (SEQ ID NO: 7) | (SEQ ID NO: 18) | (SEQ ID NO: 27) | (SEQ ID NO: 54) | (SEQ ID NO: 71) |
| | V69M | (mutations | | | | | | |
| | V78A | in heavy | | | | | | |
| | R82aS | chain | | | | | | |
| | T83R | only) | | | | | | |
| | L89V | | | | | | | |
| | F91Y | | | | | | | |
| | N297A | | | | | | | |
| 145 | D54A | Parental | ELSMQ | GFDPDAGETMYAEKFQG | SPGYDFFDY | GGDNIGSKSVH | DDSDRPS | QVWDSSNDLLI |
| | E1Q | clone 15 | (SEQ ID NO: 1) | (SEQ ID NO: 92) | (SEQ ID NO: 18) | (SEQ ID NO: 27) | (SEQ ID NO: 54) | (SEQ ID NO: 71) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 146 | D545 | Parental | ELSMQ | GFDPDSGETMYAEKFQG | SPGYDFFDY | GGDNIGSKSVH | DDSDRPS | QVWDSSNDLLI |
| | E1Q | clone 15 | (SEQ ID NO: 1) | (SEQ ID NO: 93) | (SEQ ID NO: 18) | (SEQ ID NO: 27) | (SEQ ID NO: 54) | (SEQ ID NO: 71) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 147 | D54E | Parental | ELSMQ | GFDPDEGETMYAEKFQG | SPGYDFFDY | GGDNIGSKSVH | DDSDRPS | QVWDSSNDLLI |
| | E1Q | clone 15 | (SEQ ID NO: 1) | (SEQ ID NO: 94) | (SEQ ID NO: 18) | (SEQ ID NO: 27) | (SEQ ID NO: 54) | (SEQ ID NO: 71) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 148 | G55A | Parental | ELSMQ | GFDPDDAETMYAEKFQG | SPGYDFFDY | GGDNIGSKSVH | DDSDRPS | QVWDSSNDLLI |
| | E1Q | clone 15 | (SEQ ID NO: 1) | (SEQ ID NO: 95) | (SEQ ID NO: 18) | (SEQ ID NO: 27) | (SEQ ID NO: 54) | (SEQ ID NO: 71) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 149 | E10 | Parental | ELSMQ | GFDPDDGETMYAEKFQG | SPGYDFFDY | GGDNIGSKSVH | DDSDRPS | QVWDSSNDLLI |
| | L67V | clone 15 | (SEQ ID NO: 1) | (SEQ ID NO: 7) | (SEQ ID NO: 18) | (SEQ ID NO: 27) | (SEQ ID NO: 54) | (SEQ ID NO: 71) |
| | V69M | mutated | | | | | | |
| | V78A | at: | | | | | | |
| | R82aS | E3V | | | | | | |
| | T83R | N76S | | | | | | |
| | L89V | | | | | | | |
| | F91Y | | | | | | | |
| | N297A | | | | | | | |
| 150 | D54Ah | Parental | ELSMQ | GFDPDAGETMYAEKFQG | SPGYDFFDY | GGDNIGSKSVH | DDSDRPS | QVWDSSNDLLI |
| | E1Q | clone 15 | (SEQ ID NO: 1) | (SEQ ID NO: 92) | (SEQ ID NO: 18) | (SEQ ID NO: 27) | (SEQ ID NO: 54) | (SEQ ID NO: 71) |
| | L67V | mutated | | | | | | |
| | V69M | at: | | | | | | |
| | V78A | E3V | | | | | | |
| | R82aS | N76S | | | | | | |
| | T83R | | | | | | | |
| | L89V | | | | | | | |
| | F91Y | | | | | | | |
| | N297A | | | | | | | |
| 151 | D54S | Parental | ELSMQ | GFDPDSGETMYAEKFQG | SPGYDFFDY | GGDNIGSKSVH | DDSDRPS | QVWDSSNDLLI |
| | E1Q | clone 15 | (SEQ ID NO: 1) | (SEQ ID NO: 93) | (SEQ ID NO: 18) | (SEQ ID NO: 27) | (SEQ ID NO: 54) | (SEQ ID NO: 71) |
| | L67V | mutated | | | | | | |
| | V69M | at: | | | | | | |
| | V78A | E3V | | | | | | |
| | R82aS | N76S | | | | | | |
| | T83R | | | | | | | |
| | L89V | | | | | | | |
| | F91Y | | | | | | | |
| | N297A | | | | | | | |
| 152 | D54E | Parental | ELSMQ | GFDPDEGETMYAEKFQG | SPGYDFFDY | GGDNIGSKSVH | DDSDRPS | QVWDSSNDLLI |
| | E1Q | clone 15 | (SEQ ID NO: 1) | (SEQ ID NO: 94) | (SEQ ID NO: 18) | (SEQ ID NO: 27) | (SEQ ID NO: 54) | (SEQ ID NO: 71) |
| | L67V | mutated | | | | | | |
| | V69M | at: | | | | | | |
| | V78A | E3V | | | | | | |
| | R82aS | N76S | | | | | | |
| | T83R | | | | | | | |
| | L89V | | | | | | | |
| | F91Y | | | | | | | |
| | N297A | | | | | | | |
| 153 | G55A | Parental | ELSMQ | GFDPDDAETMYAEKFQG | SPGYDFFDY | GGDNIGSKSVH | DDSDRPS | QVWDSSNDLLI |
| | E1Q | clone 15 | (SEQ ID NO: 1) | (SEQ ID NO: 95) | (SEQ ID NO: 18) | (SEQ ID NO: 27) | (SEQ ID NO: 54) | (SEQ ID NO: 71) |
| | L67V | mutated | | | | | | |
| | V69M | at: | | | | | | |
| | V78A | E3V | | | | | | |
| | R82aS | N765 | | | | | | |
| | T83R | | | | | | | |
| | L89V | | | | | | | |
| | F91Y | | | | | | | |
| | N297A | | | | | | | |
| 154 | E1Q | Parental | ELSMQ | GFDPDDGETMYAEKFQG | SPGYDFFDY | GGDNIGSKSVH | DDSDRPS | QVWDSSNDLLI |
| | N297A | clone 15 | (SEQ ID NO: 1) | (SEQ ID NO: 7) | (SEQ ID NO: 18) | (SEQ ID NO: 27) | (SEQ ID NO: 54) | (SEQ ID NO: 71) |
| | | (mutations | | | | | | |
| | | in heavy | | | | | | |
| | | chain | | | | | | |
| | | only) | | | | | | |
| 155 | L67V | Parental | ELSMQ | GFDPDDGETMYAEKFQG | SPGYDFFDY | GGDNIGSKSVH | DDSDRPS | QVWDSSNDLLI |
| | N297A | clone 15 | (SEQ ID NO: 1) | (SEQ ID NO: 7) | (SEQ ID NO: 18) | (SEQ ID NO: 27) | (SEQ ID NO: 54) | (SEQ ID NO: 71) |
| | | (mutations | | | | | | |
| | | in heavy | | | | | | |
| | | chain | | | | | | |
| | | only) | | | | | | |
| 156 | V69M | Parental | ELSMQ | GFDPDDGETMYAEKFQG | SPGYDFFDY | GGDNIGSKSVH | DDSDRPS | QVWDSSNDLLI |
| | N297A | clone 15 | (SEQ ID NO: 1) | (SEQ ID NO: 7) | (SEQ ID NO: 18) | (SEQ ID NO: 27) | (SEQ ID NO: 54) | (SEQ ID NO: 71) |
| | | (mutations | | | | | | |
| | | in heavy | | | | | | |
| | | chain | | | | | | |
| | | only) | | | | | | |
| 157 | V78A | Parental | ELSMQ | GFDPDDGETMYAEKFQG | SPGYDFFDY | GGDNIGSKSVH | DDSDRPS | QVWDSSNDLLI |
| | N297A | clone 15 | (SEQ ID NO: 1) | (SEQ ID NO: 7) | (SEQ ID NO: 18) | (SEQ ID NO: 27) | (SEQ ID NO: 54) | (SEQ ID NO: 71) |
| | | (mutations | | | | | | |
| | | in heavy | | | | | | |
| | | chain | | | | | | |
| | | only) | | | | | | |
| 158 | R82aS | Parental | ELSMQ | GFDPDDGETMYAEKFQG | SPGYDFFDY | GGDNIGSKSVH | DDSDRPS | QVWDSSNDLLI |
| | N297A | clone 15 | (SEQ NO: 1) | (SEQ ID NO: 7) | (SEQ ID NO: 18) | (SEQ ID NO: 27) | (SEQ ID NO: 54) | (SEQ ID NO: 71) |
| | | (mutations | | | | | | |
| | | in heavy | | | | | | |
| | | chain | | | | | | |
| | | only) | | | | | | |
| 159 | T83R | Parental | ELSMQ | GFDPDDGETMYAEKFQG | SPGYDFFDY | GGDNIGSKSVH | DDSDRPS | QVWDSSNDLLI |
| | N297A | clone 15 | (SEQ ID NO: 1) | (SEQ ID NO: 7) | (SEQ ID NO: 18) | (SEQ ID NO: 27) | (SEQ ID NO: 54) | (SEQ ID NO: 71) |
| | | (mutations | | | | | | |
| | | in heavy | | | | | | |
| | | chain | | | | | | |
| | | only) | | | | | | |
| 160 | L89V | Parental | ELSMQ | GFDPDDGETMYAEKFQG | SPGYDFFDY | GGDNIGSKSVH | DDSDRPS | QVWDSSNDLLI |
| | N297A | clone 15 | (SEQ ID NO: 1) | (SEQ ID NO: 7) | (SEQ ID NO: 18) | (SEQ ID NO: 27) | (SEQ ID NO: 54) | (SEQ ID NO: 71) |
| | | (mutations | | | | | | |
| | | in heavy | | | | | | |
| | | chain | | | | | | |
| | | only) | | | | | | |
| 161 | F91Y | Parental | ELSMQ | GFDPDDGETMYAEKFQG | SPGYDFFDY (SEQ | GGDNIGSKSVH | DDSDRPS | QVWDSSNDLLI |
| | N297A | clone 15 | (SEQ ID NO: 1) | (SEQ ID NO: 7) | ID NO: 18) | (SEQ ID NO: 27) | (SEQ ID NO: 54) | (SEQ ID NO: 71) |
| | | mutations | | | | | | |
| | | in heavy | | | | | | |
| | | chain | | | | | | |
| | | only) | | | | | | |
| 162 | E1Q | Parental | ELSMQ | GFDPDDGETMYAEKFQG | SPGYDFFDY (SEQ | GGDNIGSKSVH | DDSDRPS | QVWDSSNDLLI |
| | N297A | clone 15 | (SEQ ID NO: 1) | (SEQ ID NO: 7) | ID NO: 18) | (SEQ ID NO: 27) | (SEQ ID NO: 54) | (SEQ ID NO: 71) |
| | | mutated | | | | | | |
| | | at: | | | | | | |
| | | E3V | | | | | | |
| | | N76S | | | | | | |
| 163 | L67V | Parental | ELSMQ | GFDPDDGETMYAEKFQG | SPGYDFFDY (SEQ | GGDNIGSKSVH | DDSDRPS | QVWDSSNDLLI |
| | N297A | clone 15 | (SEQ ID NO: 1) | (SEQ ID NO: 7) | ID NO: 18) | (SEQ ID NO: 27) | (SEQ ID NO: 54) | (SEQ ID NO: 71) |
| | | mutated | | | | | | |
| | | at: | | | | | | |
| | | E3V | | | | | | |
| | | N76S | | | | | | |
| 164 | V69M | Parental | ELSMQ | GFDPDDGETMYAEKFQG | SPGYDFFDY (SEQ | GGDNIGSKSVH | DDSDRPS | QVWDSSNDLLI |
| | N297A | clone 15 | (SEQ ID NO: 1) | (SEQ ID NO: 7) | ID NO: 18) | (SEQ ID NO: 27) | (SEQ ID NO: 54) | (SEQ ID NO: 71) |
| | | mutated | | | | | | |
| | | at: | | | | | | |
| | | E3V | | | | | | |
| | | N769 | | | | | | |
| 165 | V78A | Parental | ELSMQ | GFDPDDGETMYAEKFQG | SPGYDFFDY (SEQ | GGDNIGSKSVH | DDSDRPS | QVWDSSNDLLI |
| | N297A | clone 15 | (SEQ ID NO: 1) | (SEQ ID NO: 7) | ID NO: 18) | (SEQ ID NO: 27) | (SEQ ID NO: 54) | (SEQ ID NO: 71) |
| | | mutated | | | | | | |
| | | at: | | | | | | |
| | | E3V | | | | | | |
| | | N76S | | | | | | |
| 166 | R82aS | Parental | ELSMQ | GFDPDDGETMYAEKFQG | SPGYDFFDY (SEQ | GGDNIGSKSVH | DDSDRPS | QVWDSSNDLLI |
| | N297A | clone 15 | (SEQ ID NO: 1) | (SEQ ID NO: 7) | ID NO: 18) | (SEQ ID NO: 27) | (SEQ ID NO: 54) | (SEQ ID NO: 71) |
| | | mutated | | | | | | |
| | | at: | | | | | | |
| | | E3V | | | | | | |
| | | N76S | | | | | | |
| 167 | T83R | Parental | ELSMQ | GFDPDDGETMYAEKFQG | SPGYDFFDY (SEQ | GGDNIGSKSVH | DDSDRES | QVWDSSNDLLI |
| | N297A | clone 15 | (SEQ ID NO: 1) | (SEQ ID NO: 7) | ID NO: 18) | (SEQ ID NO: 27) | (SEQ ID NO: 54) | (SEQ ID NO: 71) |
| | | mutated | | | | | | |
| | | at: | | | | | | |
| | | E3V | | | | | | |
| | | N765 | | | | | | |
| 168 | L89V | Parental | ELSMQ | GFDPDDGETMYAEKFQG | SPGYDFFDY (SEQ | GGDNIGSKSVH | DDSDRPS | QVWDSSNDLLI |
| | N297A | clone 15 | (SEQ ID NO: 1) | (SEQ ID NO: 7) | ID NO: 18) | (SEQ ID NO: 27) | (SEQ ID NO: 54) | (SEQ ID NO: 71) |
| | | mutated | | | | | | |
| | | at: | | | | | | |
| | | E3V | | | | | | |
| | | N76S | | | | | | |
| 169 | F91Y | Parental | ELSMQ | GFDPDDGETMYAEKFQG | SPGYDFFDY (SEQ | GGDNIGSKSVH | DDSDRPS | QVWDSSNDLLI |
| | N297A | clone 15 | (SEQ ID NO: 1) | (SEQ ID NO: 7) | ID NO: 18) | (SEQ ID NO: 27) | (SEQ ID NO: 54) | (SEQ ID NO: 71) |
| | | mutated | | | | | | |
| | | at: | | | | | | |
| | | E3V | | | | | | |
| | | N76S | | | | | | |
| 17 | Parental | Parental | ELSMQ | GFDPDDGETMYAEKFQG | SPGYDFFDY (SEQ | GGSDIGDEIVH | DDSDRPS | QVWDSSSDHLI |
| | | | (SEQ ID NO: 1) | (SEQ ID NO: 7) | ID NO: 18) | (SEQ ID NO: 36) | (SEQ ID NO: 54) | (SEQ ID NO: 72) |
| 170 | N297A | Parental | ELSMQ | GFDPDDGETMYAEKFQG | SPGYDFFDY (SEQ | GGSDIGDEIVH | DDSDRPS | QVWDSSSDHLI |
| | | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | (mutations | | | | | | |
| | | in heavy | | | | | | |
| | | chain | | | | | | |
| | | only) | | | | | | |
| 171 | D54A | Parental | ELSMQ | GFDPDAGETMYAEKFQG | SPGYDFFDY (SEQ | GGSDIGDEIVH | DDSDRES | QVWDSSSDHLI |
| | N297A | clone 17 | (SEQ ID NO: 1) | (SEQ ID NO: 92) | ID NO: 18) | (SEQ ID NO: 36) | (SEQ ID NO: 54) | (SEQ ID NO: 72) |
| | | (mutations | | | | | | |
| | | in heavy | | | | | | |
| | | chain | | | | | | |
| | | only) | | | | | | |
| 172 | D54S | Parental | ELSMQ | GFDPDSGETMYAEKFOG | SPGYDFFDY (SEQ | GGSDIGDEIVH | DDSDRPS | QVWDSSSDHLI |
| | N297A | clone 17 | (SEQ ID NO: 1) | (SEQ ID NO: 93) | ID NO: 18) | (SEQ ID NO: 36) | (SEQ ID NO: 54) | (SEQ ID NO: 72) |
| | | mutations | | | | | | |
| | | in heavy | | | | | | |
| | | chain | | | | | | |
| | | only) | | | | | | |
| 173 | D54E | Parental | ELSMQ | GFDPDEGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 94) | ID NO: 18) | (SEQ ID NO: 36) | (SEQ ID NO: 54) | (SEQ ID NO: 72) |
| | | (mutations | | | | | | |
| | | in heavy | | | | | | |
| | | chain | | | | | | |
| | | only) | | | | | | |
| 174 | G55A | Parental | ELSMQ | GFDPDDAETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 95) | ID NO: 18) | (SEQ ID NO: 36) | (SEQ ID NO: 54) | (SEQ ID NO: 72) |
| | | (mutations | | | | | | |
| | | in heavy | | | | | | |
| | | chain | | | | | | |
| | | only) | | | | | | |
| 175 | R82aS | Parental | ELSMQ | GFDPDDGETMYAEKFQG | SPGYDFFDY (SEQ | GGSDIGDEIVH | DDSDRPS | QVWDSSSDHLI |
| | T83R | clone 17 | (SEQ ID NO: 1) | (SEQ ID NO: 7) | ID NO: 18) | (SEQ ID NO: 36) | (SEQ ID NO: 54) | (SEQ ID NO: 72) |
| | L89V | (mutations | | | | | | |
| | N297A | in heavy | | | | | | |
| | | chain | | | | | | |
| | | only) | | | | | | |
| 176 | D54A | Parental | ELSMQ | GFDPDAGETMYAEKFQG | SPGYDFFDY (SEQ | GGSDIGDEIVH | DDSDRPS | QVWDSSSDHLI |
| | R82aS | clone 17 | (SEQ ID NO: 1) | (SEQ ID NO: 92) | ID NO: 18) | (SEQ ID NO: 36) | (SEQ ID NO: 54) | (SEQ ID NO: 72) |
| | T83R | (mutations | | | | | | |
| | L89V | in heavy | | | | | | |
| | N297A | chain | | | | | | |
| | | only) | | | | | | |
| 177 | D54S | Parental | ELSMQ | GFDPDSGETMYAEKFQG | SPGYDFFDY (SEQ | GGSDIGDEIVH | DDSDRPS | QVWDSSSDHLI |
| | R82aS | clone 17 | (SEQ ID NO: 1) | (SEQ ID NO: 93) | NO: 18) | (SEQ ID NO: 36) | (SEQ ID NO: 54) | (SEQ ID NO: 72) |
| | T83R | (mutations | | | | | | |
| | L89V | in heavy | | | | | | |
| | N297A | chain | | | | | | |
| | | only) | | | | | | |
| 178 | D54E | Parental | ELSMQ | GFDPDEGETMYAEKFQG | SPGYDFFDY (SEQ | GGSDIGDEIVA | DDSDRPS | QVWDSSSDHLI |
| | R82aS | clone 17 | (SEQ ID NO: 1) | (SEQ ID NO: 94) | ID NO: 18) | (SEQ ID NO: 36) | (SEQ ID NO: 54) | (SEQ ID NO: 72) |
| | T83R | (mutations | | | | | | |
| | L89V | in heavy | | | | | | |
| | N297A | chain | | | | | | |
| | | only) | | | | | | |
| 179 | G55A | Parental | ELSMQ | GFDPDDAETMYAEKFQG | SPGYDFFDY (SEQ | GGSDIGDEIVH | DDSDRPS | QVWDSSSDHLI |
| | R82aS | clone 17 | (SEQ ID NO: 1) | (SEQ ID NO: 95) | ID NO: 18) | (SEQ ID NO: 36) | (SEQ ID NO: 54) | (SEQ ID NO: 72) |
| | T83R | (mutations | | | | | | |
| | L89V | in heavy | | | | | | |
| | N297A | chain | | | | | | |
| | | only) | | | | | | |
| 180 | N297A | Parental | ELSMQ | GFDPDDGETMYAEKFQG | SPGYDFFDY (SEQ | GGSDIGDEIVH | DDSDRPS | QVWDSSSDHLI |
| | | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | mutated | | | | | | |
| | | at: | | | | | | |
| | | A78V | | | | | | |
| | | Q79E | | | | | | |
| 181 | D54A | Parental | ELSMQ | GFDPDAGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 92) | ID NO: 18) | (SEQ ID NO: 36) | (SEQ ID NO: 54) | (SEQ ID NO: 72) |
| | | mutated | | | | | | |
| | | at: | | | | | | |
| | | A78V | | | | | | |
| | | Q79E | | | | | | |
| 182 | D54S | Parental | ELSMQ | GFDPDSGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 93) | ID NO: 18) | (SEQ ID NO: 36) | (SEQ ID NO: 54) | (SEQ ID NO: 72) |
| | | mutated | | | | | | |
| | | at: | | | | | | |
| | | A78V | | | | | | |
| | | Q79E | | | | | | |
| 183 | D54E | Parental | ELSMQ | GFDPDEGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 94) | ID NO: 18) | (SEQ ID NO: 36) | (SEQ ID NO: 54) | (SEQ ID NO: 72) |
| | | mutated | | | | | | |
| | | at: | | | | | | |
| | | A78V | | | | | | |
| | | Q79E | | | | | | |
| 184 | G55A | Parental | ELSMQ | GFDPDDAETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 95) | ID NO: 18) | (SEQ ID NO: 36) | (SEQ ID NO: 54) | (SEQ ID NO: 72) |
| | | mutated | | | | | | |
| | | at: | | | | | | |
| | | A78V | | | | | | |
| | | Q79E | | | | | | |
| 185 | R82aS | Parental | ELSMQ | GFDPDDGETMYAEKFQG | SPGYDFFDY (SEQ | GGSDIGDEIVA | DDSDRPS | QVWDSSSDHLI |
| | T83R | clone 17 | (SEQ ID NO: 1) | (SEQ ID NO: 7) | ID NO: 18) | (SEQ ID NO: 36) | (SEQ ID NO: 54) | (SEQ ID NO: 72) |
| | L89V | mutated | | | | | | |
| | N297A | at: | | | | | | |
| | | A78V | | | | | | |
| | | Q79E | | | | | | |
| 186 | D54A | Parental | ELSMQ | GFDPDAGETMYAEKFQG | SPGYDFFDY (SEQ | GGSDIGDEIVH | DDSDRPS | QVWDSSSDHLI |
| | R82as | clone 17 | (SEQ ID NO: 1) | (SEQ ID NO: 92) | ID NO: 18) | (SEQ ID NO: 36) | (SEQ ID NO: 54) | (SEQ ID NO: 72) |
| | T83R | mutated | | | | | | |
| | L89V | at: | | | | | | |
| | N297A | A78V | | | | | | |
| | | Q79E | | | | | | |
| 187 | D54S | Parental | ELSMQ | GFDPDSGETMYAEKFQG | SPGYDFFDY (SEQ | GGSDIGDEIVH | DDSDRPS | QVWDSSSDHLI |
| | R82aS | clone 17 | (SEQ ID NO: 1) | (SEQ ID NO: 93) | ID NO: 18) | (SEQ ID NO: 36) | (SEQ ID NO: 54) | (SEQ ID NO: 72) |
| | T83R | mutated | | | | | | |
| | L89V | at: | | | | | | |
| | N297A | A78V | | | | | | |
| | | Q79E | | | | | | |
| 188 | D54E | Parental | ELSMQ | GFDPDEGETMYAEKFQG | SPGYDFFDY (SEQ | GGSDIGDEIVH | DDSDRPS | QVWDSSSDHLI |
| | R82as | clone 17 | (SEQ ID NO: 1) | (SEQ ID NO: 94) | ID NO: 18) | (SEQ ID NO: 36) | (SEQ ID NO: 54) | (SEQ ID NO: 72) |
| | T83R | mutated | | | | | | |
| | L89V | at: | | | | | | |
| | N297A | A78V | | | | | | |
| | | Q79E | | | | | | |
| 189 | G55A | Parental | ELSMQ | GFDPDDAETMYAEKFQG | SPGYDFFDY (SEQ | GGSDIGDEIVH | DDSDRPS | QVWDSSSDHLI |
| | R82aS | clone 17 | (SEQ ID NO: 1) | (SEQ ID NO: 95) | ID NO: 18) | (SEQ ID NO: 36) | (SEQ ID NO: 54) | (SEQ ID NO: 72) |
| | T83R | mutated | | | | | | |
| | L89V | at: | | | | | | |
| | N297A | A78V | | | | | | |
| | | Q79E | | | | | | |
| 190 | E1Q | Parental | ELSMQ | GFDPDDGETMYAEKFQG | SPGYDFFDY (SEQ | GGSDIGDEIVH | DDSDRPS | QVWDSSSDHLI |
| | L67V | clone 17 | (SEQ ID NO: 1) | (SEQ ID NO: 7) | ID NO: 18) | (SEQ ID NO: 36) | (SEQ ID NO: 54) | (SEQ ID NO: 72) |
| | V69M | (mutations | | | | | | |
| | V78A | in heavy | | | | | | |
| | R82aS | chain | | | | | | |
| | T83R | only) | | | | | | |
| | L89V | | | | | | | |
| | F91Y | | | | | | | |
| | N297A | | | | | | | |
| 191 | D54A | Parental | ELSMQ | GFDPDAGETMYAEKFQG | SPGYDFFDY (SEQ | GGSDIGDEIVH | DDSDRPS | QVWDSSSDHLI |
| | E1Q | clone 17 | (SEQ ID NO: 1) | (SEQ ID NO: 92) | ID NO: 18) | (SEQ ID NO: 36) | (SEQ ID NO: 54) | (SEQ ID NO: 72) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 192 | D54S | Parental | ELSMQ | GFDPDSGETMYAEKFQG | SPGYDFFDY (SEQ | GGSDIGDEIVH | DDSDRPS | QVWDSSSDHLI |
| | E1Q | clone 17 | (SEQ ID NO: 1) | (SEQ ID NO: 93) | ID NO: 18) | (SEQ ID NO: 36) | (SEQ ID NO: 54) | (SEQ ID NO: 72) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 193 | DS4E | Parental | ELSMQ | GFDPDEGETMYAEKFQG | SPGYDFFDY (SEQ | GGSDIGDEIVH | DDSDRPS | QVWDSSSDHLI |
| | E1Q | clone 17 | (SEQ ID NO: 1) | (SEQ ID NO: 94) | ID NO: 18) | (SEQ ID NO: 36) | (SEQ ID NO: 54) | (SEQ ID NO: 72) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 194 | G55A | Parental | ELSMQ | GFDPDDAETMYAEKFQG | SPGYDFFDY (SEQ | GGSDIGDEIVH | DDSDRPS | QVWDSSSDHLI |
| | E1Q | clone 17 | (SEQ ID NO: 1) | (SEQ ID NO: 95) | ID NO: 18) | (SEQ ID NO: 36) | (SEQ ID NO: 54) | (SEQ ID NO: 72) |
| | L67V | (mutations | | | | | | |
| | V69M | in heavy | | | | | | |
| | V78A | chain | | | | | | |
| | R82aS | only) | | | | | | |
| | T83R | | | | | | | |
| | L89V | | | | | | | |
| | F91Y | | | | | | | |
| | N297A | | | | | | | |
| 195 | E1Q | Parental | ELSMQ | GFDPDDGETMYAEKFQG | SPGYDFFDY (SEQ | GGSDIGDEIVH | DDSDRPS | QVWDSSSDHLI |
| | L67V | clone 17 | (SEQ ID NO: 1) | (SEQ ID NO: 7) | ID NO: 18) | (SEQ ID NO: 36) | (SEQ ID NO: 54) | (SEQ ID NO: 72) |
| | V69M | mutated | | | | | | |
| | V78A | at: | | | | | | |
| | R82as | A78V | | | | | | |
| | T83R | Q79E | | | | | | |
| | L89V | | | | | | | |
| | F91Y | | | | | | | |
| | N297A | | | | | | | |
| 196 | D54A | Parental | ELSMQ | GFDPDAGETMYAEKFQG | SPGYDFFDY (SEQ | GGSDIGDEIVH | DDSDRPS | QVWDSSSDHLI |
| | E1Q | clone 17 | (SEQ ID NO: 1) | (SEQ ID NO: 92) | ID NO: 18) | (SEQ ID NO: 36) | (SEQ ID NO: 54) | (SEQ ID NO: 72) |
| | L67V | mutated | | | | | | |
| | V69M | at: | | | | | | |
| | V78A | A78V | | | | | | |
| | R82aS | Q79E | | | | | | |
| | T83R | | | | | | | |
| | L89V | | | | | | | |
| | F91Y | | | | | | | |
| | N297A | | | | | | | |
| 197 | D54S | Parental | ELSMQ | GFDPDSGETMYAEKFQG | SPGYDFFDY (SEQ | GGSDIGDEIVH | DDSDRPS | QVWDSSSDHLI |
| | E1Q | clone 17 | (SEQ ID NO: 1) | (SEQ ID NO: 93) | ID NO: 18) | (SEQ ID NO: 36) | (SEQ ID NO: 54) | (SEQ ID NO: 72) |
| | L67V | mutated | | | | | | |
| | V69M | at: | | | | | | |
| | V78A | A78V | | | | | | |
| | R82aS | Q79E | | | | | | |
| | T83R | | | | | | | |
| | L89V | | | | | | | |
| | F91Y | | | | | | | |
| | N297A | | | | | | | |
| 198 | D54E | Parental | ELSMQ | GFDPDEGETMYAEKFQG | SPGYDFFDY (SEQ | GGSDIGDEIVH | DDSDRPS | QVWDSSSDHLI |
| | E1Q | clone 17 | (SEQ ID NO: 1) | (SEQ ID NO: 94) | ID NO: 18) | (SEQ ID NO: 36) | (SEQ ID NO: 54) | (SEQ ID NO: 72) |
| | L67V | mutated | | | | | | |
| | V69M | at: | | | | | | |
| | V78A | A78V | | | | | | |
| | R82aS | Q79E | | | | | | |
| | T83R | | | | | | | |
| | L89V | | | | | | | |
| | F91Y | | | | | | | |
| | N297A | | | | | | | |
| 199 | G55A | Parental | ELSMQ | GFDPDDAETMYAEKFQG | SPGYDFFDY (SEQ | GGSDIGDEIVH | DDSDRPS | QVWDSSSDHLI |
| | E1Q | clone 17 | (SEQ ID NO: 1) | (SEQ ID NO: 95) | ID NO: 18) | (SEQ ID NO: 36) | (SEQ ID NO: 54) | (SEQ ID NO: 72) |
| | L67V | mutated | | | | | | |
| | V69M | at: | | | | | | |
| | V78A | A78V | | | | | | |
| | R82aS | Q79E | | | | | | |
| | T83R | | | | | | | |
| | L89V | | | | | | | |
| | F91Y | | | | | | | |
| | N297A | | | | | | | |
| 200 | E10 | Parental | ELSMQ | GFDPDDGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | (mutations | | | | | | |
| | | in heavy | | | | | | |
| | | chain | | | | | | |
| | | only) | | | | | | |
| 201 | L67V | Parental | ELSMQ | GFDPDDGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | (mutations | | | | | | |
| | | in heavy | | | | | | |
| | | chain | | | | | | |
| | | only) | | | | | | |
| 202 | V69M | Parental | ELSMQ | GFDPDDGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | (mutations | | | | | | |
| | | in heavy | | | | | | |
| | | chain | | | | | | |
| | | only) | | | | | | |
| 203 | V78A | Parental | ELSMQ | GFDPDDGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | (mutations | | | | | | |
| | | in heavy | | | | | | |
| | | chain | | | | | | |
| | | only) | | | | | | |
| 204 | R82aS | Parental | ELSMQ | GFDPDDGETMYAEKFQG | SPGYDFFDY (SEQ | GGSDIGDEIVA | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | (mutations | | | | | | |
| | | in heavy | | | | | | |
| | | chain | | | | | | |
| | | only) | | | | | | |
| 205 | T83R | Parental | ELSMQ | GFDPDDGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | (mutations | | | | | | |
| | | in heavy | | | | | | |
| | | chain | | | | | | |
| | | only) | | | | | | |
| 206 | L89V | Parental | ELSMQ | GFDPDDGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | (mutations | | | | | | |
| | | in heavy | | | | | | |
| | | chain | | | | | | |
| | | only) | | | | | | |
| 207 | F91Y | Parental | ELSMQ | GFDPDDGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | (mutations | | | | | | |
| | | in heavy | | | | | | |
| | | chain | | | | | | |
| | | only) | | | | | | |
| 208 | E1Q | Parental | ELSMQ | GFDPDDGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | mutated | | | | | | |
| | | at: | | | | | | |
| | | A78V | | | | | | |
| | | Q79E | | | | | | |
| 209 | L67V | Parental | ELSMQ | GFDPDDGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | mutated | | | | | | |
| | | at: | | | | | | |
| | | A78V | | | | | | |
| | | Q79E | | | | | | |
| 210 | V69M | Parental | ELSMQ | GFDPDDGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | mutated | | | | | | |
| | | at: | | | | | | |
| | | A78V | | | | | | |
| | | Q79E | | | | | | |
| 211 | V78A | Parental | ELSMQ | GFDPDDGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | mutated | | | | | | |
| | | at: | | | | | | |
| | | A78V | | | | | | |
| | | Q79E | | | | | | |
| 212 | R82aS | Parental | ELSMQ | GFDPDDGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | mutated | | | | | | |
| | | at: | | | | | | |
| | | A78V | | | | | | |
| | | Q79E | | | | | | |
| 213 | T83R | Parental | ELSMQ | GFDPDDGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | mutated | | | | | | |
| | | at: | | | | | | |
| | | A78V | | | | | | |
| | | Q79E | | | | | | |
| 214 | L89V | Parental | ELSMQ | GFDPDDGETMYAEKFQG | SPGYDFFDY (SEQ | GGSDIGDEIVH | DDSDRPS | QVWDSSSDHLI |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | mutated | | | | | | |
| | | at: | | | | | | |
| | | A78V | | | | | | |
| | | Q79E | | | | | | |
| 215 | F91Y | Parental | ELSMQ | GFDPDDGETMYAEKFQG | SPGYDFFDY (SEQ | GGSDIGDEIVH | DDSDRPS | QVWDSSSDHLJ |
| | N297A | clone 17 | (SEQ ID NO: 1) | (SEQ ID NO: 7) | ID NO: 18) | (SEQ ID NO: 36) | (SEQ ID NO: 54) | (SEQ ID NO: 72) |
| | | mutated | | | | | | |
| | | at: | | | | | | |
| | | A78V | | | | | | |
| | | Q79E | | | | | | |

TABLE 3
Consensus CDR sequences.
Group 1, 2, 3, 4, 6, 12 and 13

| | |
|---|---|
| VH CDR1 | X₁X₂X₃X₄X₅ |
| | X₁ is S, E, or D; |
| | X₂ is Y, L, or S; |
| | X₃ is A, S, or F; |
| | X₄ is I or M; and |
| | X₅ is S, Q, or H. |
| VH CDR2 | X₆X₇X₈PX₉X₁₀X₁₁X₁₂X₁₃X₁₄YAX₁₅X₁₆X₁₇X₁₈G (SEQ ID NO: 97) |
| | X₆ is L or G; |
| | X₇ is V, F, or I; |
| | X₈ is D or I; |
| | X₉ is E, D, or I; |
| | X₁₀ is D, G, F, A, S, or E; |
| | X₁₁ is G or A: |
| | X₁₂ is E or T; |
| | X₁₃ is T or A: |
| | X₁₄ is I, M, or N; |
| | X₁₅ is E or Q; |
| | X₁₆ is K or R: |
| | X₁₇ is F or L; |
| | X₁₈ is R or Q. |
| VH CDR3 | X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇X₃₈ (SEQ ID NO: 98) |
| | X₁₉ is P, E, or absent; |
| | X₂₀ is G, E, or absent; |
| | X₂₁ is G, W, S, or absent; |
| | X₂₂ is I, D, S, P, or absent; |
| | X₂₃ is S, L, I, T, G, or absent; |
| | X₂₄ is P, T, Q, I, D, R, or absent; |
| | X₂₅ is G, D, or absent; |
| | X₂₆ is E, Y, P, L, D, or S; |
| | X₂₇ is E, D, A, or G; |
| | X₂₈ is S, F, A, E, Y, or W; |
| | X₂₉ is Y or F: |
| | X₃₀ is G, D, or W; |
| | X₃₁ is P, Y, L, or H; |
| | X₃₂ is Y or absent; |
| | X₃₃ is Y or absent; |
| | X₃₄ is Y or absent; |
| | X₃₅ is G or absent; |
| | X₃₆ is M or absent; |
| | X₃₇ is D or absent; |
| | X₃₈ is V or absent. |
| VL CDR1 | X₃₉X₄₀X₄₁X₄₂X₄₃X₄₄X₄₅X₄₆X₄₇X₄₈X₄₉X₅₀X₅₁X₅₂X₅₃X₅₄ (SEQ ID NO: 99) |
| | X₃₉ is T, A, or absent; |
| | X₄₀ is G or absent; |
| | X₄₁ is Q, G, V, T, or S; |
| | X₄₂ is A, G, S, or N; |
| | X₄₃ is S, N, D, T, or Y; |
| | X₄₄ is Q, Y, N, D, S, or K; |
| | X₄₅ is D, I, F, V, S, or T; |
| | X₄₆ is N or absent; |
| | X₄₇ is I or absent; |
| | X₄₈ is I, G, or R; |
| | X₄₉ is S, G, A, D, I, R, or T; |
| | X₅₀ is Y or absent; |
| | X₅₁ is N, K, I, or E; |
| | X₅₂ is Y, S, N. D, H, F, R, or G; |
| | X₅₃ is L or V; and |
| | X₅₄ is N, H, S. D, or F. |
| VL CDR2 | X₅₅X₅₆X₅₇X₅₈X₅₉X₆₀X₆₁ |
| | X₅₅ is T, D, E, Q, or R; |
| | X₅₆ is A, D, V, or N; |
| | X₅₇ is S, G, N, or R; |
| | X₅₈ is I, N, D, or K; |
| | X₅₉ is L, R, or W; |
| | X₆₀ is E or P; and |
| | X₆₁ is S, T, or L. |
| VL CDR3 | X₆₂X₆₃X₆₄X₆₅X₆₆X₆₇X₆₈X₆₉X₇₀X₇₁X₇₂ |
| | X₆₂ is Q, S, C, or G; |
| | X₆₃ is Q, V, S, T, or A; |
| | X₆₄ is Q, L, W, or Y; |
| | X₆₅ is Q, N, D, I, T, A, or G; |
| | X₆₆ is S, P, G, N, or A; |
| | X₆₇ is N, Y, I, S, N, L, or D; |
| | X₆₈ is E, P, S, I, N, H, L, or T; |
| | X₆₉ is D, T, S, or absent; |
| | X₇₀ is P, H, L, R, F, A, Q, or absent; |
| | X₇₁ is W, L, V, Y, S, A, or E; and |
| | X₇₂ is T, V, or I. |

Group 1

| | |
|---|---|
| VH CDR1 | ELSMQ (SEQ ID NO: 1) |
| VH CDR1 | GYTLTELSMQ (SEQ ID NO: 329) |
| VH CDR2 | GFDPDDX₁₀₁ETMYAEX₁₀₂X₁₀₃QG (SEQ ID NO: 102) |
| | X₁₀₁ is D or G; |
| | X₁₀₂ is K or R; and |
| | X₁₀₃ is F or L. |
| VH CDR3 | SPGYDFFDY (SEQ ID NO: 18) |
| VL CDR1 | GGX₁₀₄X₁₀₅X₁₀₆GX₁₀₇X₁₀₈X₁₀₉VX₁₁₀ (SEQ ID NO: 103) |
| | X₁₀₄ is N, D, or S: |
| | X₁₀₅ is Y, N, or D; |
| | X₁₀₆ is I or T; |
| | X₁₀₇ is S, D, I, R, or T; |
| | X₁₀₈ is K, E, or I; |
| | X₁₀₉ is S, I, R, G, N, or A; and |
| | X₁₁₀ is H, F, or N. |
| VL CDR2 | DDX₁₁₁DRPX₁₁₂ (SEQ ID NO: 104) |
| | X₁₁₁ is G, S, or R; and |
| | X₁₁₂ is S or L. |
| VL CDR3 | QVWDX₁₁₃X₁₁₄X₁₁₅DX₁₁₆X₁₁₇X₁₁₈ (SEQ ID NO: 105) |
| | X₁₁₃ is S or A; |
| | X₁₁₄ is I or S; |
| | X₁₁₅ is S, I, or N; |
| | X₁₁₆ is H, L, or Q; |
| | X₁₁₇ is V or L; and |
| | X₁₁₈ is V or I. |
| VL CDR1 | TGTSSDVGGYNYVS (SEQ ID NO: 30) |
| VL CDR2 | X₁₁₉VSX₁₂₀RPS (SEQ ID NO: 106) |
| | X₁₁₉ is E or D; and |
| | X₁₂₀ is N or K |
| VL CDR3 | QVWDSSNDLLI (SEQ ID NO: 71) |

Group 2

| | |
|---|---|
| VH CDR1 | DYFIH (SEQ ID NO: 2) |
| VH CDR2 | LVDPEDGETIYAEX₁₂₁FQG (SEQ ID NO: 107) |
| | X₁₂₁ is K or R. |
| VH CDR3 | PGGILTDPDAFDI (SEQ ID NO: 19) |
| VL CDR1 | X₁₂₂GTX₁₂₃SDVGGYNX₁₂₄VS (SEQ ID NO: 108) |
| | X₁₂₂ is T or A; |
| | X₁₂₃ is S or G; and |
| | X₁₂₄ is Y or H. |
| VL CDR2 | X₁₂₅VX₁₂₆X₁₂₇RPS (SEQ ID NO: 109) |
| | X₁₂₅ is D or E; |
| | X₁₂₆ is N or S; and |
| | X₁₂₇ is K or N. |
| VL CDR3 | SSYX₁₂₈X₁₂₉SSTX₁₃₀X₁₃₁V (SEQ ID NO: 110) |
| | X₁₂₈ is I or T; |
| | X₁₂₉ is P or S; |
| | X₁₃₀ is R, P, F, or absent; and |
| | X₁₃₁ is W or Y. |

Group 3

| | |
|---|---|
| VH CDR1 | SYAIS (SEQ ID NO: 3) |
| VH CDR2 | LVDPEDGETIYAEKFX₁₃₂G (SEQ ID NO: 111) |
| | X₁₃₂ is R or Q |
| VH CDR3 | EESYGP (SEQ ID NO: 20) |
| VL CDR1 | QASQDISNYLX₁₃₃ (SEQ ID NO: 112) |
| | X₁₃₃ is N or D |
| VL CDR2 | DASNLET (SEQ ID NO: 61) |
| VL CDR3 | QQLNSYPLT (SEQ ID NO: 80) |

Group 4

| | |
|---|---|
| VH CDR1 | EX₁₃₄SMH (SEQ ID NO: 113) |
| | X₁₃₄ is S or L |
| VH CDR2 | LVDPEDGETIYAQKFQG (SEQ ID NO: 14) |
| VH CDR3 | EEWSGDGDDAFDI (SEQ ID NO: 21) |
| VL CDR1 | SGSSSNIGSYSVS (SEQ ID NO: 46) |
| VL CDR2 | DX₁₃₅NKRPS (SEQ ID NO: 114) |
| | X₁₃₅ is N or D |
| VL CDR3 | GTWDSSLSAWV (SEQ ID NO: 81). |

Group 6

| | |
|---|---|
| VH CDR1 | SYAIS (SEQ ID NO: 3) |
| VH CDR2 | GIIPIFGTANYAQKFQG (SEQ ID NO: 15) |
| VH CDR3 | SPLRGSGWYWHYYYGMDV (SEQ ID NO: 22) |
| VL CDR1 | GGNX₁₃₆IX₁₃₇X₁₃₈KX₁₃₉VH (SEQ ID NO: 115) |
| | X₁₃₆ is N or K; |
| | X₁₃₇ is R or G; |
| | X₁₃₈ is A, S, R, or T; and |
| | X₁₃₉ is H or S. |
| VL CDR2 | X₁₄₀DX₁₄₁X₁₄₂RPS (SEQ ID NO: 116) |
| | X₁₄₀ is Q or R; |
| | X₁₄₁ is S or R: and |
| | X₁₄₂ is N or K. |
| VL CDR3 | QX₁₄₃WX₁₄₄SX₁₄₅TX₁₄₆V (SEQ ID NO: 117) |
| | X₁₄₃ is A or V; |
| | X₁₄₄ is D or G; |
| | X₁₄₅ is S or N; and |
| | X₁₄₆ is V, A, or E. |

TABLE 4
Heavy chain, heavy chain variable region, light chain, and light chain variable region sequences.

| Clone | | | | | |
|---|---|---|---|---|---|
| No. | Group | Heavy Chain | VH | Light Chain | VL |
| 1 | 1 | EVQLVQSGAEVKKPGASVKVSCKVSGYTLTE | EVQLVQSGAEVKKPGASVKVSCKVSGY | SYVLTQPPSVSVAPGKTARITCGGN | SYVLTQPPSVSVAPGKTARITCGGNY |
| | | LSMQWVRQAPGKGLEWMGGFDPDDGETMYAE | TLTELSMQWVRQAPGKGLEWMGGFDPD | YIGSKSVHWYQQKPGQAPVLVVYDD | IGSKSVHWYQQKPGQAPVLVVYDDGD |
| | | KFQGRLTVTEDTSTDTVYMELRSLTSEDTAL | DGETMYAEKFQGRLTVTEDTSTDTVYM | GDRPSGIPARFSGSNSGNTATLTIS | RPSGIPARFSGSNSGNTATLTISRVE |
| | | YFCATSPGYDFFDYWGQGTLVTVSSASTKGP | ELRSLTSEDTALYFCATSPGYDFFDYW | RVEAGDEADYYCQVWDSISDHVVFG | AGDEADYYCQVWDSISDHVVFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 264) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 209) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 2 | 1 | EVQLVQSGAEVKKPGASVKVSCKVSGYTLTE | EVQLVQSGAEVKKPGASVKVSCKVSGY | SYELTQPPSVSVAPGKTARITCGGD | SYELTQPPSVSVAPGKTARITCGGDN |
| | | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRVE |
| | | YFCATSPGYDFFDYWGQGTLVTVSSASTKGP | ELRSLTSEDTALYFCATSPGYDFFDYW | RVEAGDEADYYCQVWDSSIDHLIFG | AGDEADYYCQVWDSSIDHLIFGGGTK |
| | | SVFPLAPSSKSTSGGTAALGCLVKDYFPEPV | GQGTLVTVSS (SEQ ID NO: 165) | GGTKVTVLGQPKAAPSVTLFPPSSE | VTVL (SEQ ID NO: 265) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 210) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 3 | 1 | EVQLVQSGAEVKKPGASVKVSCKVSGYTLTE | EVQLVQSGAEVKKPGASVKVSCKVSGY | SYVLTQPPSVSVAPGQTARITCGGN | SYVLTQPPSVSVAPGQTARITCGGNN |
| | | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | | RFQGRLTVTEDTSTDTVYMELRSLTSEDTAL | DGETMYAERFQGRLTVTEDTSTDTVYM | SDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 166) | GGTKLTVLGQPKAAPSVTLFPPSSE | LTVL (SEQ ID NO: 266) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 211) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 120) | | | |
| 4 | 1 | EVQLVQSGAEVKKPGASVKVSCKVSGYTLTE | EVQLVQSGAEVKKPGASVKVSCKVSGY | SYVLTQPPSVSVAPGQTARITCGGN | SYVLTQPPSVSVAPGQTARITCGGNN |
| | | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVIVSS (SEQ ID NO: 165) | GGTKLTVLGQPKAAPSVTLFPPSSE | LIVL (SEQ ID NO: 266) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTRPREEQYNSTYRVVSVLTVL | | NO: 211) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 5 | 1 | EVQLVQSGAEVKKPGASVKVSCKVSGYTLTE | EVQLVQSGAEVKKPGASVKVSCKVSGY | SYVLTQPPSVSVAPGQTASITCVGT | SYVLTQPPSVSVAPGQTASITCVGTN |
| | | LSMQWVRQAPGKGLEWMGGFDPDDGETMYAE | TLTELSMQWVRQAPGKGLEWMGGFDPD | NFGSKNVHWYQQRPGQAPVLVVYDD | FGSKNVHWYQQRPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRVE |
| | | YFCATSPGYDFFDYWGQGTLVTVSSASTKGP | ELRSLTSEDTALYFCATSPGYDFFDYW | RVEAGDEADYYCQVWDSSSDHLIFG | AGDEADYYCQVWDSSSDHLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 267) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 212) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSISLSPG (SEQ ID NO: 119) | | | |
| 6 | 1 | EVQLVQSGAEVKKPGASVKVSCKVSGYTLTE | EVQLVQSGAEVKKPGASVKVSCKVSGY | QSALTQPASVSGSPGQSITISCTGT | QSALTQPASVSGSPGQSITISCTGTS |
| | | LSMQWVRQAPGKGLEWMGGFDPDDGETMYAE | TLTELSMQWVRQAPGKGLEWMGGFDPD | SSDVGGYNYVSWYQQHPGKAPKLMI | SDVGGYNYVSWYQQHPGKAPKLMIYE |
| | | KFQGRLTVTEDTSTDTVYMELRSLTSEDTAL | DGETMYAEKFQGRLTVTEDTSTDTVYM | YEVSNRPSGVSNRFSGSNSGNTATL | VSNRPSGVSNRFSGSNSGNTATLTIN |
| | | YFCATSPGYDFFDYWGQGTLVTVSSASTKGP | ELRSLTSEDTALYFCATSPGYDFFDYW | TINRVEAGDEADYYCQVWDSSNDLL | RVEAGDEADYYCQVWDSSNDLLIFGG |
| | | SVFPLAPSSKSTSGGTAALGCLVKDYFPEPV | GQGTLVTVSS (SEQ ID NO: 165) | IFGGGTKLTVLGQPKAAPSVTLFPP | GTKLTVL (SEQ ID NO: 268) |
| | | TVSWNSGALTSGVHTFPAVLQSSGLYSLSSV | | SSEELQANKATLVCLISDFYPGAVT | |
| | | VTVPSSSLGTQTYICNVNHKPSNTKVDKRVE | | VAWKADSSPVKAGVETTTPSKQSNN | |
| | | PKSCDKTHTCPPCPAPELLGGPSVFLFPPKP | | KYAASSYLSLTPEQWKSHRSYSCQV | |
| | | KDTLMISRTPEVTCVVVDVSHEDPEVKFNWY | | THEGSTVEKTVAPTECS (SEQ ID | |
| | | VDGVEVHNAKTKPREEQYNSTYRVVSVLTVL | | NO: 213) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSISLSPG (SEQ ID NO: 119) | | | |
| 7 | 1 | EVQLVQSGAEVKKPGASVKVSCKVSGYTLTE | EVQLVQSGAEVKKPGASVKVSCKVSGY | SYVLTQPPSVSVAPGQTASITCVGT | SYVLTQPPSVSVAPGQTASITCVGTN |
| | | LSMQWVRQAPGKGLEWMGGEDPDDGETMYAE | TLTELSMQWVRQAPGKGLEWMGGFDPD | NFGSKNVHWYQQRPGQAPVLVVYDD | FGSKNVHWYQQRPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 269) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 214) | |
| | | HQDWLNGKEYKCKVSNKALPAPIERTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 8 | 1 | QVQLVQSGAEVKKPGSSVKVSCKVSGYTLTE | QVQLVQSGAEVKKPGSSVKVSCKVSGY | SYELTQPPSVSVAPGQTARITCGGN | SYELTQPPSVSVAPGQTARITCGGNN |
| | | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGRTATLTIS | RPSGIPERFSGSNSGRTATLTISRVE |
| | | YFCATSPGYDFFDYWGQGTLVTVSSASTKGP | ELRSLTSEDTALYFCATSPGYDFFDYW | RVEAGDEADYHCQVWDSSSDHLVFG | AGDEADYHCQVWDSSSDHLVFGGGTK |
| | | SVFPLAPSSKSTSGGTAALGCLVKDYFPEPV | GQGTLVTVSS (SEQ ID NO: 167) | GGTKLTVLGQPKAAPSVTLFPPSSE | LTVL (SEQ ID NO: 270) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 215) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 121) | | | |
| 9 | 1 | EVQLVQSGAEVKKPGASVKVSCKVSGYTLTE | EVQLVQSGAEVKKPGASVKVSCKVSGY | SYVLTQPPSVSVAPGQTARIPCGGN | SYVLTQPPSVSVAPGQTARIPCGGNN |
| | | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDRD |
| | | KFQGRLTVTEDTSTDTVYMELRSLTSEDTAL | DGETMYAEKFQGRLIVTEDTSTDTVYM | RDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 271) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 216) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 10 | 1 | EVQLVQSGAEVKKPGASVKVSCKVSGYTLTE | EVQLVQSGAEVKKPGASVKVSCKVSGY | SYELTQPPSVSVAPGQTARIPCGGN | SYELTQPPSVSVAPGQTARIPCGGNN |
| | | LSMQWVRQAPGKGLEWMGGFDPDDGETMYAE | TLTELSMQWVRQAPGKGLEWMGGFDPD | NIGSEGVHWYQQKPGQAPVLVVYDD | IGSEGVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 272) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVI | | NO: 217) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | QKSLSLSPG (SEQ ID NO: 119) | | | |
| 11 | 1 | EVRLVQSGAEVKKPGASVKVSCKVSGYTLTE | EVRLVQSGAEVKKPGASVKVSCKVSGY | QSALTQPRSVSGSPGQSVTISCTGT | QSALTQPRSVSGSPGQSVTISCTGTS |
| | | LSMQWVRQAPGKGLEWMGGFDPDDGETMYAE | TLTELSMQWVRQAPGKGLEWMGGFDPD | SSDVGGYNYVSWYQQHPGKAPKLMI | SDVGGYNYVSWYQQHPGKAPKLMIYD |
| | | KFQGRLTVTEDTSTDTVYMELRSLTSEDTAL | DGETMYAEKFQGRLTVTEDTSTDTVYM | YDVSKRPSGVPDRFSGSNSGNTATL | VSKRPSGVPDRESGSNSGNTATLTIN |
| | | YFCATSPGYDFFDYWGQGTLVTVSSASTKGP | ELRSLTSEDTALYFCATSPGYDFFDYW | TINRVEAGDEADYYCQVWDSSNDLL | RVEAGDEADYYCQVWDSSNDLLIFGG |
| | | SVFPLAPSSKSTSGGTAALGCLVKDYFPEPV | GQGTLVTVSS (SEQ ID NO: 168) | IFGGGTKLTVLGQPKAAPSVTLFPP | GTKLIVL (SEQ ID NO: 273) |
| | | TVSWNSGALTSGVHTFPAVLQSSGLYSLSSV | | SSEELQANKATLVCLISDFYPGAVT | |
| | | VTVPSSSIGTQTYICNVNHKPSNTKVDKRVE | | VAWKADSSPVKAGVETTTPSKQSNN | |
| | | PKSCDKTHTCPPCPAPELLGGPSVFLFPPKP | | KYAASSYLSLTPEQWKSHRSYSCQV | |
| | | KDTLMISRTPEVTCVVVDVSHEDPEVKFNWY | | THEGSTVEKTVAPTECS (SEQ ID | |
| | | VDGVEVHNAKTKPREEQYNSTYRVVSVLTVL | | NO: 218) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKITVDKSRWQQGNVFSCSVMHEALH | | | |
| | | NHYTQRSLSLSPG (SEQ ID NO: 122) | | | |
| 12 | 1 | EVQLVQSGAEVKKPGASVKVSCKVSGYTLTE | EVQLVQSGAEVKKPGASVKVSCKVSGY | SYVLTQPPSVSVAPGQTARVSCGGN | SYVLTQPPSVSVAPGQTARVSCGGNN |
| | | LSMQWVRQAPGKGLEWMGGFDPDGGETMYAE | TLTELSMQWVRQAPGKGLEWMGGFDPD | NIGSKSVNWYRQKPGQAPVLVIYDD | IGSKSVNWYRQKPGQAPVIVIYDDSD |
| | | KFQGRLTVTEDTSTDTVYMELRSLTSEDTAL | GGETMYAEKFQGRLTVTEDTSTDTVYM | SDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGQLVKDYFPEPV | GQGTLVTVSS (SEQ ID NO: 169) | GGTKLTVLGQPKAAPSVTLFPPSSE | LIVL (SEQ ID NO: 274) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PRSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 219) | |
| | | HQDWLNGKEYKCKVSNKALPAPIEKTISKAK | | | |
| | | GQPREPQVYTIPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 123) | | | |
| 13 | 1 | QVQLVQSGAEVKKPGASVKVSCKVSGYTLTE | QVQLVQSGAEVKKPGASVKVSCKVSGY | SYVLTQPPSVSVAPGKTARITCGGN | SYVLTQPPSVSVAPGKTARITCGGNN |
| | | LSMQWVRQAPGKGLEWMGGFDPDDGETMYAE | TLTELSMQWVRQAPGKGLEWMGGFDPD | NIGSKSVFWYQQKPGQAPVLVVYDD | IGSKSVFWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISGTQ |
| | | YFCATSPGYDFFDYWGQGTLVTVSSASTKGP | ELRSLTSEDTALYFCATSPGYDFFDYW | GTQAMDEADYYCQVWDSSSDHLVFG | AMDEADYYCQVWDSSSDHLVFGGGTK |
| | | SVFPLAPSSKSTSGGTAALGCLVKDYFPEPV | GQGTLVTVSS (SEQ ID NO: 170) | GGTKLTVLGQPKAAPSVTLFPPSSE | LTVL (SEQ ID NO: 275) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 220) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 124) | | | |
| 14 | 1 | EVQLVQSGAEVKKPGASVKVSCKVSGYTLTE | EVQLVQSGAEVKKPGASVKVSCKVSGY | SYVLTQPPSVSVAPGKTARITCGGY | SYVLTQPPSVSVAPGKTARITCGGYN |
| | | LSMQWVRQAPGKGLEWMGGFDPDDGETMYAE | TLTELSMQWVRQAPGKGLEWMGGFDPD | NIGSKFVHWYRQRPGQAPVLVVYDD | IGSKFVHWYRQRPGQAPVLVVYDDRD |
| | | KFQGRLAVTEDTSTDTVYMELRSLTSEDTAL | DGETMYAEKFQGRLAVTEDTSTDTVYM | RDRPSGIPERFSGSNSGNTATLTIS | RPSGIPERFSGSNSGNTATLTISRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 171) | GGTKLTVLGQPKAAPSVTLFPPSSE | LTVL (SEQ ID NO: 276) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 221) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 125) | | | |
| 15 | 1 | EVQLVQSGAEVKKPGASVKVSCKVSGYTLTE | EVQLVQSGAEVKKPGASVKVSCKVSGY | SYELTQPPSVSVAPGKTARITCGGD | SYELTQPPSVSVAPGKTARITCGGDN |
| | | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 277) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 222) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 16 | 1 | EVQLVQSGAEVKKPGASVKVSCKVSGYTLTE | EVQLVQSGAEVKKPGASVKVSCKVSGY | SYVLTQPPSVSVAPGKTARITCGGN | SYVLTQPPSVSVAPGKTARITCGGNN |
| | | LSMQWVRQAPGKGLEWMGGFDPDDGETMYAE | TLTELSMQWVRQAPGKGLEWMGGFDPD | NTGSKSVHWYQQKPGQAPVLVVYDD | TGSKSVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 278) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTRPREEQYNSTYRVVSVLTVL | | NO: 223) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 17 | 1 | EVQLVQSGAEVKKPGASVKVSCKVSGYTLTE | EVQLVQSGAEVKKPGASVKVSCKVSGY | SYVLTQPPSVSVAPGQTARITQGGS | SYVLTQPPSVSVAPGQTARITCGGSD |
| | | LSMQWVRQAPGKGLEWMGGFDPDDGETMYAE | TLTELSMQWVRQAPGKGLEWMGGFDPD | DIGDEIVHWYQQKPGQAPVLVVYDD | IGDEIVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRAQ |
| | | YFCATSPGYDFFDYWGQGTLVTVSSASTKGP | ELRSLTSEDTALYFCATSPGYDFFDYW | RAQAGDEADYYCQVWDSSSDHLIFG | AGDEADYYCQVWDSSSDHLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 279) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTIMISRTPEVTCVVVDVSHEDPEVKFNWY | | GSTVEKTVAPTECS (SEQ ID | |
| | | VDGVEVHNAKTRPREEQYNSTYRVVSVLTVL | | NO: 224) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 18 | 1 | EVQLVQSGAEVKKPGASVKVSCKVSGYTLTE | EVQLVQSGAEVKKPGASVKVSCKVSGY | SYVLTQPPSVSVAPGKTARITCGGN | SYVLTQPPSVSVAPGKTARITCGGNN |
| | | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | | KFQGRLTVTEDTSTDTVYMELRSLTSEDTAL | DGETMYAEKFQGRLTVTEDTSTDTVYM | SDRPLGIPERFSGSNSGNTATLTIN | RPLGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 280) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVI | | NO: 225) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 19 7 | 1 | EVQLVQSGAEVKKPGASVKVSCKVSGYTLTE | EVQLVQSGAEVKKPGASVKVSCKVSGY | SYVLTQPLSVSVALGQTARITCGGS | SYVLTQPLSVSVALGQTARITCGGSN |
| | | LSMQWVRQAPGKGLEWMGGFDPDDGETMYAE | TLTELSMQWVRQAPGKGLEWMGGFDPD | NIGDIRVHWYQQKPGQAPVLVVYDD | IGDIRVHWYQQRPGQAPVLVVYDDSD |
| | | KFQGRLTVTEDTPTDTVYMELRSLTSEDTAL | DGETMYAEKFQGRLTVTEDTPTDTVYM | SDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 172) | GGTKLTVLGQPKAAPSVTLFPPSSE | LTVI (SEQ ID NO: 281) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 226) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 126) | | | |
| 20 | 1 | EVQLVQSGAEVKKPGASVKVSCKVSGYTLTE | EVQLVQSGAEVKKPGASVKVSCKVSGY | SYVLTQPPSVSVAPGKTARITCGGN | SYVLTQPPSVSVAPGKTARITCGGNN |
| | | LSMQWVRQAPGKGLEWMGGFDPDDGETMYAE | TLTELSMQWVRQAPGKGLEWMGGFDPD | NIGSKGVHWYQQKPGQAPVLVVYDD | IGSKGVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 282) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 227) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 21 | 1 | EVQLVQSGAEVKKPGASVKVSCEVSGYTLTE | EVQLVQSGAEVKKPGASVKVSCEVSGY | SYVLTQPPSVSVAPGQTARVSCGGN | SYVLTQPPSVSVAPGQTARVSCGGNN |
| | | LSMQWVRQAPGKGLEWMGGFDPDDGETMYAE | TLTELSMQWVRQAPGKGLEWMGGFDPD | NIGSKSVNWYRQKPGQAPVLVIYDD | IGSKSVNWYRQKPGQAPVLVIYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 173) | GGTKLTVLGQPKAAPSVTLFPPSSE | LTVL (SEQ ID NO: 283) |
| | | TVSWNSGALTSGVHTFPAVLQSSGLYSLSSV | | ELQANKATLVCLISDFYPGAVTVAW | |
| | | VTVPSSSIGTQTYICNVNHKPSNTKVDKRVE | | KADSSPVKAGVETTTPSKQSNNKYA | |
| | | PRSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 228) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQRSLSLSPG (SEQ ID NO: 127) | | | |
| 22 | 1 | EVQLVQSGAEVKKPGASVKVSCKVSGYTLTE | EVQLVQSGAEVKKPGASVKVSCKVSGY | SYVLTQPPSVSVAPGKTARITCGGN | SYVLTQPPSVSVAPGKTARITCGGNN |
| | | LSMQWVRQAPGKGLEWMGGFDPDDGETMYAE | TLTELSMQWVRQAPGKGLEWMGGFDPD | NIGSKNVHWYQQKPGQAPVLVVYDD | IGSKNVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LIVL (SEQ ID NO: 284) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 229) | |
| | | HQDWLNGKEYKCKVSNKALPAPIEKTISKAK | | | |
| | | GQPREPQVYTIPPSREEMTKNQVSLTCLVKG | | | |
| | | FYPSDIAVEWESNGQPENNYKTTPPVLDSDG | | | |
| | | SFFLYSKITVDKSRWQQGNVFSCSVMHEALH | | | |
| | | NHYTQKSLSLSPG (SEQ ID NO: 119) | | | |
| 23 | 1 | EVQLVQSGAEVKKPGASVKVSCKVSGYTLTE | EVQLVQSGAEVKKPGASVKVSCKVSGY | SYVLTQPPSVSVAPGQTARITCGGN | SYVLTQPPSVSVAPGQTARITCGGNN |
| | | LSMQWVRQAPGKGLEWMGGFDPDDGETMYAE | TITELSMQWVRQAPGKGLEWMGGFDPD | NIGIKSVHWYQQKPGQAPVLVVYDD | IGIKSVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 285) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 230) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 24 | 1 | EVQLVQSGAEVKKPGASVKVSCKVSGYTLTE | EVQLVQSGAEVKKPGASVKVSCKVSGY | SYVLTQPPSVSVAPGKTARITCGGN | SYVLTQPPSVSVAPGKTARITCGGNN |
| | | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | | KLQGRLTVTEDTSTDTVYMELRSLTSEDTAL | DGETMYAEKLQGRLTVTEDTSTDTVYM | SDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 174) | GGTKLTVLGQPKAAPSVTLFPPSSE | LTVL (SEQ ID NO: 286) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 231) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 128) | | | |
| 25 | 1 | EVQLVQSGAEVKKPGASVKVSCKVSGYTLTE | EVQLVQSGAEVKKPGASVKVSCKVSGY | SYELTQPPSVAVAPGQTARITQGGN | SYELTQPPSVAVAPGQTARITCGGNN |
| | | LSMQWVRQAPGKGLEWMGGFDPDDGETMYAE | TLTELSMQWVRQAPGKGLEWMGGFDPD | NIGRKAVHWYQQKPGQAPVLVVYDD | IGRKAVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 287) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVI | | NO: 232) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 26 | 1 | EVQLVQSGAEVKKPGASVKVSCKVSGYTLTE | EVQLVQSGAEVKKPGASVKVSCKVSGY | SYVLTQPPSVSVAPGQTARITCGGN | SYVLTQPPSVSVAPGQTARITCGGNN |
| | | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRVE |
| | | YFCATSPGYDFFDYWGQGTLVTVSSASTKGP | ELRSLTSEDTALYFCATSPGYDFFDYW | RVEAGDEADYYCQVWDSSIDHLIFG | AGDEADYYCQVWDSSIDHLIFGGGTK |
| | | SVFPLAPSSKSTSGGTAALGCLVKDYFPEPV | GQGTLVTVSS (SEQ ID NO: 165) | GGTKVTVLGQPKAAPSVTLFPPSSE | VTVL (SEQ ID NO: 288) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTRPREEQYNSTYRVVSVLTVL | | NO: 233) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 27 | 1 | EVQLVQSGAEVKKPGASVKVSCKVSGYTLTE | EVQLVQSGAEVKKPGASVKVSCKVSGY | SYVLTQPPSVSVAPGQTARITCGGN | SYVLTQPPSVSVAPGQTARITCGGNN |
| | | LSMQWVRQAPGKGLEWMGGFDPDDGETMYAE | TLTELSMQWVRQAPGKGLEWMGGFDPD | NIGIKSVHWYQQKPGQAPVLVVYDD | IGIKSVHWYQQKPGQAPVLVVYDDRD |
| | | KFQGRLTVTEDTSTDTVYMELRSLTSEDTAL | DGETMYAEKFQGRLTVTEDTSTDTVYM | RDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 289) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTRPREEQYNSTYRVVSVLTVL | | NO: 234) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 28 | 1 | EVQLVQSGAEVKKPGASVKVSCKVSGYTLTE | EVQLVQSGAEVKKPGASVKVSCKVSGY | SYELTQPPSVSVAPGQTARITCGGN | SYELTQPPSVSVAPGQTARITCGGNN |
| | | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRVE |
| | | YFCATSPGYDFFDYWGQGTLVTVSSASTKGP | ELRSLTSEDTALYFCATSPGYDFFDYW | RVEAGDEADYYCQVWDADSDQLIFG | AGDEADYYCQVWDADSDQLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 290) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 235) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 29 | 1 | EVQLVQSGAEVKKPGASVKVSCKVSGYTLTE | EVQLVQSGAEVKKPGASVKVSCKVSGY | SYVLTQPPSVSVAPGKTARITQGGN | SYVLTQPPSVSVAPGKTARITCGGNN |
| | | LSMQWVRQAPGKGLEWMGGFDPDDGETMYAE | TLTELSMQWVRQAPGKGLEWMGGFDPD | NIGSKSVHWYQQKPGQAPVLVVYDD | IGSKSVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIS | RPSGIPERFSGSNSGNTATLTISRVE |
| | | YFCATSPGYDFFDYWGQGTLVTVSSASTKGP | ELRSLTSEDTALYFCATSPGYDFFDYW | RVEAGDEADYYCQVWDSSSDLLIFG | AGDEADYYCQVWDSSSDLLIFGGGAK |
| | | SVFPLAPSSKSTSGGTAALGCLVKDYFPEPV | GQGTLVTVSS (SEQ ID NO: 165) | GGAKLTVLGQPKAAPSVTLFPPSSE | LTVL (SEQ ID NO: 291) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 236) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 30 | 1 | EVQLVQSGAEVKKPGASVKVSCKVSGYTLTE | EVQLVQSGAEVKKPGASVKVSCKVSGY | SYVLTQPPSVSVAPGKTATITCGGN | SYVLTQPPSVSVAPGKTATITCGGNN |
| | | LSMQWVRQAPGKGLEWMGGFDPDDGETMYAE | TLTELSMQWVRQAPGKGLEWMGGFDPD | NIGTKSVHWYQQKPGQAPVLVVYDD | IGTKSVHWYQQKPGQAPVLVVYDDSD |
| | | KFQGRLTVTEDTSTDTVYMELRSLTSEDTAL | DGETMYAEKFQGRLTVTEDTSTDTVYM | SDRPSGIPERFSGSNSGNTATLTIN | RPSGIPERFSGSNSGNTATLTINRVE |
| | | YFCATSPGYDFFDYWGQGTLVTVSSASTKGP | ELRSLTSEDTALYFCATSPGYDFFDYW | RVEAGDEADYYCQVWDSSNDLLIFG | AGDEADYYCQVWDSSNDLLIFGGGTK |
| | | SVFPLAPSSKSTSGGTAALGCLVKDYFPEPV | GQGTLVTVSS (SEQ ID NO: 165) | GGTKLTVLGQPKAAPSVTLFPPSSE | LTVL (SEQ ID NO: 292) |
| | | TVSWNSGALTSGVHTFPAVLQSSGLYSLSSV | | ELQANKATLVCLISDFYPGAVTVAW | |
| | | VTVPSSSLGTQTYICNVNHKPSNTKVDKRVE | | KADSSPVKAGVETTTPSKQSNNKYA | |
| | | PKSCDKTHTCPPCPAPELLGGPSVFLFPPKP | | ASSYLSLTPEQWKSHRSYSCQVTHE | |
| | | KDTLMISRTPEVTCVVVDVSHEDPEVKFNWY | | GSTVEKTVAPTECS (SEQ ID | |
| | | VDGVEVHNAKTKPREEQYNSTYRVVSVLTVL | | NO: 237) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKLTVDKSRWQQGNVFSCSVMHEALH | | | |
| | | NHYTQKSLSLSPG (SEQ ID NO: 119) | | | |
| 31 | 2 | QVQLVQSGAEVKKPGATVKISCKVSGYSFTD | QVQLVQSGAEVKKPGATVKISCKVSGY | QSALTQPASVSGSPGQSVTISCTGT | QSALTQPASVSGSPGQSVTISCTGTS |
| | | YFIHWVQQAPGKGLAWVGLVDPEDGETIYAE | SFTDYFIHWVQQAPGKGLAWVGLVDPE | SSDVGGYNYVSWYQHHPGKAPKLMI | SDVGGYNYVSWYQHHPGKAPKLMIYD |
| | | KFQGRVTITADTSTDTAYMELSSLRSEDTAV | DGETIYAEKFQGRVTITADTSTDTAYM | YDVNKRPSGVPDRFSGSKSGNTASL | VNKRPSGVPDRFSGSKSGNTASLTIS |
| | | YYCATPGGILTDPDAFDIWGQGTMVTVSSAS | ELSSLRSEDTAVYYCATPGGILTDPDA | TISGLQAEDEADYYCSSYIPSSTRW | GLQAEDEADYYCSSYIPSSTRWVFGG |
| | | TKGPSVFPLAPSSKSTSGGTAALGCLVKDYF | FDIWGQGTMVTVSS (SEQ ID | VFGGGTKLTVLGQPKAAPSVTLFPP | GTKLTVL (SEQ ID NO: 293) |
| | | PEPVTVSWNSGALTSGVHTFPAVLQSSGLYS | NO: 175) | SSEELQANKATLVCLISDFYPGAVT | |
| | | LSSVVTVPSSSLGTQTYICNVNHKPSNTKVD | | VAWKADSSPVKAGVETTTPSKQSNN | |
| | | KRVEPKSCDKTHTCPPCPAPELLGGPSVFLF | | KYAASSYLSLTPEQWKSHRSYSCQV | |
| | | PPKPKDTLMISRTPEVTCVVVDVSHEDPEVK | | THEGSTVEKTVAPTECS (SEQ ID | |
| | | FNWYVDGVEVHNAKTKPREEQYNSTYRVVSV | | NO: 238) | |
| | | LTVLHQDWLNGKEYKCKVSNKALPAPIEKTI | | | |
| | | SKAKGQPREPQVYTLPPSREEMTKNQVSLTC | | | |
| | | LVKGFYPSDIAVEWESNGQPENNYKTTPPVL | | | |
| | | DSDGSFFLYSKLTVDKSRWQQGNVFSCSVMH | | | |
| | | EALHNHYTQKSLSLSPG (SEQ ID | | | |
| | | NO: 129) | | | |
| 32 | 2 | QVQLVQSGAEVKKPGATVKISCKVSGYSETD | QVQLVQSGAEVKKPGATVKISCKVSGY | QSALTQPASVSGSPGQSITISCTGT | QSALTQPASVSGSPGQSITISCTGTS |
| | | YFIHWVQQAPGKGLAWVGLVDPEDGETIYAE | SFTDYFIHWVQQAPGKGLAWVGLVDPE | SSDVGGYNYVSWYQQHPGKAPKLMI | SDVGGYNYVSWYQQHPGKAPKLMIYE |
| | | KFQGRVTITADTSTDTAYMELSSLRSEDTAV | DGETIYAEKFQGRVTITADTSTDTAYM | YEVSNRPSGVSNRFSGSKSGNTASL | VSNRPSGVSNRFSGSKSGNTASITIS |
| | | YYCATPGGILTDPDAFDIWGQGTMVTVSSAS | ELSSLRSEDTAVYYCATPGGILTDPDA | TISGLQAEDEADYYCSSYTSSSTPY | GLQAEDEADYYCSSYTSSSTPYVEGT |
| | | TKGPSVFPLAPSSKSTSGGTAALGCLVKDYF | FDIWGQGTMVTVSS (SEQ ID | VFGTGTKVTVLGQPKAAPSVTLFPP | GTKVTVL (SEQ ID NO: 294) |
| | | PEPVTVSWNSGALTSGVHTFPAVLQSSGLYS | NO: 175) | SSEELQANKATLVCLISDFYPGAVT | |
| | | LSSVVTVPSSSLGTQTYICNVNHKPSNTKVD | | VAWKADSSPVKAGVETTTPSKQSNN | |
| | | KRVEPKSCDKTHTCPPCPAPELLGGPSVFLF | | KYAASSYLSLTPEQWKSHRSYSCQV | |
| | | PPKPKDTIMISRTPEVTCVVVDVSHEDPEVK | | THEGSTVEKTVAPTECS (SEQ ID | |
| | | FNWYVDGVEVHNAKTKPREEQYNSTYRVVSV | | NO: 239) | |
| | | LTVLHQDWLNGKEYKCKVSNKALPAPIEKTI | | | |
| | | SKAKGQPREPQVYTLPPSREEMTKNQVSLTC | | | |
| | | LVKGFYPSDIAVEWESNGQPENNYKTTPPVL | | | |
| | | DSDGSFFLYSKLTVDKSRWQQGNVFSCSVMH | | | |
| | | EALHNHYTQKSLSLSPG (SEQ ID | | | |
| | | NO: 129) | | | |
| 33 | 2 | QVQLVQSGAEVKKPGATVKISCKVSGYSFTD | QVQLVQSGAEVKKPGATVKISCKVSGY | QSALTQPRSVSGSPGQSVTISCAGT | QSALTQPRSVSGSPGQSVTISCAGTG |
| | | YFIHWVQQAPGKGLAWVGLVDPEDGETIYAE | SFTDYFIHWVQQAPGKGLAWVGLVDPE | GSDVGGYNHVSWYQHHPGKAPKLMI | SDVGGYNHVSWYQHHPGKAPKLMIYD |
| | | KFQGRVTITADTSTDTAYMELSSLRSEDTAV | DGETIYAEKFQGRVTITADTSTDTAYM | YDVNKRPSGVPDRFSGSKSGNTASL | VNKRPSGVPDRFSGSKSGNTASLTIS |
| | | YYCATPGGILTDPDAFDIWGQGTMVTVSSAS | ELSSLRSEDTAVYYCATPGGILTDPDA | TISGLQAEDEADYYCSSYIPSSTRW | GLQAEDEADYYCSSYIPSSTRWVEGG |
| | | TKGPSVFPLAPSSKSTSGGTAALGCLVKDYF | FDIWGQGIMVTVSS (SEQ ID | VFGGGTKLTVLGQPKAAPSVTLFPP | GTKLTVL (SEQ ID NO: 295) |
| | | PEPVTVSWNSGALTSGVHTFPAVLQSSGLYS | NO: 175) | SSEELQANKATLVCLISDFYPGAVT | |
| | | LSSVVTVPSSSLGTQTYICNVNHKPSNTKVD | | VAWKADSSPVKAGVETTTPSKQSNN | |
| | | KRVEPKSCDKTHTCPPCPAPELLGGPSVFLF | | KYAASSYLSLTPEQWKSHRSYSCQV | |
| | | PPKPKDTLMISRTPEVTCVVVDVSHEDPEVK | | THEGSTVEKTVAPTECS (SEQ ID | |
| | | FNWYVDGVEVHNAKTKPREEQYNSTYRVVSV | | NO: 240) | |
| | | LTVLHQDWLNGKEYKCKVSNKALPAPIEKTI | | | |
| | | SKAKGQPREPQVYTLPPSREEMTKNQVSLTC | | | |
| | | LVKGFYPSDIAVEWESNGQPENNYKTTPPVL | | | |
| | | DSDGSFFLYSKLTVDKSRWQQGNVFSCSVMH | | | |
| | | EALHNHYTQKSISLSPG (SEQ ID | | | |
| | | NO: 129) | | | |
| 34 | 2 | QVQLVQSGAEVKKPGATVKISCKVSGYSFTD | QVQLVQSGAEVKKPGATVKISCKVSGY | QSALTQPASVSGSPGQSITISCTGT | QSALTQPASVSGSPGQSITISCTGTS |
| | | YFIHWVQQAPGKGLAWVGLVDPEDGETIYAE | SFTDYFIHWVQQAPGKGLAWVGLVDPE | SSDVGGYNYVSWYQQHPGKAPKLMI | SDVGGYNYVSWYQQHPGKAPKLMIYE |
| | | KFQGRVTITADTSTDTAYMELSSLRSEDTAV | DGETIYAEKEQGRVTITADTSTDTAYM | YEVSNRPSGVSNRFSGSKSGNTASL | VSNRPSGVSNRFSGSKSGNTASLTIS |
| | | YYCATPGGILTDPDAFDIWGQGTMVTVSSAS | ELSSLRSEDTAVYYCATPGGILTDPDA | TISGLQAEDEADYYCSSYTSSSTYV | GLQAEDEADYYCSSYTSSSTYVFGTG |
| | | TKGPSVFPLAPSSKSTSGGTAALGCLVKDYF | FDIWGQGTMVTVSS (SEQ ID | FGTGTKVTVLGQPKAAPSVTLFPPS | TKVTVL (SEQ ID NO: 296) |
| | | PEPVTVSWNSGALTSGVHTFPAVLQSSGLYS | NO: 175) | SEELQANKATLVCLISDFYPGAVTV | |
| | | LSSVVTVPSSSLGTQTYICNVNHKPSNTKVD | | AWKADSSPVKAGVETTTPSKQSNNK | |
| | | KRVEPKSCDKTHTCPPCPAPELLGGPSVFLF | | YAASSYLSLTPEQWKSHRSYSCQVT | |
| | | PPKPKDTLMISRTPEVTCVVVDVSHEDPEVK | | HEGSTVEKTVAPTECS (SEQ ID | |
| | | FNWYVDGVEVHNAKTRPREEQYNSTYRVVSV | | NO: 241) | |
| | | LTVLHQDWLNGKEYKCKVSNKALPAPIEKTI | | | |
| | | SKAKGQPREPQVYTLPPSREEMTKNQVSLTC | | | |
| | | LVKGFYPSDIAVEWESNGQPENNYKTTPPVL | | | |
| | | DSDGSFFLYSKLTVDKSRWQQGNVFSCSVMH | | | |
| | | EALHNHYTQKSLSLSPG (SEQ ID | | | |
| | | NO: 129) | | | |
| 35 | 2 | QVQLVQSGAEVKKPGATVKISCKVSGYSFTD | QVQLVQSGAEVKKPGATVKISCKVSGY | QSALTQPASVSGSPGQSITISCTGT | QSALTQPASVSGSPGQSITISCTGTS |
| | | YFIHWVQQAPGKGLAWVGLVDPEDGETIYAE | SFTDYFIHWVQQAPGKGLAWVGLVDPE | SSDVGGYNYVSWYQQHPGKAPKLMI | SDVGGYNYVSWYQQHPGKAPKLMIYE |
| | | RFQGRVTITADTSTDTAYMELSSLRSEDTAV | DGETIYAERFQGRVTITADTSTDTAYM | YEVSNRPSGVSNRFSGSKSGNTASL | VSNRPSGVSNRFSGSKSGNTASITIS |
| | | YYCATPGGILTDPDAFDIWGQGTMVTVSSAS | ELSSLRSEDTAVYYCATPGGILTDPDA | TISGLQAEDEADYYCSSYTSSSTPY | GLQAEDEADYYCSSYTSSSTPYVFGT |
| | | TKGPSVFPLAPSSKSTSGGTAALGCLVKDYF | FDIWGQGTMVTVSS (SEQ ID | VFGTGTKVTVLGQPKAAPSVTLFPP | GTKVTVL (SEQ ID NO: 294) |
| | | PEPVTVSWNSGALTSGVHTFPAVLQSSGLYS | NO: 176) | SSEELQANKATLVCLISDFYPGAVT | |
| | | LSSVVTVPSSSLGTQTYICNVNHKPSNTKVD | | VAWKADSSPVKAGVETTTPSKQSNN | |
| | | KRVEPKSCDKTHTCPPCPAPELLGGPSVFLF | | KYAASSYLSLTPEQWKSHRSYSCQV | |
| | | PPKPKDTLMISRTPEVTCVVVDVSHEDPEVK | | THEGSTVEKTVAPTECS (SEQ ID | |
| | | FNWYVDGVEVHNAKTKPREEQYNSTYRVVSV | | NO: 239) | |
| | | LTVLHQDWLNGKEYKCKVSNKALPAPIEKTI | | | |
| | | SKAKGQPREPQVYTLPPSREEMTKNQVSLTC | | | |
| | | LVKGFYPSDIAVEWESNGQPENNYKTTPPVL | | | |
| | | DSDGSFFLYSKLTVDKSRWQQGNVFSCSVMH | | | |
| | | EALHNHYTQKSLSLSPG (SEQ ID | | | |
| | | NO: 130) | | | |
| 36 | 2 | QVQLVQSGAEVKKPGATVKISCKVSGYSFTD | QVQLVQSGAEVKKPGATVKISCKVSGY | QSALTQPRSVSGSPGQSVTISCAGT | QSALTQPRSVSGSPGQSVTISCAGTG |
| | | YFIHWVQQAPGKGLAWVGLVDPEDGETIYAE | SFTDYFIHWVQQAPGKGLAWVGLVDPE | GSDVGGYNYVSWYQHHPGKAPKLMI | SDVGGYNYVSWYQHHPGKAPKLMIYD |
| | | KFQGRVTITADTSTDTAYMELSSLRSEDTAV | DGETIYAEKFQGRVTITADTSTDTAYM | YDVNKRPSGVPDRFSGSKSGNTASL | VNKRPSGVPDRFSGSKSGNTASLTIS |
| | | YYCATPGGILTDPDAFDIWGQGTMVTVSSAS | ELSSLRSEDTAVYYCATPGGILTDPDA | TISGLQAEDEADYYCSSYTSSSTFY | GLQAEDEADYYCSSYTSSSTFYVFGT |
| | | TKGPSVFPLAPSSKSTSGGTAALGCLVKDYF | FDIWGQGTMVTVSS (SEQ ID | VFGTGTKVTVLGQPKAAPSVTLFPP | GTKVTVL (SEQ ID NO: 297) |
| | | PEPVTVSWNSGALTSGVHTFPAVLQSSGLYS | NO: 175) | SSEELQANKATLVCLISDFYPGAVT | |
| | | LSSVVTVPSSSLGTQTYICNVNHKPSNTKVD | | VAWKADSSPVKAGVETTTPSKQSNN | |
| | | KRVEPKSCDKTHTCPPCPAPELLGGPSVFLF | | KYAASSYLSLTPEQWKSHRSYSCQV | |
| | | PPKPKDTLMISRTPEVTCVVVDVSHEDPEVK | | THEGSTVEKTVAPTECS (SEQ ID | |
| | | FNWYVDGVEVHNAKTKPREEQYNSTYRVVSV | | NO: 242) | |
| | | LTVLHQDWLNGKEYKCKVSNKALPAPIEKTI | | | |
| | | SKAKGQPREPQVYTLPPSREEMTKNQVSLTC | | | |
| | | LVKGFYPSDIAVEWESNGQPENNYKTTPPVL | | | |
| | | DSDGSFFLYSKLTVDKSRWQQGNVFSCSVMH | | | |
| | | EALHNHYTQKSLSLSPG (SEQ ID | | | |
| | | NO: 129) | | | |
| 37 | 2 | QVQLVQSGAEVKKPGATVKISCKVSGYSFTD | QVQLVQSGAEVKKPGATVKISCKVSGY | QSALTQPASVSGSPGQSITISCTGT | QSALTQPASVSGSPGQSITISCTGTS |
| | | YFIHWVQQAPGKGLAWVGLVDPEDGETIYAE | SFTDYFIHWVQQAPGKGLAWVGLVDPE | SSDVGGYNYVSWYQQHPGKAPKLMI | SDVGGYNYVSWYQQHPGKAPKLMIYD |
| | | KFQGRVTITADTSTDTAYMELSSLRSEDTAV | DGETIYAEKFQGRVTITADTSTDTAYM | YDVSNRPSGVSNRFSGSKSGNTASL | VSNRPSGVSNRFSGSKSGNTASLTIS |
| | | YYCATPGGILTDPDAFDIWGQGTMVTVSSAS | ELSSLRSEDTAVYYCATPGGILTDPDA | TISGLQAEDEADYYCSSYTSSSTFY | GLQAEDEADYYCSSYTSSSTFYVFGT |
| | | TKGPSVFPLAPSSKSTSGGTAALGCLVKDYF | FDIWGQGTMVTVSS (SEQ ID | VFGTGTKVTVLGQPKAAPSVTLFPP | GTKVTVL (SEQ ID NO: 298) |
| | | PEPVTVSWNSGALTSGVHTFPAVLQSSGLYS | NO: 175) | SSEELQANKATLVCLISDEYPGAVT | |
| | | LSSVVTVPSSSLGTQTYICNVNHKPSNTKVD | | VAWKADSSPVKAGVETTTPSKQSNN | |
| | | KRVEPKSCDKTHTCPPCPAPELLGGPSVFLF | | KYAASSYLSLTPEQWKSHRSYSCQV | |
| | | PPKPKDTLMISRTPEVTCVVVDVSHEDPEVK | | THEGSTVEKTVAPTECS (SEQ ID | |
| | | FNWYVDGVEVHNAKTKPREEQYNSTYRVVSV | | NO: 243) | |
| | | LTVLHQDWLNGKEYKCKVSNKALPAPIEKTI | | | |
| | | SKAKGQPREPQVYTLPPSREEMTKNQVSLTC | | | |
| | | LVKGFYPSDIAVEWESNGQPENNYKTTPPVL | | | |
| | | DSDGSFFLYSKLTVDKSRWQQGNVFSCSVMH | | | |
| | | EALHNHYTQKSLSLSPG (SEQ ID | | | |
| | | NO: 129) | | | |
| 38 | 3 | EVQPVQSGAEVKKPGSSVKVSCKASGGTFSS | EVQPVQSGAEVKKPGSSVKVSCKASGG | DIQLTQSPSSLSASVGDRVTITCQA | DIQLTQSPSSLSASVGDRVTITCQAS |
| | | YAISWVRQAPGQGLEWMGLVDPEDGETIYAE | TFSSYAISWVRQAPGQGLEWMGLVDPE | SQDISNYLNWYQQKPGKAPKLLIYD | QDISNYLNWYQQKPGKAPKLLIYDAS |
| | | KFRGRVTITADTSTDTAYMELSSLRSEDTAV | DGETIYAEKFRGRVTITADTSTDTAYM | ASNLETGVPSRFSGSGSGTEFTLTI | NLETGVPSRFSGSGSGTEFTLTISSL |
| | | YYCATEESYGPWGQGTLVTVSSASTKGPSVF | ELSSLRSEDTAVYYCATEESYGPWGQG | SSLQPEDFATYYCQQLNSYPLTEGG | QPEDFATYYCQQLNSYPLTFGGGTKL |
| | | PLAPSSKSTSGGTAALGCLVKDYFPEPVTVS | TLVTVSS (SEQ ID NO: 177) | GTKLEIKRTVAAPSVFIFPPSDEQL | EIK (SEQ ID NO: 299) |
| | | WNSGALTSGVHTFPAVLQSSGLYSLSSVVTV | | KSGTASVVCLLNNFYPREAKVQWKV | |
| | | PSSSLGTQTYICNVNHKPSNTKVDKRVEPKS | | DNALQSGNSQESVTEQDSKDSTYSL | |
| | | CDKTHTCPPCPAPELLGGPSVFLFPPKPKDT | | SSTLTLSKADYEKHKVYACEVTHQG | |
| | | LMISRTPEVTCVVVDVSHEDPEVKFNWYVDG | | LSSPVTKSENRGEC (SEQ ID | |
| | | VEVHNAKTKPREEQYNSTYRVVSVLTVLHQD | | NO: 244) | |
| | | WLNGKEYKCKVSNKALPAPIEKTISKAKGQP | | | |
| | | REPQVYTIPPSREEMTKNQVSLTCLVKGFYP | | | |
| | | SDIAVEWESNGQPENNYKTTPPVLDSDGSFF | | | |
| | | LYSKLTVDKSRWQQGNVFSCSVMHEALHNHY | | | |
| | | TQKSLSLSPG (SEQ ID NO: 131) | | | |
| 39 | 3 | EVQLVQSGAEVKKPGSSVKVSCKASGGTFSS | EVQLVQSGAEVKKPGSSVKVSCKASGG | DIQLTQSPSSLSASVGDRVTITCQA | DIQLTQSPSSLSASVGDRVTITCQAS |
| | | YAISWVRQAPGQGLEWMGLVDPEDGETIYAE | TFSSYAISWVRQAPGQGLEWMGLVDPE | SQDISNYLDWYQQKPGKAPKLLIYD | QDISNYLDWYQQKPGKAPKLLIYDAS |
| | | KFQGRVTITADTSTDTAYMELSSLRSEDTAV | DGETIYAEKFQGRVTITADTSTDTAYM | ASNLETGVPSRFSGSGSGTEFTLTI | NLETGVPSRFSGSGSGTEFTLTISSL |
| | | YYCATEESYGPWGQGTLVTVSSASTKGPSVF | ELSSLRSEDTAVYYCATEESYGPWGQG | SSLQPEDFATYYCQQLNSYPLTEGG | QPEDFATYYCQQLNSYPLTFGGGTKL |
| | | PLAPSSKSTSGGTAALGCLVKDYFPEPVTVS | TLVTVSS (SEQ ID NO: 178) | GTKLEIKRTVAAPSVFIFPPSDEQL | EIK (SEQ ID NO: 300) |
| | | WNSGALTSGVHTFPAVLQSSGLYSLSSVVTV | | KSGTASVVCLLNNFYPREAKVQWKV | |
| | | PSSSLGTQTYICNVNHKPSNTKVDKRVEPKS | | DNALQSGNSQESVTEQDSKDSTYSL | |
| | | CDKTHTCPPCPAPELLGGPSVFLFPPKPKDT | | SSTLTLSKADYEKHKVYACEVTHQG | |
| | | LMISRTPEVTCVVVDVSHEDPEVKFNWYVDG | | LSSPVTKSENRGEC (SEQ ID | |
| | | VEVHNAKTKPREEQYNSTYRVVSVLTVLHQD | | NO: 245) | |
| | | WLNGKEYKCKVSNKALPAPIEKTISKAKGQP | | | |
| | | REPQVYTIPPSREEMTKNQVSLTCLVKGFYP | | | |
| | | SDIAVEWESNGQPENNYKTTPPVLDSDGSFF | | | |
| | | LYSKLTVDKSRWQQGNVFSCSVMHEALHNHY | | | |
| | | TQKSLSLSPG (SEQ ID NO: 132) | | | |
| 40 | 4 | EVQLVQSGAEVKKPGASVKVSCKVSGYTLTE | EVQLVQSGAEVKKPGASVKVSCKVSGY | QSVLTQPPSVSAAPGQKVTISCSGS | QSVLTQPPSVSAAPGQKVTISCSGSS |
| | | SSMHWVRQAPGKGLEWMGLVDPEDGETIYAQ | TLTESSMHWVRQAPGKGLEWMGLVDPE | SSNIGSYSVSWYQQLPGTAPKLLIY | SNIGSYSVSWYQQLPGTAPKLLIYDN |
| | | KFQGRVTMTEDTSTDTAYMELSSLRSEDTAV | DGETIYAQKFQGRVTMTEDTSTDTAYM | DNNKRPSGIPDRFSGSNSGTSATLG | NKRPSGIPDRFSGSNSGTSATLGITG |
| | | YYCAREEWSGDGDDAFDIWGQGTMVTVSSAS | ELSSLRSEDTAVYYCAREEWSGDGDDA | ITGLQTGDEADYYCGTWDSSLSAWV | LQTGDEADYYCGTWDSSLSAWVFGGG |
| | | TKGPSVFPLAPSSKSTSGGTAALGCLVKDYF | FDIWGQGTMVTVSS (SEQ ID | FGGGTKLTVLGQPKAAPSVTLFPPS | TKLIVL (SEQ ID NO: 301) |
| | | PEPVTVSWNSGALTSGVHTFPAVLQSSGLYS | NO: 179) | SEELQANKATLVCLISDFYPGAVTV | |
| | | LSSVVTVPSSSIGTQTYICNVNHKPSNTKVD | | AWKADSSPVKAGVETTTPSKQSNNK | |
| | | KRVEPKSCDKTHTCPPCPAPELLGGPSVFLF | | YAASSYLSLTPEQWKSHRSYSCQVT | |
| | | PPKPKDTLMISRTPEVTCVVVDVSHEDPEVK | | HEGSTVEKTVAPTECS (SEQ ID | |
| | | FNWYVDGVEVHNAKTKPREEQYNSTYRVVSV | | NO: 246) | |
| | | LTVLHQDWLNGKEYKCKVSNKALPAPIEKTI | | | |
| | | SKAKGQPREPQVYTIPPSREEMTKNQVSLTC | | | |
| | | LVKGFYPSDIAVEWESNGQPENNYKTTPPVL | | | |
| | | DSDGSFFLYSKLTVDKSRWQQGNVFSCSVMH | | | |
| | | EALANHYTQKSLSLSPG (SEQ ID | | | |
| | | NO: 133) | | | |
| 41 | 4 | EVQLVQSGAEVKKPGASVKVSCKVSGYTLTE | EVQLVQSGAEVKKPGASVKVSCKVSGY | QSVLTQPPSVSAAPGQKVTISCSGS | QSVLTQPPSVSAAPGQKVTISCSGSS |
| | | LSMHWVRQAPGKGLEWMGLVDPEDGETIYAQ | TLTELSMHWVRQAPGKGLEWMGLVDPE | SSNIGSYSVSWYQQLPGTAPKLLIY | SNIGSYSVSWYQQLPGTAPKLLIYDN |
| | | KFQGRVTMTEDTSTDTAYMELSSLRSEDTAV | DGETIYAQKFQGRVTMTEDTSTDTAYM | DNNKRPSGIPDRFSGSNSGTSATLG | NKRPSGIPDRFSGSNSGTSATLGITG |
| | | YYCAREEWSGDGDDAFDIWGQGTMVTVSSAS | ELSSLRSEDTAVYYCAREEWSGDGDDA | ITGLQTGDEADYYCGTWDSSLSAWV | LQTGDEADYYCGTWDSSLSAWVFGGG |
| | | TKGPSVFPLAPSSKSTSGGTAALGCLVKDYF | FDIWGQGTMVTVSS (SEQ ID | FGGGTKLTVLGQPKAAPSVTLFPPS | TKLTVL (SEQ ID NO: 301) |
| | | PEPVTVSWNSGALTSGVHTFPAVLQSSGLYS | NO: 180) | SEELQANKATLVCLISDFYPGAVTV | |
| | | LSSVVTVPSSSLGTQTYICNVNHKPSNTKVD | | AWKADSSPVKAGVETTTPSKQSNNK | |
| | | KRVEPKSCDKTHTCPPCPAPELLGGPSVFLF | | YAASSYLSLTPEQWKSHRSYSCQVT | |
| | | PPKPKDTLMISRTPEVTCVVVDVSHEDPEVK | | HEGSTVEKTVAPTECS (SEQ ID | |
| | | FNWYVDGVEVHNAKTKPREEQYNSTYRVVSV | | NO: 246) | |
| | | LTVLHQDWLNGKEYKCKVSNKALPAPIEKTI | | | |
| | | SKAKGQPREPQVYTLPPSREEMTKNQVSLTC | | | |
| | | LVKGFYPSDIAVEWESNGQPENNYKTTPPVL | | | |
| | | DSDGSFFLYSKLTVDKSRWQQGNVFSCSVMH | | | |
| | | EALHNHYTQKSLSLSPG (SEQ ID | | | |
| | | NO: 134) | | | |
| 42 | 4 | EVQLVQSGAEVKKPGASVKVSCKVSGYTLTE | EVQLVQSGAEVKKPGASVKVSCKVSGY | QSVLTQPPSVSAAPGQKVTISCSGS | QSVLTQPPSVSAAPGQKVTISCSGSS |
| | | LSMHWVRQAPGKGLEWMGLVDPEDGETIYAç | TLTELSMHWVRQAPGKGLEWMGLVDPE | SSNIGSYSVSWYQQLPGTAPKLLIY | SNIGSYSVSWYQQLPGTAPKLLIYDD |
| | | KFQGRVTMTEDTSTDTAYMELSSLRSEDTAV | DGETIYAQKFQGRVTMTEDTSTDTAYM | DDNKRPSGIPDRFSGSNSGTSATLS | NKRPSGIPDRFSGSNSGTSATLSITG |
| | | YYCAREEWSGDGDDAFDIWGQGTMVTVSSAS | ELSSLRSEDTAVYYCAREEWSGDGDDA | ITGLQTGDEADYYCGTWDSSLSAWV | LQTGDEADYYCGTWDSSLSAWVFGGG |
| | | TKGPSVFPLAPSSKSTSGGTAALGCLVKDYF | FDIWGQGTMVTVSS (SEQ ID | FGGGTKLTVLGQPKAAPSVTLFPPS | TKLTVL (SEQ ID NO: 302) |
| | | PEPVTVSWNSGALTSGVHTFPAVLQSSGLYS | NO: 180) | SEELQANKATLVCLISDFYPGAVTV | |
| | | LSSVVTVPSSSLGTQTYICNVNHKPSNTKVD | | AWKADSSPVKAGVETTTPSKQSNNK | |
| | | KRVEPKSCDKTHTCPPCPAPELLGGPSVFLF | | YAASSYLSLTPEQWKSHRSYSCQVT | |
| | | PPKPKDTLMISRTPEVTCVVVDVSHEDPEVK | | HEGSTVEKTVAPTECS (SEQ ID | |
| | | FNWYVDGVEVHNAKTKPREEQYNSTYRVVSV | | NO: 247) | |
| | | LTVLHQDWLNGKEYKCKVSNKALPAPIEKTI | | | |
| | | SKAKGQPREPQVYTLPPSREEMTKNQVSLTC | | | |
| | | LVKGFYPSDIAVEWESNGQPENNYKTTPPVI | | | |
| | | DSDGSFFLYSKLTVDKSRWQQGNVESCSVMH | | | |
| | | EALHNHYTQKSISLSPG (SEQ ID | | | |
| | | NO: 134) | | | |
| 43 | 6 | EVQLVQSGAEVKKPGASVKVSCKASGGTFSS | EVQLVQSGAEVKKPGASVKVSCKASGG | SYELTQPPSVSVAPGKTARVTCGGN | SYELTQPPSVSVAPGKTARVTCGGNN |
| | | YAISWVRQAPGQGLEWMGGIIPIFGTANYAQ | TFSSYAISWVRQAPGQGLEWMGGIIPI | NIRAKHVHWYQQKPGQSPVLVIYQD | IRAKHVHWYQQKPGQSPVLVIYQDSK |
| | | KFQGRVTITADESTSTAYMELSSLRSEDTAV | FGTANYAQKFQGRVTITADESTSTAYM | SKRPSGIPERFSGSNSGNTATLTIS | RPSGIPERFSGSNSGNTATLTISGTQ |
| | | YYCASSPLRGSGWYWHYYYGMDVWGQGTTVT | ELSSLRSEDTAVYYCASSPLRGSGWYW | GTQAMDEADYYCQAWDSSTVVFGGG | AMDEADYYCQAWDSSTVVFGGGTKLT |
| | | VSSASTKGPSVFPLAPSSKSTSGGTAALGCL | HYYYGMDVWGQGTTVTVSS (SEQ ID | TKLTVLGQPKAAPSVTLFPPSSEEL | VI (SEQ ID NO: 303) |
| | | VKDYFPEPVTVSWNSGALTSGVHTFPAVLQS | NO: 181) | QANKATLVCLISDFYPGAVTVAWKA | |
| | | SGLYSLSSVVTVPSSSLGTQTYICNVNHKPS | | DSSPVKAGVETTTPSKQSNNKYAAS | |
| | | NTKVDKRVEPKSCDKTHTCPPCPAPELLGGP | | SYLSLTPEQWKSHRSYSCQVTHEGS | |
| | | SVFLFPPKPKDTLMISRTPEVTCVVVDVSHE | | TVEKTVAPTECS (SEQ ID | |
| | | DPEVKFNWYVDGVEVHNAKTKPREEQYNSTY | | NO: 248) | |
| | | RVVSVLTVLHQDWLNGKEYKCKVSNKALPAP | | | |
| | | IEKTISKAKGQPREPQVYTLPPSREEMTKNQ | | | |
| | | VSLTCLVKGFYPSDIAVEWESNGQPENNYKT | | | |
| | | TPPVLDSDGSFFLYSKLTVDKSRWQQGNVFS | | | |
| | | CSVMHEALHNHYTQKSLSLSPG (SEQ ID | | | |
| | | NO: 135) | | | |
| 44 | 6 | EVQLVQSGAEVKKPGSSVKVSCKASGGTFSS | EVQLVQSGAEVKKPGSSVKVSCKASGG | SYVLTQPPSVSAAPGKTARISCGGN | SYVLTQPPSVSAAPGKTARISCGGNN |
| | | YAISWVRQAPGQGLEWMGGIIPIFGTANYAQ | TFSSYAISWVRQAPGQGLEWMGGIIPI | NIRSKSVHWYQQKAGQAPVLVIYQD | IRSKSVHWYQQKAGQAPVLVIYQDSK |
| | | KFQGRVTITADESTSTAYMELSSLRSEDTAV | FGTANYAQKFQGRVTITADESTSTAYM | SKRPSGIPERFSGSNSGNTATLTIS | RPSGIPERFSGSNSGNTATLTISGTQ |
| | | YYCASSPLRGSGWYWHYYYGMDVWGQGTTVT | ELSSLRSEDTAVYYCASSPLRGSGWYW | GTQAMDEADYYCQAWDSNTVVFGGG | AMDEADYYCQAWDSNTVVFGGGTKLT |
| | | VSSASTKGPSVFPLAPSSKSTSGGTAALGCL | HYYYGMDVWGQGTTVTVSS (SEQ ID | TKLTVLGQPKAAPSVTLFPPSSEEL | VI (SEQ ID NO: 304) |
| | | VKDYFPEPVTVSWNSGALTSGVHTFPAVLQS | NO: 182) | QANKATLVCLISDFYPGAVTVAWKA | |
| | | SGLYSLSSVVTVPSSSLGTQTYICNVNHKPS | | DSSPVKAGVETTTPSKQSNNKYAAS | |
| | | NTKVDKRVEPKSCDKTHTCPPCPAPELLGGP | | SYLSLTPEQWKSHRSYSCQVTHEGS | |
| | | SVFLFPPKPKDTLMISRTPEVTCVVVDVSHE | | TVEKTVAPTECS (SEQ ID | |
| | | DPEVKFNWYVDGVEVHNAKTKPREEQYNSTY | | NO: 249) | |
| | | RVVSVLTVLHQDWLNGKEYKCKVSNKALPAP | | | |
| | | IEKTISKAKGQPREPQVYTLPPSREEMTKNQ | | | |
| | | VSLTCLVKGFYPSDIAVEWESNGQPENNYKT | | | |
| | | TPPVLDSDGSFFLYSKLTVDKSRWQQGNVFS | | | |
| | | CSVMHEALHNHYTQKSLSLSPG (SEQ ID | | | |
| | | NO: 136) | | | |
| 45 | 6 | EVQLVQSGAEVKKPGSSVKVSCKASGGTFSS | EVQLVQSGAEVKKPGSSVKVSCKASGG | SYVLTQPLSVSVAPGQTARITCGGN | SYVLTQPLSVSVAPGQTARITCGGNN |
| | | YAISWVRQAPGQGLEWMGGIIPIFGTANYAQ | TFSSYAISWVRQAPGQGLEWMGGIIPI | NIGRKSVHWYQQKPGQAPVLVIYRD | IGRKSVHWYQQKPGQAPVLVIYRDSN |
| | | KFQGRVTITADESTSTAYMELSSLRSEDTAV | FGTANYAQKFQGRVTITADESTSTAYM | SNRPSGIPERFSGSNSGNTATLTIS | RPSGIPERFSGSNSGNTATLTISRAQ |
| | | YYCASSPLRGSGWYWHYYYGMDVWGQGTTVT | ELSSLRSEDTAVYYCASSPLRGSGWYW | RAQAGDEADYYCQVWDSSTVVFGGG | AGDEADYYCQVWDSSTVVFGGGTKLT |
| | | VSSASTKGPSVFPLAPSSKSTSGGTAALGCL | HYYYGMDVWGQGTTVTVSS (SEQ ID | TKLTVLGQPKAAPSVTLFPPSSEEL | VL (SEQ ID NO: 305) |
| | | VKDYFPEPVTVSWNSGALTSGVHTFPAVLQS | NO: 182) | QANKATLVCLISDFYPGAVTVAWKA | |
| | | SGLYSLSSVVTVPSSSLGTQTYICNVNHKPS | | DSSPVKAGVETTTPSKQSNNKYAAS | |
| | | NTKVDKRVEPKSCDKTHTCPPCPAPELLGGP | | SYLSLTPEQWKSHRSYSCQVTHEGS | |
| | | SVFLFPPKPKDTLMISRTPEVTCVVVDVSHE | | TVEKTVAPTECS (SEQ ID | |
| | | DPEVKFNWYVDGVEVHNAKTKPREEQYNSTY | | NO: 250) | |
| | | RVVSVLTVLHQDWLNGKEYKCKVSNKALPAP | | | |
| | | IEKTISKAKGQPREPQVYTLPPSREEMTKNQ | | | |
| | | VSLTCLVKGFYPSDIAVEWESNGQPENNYKT | | | |
| | | TPPVLDSDGSFFLYSKLTVDKSRWQQGNVFS | | | |
| | | CSVMHEALHNHYTQKSLSLSPG (SEQ ID | | | |
| | | NO: 136) | | | |
| 46 | 6 | QVQLVQSGAEVKKPGSSVKVSCKASGGTFSS | QVQLVQSGAEVKKPGSSVKVSCKASGG | SYELTQPPSVSVAPGKTARITCGGN | SYELTQPPSVSVAPGKTARITCGGNN |
| | | YAISWVRQAPGQGLEWMGGIIPIFGTANYAQ | TFSSYAISWVRQAPGQGLEWMGGIIPI | NIRSKSVHWYQQKPGQAPVLVVYQD | IRSKSVHWYQQKPGQAPVLVVYQDSK |
| | | KFQGRVTITADESTSTAYMELSSLRSEDTAV | FGTANYAQKFQGRVTITADESTSTAYM | SKRPSGIPERFSGSNSGNTATLTIS | RPSGIPERFSGSNSGNTATLTISGTQ |
| | | YYCASSPLRGSGWYWHYYYGMDVWGQGTTVT | ELSSLRSEDTAVYYCASSPLRGSGWYW | GTQAMDEADYYCQVWDSSTAVFGGG | AMDEADYYCQVWDSSTAVFGGGTKLT |
| | | VSSASTKGPSVFPLAPSSKSTSGGTAALGCL | HYYYGMDVWGQGTTVTVSS (SEQ ID | TKLTVLGQPKAAPSVTLFPPSSEEL | VL (SEQ ID NO: 306) |
| | | VKDYFPEPVTVSWNSGALTSGVHTFPAVLQS | NO: 183) | QANKATLVCLISDFYPGAVTVAWKA | |
| | | SGLYSISSVVTVPSSSLGTQTYICNVNHKPS | | DSSPVKAGVETTTPSKQSNNKYAAS | |
| | | NTKVDKRVEPKSCDKTHTCPPCPAPELLGGP | | SYLSLTPEQWKSHRSYSCQVTHEGS | |
| | | SVFLFPPKPKDTLMISRTPEVTCVVVDVSHE | | TVEKTVAPTECS (SEQ ID | |
| | | DPEVRFNWYVDGVEVHNAKTKPREEQYNSTY | | NO: 251) | |
| | | RVVSVLTVLHQDWLNGKEYKCKVSNKALPAP | | | |
| | | IEKTISKAKGQPREPQVYTLPPSREEMTKNQ | | | |
| | | VSLTCLVKGFYPSDIAVEWESNGQPENNYKT | | | |
| | | TPPVLDSDGSFFLYSKLTVDKSRWQQGNVFS | | | |
| | | CSVMHEALHNHYTQKSLSLSPG (SEQ ID | | | |
| | | NO: 137) | | | |
| 47 | 6 | EVQLVQSGAEVKKPGSSVKVSCKASGGTFSS | EVQLVQSGAEVKKPGSSVKVSCKASGG | SYELTQPPSVSVAPGKTARITCGGN | SYELTQPPSVSVAPGKTARITCGGNK |
| | | YAISWVRçAPGQGLEWMGGIIPIFGTANYAQ | TFSSYAISWVRQAPGQGLEWMGGIIPI | KIGTKSVHWYQQKPGQAPVLVIYRD | IGTKSVHWYQQKPGQAPVLVIYRDSN |
| | | KFQGRVTITADESTSTAYMELSSLRSEDTAV | FGTANYAQKFQGRVTITADESTSTAYM | SNRPSGIPERFSGSNSGNTATLTIS | RPSGIPERFSGSNSGNTATLTISRAQ |
| | | YYCASSPLRGSGWYWHYYYGMDVWGQGTTVT | ELSSLRSEDTAVYYCASSPLRGSGWYW | RAQAGDEADYYCQVWDSSTVVFGGG | AGDEADYYCQVWDSSTVVFGGGTKLT |
| | | VSSASTKGPSVFPLAPSSKSTSGGTAALGCL | HYYYGMDVWGQGTTVTVSS (SEQ ID | TKLTVLGQPKAAPSVTLFPPSSEEL | VL (SEQ ID NO: 307) |
| | | VKDYFPEPVTVSWNSGALTSGVHTFPAVLQS | NO: 182) | QANKATLVCLISDFYPGAVTVAWKA | |
| | | SGLYSLSSVVTVPSSSLGTQTYICNVNHKPS | | DSSPVKAGVETTTPSKQSNNKYAAS | |
| | | NTKVDKRVEPKSCDKTHTCPPCPAPELLGGP | | SYLSLTPEQWKSHRSYSCQVTHEGS | |
| | | SVFLFPPKPKDTLMISRTPEVTCVVVDVSHE | | TVEKTVAPTECS (SEQ ID | |
| | | DPEVKFNWYVDGVEVHNAKTKPREEQYNSTY | | NO: 252) | |
| | | RVVSVLTVLHQDWLNGKEYKCKVSNKALPAP | | | |
| | | IEKTISKAKGQPREPQVYTLPPSREEMTKNQ | | | |
| | | VSLTCLVKGFYPSDIAVEWESNGQPENNYKT | | | |
| | | TPPVLDSDGSFFLYSKLTVDKSRWQQGNVFS | | | |
| | | CSVMHEALHNHYTQKSLSLSPG (SEQ ID | | | |
| | | NO: 136) | | | |
| 48 | 6 | EVQLVQSGAEVKKPGSSVKVSCKASGGTFSS | EVQLVQSGAEVKKPGSSVKVSCKASGG | SYVLTQPPSVSVAPGKTARITCGGN | SYVLTQPPSVSVAPGKTARITCGGNN |
| | | YAISWVRçAPGQGLEWMGGIIPIFGTANYAQ | TFSSYAISWVRQAPGQGLEWMGGIIPI | NIGSKSVHWYQQKPGQAPVLVIYQD | IGSKSVHWYQQKPGQAPVLVIYQDRK |
| | | KFQGRVTITADESTSTAYMELSSLRSEDTAV | FGTANYAQKFQGRVTITADESTSTAYM | RKRPSGIPERFSGSNSGNTATLTIS | RPSGIPERFSGSNSGNTATLTISGTQ |
| | | YYCASSPLRGSGWYWHYYYGMDVWGQGTTVT | ELSSLRSEDTAVYYCASSPLRGSGWYW | GTQAMDEADYYCQAWDSSTAVFGGG | AMDEADYYCQAWDSSTAVFGGGTKLT |
| | | VSSASTKGPSVFPLAPSSKSTSGGTAALGCL | HYYYGMDVWGQGTTVTVSS (SEQ ID | TKLTVLGQPKAAPSVTLFPPSSEEL | VL (SEQ ID NO: 308) |
| | | VKDYFPEPVTVSWNSGALTSGVHTFPAVLQS | NO: 182) | QANKATLVCLISDFYPGAVTVAWKA | |
| | | SGLYSLSSVVTVPSSSLGTQTYICNVNHKPS | | DSSPVKAGVETTTPSKQSNNKYAAS | |
| | | NTKVDKRVEPKSCDKTHTCPPCPAPELLGGP | | SYLSLTPEQWKSHRSYSCQVTHEGS | |
| | | SVFLFPPKPKDTLMISRTPEVTCVVVDVSHE | | TVEKTVAPTECS (SEQ ID | |
| | | DPEVKFNWYVDGVEVHNAKTKPREEQYNSTY | | NO: 253) | |
| | | RVVSVLTVLHQDWLNGKEYKCKVSNKALPAP | | | |
| | | IEKTISKAKGQPREPQVYTLPPSREEMTKNQ | | | |
| | | VSLTCLVKGFYPSDIAVEWESNGQPENNYKT | | | |
| | | TPPVLDSDGSFFLYSKLTVDKSRWQQGNVFS | | | |
| | | CSVMHEALHNHYTQKSLSLSPG (SEQ ID | | | |
| | | NO: 136) | | | |
| 49 | 6 | EVQLVQSGAEVKKPGSSVKVSCKASGGTFSS | EVQLVQSGAEVKKPGSSVKVSCKASGG | SYELTQPPSVSVAPGKTARITCGGN | SYELTQPPSVSVAPGKTARITCGGNN |
| | | YAISWVRçAPGQGLEWMGGIIPIFGTANYAQ | TFSSYAISWVRQAPGQGLEWMGGIIPI | NIRAKHVHWYQQKPGQSPVLVIYQD | IRAKHVHWYQQKPGQSPVLVIYQDSK |
| | | KFQGRVTITADESTSTAYMELSSLRSEDTAV | FGTANYAQKFQGRVTITADESTSTAYM | SKRPSGIPERFSGSNSGSTATLTIS | RPSGIPERFSGSNSGSTATLTISRVE |
| | | YYCASSPLRGSGWYWHYYYGMDVWGQGTTVT | ELSSLRSEDTAVYYCASSPLRGSGWYW | RVEAGDEADYYCQVWDSSTVVFGGG | AGDEADYYCQVWDSSTVVFGGGTKLT |
| | | VSSASTKGPSVFPLAPSSKSTSGGTAALGCL | HYYYGMDVWGQGTTVTVSS (SEQ ID | TKLTVLGQPKAAPSVTLFPPSSEEL | VL (SEQ ID NO: 309) |
| | | VKDYFPEPVTVSWNSGALTSGVHTFPAVLQS | NO: 182) | QANKATLVCLISDFYPGAVTVAWKA | |
| | | SGLYSLSSVVTVPSSSLGTQTYICNVNHKPS | | DSSPVKAGVETTTPSKQSNNKYAAS | |
| | | NTKVDKRVEPKSCDKTHTCPPCPAPELLGGP | | SYLSLTPEQWKSHRSYSCQVTHEGS | |
| | | SVFLFPPKPKDTLMISRTPEVTCVVVDVSHE | | TVEKTVAPTECS (SEQ ID | |
| | | DPEVKFNWYVDGVEVHNAKTKPREEQYNSTY | | NO: 254) | |
| | | RVVSVLTVLHQDWLNGKEYKCKVSNKALPAP | | | |
| | | IEKTISKAKGQPREPQVYTLPPSREEMTKNQ | | | |
| | | VSLTCLVKGFYPSDIAVEWESNGQPENNYKT | | | |
| | | TPPVLDSDGSFFLYSKLTVDKSRWQQGNVFS | | | |
| | | CSVMHEALHNHYTQKSLSLSPG (SEQ ID | | | |
| | | NO: 136) | | | |
| 50 | 6 | QMQLVQSGAEVKKPGSSVKVSCKASGGTFSS | QMQLVQSGAEVKKPGSSVKVSCKASGG | SYVLTQPPSVSVAPGKTARITCGGN | SYVLTQPPSVSVAPGKTARITCGGNN |
| | | YAISWVRçAPGQGLEWMGGIIPIFGTANYAQ | TFSSYAISWVRQAPGQGLEWMGGIIPI | NIGTKSVHWYQQKPGQSPVLVIYQD | IGTKSVHWYQQKPGQSPVLVIYQDRK |
| | | KFQGRVTITADESTSTAYMELSSLRSEDTAV | FGTANYAQKFQGRVTITADESTSTAYM | RKRPSGIPERFSGSNSGNTATLTIS | RPSGIPERFSGSNSGNTATLTISGTQ |
| | | YYCASSPLRGSGWYWHYYYGMDVWGQGTTVT | ELSSLRSEDTAVYYCASSPLRGSGWYW | GTQAMDEADYYCQVWDSSTEVFGGG | AMDEADYYCQVWDSSTEVFGGGTKLT |
| | | VSSASTKGPSVFPLAPSSKSTSGGTAALGCL | HYYYGMDVWGQGTTVTVSS (SEQ ID | TKLTVLGQPKAAPSVTLFPPSSEEL | VL (SEQ ID NO: 310) |
| | | VKDYFPEPVTVSWNSGALTSGVHTFPAVLQS | NO: 184) | QANKATLVCLISDFYPGAVTVAWKA | |
| | | SGLYSLSSVVTVPSSSIGTQTYICNVNHKPS | | DSSPVKAGVETTTPSKQSNNKYAAS | |
| | | NTKVDKRVEPKSCDKTHTCPPCPAPELLGGP | | SYLSLTPEQWKSHRSYSCQVTHEGS | |
| | | SVFLFPPKPKDTLMISRTPEVTCVVVDVSHE | | TVEKTVAPTECS (SEQ ID | |
| | | DPEVKFNWYVDGVEVHNAKTKPREEQYNSTY | | NO: 255) | |
| | | RVVSVLTVLHQDWLNGKEYKCKVSNKALPAP | | | |
| | | IEKTISKAKGQPREPQVYTLPPSREEMTKNQ | | | |
| | | VSLTCLVKGFYPSDIAVEWESNGQPENNYKT | | | |
| | | TPPVLDSDGSFFLYSKLTVDKSRWQQGNVFS | | | |
| | | CSVMHEALANHYTQKSLSLSPG (SEQ ID | | | |
| | | NO: 138) | | | |
| 51 | 6 | EVQLVQSGAEVKKPGSSVKVSCKASGGTFSS | EVQLVQSGAEVKKPGSSVKVSCKASGG | SYELTQPPSVSVAPGKTARITCGGN | SYELTQPPSVSVAPGKTARITCGGNK |
| | | YAISWVRçAPGQGLEWMGGIIPIFGTANYAQ | TFSSYAISWVRQAPGQGLEWMGGTIPI | KIGTKSVHWYQQKPGQAPVLVIYQD | IGTKSVHWYQQKPGQAPVLVIYQDSK |
| | | KFQGRVTITADESTSTAYMELSSLRSEDTAV | FGTANYAQKFQGRVTITADESTSTAYM | SKRPSGIPERFSGSNSGNTATLTIS | RPSGIPERFSGSNSGNTATLTISGTQ |
| | | YYCASSPLRGSGWYWHYYYGMDVWGQGTTVT | ELSSLRSEDTAVYYCASSPLRGSGWYW | GTQAMDEADYYCQAWGSSTVVFGGG | AMDEADYYCQAWGSSTVVFGGGTKLT |
| | | VSSASTKGPSVFPLAPSSKSTSGGTAALGCL | HYYYGMDVWGQGTTVTVSS (SEQ ID | TKLTVLGQPKAAPSVTLFPPSSEEL | VL (SEQ ID NO: 311) |
| | | VKDYFPEPVTVSWNSGALTSGVHTFPAVLQS | NO: 182) | QANKATLVCLISDFYPGAVTVAWKA | |
| | | SGLYSLSSVVTVPSSSLGTQTYICNVNHKPS | | DSSPVKAGVETTTPSKQSNNKYAAS | |
| | | NTKVDKRVEPKSCDKTHTCPPCPAPELLGGP | | SYLSLTPEQWKSHRSYSCQVTHEGS | |
| | | SVFLFPPKPKDTLMISRTPEVTCVVVDVSHE | | TVEKTVAPTECS (SEQ ID | |
| | | DPEVKFNWYVDGVEVHNAKTKPREEQYNSTY | | NO: 256) | |
| | | RVVSVLTVLHQDWLNGKEYKCKVSNKALPAP | | | |
| | | IEKTISKAKGQPREPQVYTLPPSREEMTKNQ | | | |
| | | VSLTCLVKGFYPSDIAVEWESNGQPENNYKT | | | |
| | | TPPVLDSDGSFFLYSKLTVDKSRWQQGNVFS | | | |
| | | CSVMHEALHNHYTQKSLSLSPG (SEQ ID | | | |
| | | NO: 136) | | | |
| 52 | 12 | EVQLVQSGAEVKKPGASVKVSCKVSGYTLTE | EVQLVQSGAEVKKPGASVKVSCKVSGY | DIQLTQSPSSLSASVGDRVTITCQA | DIQLTQSPSSLSASVGDRVTITCQAS |
| | | LSMHWVRQAPGKGLEWMGLVDPEDGETIYAE | TLTELSMHWVRQAPGKGLEWMGLVDPE | SQDISNYLNWYQQKPGKAPKLLIYD | QDISNYLNWYQQKPGKAPKLLIYDAS |
| | | KFQGRVTITADTSTDTAYMELSSPRSEDTAV | DGETIYAEKFQGRVTITADTSTDTAYM | ASNLETGVPSRFSGSGSGTDFTFTI | NLETGVPSRFSGSGSGTDFTFTISSI |
| | | YYCATTIGDDYFDYWGQGTLVTVSSASTKGP | ELSSPRSEDTAVYYCATTIGDDYFDYW | SSLQPEDIATYYCQQYDNLPLTFGG | QPEDIATYYCQQYDNLPLTFGGGTKL |
| | | SVFPLAPSSKSTSGGTAALGCLVKDYFPEPV | GQGTLVTVSS (SEQ ID NO: 185) | GTKLEIKRTVAAPSVFIFPPSDEQL | EIK (SEQ ID NO: 312) |
| | | TVSWNSGALTSGVHTFPAVLQSSGLYSLSSV | | KSGTASVVCLLNNFYPREAKVQWKV | |
| | | VTVPSSSIGTQTYICNVNHKPSNTKVDKRVE | | DNALQSGNSQESVTEQDSKDSTYSL | |
| | | PRSCDRTHTCPPCPAPELLGGPSVFLFPPKP | | SSTLTLSKADYEKHKVYACEVTHQG | |
| | | KDTLMISRTPEVTCVVVDVSHEDPEVKFNWY | | LSSPVTKSFNRGEC (SEQ ID | |
| | | VDGVEVHNAKTKPREEQYNSTYRVVSVLTVL | | NO: 257) | |
| | | HQDWLNGKEYKCKVSNKALPAPIEKTISKAK | | | |
| | | GQPREPQVYTLPPSREEMTKNQVSLTCLVKG | | | |
| | | FYPSDIAVEWESNGQPENNYKTTPPVLDSDG | | | |
| | | SFFLYSKITVDKSRWQQGNVFSCSVMHEALH | | | |
| | | NHYTQKSLSLSPG (SEQ ID NO: 139) | | | |
| 53 | 13 | QVQLVQSGAEVKKPGASVKVSCKVSGYTLTE | QVQLVQSGAEVKKPGASVKVSCKVSGY | QSALTQPRSVSGSPGQSVTISCTGT | QSALTQPRSVSGSPGQSVTISCTGTN |
| | | LSMHWVRQAPGKGLEWMGGFDPEDGETIYAQ | TLTELSMHWVRQAPGKGLEWMGGFDPE | NTDVGAYIDVSWYQQHPGKAPKLII | TDVGAYIDVSWYQQHPGKAPKLIIYD |
| | | KFQGRVTMTEDTSTDTAYMELSSLRSEDTAV | DGETIYAQKFQGRVTMTEDTSTDTAYM | YDVSKWPSGVPDRFSGSKSGNTASL | VSKWPSGVPDRFSGSKSGNTASITIS |
| | | YYCATDIQGLAEFDYWGQGTLVTVSSASTKG | ELSSLRSEDTAVYYCATDIQGLAEFDY | TISGLQAEDEADYYCCSYAGNHSFS | GLQAEDEADYYCCSYAGNHSFSFGGG |
| | | PSVFPLAPSSKSTSGGTAALGCLVKDYFPEP | WGQGTLVTVSS (SEQ ID NO: 186) | FGGGTKVTVLGQPKAAPSVTLFPPS | TKVTVL (SEQ ID NO: 313) |
| | | VTVSWNSGALTSGVHTFPAVLQSSGLYSLSS | | SEELQANKATLVCLISDEYPGAVTV | |
| | | VVTVPSSSLGTQTYICNVNHKPSNTKVDKRV | | AWKADSSPVKAGVETTTPSKQSNNK | |
| | | EPKSCDKTHTCPPCPAPELLGGPSVFLFPPK | | YAASSYLSLTPEQWKSHRSYSCQVT | |
| | | PKDTLMISRTPEVTCVVVDVSHEDPEVKFNW | | HEGSTVEKTVAPTECS (SEQ ID | |
| | | YVDGVEVHNAKTKPREEQYNSTYRVVSVLTV | | NO: 258) | |
| | | LHQDWLNGKEYKCKVSNKALPAPIEKTISKA | | | |
| | | KGQPREPQVYTIPPSREEMTKNQVSLTCLVK | | | |
| | | GFYPSDIAVEWESNGQPENNYKTTPPVLDSD | | | |
| | | GSFFLYSKITVDKSRWQQGNVFSCSVMHEAL | | | |
| | | HNHYTQKSLSLSPG (SEQ ID NO: 140) | | | |
| 54 | 14 | QVTLKESGPGILKPSQTLSLTCSFSGFSLST | QVTLKESGPGILKPSQTLSLTCSFSGF | DIVLTQSPASLAVSLGQRATISCKA | DIVLTQSPASLAVSLGQRATISCKAS |
| | | SGMGVGWIRQPSGKGLEWLAHIWWDDDKYYN | SLSTSGMGVGWIRQPSGKGLEWLAHIW | SQSVDYDGDSFMNWYQQKPGQPPKL | QSVDYDGDSFMNWYQQKPGQPPKLLI |
| | | PYLKNQVTISKDTSRNQVFLKITSVDTADTA | WDDDKYYNPYLKNQVTISKDTSRNQVF | LIYTASILESGIPARFSGSGSGTDF | YTASILESGIPARFSGSGSGTDFTLN |
| | | TYYCARSAYGSTYGYWGQGTTLTVSSASTKG | LKITSVDTADTATYYCARSAYGSTYGY | TLNIHPVEEEDAATYYCQQSNEDPW | IHPVEEEDAATYYCQQSNEDPWTFGG |
| | | PSVFPLAPSSKSTSGGTAALGCLVKDYFPEP | WGQGTTLIVSS (SEQ ID NO: 187) | TFGGGTKLEIKRTVAAPSVFIFPPS | GTKLEIK (SEQ ID NO: 314) |
| | | VTVSWNSGALTSGVHTFPAVLQSSGLYSLSS | | DEQLKSGTASVVCLLNNFYPREAKV | |
| | | VVTVPSSSLGTQTYICNVNHKPSNTKVDKRV | | QWKVDNALQSGNSQESVTEQDSKDS | |
| | | EPKSCDKTHTCPPCPAPELLGGPSVFLFPPK | | TYSLSSTLTLSKADYEKHKVYACEV | |
| | | PKDTLMISRTPEVTCVVVDVSHEDPEVKFNW | | THQGLSSPVTKSFNRGEC (SEQ | |
| | | YVDGVEVHNAKTKPREEQYNSTYRVVSVLTV | | ID NO: 259) | |
| | | LHQDWLNGKEYKCKVSNKALPAPIEKTISKA | | | |
| | | KGQPREPQVYTLPPSREEMTKNQVSLTCLVK | | | |
| | | GFYPSDIAVEWESNGQPENNYKTTPPVLDSD | | | |
| | | GSFFLYSKLTVDKSRWQQGNVFSCSVMHEAL | | | |
| | | HNHYTQKSLSLSPG (SEQ ID NO: 141) | | | |

TABLE 5
Heavy chain, heavy chain variable region, light chain, and light chain variable region sequences of three parent clones and
variants thereof.

| Clone | | | | |
|---|---|---|---|---|
| No. | Heavy chain sequence | VH | Light chain sequence | VL |
| 6 | EVQLVQSGAEVKKPGASVKVSCKVSGYTITELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 165) | TLFPPSSEELQANKATLVCLISDF | NO: 268) |
| | VDVSHEDPEVKFNWYVDGVEVHNAKTKPREEQYNSTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 119) | | | |
| 55 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSISSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDRTHTCPPCPAPELLGGPSVFLFPPKPKDTLMISRTPEVTCVV | (SEQ ID NO: 165) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 142) | | | |
| 56 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDAGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDAGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 188) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 143) | | | |
| 57 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDSGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDSGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 189) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 144) | | | |
| 58 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDEGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDEGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVATFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 190) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 145) | | | |
| 59 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDAETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDAETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 191) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTD | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 146) | | | |
| 60 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELSSLRSEDTAVYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 192) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIERTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 147) | | | |
| 61 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDAGETMYAEKFQGRLTVTEDTSTDTVYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYFCATSPGYDFFDYWGQGTLVTVSSASTKGPSVFPLAPS | GGFDPDAGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELSSLRSEDTAVYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 193) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 148) | | | |
| 62 | EVQLVQSGAEVKKPGASVKVSCKVSGYTITELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDSGETMYAEKFQGRLTVTEDTSTDTVYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYFCATSPGYDFFDYWGQGTLVTVSSASTKGPSVFPLAPS | GGFDPDSGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELSSLRSEDTAVYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 194) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 149) | | | |
| 63 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDEGETMYAEKFQGRLTVTEDTSTDTVYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYFCATSPGYDFFDYWGQGTLVTVSSASTKGPSVFPLAPS | GGFDPDEGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELSSLRSEDTAVYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 195) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 150) | | | |
| 64 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDAETMYAEKFQGRLTVTEDTSTDTVYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYFCATSPGYDFFDYWGQGTLVTVSSASTKGPSVFPLAPS | GGFDPDDAETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELSSLRSEDTAVYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 196) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 151) | | | |
| 65 | EVQLVQSGAEVKKPGASVKVSCKVSGYTLTELSMQWVRçAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 165) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 142) | | | |
| 66 | EVQLVQSGAEVKKPGASVKVSCKVSGYTITELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDAGETMYAEKFQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDAGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 188) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKITP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 143) | | | |
| 67 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDSGETMYAEKFQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDSGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSIGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDRTHTCPPCPAPELLGGPSVFLFPPKPKDTLMISRTPEVTCVV | (SEQ ID NO: 189) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 144) | | | |
| 68 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDEGETMYAEKFQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDEGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 190) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SISLSPG (SEQ ID NO: 145) | | | |
| 69 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDAETMYAEKEQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDAETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 191) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 146) | | | |
| 70 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTVYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYFCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELSSLRSEDTAVYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 192) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVESCSVMHEALHNHYTQK | | S (SEQ ID NO: 260) | |
| | SLSLSPG (SEQ ID NO: 147) | | | |
| 71 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDAGETMYAEKFQGRLTVTEDTSTDTVYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYFCATSPGYDFFDYWGQGTLVTVSSASTKGPSVFPLAPS | GGFDPDAGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELSSLRSEDTAVYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 193) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 148) | | | |
| 72 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDSGETMYAEKFQGRLTVTEDTSTDTVYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYFCATSPGYDFFDYWGQGTLVTVSSASTKGPSVFPLAPS | GGFDPDSGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELSSLRSEDTAVYFC | ATLTISRVEAGDEADYYCQVWDSS | ATLTISRVEAGDEADYYCQVWDSS |
| | SSGLYSISSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 194) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 149) | | | |
| 73 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDEGETMYAEKFQGRLTVTEDTSTDTVYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYFCATSPGYDFFDYWGQGTLVTVSSASTKGPSVFPLAPS | GGFDPDEGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELSSLRSEDTAVYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 195) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 150) | | | |
| 74 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDAETMYAEKFQGRLTVTEDTSTDTVYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYFCATSPGYDFFDYWGQGTLVTVSSASTKGPSVFPLAPS | GGFDPDDAETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELSSLRSEDTAVYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 196) | TLFPPSSEELQANKATLVCLISDF | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 151) | | | |
| 75 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 165) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 142) | | | |
| 76 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDAGETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDAGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 188) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVESCSVMHEALHNHYTQK | | (SEQ ID NO: 261) | |
| | SLSLSPG (SEQ ID NO: 143) | | | |
| 77 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDSGETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDSGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 189) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 144) | | | |
| 78 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDEGETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDEGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSISSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 190) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 145) | | | |
| 79 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDAETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVEPLAPS | GGFDPDDAETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 191) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 146) | | | |
| 80 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 192) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 147) | | | |
| 81 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDAGETMYAEKFQGRLTVTEDTSTDTVYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYFCATSPGYDFFDYWGQGTLVTVSSASTKGPSVFPLAPS | GGFDPDAGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVATEPAVLQ | DTSTDTVYMELSSLRSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 193) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 148) | | | |
| 82 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDSGETMYAEKFQGRLTVTEDTSTDTVYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYFCATSPGYDFFDYWGQGTLVTVSSASTKGPSVFPLAPS | GGFDPDSGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 194) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 149) | | | |
| 83 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDEGETMYAEKFQGRLTVTEDTSTDTVYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYFCATSPGYDFFDYWGQGTLVTVSSASTKGPSVFPLAPS | GGFDPDEGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 195) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 150) | | | |
| 84 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDAETMYAEKFQGRLTVTEDTSTDTVYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYFCATSPGYDFFDYWGQGTLVTVSSASTKGPSVFPLAPS | GGFDPDDAETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 196) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 151) | | | |
| 85 | QVQLVQSGAEVKKPGASVKVSCKVSGYTITELSMQWVRQAPGKG | QVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRVTMTEDTSTDTAYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYYCATSPGYDFFDYWGQGTLVTVSSASTKGPSVFPLAPS | GGFDPDDGETMYAEKFQGRVTMTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTAYMELSSLRSEDTAVYYC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 197) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 152) | | | |
| 86 | QVQLVQSGAEVKKPGASVKVSCKVSGYTLTELSMQWVRQAPGKG | QVQLVQSGAEVKKPGASVKVSCKV | QSALTQPASVSGSPGQSITISCTG | QSALTQPASVSGSPGQSITISCTG |
| | LEWMGGFDPDAGETMYAEKFQGRVTMTEDTSTDTAYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYYCATSPGYDFFDYWGQGTLVTVSSASTKGPSVFPLAPS | GGFDPDAGETMYAEKFQGRVTMTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTAYMELSSLRSEDTAVYYC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 198) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTI | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 153) | | | |
| 37 | QVQLVQSGAEVKKPGASVKVSCKVSGYTLTELSMQWVRQAPGKG | QVQLVQSGAEVKKPGASVKVSCKV | QSALTQPASVSGSPGQSITISCTG | QSALTQPASVSGSPGQSITISCTG |
| | LEWMGGFDPDSGETMYAEKFQGRVTMTEDTSTDTAYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYYCATSPGYDFFDYWGQGTLVTVSSASTKGPSVFPLAPS | GGFDPDSGETMYAEKFQGRVTMTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTAYMELSSLRSEDTAVYYC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 199) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 154) | | | |
| 88 | QVQLVQSGAEVKKPGASVKVSCKVSGYTLTELSMQWVRQAPGKG | QVQLVQSGAEVKKPGASVKVSCKV | QSALTQPASVSGSPGQSITISCTG | QSALTQPASVSGSPGQSITISCTG |
| | LEWMGGFDPDEGETMYAEKFQGRVTMTEDTSTDTAYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYYCATSPGYDFFDYWGQGTLVTVSSASTKGPSVFPLAPS | GGFDPDEGETMYAEKFQGRVTMTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTAYMELSSLRSEDTAVYYC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 200) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 155) | | | |
| 89 | QVQLVQSGAEVKKPGASVKVSCKVSGYTLTELSMQWVRQAPGKG | QVQLVQSGAEVKKPGASVKVSCKV | QSALTQPASVSGSPGQSITISCTG | QSALTQPASVSGSPGQSITISCTG |
| | LEWMGGFDPDDAETMYAEKFQGRVTMTEDTSTDTAYMELSSLRS | SGYTLTELSMQWVRQAPGKGLEWM | TSSDVGGYNYVSWYQQHPGKAPKL | TSSDVGGYNYVSWYQQHPGKAPKL |
| | EDTAVYYCATSPGYDFFDYWGQGTLVTVSSASTKGPSVFPLAPS | GGFDPDDAETMYAEKFQGRVTMTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTAYMELSSLRSEDTAVYYC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 201) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKITP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 156) | | | |
| 90 | QVQLVQSGAEVKKPGASVKVSCKVSGYTLTELSMQWVRQAPGKG | QVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRVTMTEDTSTDTAYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYYCATSPGYDFFDYWGQGTLVTVSSASTKGPSVFPLAPS | GGFDPDDGETMYAEKFQGRVTMTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTAYMELSSLRSEDTAVYYC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 197) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 152) | | | |
| 91 | QVQLVQSGAEVKKPGASVKVSCKVSGYTLTELSMQWVRQAPGKG | QVQLVQSGAEVKKPGASVKVSCKV | SYVLTQPPSVSVAPGKTARITCTG | SYVLTQPPSVSVAPGKTARITCTG |
| | LEWMGGFDPDAGETMYAEKFQGRVTMTEDTSTDTAYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYYCATSPGYDFFDYWGQGTLVTVSSASTKGPSVFPLAPS | GGFDPDAGETMYAEKFQGRVTMTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTAYMELSSLRSEDTAVYYC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 198) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 153) | | | |
| 92 | QVQLVQSGAEVKKPGASVKVSCKVSGYTLTELSMQWVRQAPGKG | QVQLVQSGAEVKKPGASVKVSCKV | SYVLTQPPSVSVAPGKTARITCTG | SYVLTQPPSVSVAPGKTARITCTG |
| | LEWMGGFDPDSGETMYAEKFQGRVTMTEDTSTDTAYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYYCATSPGYDFFDYWGQGTLVTVSSASTKGPSVFPLAPS | GGFDPDSGETMYAEKFQGRVTMTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTAYMELSSLRSEDTAVYYC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 199) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 154) | | | |
| 93 | QVQLVQSGAEVKKPGASVKVSCKVSGYTLTELSMQWVRQAPGKG | QVQLVQSGAEVKKPGASVKVSCKV | SYVLTQPPSVSVAPGKTARITCTG | SYVLTQPPSVSVAPGKTARITCTG |
| | LEWMGGFDPDEGETMYAEKFQGRVTMTEDTSTDTAYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYYCATSPGYDFFDYWGQGTLVTVSSASTKGPSVFPLAPS | GGFDPDEGETMYAEKFQGRVTMTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTAYMELSSLRSEDTAVYYC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 200) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 155) | | | |
| 94 | QVQLVQSGAEVKKPGASVKVSCKVSGYTLTELSMQWVRçAPGKG | QVQLVQSGAEVKKPGASVKVSCKV | SYVLTQPPSVSVAPGKTARITCTG | SYVLTQPPSVSVAPGKTARITCTG |
| | LEWMGGFDPDDAETMYAEKFQGRVTMTEDTSTDTAYMELSSLRS | SGYTLTELSMQWVRQAPGKGLEWM | TSSDVGGYNYVSWYQQKPGQAPVL | TSSDVGGYNYVSWYQQKPGQAPVL |
| | EDTAVYYCATSPGYDFFDYWGQGTLVTVSSASTKGPSVFPLAPS | GGFDPDDAETMYAEKFQGRVTMTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTAYMELSSLRSEDTAVYYC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 201) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 156) | | | |
| 95 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRVTMTEDTSTDTAYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYYCATSPGYDFFDYWGQGTLVTVSSASTKGPSVFPLAPS | GGFDPDDGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 197) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 152) | | | |
| 96 | QVQLVQSGAEVRKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDAGETMYAEKFQGRVTMTEDTSTDTAYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYYCATSPGYDFFDYWGQGTLVTVSSASTKGPSVFPLAPS | GGFDPDAGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 198) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 153) | | | |
| 97 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDSGETMYAEKFQGRVTMTEDTSTDTAYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYYCATSPGYDFFDYWGQGTLVTVSSASTKGPSVFPLAPS | GGFDPDSGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 199) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 154) | | | |
| 98 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDEGETMYAEKFQGRVTMTEDTSTDTAYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYYCATSPGYDFFDYWGQGTLVTVSSASTKGPSVFPLAPS | GGFDPDEGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTEPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 200) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 155) | | | |
| 99 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDDAETMYAEKFQGRVTMTEDTSTDTAYMELSSLRS | SGYTLTELSMQWVRQAPGKGLEWM | NNIGSKSVHWYQQKPGQAPVLVVY | NNIGSKSVHWYQQKPGQAPVLVVY |
| | EDTAVYYCATSPGYDFFDYWGQGTLVTVSSASTKGPSVFPLAPS | GGFDPDDAETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 201) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 156) | | | |
| 100 | QVQLVQSGAEVKKPGASVKVSCKVSGYTLTELSMQWVRQAPGKG | QVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 170) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 157) | | | |
| 101 | EVQLVQSGAEVKKPGASVKVSCKVSGYTITELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRVTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRVTVTE | MIYEVSNRPSGVSNRFSGSNSGNT | MIYEVSNRPSGVSNRFSGSNSGNT |
| | SKSTSGGTAALGCLVRDYFPEPVTVSWNSGALTSGVHTEPAVLQ | DTSTDTVYMELRSLTSEDTALYFC | ATLTINRVEAGDEADYYCQVWDSS | ATLTINRVEAGDEADYYCQVWDSS |
| | SSGLYSISSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 202) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 158) | | | |
| 102 | EVQLVQSGAEVRKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTMTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRLTMTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVERKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 203) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 159) | | | |
| 103 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTAYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTAYMELRSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 204) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 160) | | | |
| 104 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELSSLTS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELSSLTSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 205) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 161) | | | |
| 105 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKEQGRLTVTEDTSTDTVYMELRSLRS | SGYTLTELSMQWVRQAPGKGLEWM | TSSDVGGYNYVSWYQQHPGKAPKL | TSSDVGGYNYVSWYQQHPGKAPKL |
| | EDTALYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLRSEDTALYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 206) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKITVDKSRWQQGNVFSCSVMHEALHNHYTQK | | S (SEQ ID NO: 213) | |
| | SLSLSPG (SEQ ID NO: 162) | | | |
| 106 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTAVYFCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTAVYFC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 207) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 163) | | | |
| 107 | EVQLVQSGAEVKKPGASVKVSCKVSGYTLTELSMQWVRQAPGKG | EVQLVQSGAEVKKPGASVKVSCKV | QSALTQPASVSGSPGQSITISCTG | QSALTQPASVSGSPGQSITISCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQHPGKAPKL | TSSDVGGYNYVSWYQQHPGKAPKL |
| | EDTALYYCATSPGYDFFDYWGQGTLVTVSSASTKGPSVFPLAPS | GGFDPDDGETMYAEKFQGRLTVTE | MIYEVSNRPSGVSNRFSGSNSGNT | MIYEVSNRPSGVSNRFSGSNSGNT |
| | SKSTSGGTAALGCLVKDYFPEPVTVSWNSGALTSGVHTFPAVLQ | DTSTDTVYMELRSLTSEDTALYYC | ATLTINRVEAGDEADYYCQVWDSS | ATLTINRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 208) | TLFPPSSEELQANKATLVCLISDF | NO: 268) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 213) | |
| | SLSLSPG (SEQ ID NO: 164) | | | |
| 108 | QVQLVQSGAEVKKPGASVKVSCKVSGYTITELSMQWVRQAPGKG | QVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 170) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 157) | | | |
| 109 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRVTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDGETMYAEKFQGRVTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 202) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 158) | | | |
| 110 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLIMTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDGETMYAEKFQGRLTMTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 203) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 159) | | | |
| 111 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTAYMELRSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTAYMELRSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 204) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 160) | | | |
| 112 | EVQLVQSGAEVKKPGASVKVSCKVSGYTITELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTVYMELSSLTS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELSSLTSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 205) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKITP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 161) | | | |
| 113 | EVQLVQSGAEVRKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTVYMELRSLRS | SGYTLTELSMQWVRQAPGKGLEWM | TSSDVGGYNYVSWYQQKPGQAPVL | TSSDVGGYNYVSWYQQKPGQAPVL |
| | EDTALYFCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLRSEDTALYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDRTHTCPPCPAPELLGGPSVFLFPPKPKDTLMISRTPEVTCVV | (SEQ ID NO: 206) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 162) | | | |
| 114 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTVYMELRSLTS | SGYTLTELSMQWVRQAPGKGLEWM | TSSDVGGYNYVSWYQQKPGQAPVL | TSSDVGGYNYVSWYQQKPGQAPVL |
| | EDTAVYFCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTAVYFC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 207) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 163) | | | |
| 115 | EVQLVQSGAEVKKPGASVKVSCKVSGYTLTELSMQWVRQAPGKG | EVQLVQSGAEVKKPGASVKVSCKV | SYVLTQPPSVSVAPGKTARITCTG | SYVLTQPPSVSVAPGKTARITCTG |
| | LEWMGGFDPDDGETMYAEKFQGRLTVTEDTSTDTVYMELRSLIS | SGYTLTELSMQWVRQAPGKGLEWM | TSSDVGGYNYVSWYQQKPGQAPVL | TSSDVGGYNYVSWYQQKPGQAPVL |
| | EDTALYYCATSPGYDFFDYWGQGTLVTVSSASTKGPSVFPLAPS | GGFDPDDGETMYAEKFQGRLTVTE | VVYEVSNRPSGIPERFSGSNSGNT | VVYEVSNRPSGIPERFSGSNSGNT |
| | SKSTSGGTAALGCLVKDYFPEPVTVSWNSGALTSGVHTFPAVLQ | DTSTDTVYMELRSLTSEDTALYYC | ATLTISRVEAGDEADYYCQVWDSS | ATLTISRVEAGDEADYYCQVWDSS |
| | SSGLYSLSSVVTVPSSSLGTQTYICNVNHKPSNTKVDKRVEPKS | ATSPGYDFFDYWGQGTLVTVSS | NDLLIFGGGTKLTVLGQPKAAPSV | NDLLIFGGGTKLTVL (SEQ ID |
| | CDKTHTCPPCPAPELLGGPSVFLFPPKPKDTLMISRTPEVTCVV | (SEQ ID NO: 208) | TLFPPSSEELQANKATLVCLISDE | NO: 315) |
| | VDVSHEDPEVKFNWYVDGVEVHNAKTKPREEQYASTYRVVSVLT | | YPGAVTVAWKADSSPVKAGVETTT | |
| | VLHQDWLNGKEYKCKVSNKALPAPIEKTISKAKGQPREPQVYTL | | PSKQSNNKYAASSYLSLTPEQWKS | |
| | PPSREEMTKNQVSLTCLVKGFYPSDIAVEWESNGQPENNYKTTP | | HRSYSCQVTHEGSTVEKTVAPTEC | |
| | PVLDSDGSFFLYSKLTVDKSRWQQGNVFSCSVMHEALHNHYTQK | | S (SEQ ID NO: 260) | |
| | SLSLSPG (SEQ ID NO: 164) | | | |
| 116 | QVQLVQSGAEVKKPGASVRVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLIVL (SEQ ID |
| | CDKTHTCPPCPAPELLGGPSVFLFPPKPKDTLMISRTPEVTCVV | (SEQ ID NO: 170) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 157) | | | |
| 117 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRVTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDGETMYAEKFQGRVTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLIVL (SEQ ID |
| | CDKTHTCPPCPAPELLGGPSVFLFPPKPKDTLMISRTPEVTCVV | (SEQ ID NO: 202) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 158) | | | |
| 118 | EVQLVQSGAEVKKPGASVKVSCKVSGYTITELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTMTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDGETMYAEKFQGRLTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLIVL (SEQ ID |
| | CDKTHTCPPCPAPELLGGPSVFLFPPKPKDTLMISRTPEVTCVV | (SEQ ID NO: 203) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 159) | | | |
| 119 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTAYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 204) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 160) | | | |
| 120 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELSSLTS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLIVL (SEQ ID |
| | CDKTHTCPPCPAPELLGGPSVFLFPPKPKDTLMISRTPEVTCVV | (SEQ ID NO: 205) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 161) | | | |
| 121 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLRS | SGYTLTELSMQWVRQAPGKGLEWM | NNIGSKSVHWYQQKPGQAPVLVVY | NNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLRSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLIVL (SEQ ID |
| | CDKTHTCPPCPAPELLGGPSVFLFPPKPKDTLMISRTPEVTCVV | (SEQ ID NO: 206) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 162) | | | |
| 122 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTAVYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLIVL (SEQ ID |
| | CDKTHTCPPCPAPELLGGPSVFLFPPKPKDTLMISRTPEVTCVV | (SEQ ID NO: 207) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 163) | | | |
| 123 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLTS | SGYTLTELSMQWVRQAPGKGLEWM | NNIGSKSVHWYQQKPGQAPVLVVY | NNIGSKSVHWYQQKPGQAPVLVVY |
| | EDTALYYCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLIVL (SEQ ID |
| | CDKTHTCPPCPAPELLGGPSVFLFPPKPKDTLMISRTPEVTCVV | (SEQ ID NO: 208) | PPSSEELQANKATLVCLISDFYPG | NO: 316) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 261) | |
| | SLSLSPG (SEQ ID NO: 164) | | | |
| 15 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDDGETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TINRVEAGDEADYYCQVWDSSNDL | TINRVEAGDEADYYCQVWDSSNDL |
| | SSGLYSISSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 165) | PPSSEELQANKATLVCLISDFYPG | NO: 277) |
| | VDVSHEDPEVKFNWYVDGVEVHNAKTKPREEQYNSTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 119) | | | |
| 124 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDDGETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 165) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 142) | | | |
| 125 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDAGETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDAGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 188) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 143) | | | |
| 126 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDSGETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDSGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 189) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 144) | | | |
| 127 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDEGETMYAEKEQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDEGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 190) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 145) | | | |
| 128 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDDAETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDAETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 191) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 146) | | | |
| 129 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDDGETMYAEKFQGRLTVTEDTSTDTVYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 192) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 147) | | | |
| 130 | EVQLVQSGAEVKKPGASVKVSCKVSGYTITELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDAGETMYAEKFQGRLTVTEDTSTDTVYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYFCATSPGYDFFDYWGQGTLVTVSSASTKGPSVFPLAPS | GGFDPDAGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 193) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 148) | | | |
| 131 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDSGETMYAEKFQGRLTVTEDTSTDTVYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYFCATSPGYDFFDYWGQGTLVTVSSASTKGPSVFPLAPS | GGFDPDSGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 194) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VIHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 149) | | | |
| 132 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDEGETMYAEKFQGRLTVTEDTSTDTVYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYFCATSPGYDFFDYWGQGTLVTVSSASTKGPSVFPLAPS | GGFDPDEGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 195) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 150) | | | |
| 133 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDDAETMYAEKFQGRLTVTEDTSTDTVYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYFCATSPGYDFFDYWGQGTLVTVSSASTKGPSVFPLAPS | GGFDPDDAETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 196) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 151) | | | |
| 134 | EVQLVQSGAEVKKPGASVKVSCKVSGYTITELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 165) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 142) | | | |
| 135 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDAGETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDAGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 188) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 143) | | | |
| 136 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDSGETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDSGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 189) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 144) | | | |
| 137 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDEGETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDEGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVATFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 190) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKITVDKSRWQQGNVFSCSVMHEALHNHYTQK | | (SEQ ID NO: 262) | |
| | SLSLSPG (SEQ ID NO: 145) | | | |
| 138 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDAETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDAETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 191) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVESCSVMHEALHNHYTQK | | (SEQ ID NO: 262) | |
| | SLSLSPG (SEQ ID NO: 146) | | | |
| 139 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 192) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 147) | | | |
| 140 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDAGETMYAEKFQGRLTVTEDTSTDTVYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYFCATSPGYDFFDYWGQGTLVTVSSASTKGPSVFPLAPS | GGFDPDAGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSNDL | TISRVEAGDEADYYCQVWDSSNDL |
| | SSGLYSISSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 193) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 148) | | | |
| 141 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDSGETMYAEKFQGRLTVTEDTSTDTVYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYFCATSPGYDFFDYWGQGTLVTVSSASTKGPSVFPLAPS | GGFDPDSGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 194) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 149) | | | |
| 142 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDEGETMYAEKFQGRLTVTEDTSTDTVYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYFCATSPGYDFFDYWGQGTLVTVSSASTKGPSVFPLAPS | GGFDPDEGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 195) | PPSSEELQANKATLVCLISDFYPG | NO: 317) |
| | VDVSHEDPEVKFNWYVDGVEVHNAKTRPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 150) | | | |
| 143 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDAETMYAEKFQGRLTVTEDTSTDTVYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYFCATSPGYDFFDYWGQGTLVTVSSASTKGPSVFPLAPS | GGFDPDDAETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 196) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 151) | | | |
| 144 | QVQLVQSGAEVKKPGASVKVSCKVSGYTLTELSMQWVRQAPGKG | QVQLVQSGAEVKKPGASVKVSCKV | SYELTQPPSVSVAPGKTARITCGG | SYELTQPPSVSVAPGKTARITCGG |
| | LEWMGGFDPDDGETMYAEKFQGRVTMTEDTSTDTAYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYYCATSPGYDFFDYWGQGTLVTVSSASTKGPSVFPLAPS | GGFDPDDGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 197) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 152) | | | |
| 145 | QVQLVQSGAEVKKPGASVKVSCKVSGYTLTELSMQWVRQAPGKG | QVQLVQSGAEVKKPGASVKVSCKV | SYELTQPPSVSVAPGKTARITCGG | SYELTQPPSVSVAPGKTARITCGG |
| | LEWMGGFDPDAGETMYAEKFQGRVTMTEDTSTDTAYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYYCATSPGYDFFDYWGQGTLVTVSSASTKGPSVFPLAPS | GGFDPDAGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 198) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQ | | (SEQ ID NO: 222) | |
| | SLSLSPG (SEQ ID NO: 153) | | | |
| 146 | QVQLVQSGAEVKKPGASVKVSCKVSGYTLTELSMQWVRQAPGKG | QVQLVQSGAEVKKPGASVKVSCKV | SYELTQPPSVSVAPGKTARITCGG | SYELTQPPSVSVAPGKTARITCGG |
| | LEWMGGFDPDSGETMYAEKFQGRVTMTEDTSTDTAYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYYCATSPGYDFFDYWGQGTLVTVSSASTKGPSVFPLAPS | GGFDPDSGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TINRVEAGDEADYYCQVWDSSNDL | TINRVEAGDEADYYCQVWDSSNDL |
| | SSGLYSISSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDRTHTCPPCPAPELLGGPSVFLFPPKPKDTLMISRTPEVTCVV | (SEQ ID NO: 199) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 154) | | | |
| 147 | QVQLVQSGAEVKKPGASVKVSCKVSGYTLTELSMQWVRQAPGKG | QVQLVQSGAEVKKPGASVKVSCKV | SYELTQPPSVSVAPGKTARITCGG | SYELTQPPSVSVAPGKTARITCGG |
| | LEWMGGFDPDEGETMYAEKFQGRVTMTEDTSTDTAYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYYCATSPGYDFFDYWGQGTLVTVSSASTKGPSVEPLAPS | GGFDPDEGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 200) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 155) | | | |
| 148 | QVQLVQSGAEVKKPGASVKVSCKVSGYTLTELSMQWVRQAPGKG | QVQLVQSGAEVKKPGASVKVSCKV | SYELTQPPSVSVAPGKTARITCGG | SYELTQPPSVSVAPGKTARITCGG |
| | LEWMGGFDPDDAETMYAEKFQGRVTMTEDTSTDTAYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYYCATSPGYDFFDYWGQGTLVTVSSASTKGPSVFPLAPS | GGFDPDDAETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 201) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 156) | | | |
| 149 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRVTMTEDTSTDTAYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYYCATSPGYDFFDYWGQGTLVTVSSASTKGPSVFPLAPS | GGFDPDDGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 197) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 152) | | | |
| 150 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDAGETMYAEKFQGRVTMTEDTSTDTAYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYYCATSPGYDFFDYWGQGTLVTVSSASTKGPSVFPLAPS | GGFDPDAGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 198) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTD | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 153) | | | |
| 151 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDSGETMYAEKFQGRVTMTEDTSTDTAYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYYCATSPGYDFFDYWGQGTLVTVSSASTKGPSVFPLAPS | GGFDPDSGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 199) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 154) | | | |
| 152 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDEGETMYAEKFQGRVTMTEDTSTDTAYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYYCATSPGYDFFDYWGQGTLVTVSSASTKGPSVFPLAPS | GGFDPDEGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 200) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 155) | | | |
| 153 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDDAETMYAEKFQGRVTMTEDTSTDTAYMELSSLRS | SGYTLTELSMQWVRQAPGKGLEWM | DNIGSKSVHWYQQKPGQAPVLVVY | DNIGSKSVHWYQQKPGQAPVLVVY |
| | EDTAVYYCATSPGYDFFDYWGQGTLVTVSSASTKGPSVFPLAPS | GGFDPDDAETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 201) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 156) | | | |
| 154 | QVQLVQSGAEVRKPGASVKVSCKVSGYTLTELSMQWVRQAPGKG | QVQLVQSGAEVKKPGASVKVSCKV | SYELTQPPSVSVAPGKTARITCGG | SYELTQPPSVSVAPGKTARITCGG |
| | LEWMGGFDPDDGETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 170) | PPSSEELQANKATLVCLISDEYPG | NO: 277) |
| | VDVSHEDPEVKFNWYVDGVEVHNAKTKPREEQYASTYRVVSVLT | | AVTVAWKADSSPVKAGVETTTPSK | |
| | VIHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 157) | | | |
| 155 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDDGETMYAEKFQGRVTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRVTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 202) | PPSSEELQANKATLVCLISDEYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 158) | | | |
| 156 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDDGETMYAEKFQGRLTMTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRLTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 203) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 159) | | | |
| 157 | EVQLVQSGAEVKKPGASVKVSCKVSGYTITELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDDGETMYAERFQGRLTVTEDTSTDTAYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELRSLTSEDTALYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 204) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKITP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 160) | | | |
| 158 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDDGETMYAEKFQGRLTVTEDTSTDTVYMELSSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLTSEDTALYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDRTHTCPPCPAPELLGGPSVFLFPPKPKDTLMISRTPEVTCVV | (SEQ ID NO: 205) | PPSSEELQANKATLVCLISDEYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 161) | | | |
| 159 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDDGETMYAEKFQGRLTVTEDTSTDTVYMELRSLRS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLRSEDTALYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLE | LIEGGGTKLTVL (SEQ ID |
| | CDKTHTCPPCPAPELLGGPSVFLFPPKPKDTLMISRTPEVTCVV | (SEQ ID NO: 206) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 162) | | | |
| 160 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDDGETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTAVYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTAVYFC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 207) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 163) | | | |
| 161 | EVQLVQSGAEVKKPGASVKVSCKVSGYTLTELSMQWVRQAPGKG | EVQLVQSGAEVKKPGASVKVSCKV | SYELTQPPSVSVAPGKTARITCGG | SYELTQPPSVSVAPGKTARITCGG |
| | LEWMGGFDPDDGETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYYCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYYC | TINRVEAGDEADYYCQVWDSSNDL | TINRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 208) | PPSSEELQANKATLVCLISDFYPG | NO: 277) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 222) | |
| | SLSLSPG (SEQ ID NO: 164) | | | |
| 162 | QVQLVQSGAEVKKPGASVKVSCKVSGYTLTELSMQWVRQAPGKG | QVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 170) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 157) | | | |
| 163 | EVQLVQSGAEVKKPGASVKVSCKVSGYTITELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRVTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRVTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSISSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 202) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | SEQ ID NO: 262) | |
| | SLSLSPG (SEQ ID NO: 158) | | | |
| 164 | EVQLVQSGAEVRKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTMTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRLTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 203) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 159) | | | |
| 165 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTAYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELRSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 204) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 160) | | | |
| 166 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELSSLTS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLTSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 205) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 161) | | | |
| 167 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLRS | SGYTLTELSMQWVRQAPGKGLEWM | DNIGSKSVHWYQQKPGQAPVLVVY | DNIGSKSVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLRSEDTALYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 206) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 162) | | | |
| 168 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTAVYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGQLVKDYFPEPVTVSWNSGALTSGVHTFPAVLQ | DTSTDTVYMELRSLTSEDTAVYFC | TISRVEAGDEADYYCQVWDSSNDL | TISRVEAGDEADYYCQVWDSSNDL |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 207) | PPSSEELQANKATLVCLISDFYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 163) | | | |
| 169 | EVQLVQSGAEVKKPGASVKVSCKVSGYTLTELSMQWVRQAPGKG | EVQLVQSGAEVKKPGASVKVSCKV | SYVLTQPPSVSVAPGKTARITCGG | SYVLTQPPSVSVAPGKTARITCGG |
| | LEWMGGFDPDDGETMYAEKFQGRLTVTEDTSTDTVYMELRSLTS | SGYTLTELSMQWVRQAPGKGLEWM | DNIGSKSVHWYQQKPGQAPVLVVY | DNIGSKSVHWYQQKPGQAPVLVVY |
| | EDTALYYCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYYC | TISRVEAGDEADYYCQVWDSSNDL | TISRVEAGDEADYYCQVWDSSNDL |
| | SSGLYSISSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 208) | PPSSEELQANKATLVCLISDEYPG | NO: 317) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 262) | |
| | SLSLSPG (SEQ ID NO: 164) | | | |
| 17 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 165) | PPSSEELQANKATLVCLISDFYPG | NO: 279) |
| | VDVSHEDPEVKFNWYVDGVEVHNAKTKPREEQYNSTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 119) | | | |
| 170 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 165) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 142) | | | |
| 171 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDAGETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDAGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVATEPAVLQ | DTSTDTVYMELRSLTSEDTALYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 188) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 143) | | | |
| 172 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDSGETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDSGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 189) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 144) | | | |
| 173 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDEGETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDEGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 190) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 145) | | | |
| 174 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDAETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDAETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 191) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 146) | | | |
| 175 | EVQLVQSGAEVKKPGASVKVSCKVSGYTITELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 192) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 147) | | | |
| 176 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDAGETMYAEKFQGRLTVTEDTSTDTVYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYFCATSPGYDFFDYWGQGTLVTVSSASTKGPSVFPLAPS | GGFDPDAGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 193) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 148) | | | |
| 177 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDSGETMYAEKFQGRLTVTEDTSTDTVYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYFCATSPGYDFFDYWGQGTLVTVSSASTKGPSVFPLAPS | GGFDPDSGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 194) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 149) | | | |
| 178 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDEGETMYAEKFQGRLTVTEDTSTDTVYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYFCATSPGYDFFDYWGQGTLVTVSSASTKGPSVFPLAPS | GGFDPDEGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 195) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 150) | | | |
| 179 | EVQLVQSGAEVKKPGASVKVSCKVSGYTLTELSMQWVRQAPGKG | | | |
| | LEWMGGFDPDDAETMYAEKFQGRLTVTEDTSTDTVYMELSSLRS | EVQLVQSGAEVKKPGASVKVSCKV | SYVLTQPPSVSVAPGQTARITCGG | SYVLTQPPSVSVAPGQTARITCGG |
| | EDTAVYFCATSPGYDFFDYWGQGTLVTVSSASTKGPSVFPLAPS | SGYTLTELSMQWVRQAPGKGLEWM | SDIGDEIVHWYQQKPGQAPVLVVY | SDIGDEIVHWYQQKPGQAPVLVVY |
| | SKSTSGGTAALGCLVKDYFPEPVTVSWNSGALTSGVHTFPAVLQ | GGFDPDDAETMYAEKFQGRLTVTE | DDSDRPSGIPERFSGSNSGNTATL | DDSDRPSGIPERFSGSNSGNTATL |
| | SSGLYSLSSVVTVPSSSLGTQTYICNVNHKPSNTKVDKRVEPKS | DTSTDTVYMELSSLRSEDTAVYFC | TISRAQAGDEADYYCQVWDSSSDH | TISRAQAGDEADYYCQVWDSSSDH |
| | CDKTHTCPPCPAPELLGGPSVFLFPPKPKDTLMISRTPEVTCVV | ATSPGYDFFDYWGQGTLVTVSS | LIFGGGTKLTVLGQPKAAPSVTLF | LIFGGGTKLTVL (SEQ ID |
| | VDVSHEDPEVKFNWYVDGVEVHNAKTKPREEQYASTYRVVSVLT | (SEQ ID NO: 196) | PPSSEELQANKATLVCLISDFYPG | NO: 279) |
| | VLHQDWLNGKEYKCKVSNKALPAPIEKTISKAKGQPREPQVYTL | | AVTVAWKADSSPVKAGVETTTPSK | |
| | PPSREEMTKNQVSLTCLVKGFYPSDIAVEWESNGQPENNYKITP | | QSNNKYAASSYLSLTPEQWKSHRS | |
| | PVLDSDGSFFLYSKLTVDKSRWQQGNVFSCSVMHEALHNHYTQK | | YSCQVTHEGSTVEKTVAPTECS | |
| | SLSLSPG (SEQ ID NO: 151) | | (SEQ ID NO: 224) | |
| 180 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDRTHTCPPCPAPELLGGPSVFLFPPKPKDTLMISRTPEVTCVV | (SEQ ID NO: 165) | PPSSEELQANKATLVCLISDEYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 142) | | | |
| 181 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDAGETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDAGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 188) | PPSSEELQANKATLVCLISDEYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 143) | | | |
| 182 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDSGETMYAEKFQGRLTVTEDTSTDTVYMELRSLIS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDSGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 189) | PPSSEELQANKATLVCLISDEYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 144) | | | |
| 183 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDEGETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDEGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 190) | PPSSEELQANKATLVCLISDEYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 145) | | | |
| 184 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDAETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDAETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 191) | PPSSEELQANKATLVCLISDEYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 146) | | | |
| 185 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSSDH | TISRVEAGDEADYYCQVWDSSSDH |
| | SSGLYSISSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 192) | PPSSEELQANKATLVCLISDEYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 147) | | | |
| 186 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDAGETMYAEKFQGRLTVTEDTSTDTVYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYFCATSPGYDFFDYWGQGTLVTVSSASTKGPSVFPLAPS | GGFDPDAGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 193) | PPSSEELQANKATLVCLISDEYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 148) | | | |
| 187 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDSGETMYAEKFQGRLTVTEDTSTDTVYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYFCATSPGYDFFDYWGQGTLVTVSSASTKGPSVFPLAPS | GGFDPDSGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 194) | PPSSEELQANKATLVCLISDEYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 149) | | | |
| 188 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDEGETMYAEKFQGRLTVTEDTSTDTVYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYFCATSPGYDFFDYWGQGTLVTVSSASTKGPSVFPLAPS | GGFDPDEGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 195) | PPSSEELQANKATLVCLISDEYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 150) | | | |
| 189 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDAETMYAEKFQGRLTVTEDTSTDTVYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYFCATSPGYDFFDYWGQGTLVTVSSASTKGPSVFPLAPS | GGFDPDDAETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLRSEDTAVYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 196) | PPSSEELQANKATLVCLISDEYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 151) | | | |
| 190 | QVQLVQSGAEVKKPGASVKVSCKVSGYTLTELSMQWVRçAPGKG | QVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRVTMTEDTSTDTAYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYYCATSPGYDFFDYWGQGTLVTVSSASTKGPSVFPLAPS | GGFDPDDGETMYAEKFQGRVTMTE | DDSDRPSGIPERFSGSNSGNTATL | DDSDRPSGIPERFSGSNSGNTATI |
| | SKSTSGGTAALGCLVKDYFPEPVTVSWNSGALTSGVHTFPAVLQ | DTSTDTAYMELSSLRSEDTAVYYC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 197) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 152) | | | |
| 191 | QVQLVQSGAEVKKPGASVKVSCKVSGYTLTELSMQWVRQAPGKG | QVQLVQSGAEVKKPGASVKVSCKV | SYVLTQPPSVSVAPGQTARITCGG | SYVLTQPPSVSVAPGQTARITCGG |
| | LEWMGGFDPDAGETMYAEKFQGRVTMTEDTSTDTAYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYYCATSPGYDFFDYWGQGTLVTVSSASTKGPSVFPLAPS | GGFDPDAGETMYAEKFQGRVTMTE | DDSDRPSGIPERFSGSNSGNTATL | DDSDRPSGIPERFSGSNSGNTATI |
| | SKSTSGGTAALGCLVKDYFPEPVTVSWNSGALTSGVHTFPAVLQ | DTSTDTAYMELSSLRSEDTAVYYC | TISRAQAGDEADYYCQVWDSSSDH | TISRAQAGDEADYYCQVWDSSSDH |
| | SSGLYSISSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDRTHTCPPCPAPELLGGPSVFLFPPKPKDTLMISRTPEVTCVV | (SEQ ID NO: 198) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 153) | | | |
| 192 | QVQLVQSGAEVKKPGASVKVSCKVSGYTLTELSMQWVRQAPGKG | QVQLVQSGAEVKKPGASVKVSCKV | SYVLTQPPSVSVAPGQTARITCGG | SYVLTQPPSVSVAPGQTARITCGG |
| | LEWMGGFDPDSGETMYAEKFQGRVTMTEDTSTDTAYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYYCATSPGYDFFDYWGQGTLVTVSSASTKGPSVEPLAPS | GGFDPDSGETMYAEKFQGRVTMTE | DDSDRPSGIPERFSGSNSGNTATL | DDSDRPSGIPERFSGSNSGNTATI |
| | SKSTSGGTAALGCLVKDYFPEPVTVSWNSGALTSGVHTFPAVLQ | DTSTDTAYMELSSLRSEDTAVYYC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 199) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 154) | | | |
| 193 | QVQLVQSGAEVKKPGASVKVSCKVSGYTLTELSMQWVRQAPGKG | QVQLVQSGAEVKKPGASVKVSCKV | SYVLTQPPSVSVAPGQTARITCGG | SYVLTQPPSVSVAPGQTARITCGG |
| | LEWMGGFDPDEGETMYAEKFQGRVTMTEDTSTDTAYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYYCATSPGYDFFDYWGQGTLVTVSSASTKGPSVFPLAPS | GGFDPDEGETMYAEKFQGRVTMTE | DDSDRPSGIPERFSGSNSGNTATL | DDSDRPSGIPERFSGSNSGNTATI |
| | SKSTSGGTAALGCLVKDYFPEPVTVSWNSGALTSGVHTFPAVLQ | DTSTDTAYMELSSLRSEDTAVYYC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 200) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 155) | | | |
| 194 | QVQLVQSGAEVKKPGASVKVSCKVSGYTLTELSMQWVRQAPGKG | QVQLVQSGAEVKKPGASVKVSCKV | SYVLTQPPSVSVAPGQTARITCGG | SYVLTQPPSVSVAPGQTARITCGG |
| | LEWMGGFDPDDAETMYAEKFQGRVTMTEDTSTDTAYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYYCATSPGYDFFDYWGQGTLVTVSSASTKGPSVFPLAPS | GGFDPDDAETMYAEKFQGRVTMTE | DDSDRPSGIPERFSGSNSGNTATL | DDSDRPSGIPERFSGSNSGNTATI |
| | SKSTSGGTAALGCLVKDYFPEPVTVSWNSGALTSGVHTFPAVLQ | DTSTDTAYMELSSLRSEDTAVYYC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 201) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 156) | | | |
| 195 | QVQLVQSGAEVKKPGASVKVSCKVSGYTLTELSMQWVRQAPGKG | QVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRVTMTEDTSTDTAYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYYCATSPGYDFFDYWGQGTLVTVSSASTKGPSVFPLAPS | GGFDPDDGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 197) | PPSSEELQANKATLVCLISDFYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 152) | | | |
| 196 | QVQLVQSGAEVKKPGASVKVSCKVSGYTLTELSMQWVRQAPGKG | QVQLVQSGAEVKKPGASVKVSCKV | SYVLTQPPSVSVAPGQTARITCGG | SYVLTQPPSVSVAPGQTARITCGG |
| | LEWMGGFDPDAGETMYAEKFQGRVTMTEDTSTDTAYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYYCATSPGYDFFDYWGQGTLVTVSSASTKGPSVFPLAPS | GGFDPDAGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 198) | PPSSEELQANKATLVCLISDFYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 153) | | | |
| 197 | QVQLVQSGAEVKKPGASVKVSCKVSGYTLTELSMQWVRQAPGKG | QVQLVQSGAEVKKPGASVKVSCKV | SYVLTQPPSVSVAPGQTARITCGG | SYVLTQPPSVSVAPGQTARITCGG |
| | LEWMGGFDPDSGETMYAEKFQGRVTMTEDTSTDTAYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYYCATSPGYDFFDYWGQGTLVTVSSASTKGPSVFPLAPS | GGFDPDSGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 199) | PPSSEELQANKATLVCLISDFYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 154) | | | |
| 198 | QVQLVQSGAEVKKPGASVKVSCKVSGYTITELSMQWVRQAPGKG | QVQLVQSGAEVKKPGASVKVSCKV | SYVLTQPPSVSVAPGQTARITCGG | SYVLTQPPSVSVAPGQTARITCGG |
| | LEWMGGFDPDEGETMYAEKFQGRVTMTEDTSTDTAYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYYCATSPGYDFFDYWGQGTLVTVSSASTKGPSVFPLAPS | GGFDPDEGETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 200) | PPSSEELQANKATLVCLISDFYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 155) | | | |
| 199 | QVQLVQSGAEVKKPGASVKVSCKVSGYTLTELSMQWVRQAPGKG | QVQLVQSGAEVKKPGASVKVSCKV | SYVLTQPPSVSVAPGQTARITCGG | SYVLTQPPSVSVAPGQTARITCGG |
| | LEWMGGFDPDDAETMYAEKFQGRVTMTEDTSTDTAYMELSSLRS | SGYTLTELSMQWVRQAPGKGLEWM | SDIGDEIVHWYQQKPGQAPVLVVY | SDIGDEIVHWYQQKPGQAPVLVVY |
| | EDTAVYYCATSPGYDFFDYWGQGTLVTVSSASTKGPSVFPLAPS | GGFDPDDAETMYAEKFQGRVTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELSSLRSEDTAVYYC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 201) | PPSSEELQANKATLVCLISDFYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 156) | | | |
| 200 | QVQLVQSGAEVKKPGASVKVSCKVSGYTLTELSMQWVRQAPGKG | QVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 170) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 157) | | | |
| 201 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRVTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRVTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 202) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 158) | | | |
| 202 | EVQLVQSGAEVKKPGASVKVSCKVSGYTITELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTMTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRLTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 203) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 159) | | | |
| 203 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTAYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELRSLTSEDTALYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 204) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 160) | | | |
| 204 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELSSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLTSEDTALYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 205) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 161) | | | |
| 205 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELRSLRS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLRSEDTALYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 206) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 162) | | | |
| 206 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTAVYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTAVYFC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 207) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIERTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 163) | | | |
| 207 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYYCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYYC | TISRAQAGDEADYYCQVWDSSSDH | TISRAQAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 208) | PPSSEELQANKATLVCLISDFYPG | NO: 279) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 224) | |
| | SLSLSPG (SEQ ID NO: 164) | | | |
| 208 | QVQLVQSGAEVKKPGASVKVSCKVSGYTLTELSMQWVRQAPGKG | QVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 170) | PPSSEELQANKATLVCLISDFYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 157) | | | |
| 209 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRVTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRVTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 202) | PPSSEELQANKATLVCLISDFYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 158) | | | |
| 210 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTMTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRLTMTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 203) | PPSSEELQANKATLVCLISDFYPG | NO: 318) |
| | VDVSHEDPEVKFNWYVDGVEVHNAKTRPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 159) | | | |
| 211 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTAYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTAYMELRSLTSEDTALYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 204) | PPSSEELQANKATLVCLISDFYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 160) | | | |
| 212 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELSSLTS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELSSLTSEDTALYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 205) | PPSSEELQANKATLVCLISDFYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 161) | | | |
| 213 | EVQLVQSGAEVKKPGASVKVSCKVSGYTLTELSMQWVRçAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELRSLRS | SGYTLTELSMQWVRQAPGKGLEWM | SDIGDEIVHWYQQKPGQAPVLVVY | SDIGDEIVHWYQQKPGQAPVLVVY |
| | EDTALYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLRSEDTALYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 206) | PPSSEELQANKATLVCLISDFYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 162) | | | |
| 214 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTAVYFCATSPGYDFFDYWGQGTLVTVSSASTKGPSVFPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTAVYFC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 207) | PPSSEELQANKATLVCLISDFYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 163) | | | |
| 215 | EVQLVQSGAEVKKPGASVKVSCKVSGYTLTELSMQWVRQAPGKG | EVQLVQSGAEVKKPGASVKVSCKV | SYVLTQPPSVSVAPGQTARITCGG | SYVLTQPPSVSVAPGQTARITCGG |
| | LEWMGGFDPDDGETMYAEKFQGRLTVTEDTSTDTVYMELRSLTS | SGYTLTELSMQWVRQAPGKGLEWM | SDIGDEIVHWYQQKPGQAPVLVVY | SDIGDEIVHWYQQKPGQAPVLVVY |
| | EDTALYYCATSPGYDFFDYWGQGTLVTVSSASTKGPSVEPLAPS | GGFDPDDGETMYAEKFQGRLTVTE | DDSDRPSGIPERFSGSNSGNTATL | DDSDRPSGIPERFSGSNSGNTATL |
| | SKSTSGGTAALGCLVKDYFPEPVTVSWNSGALTSGVHTFPAVLQ | DTSTDTVYMELRSLTSEDTALYYC | TISRVEAGDEADYYCQVWDSSSDH | TISRVEAGDEADYYCQVWDSSSDH |
| | SSGLYSLSSVVTVPSSSLGTQTYICNVNHKPSNTKVDKRVEPKS | ATSPGYDFFDYWGQGTLVTVSS | LIFGGGTKLTVLGQPKAAPSVTLF | LIFGGGTKLTVL (SEQ ID |
| | CDKTHTCPPCPAPELLGGPSVFLFPPKPKDTLMISRTPEVTCVV | (SEQ ID NO: 208) | PPSSEELQANKATLVCLISDFYPG | NO: 318) |
| | VDVSHEDPEVKFNWYVDGVEVHNAKTKPREEQYASTYRVVSVLT | | AVTVAWKADSSPVKAGVETTTPSK | |
| | VLHQDWLNGKEYKCKVSNKALPAPIEKTISKAKGQPREPQVYTL | | QSNNKYAASSYLSLTPEQWKSHRS | |
| | PPSREEMTKNQVSLTCLVKGFYPSDIAVEWESNGQPENNYKTTP | | YSCQVTHEGSTVEKTVAPTECS | |
| | PVLDSDGSFFLYSKLTVDKSRWQQGNVFSCSVMHEALHNHYTQK | | (SEQ ID NO: 263) | |
| | SLSLSPG (SEQ ID NO: 164) | | | |

### Example 2: Binding Affinity

The amino acid sequence of recombinant MBP-mutCALR Type 1 fusion protein used in Experiments 1 and 2 is:

(SEQ ID NO: 335)
HHHHHHMKIEEGKLVIWINGDKGYNGLAEVGKKFEKDTGIKVTVEHPDKLEEKFPQVAATGDGP
DIIFWAHDRFGGYAQSGLLAEITPDKAFQDKLYPFTWDAVRYNGKLIAYPIAVEALSLIYNKDL
LPNPPKTWEEIPALDKELKAKGKSALMENLQEPYFTWPLIAADGGYAFKYENGKYDIKDVGVDN
AGAKAGLTFLVDLIKNKHMNADTDYSIAEAAFNKGETAMTINGPWAWSNIDTSKVNYGVTVLPT
FKGQPSKPEVGVLSAGINAASPNKELAKEFLENYLLTDEGLEAVNKDKPLGAVALKSYEEELAK
DPRIAATMENAQKGEIMPNIPQMSAFWYAVRTAVINAASGRQTVDEALKDAQTRSSSLEVLFQG
PGLNDIFEAQKIEWHEENLYFQGEPAVYFKEQFLDGDGWTSRWIEKHKSDFGKFVLSSGKFYGD
EEKDKGLQTSQDARFYALSASFEPFSNKGQTLVVQFTVKHEQNIDCGGGYVKLFPNSLDQTDMH
GDSEYNIMFGPDICGPGTKKVHVIFNYKGKNVLINKDIRCKDDEFTHLYTLIVRPDNTYEVKID
NSQVESGSLEDDWDFLPPKKIKDPDASKPEDWDERAKIDDPTDSKPEDWDKPEHIPDPDAKKPE
DWDEEMDGEWEPPVIQNPEYKGEWKPRQIDNPDYKGTWIHPEIDNPEYSPDPSIYAYDNFGVLG
LDLWQVKSGTIFDNFLITNDEAYAEEFGNETWGVTKAAEKQMKDKQDEEQRTRRMMRTKMRMRR
MRRTRRKMRRKMSPARPRISCREACLQGWTEA

Experiment 1: Recombinant MBP-mutCALR Type 1 fusion protein was custom made at Cepter (cepterbiopartners.com). Data for the interaction of antibodies with recombinant MBP-mutCALR Type 1 fusion protein were collected at 25° C. on a Biacore 8K instrument (Cytiva, Marlborough, Mass.), and all reagents were obtained from Cytiva unless otherwise specified. The experiments were conducted using Tris-buffered saline (pH 7.2), 0.005% Surfactant P20, 1 mM CaCl₂) as running buffer. To prepare the capture surfaces, anti-human IgG (Fc) antibodies (Product #29234600) was amine-coupled onto a CM4 chip (Product #BR100534) under standard conditions using an Amine Coupling Kit (Product #BR100050). Anti-mutCALR antibodies were captured onto the chip surface by injecting over flow cell 2 only at a flow rate of 30 μL/min for 30 seconds. Typical capture levels were in the range of 10-30RU. Recombinant MBP-mutCALR Type 1 fusion protein was prepared at nominal concentrations of 0, 3.1, 9.3, 27.8, 83.3, and 250 nM in the running buffer and was injected over both flow cells 1 and 2 for 210 seconds at a flow rate of 50 μL/min, followed by a 510-second dissociation phase at the same flow rate. The kinetics parameters were obtained by applying 1:1 binding model to fit the data from multiple-cycle injection experiments using Biacore Insight Evaluation software. MBP protein at 250 nM concentration was injected as a control, which showed no binding to anti-mutCALR antibodies.

TABLE 6
Binding affinities and kinetic association and dissociation rate constants
to mutCALR for the indicated antibodies for Experiment 1.

| Clone No. | kₐ (1/Ms) | k_{d} (1/s) | KD (M) |
|---|---|---|---|
| 38 | 1.28E+06 | 2.68E−03 | 2.10E−09 |
| 1 | 1.38E+06 | 1.96E−02 | 1.42E−08 |
| 2 | 1.63E+06 | 1.23E−02 | 7.58E−09 |
| 4 | 1.63E+06 | 7.04E−03 | 4.33E−09 |
| 5 | 9.35E+05 | 4.05E−03 | 4.33E−09 |
| 31 | ND | ND | ND |
| 32 | 1.36E+06 | 7.68E−03 | 5.63E−09 |
| 6 | 5.97E+05 | 1.53E−03 | 2.57E−09 |
| 53 | 2.29E+05 | 2.35E−03 | 1.02E−08 |
| 33 | 7.75E+05 | 1.26E−02 | 1.62E−08 |
| 39 | 3.22E+05 | 1.80E−03 | 5.59E−09 |
| 52 | 6.99E+06 | 1.23E−02 | 1.76E−09 |
| 7 | 1.59E+06 | 6.09E−03 | 3.83E−09 |
| 8 | 1.75E+06 | 8.02E−03 | 4.58E−09 |
| 40 | 5.17E+06 | 5.38E−03 | 1.04E−09 |
| 9 | 3.02E+06 | 9.04E−03 | 2.99E−09 |
| 10 | 2.11E+06 | 2.92E−03 | 1.38E−09 |
| 11 | 5.14E+05 | 5.24E−03 | 1.02E−08 |
| 34 | 2.27E+06 | 5.81E−03 | 2.56E−09 |
| 12 | 5.89E+05 | 2.40E−03 | 4.08E−09 |
| 13 | 2.99E+06 | 3.01E−02 | 1.01E−08 |
| 35 | 2.13E+06 | 5.99E−03 | 2.82E−09 |
| 36 | 6.95E+05 | 2.05E−03 | 2.95E−09 |
| 37 | 2.15E+05 | 2.12E−03 | 9.88E−09 |
| 14 | ND | ND | ND |
| 41 | 1.66E+06 | 8.11E−03 | 4.88E−09 |
| 15 | 3.57E+07 | 2.15E−01 | 6.03E−09 |
| 16 | 2.84E+06 | 3.54E−02 | 1.25E−08 |
| 17 | 1.56E+06 | 3.47E−03 | 2.22E−09 |
| 18 | 2.37E+06 | 6.28E−03 | 2.66E−09 |
| 3 | 2.08E+06 | 7.16E−03 | 3.45E−09 |
| 19 | 1.46E+06 | 1.32E−02 | 9.04E−09 |
| 20 | 1.12E+06 | 4.98E−03 | 4.46E−09 |
| 21 | 2.57E+06 | 6.64E−03 | 2.58E−09 |
| 22 | 2.29E+05 | 2.89E−03 | 1.26E−08 |
| 23 | 7.85E+05 | 3.43E−03 | 4.37E−09 |
| 42 | 6.49E+05 | 4.52E−03 | 6.97E−09 |
| 24 | 2.61E+05 | 1.99E−03 | 7.62E−09 |
| 25 | 5.85E+05 | 3.89E−03 | 6.64E−09 |
| 26 | 4.78E+05 | 4.71E−03 | 9.84E−09 |
| 27 | 3.80E+05 | 3.66E−03 | 9.63E−09 |
| 28 | 4.60E+05 | 8.97E−03 | 1.95E−08 |
| 29 | 6.88E+05 | 5.61E−03 | 8.16E−09 |
| 30 | 2.31E+05 | 2.95E−03 | 1.28E−08 |
| 43 | 1.87E+05 | 6.20E−04 | 3.32E−09 |
| 44 | 2.63E+05 | 1.01E−03 | 3.84E−09 |
| 45 | 2.14E+05 | 1.10E−03 | 5.13E−09 |
| 46 | 2.83E+05 | 1.56E−03 | 5.50E−09 |
| 47 | 2.13E+05 | 7.72E−04 | 3.62E−09 |
| 48 | 1.81E+05 | 2.97E−04 | 1.64E−09 |
| 49 | 1.85E+05 | 6.69E−04 | 3.61E−09 |
| 50 | 2.49E+05 | 1.04E−03 | 4.20E−09 |
| 51 | 2.35E+05 | 1.09E−03 | 4.64E−09 |

ND, not determined.

Experiment 2: Recombinant MBP-mutCALR Type 1 fusion protein was custom made at Cepter (cepterbiopartners.com). Data for the interaction of antibodies with recombinant mutCALR protein were collected at 25° C. on a Biacore 8K instrument (Cytiva, Marlborough, Mass.), and all reagents were obtained from Cytiva unless otherwise specified. The experiments were conducted using Tris-buffered saline (pH 7.2), 0.005% Surfactant P20, 1 mM CaCl₂) as running buffer. To prepare the capture surfaces, anti-human IgG (Fc) antibodies (Product #29234600) was amine-coupled onto a CM4 chip (Product #BR100534) under standard conditions using an Amine Coupling Kit (Product #BR100050). Anti-mutCALR antibodies were captured onto the chip surface by injecting over flow cell 2 only at a flow rate of 10 μL/min for 30 seconds. Typical capture levels were in the range of 15-25RU. Recombinant MBP-mutCALR Type 1 fusion protein was prepared at nominal concentrations of 0, 0.75, 2.22, 6.67, 20, and 60 nM and injected over both flow cells 1 and 2 for 150 seconds at a flow rate of 69 L/min, followed by a 230-second dissociation phase at the same flow rate. The kinetics parameters were obtained by applying 1:1 binding model to fit the data from multiple-cycle injection experiments using Biacore Insight Evaluation software.

TABLE 7
Binding affinities and kinetic association and dissociation
rate constants to mutCALR for the indicated parental
and variant antibodies for Experiment 2.

| Clone No. | kₐ (1/Ms) | k_{d} (1/s) | KD (M) |
|---|---|---|---|
| 32 | 1.84E+06 | 3.14E−03 | 1.71E−09 |
| 6 | 2.29E+06 | 1.23E−03 | 5.38E−10 |
| 53 | 6.36E+06 | 1.08E−01 | 1.70E−08 |
| 52 | 1.79E+06 | 2.30E−03 | 1.29E−09 |
| 40 | 3.44E+06 | 1.21E−03 | 3.52E−10 |
| 36 | 1.36E+06 | 1.66E−03 | 1.22E−09 |
| 15 | 2.44E+06 | 2.49E−03 | 1.02E−09 |
| 17 | 5.12E+06 | 1.27E−03 | 2.49E−10 |
| 51 | 6.36E+05 | 5.24E−04 | 8.23E−10 |
| 64 | 3.89E+06 | 2.48E−03 | 6.38E−10 |
| 70 | 9.87E+05 | 9.53E−04 | 9.66E−10 |
| 55 | 5.32E+06 | 9.11E−04 | 1.71E−10 |
| 75 | 6.12E+06 | 3.90E−03 | 6.37E−10 |
| 68 | 1.44E+06 | 1.66E−03 | 1.15E−09 |
| 74 | 1.29E+06 | 1.53E−03 | 1.19E−09 |
| 69 | 9.94E+06 | 5.27E−03 | 5.30E−10 |
| 79 | 4.53E+06 | 6.31E−03 | 1.39E−09 |
| 58 | 6.76E+06 | 2.32E−03 | 3.43E−10 |
| 84 | 8.92E+05 | 1.80E−03 | 2.01E−09 |
| 170 | 4.27E+06 | 1.79E−03 | 4.19E−10 |
| 63 | 2.83E+06 | 6.91E−04 | 2.44E−10 |
| 65 | 4.60E+06 | 2.31E−03 | 5.02E−10 |
| 124 | 1.23E+06 | 1.28E−03 | 1.04E−09 |
| 179 | 1.00E+07 | 6.81E−03 | 4.55E−10 |
| 138 | 7.86E+06 | 6.88E−03 | 8.75E−10 |
| 134 | 1.27E+06 | 1.42E−03 | 1.12E−09 |
| 180 | 1.00E+07 | 1.27E−01 | 1.98E−10 |
| 189 | 1.00E+07 | 1.53E−02 | 4.22E−10 |
| 174 | 7.80E+06 | 4.10E−03 | 5.26E−10 |
| 128 | 1.29E+06 | 2.07E−03 | 1.61E−09 |
| 184 | 4.28E+05 | 6.73E−04 | 1.57E−09 |
| 178 | 1.02E+06 | 1.23E−03 | 1.21E−09 |
| 173 | 5.35E+06 | 3.49E−03 | 6.53E−10 |
| 61 | 4.13E+06 | 3.69E−03 | 8.93E−10 |
| 183 | 1.23E+06 | 1.24E−03 | 1.00E−09 |
| 56 | 6.90E+05 | 2.12E−03 | 3.08E−09 |
| 66 | 3.54E+06 | 8.36E−03 | 2.36E−09 |
| 83 | 5.62E+06 | 9.92E−03 | 1.76E−09 |
| 133 | 8.51E+06 | 7.02E−03 | 8.26E−10 |
| 143 | 5.74E+06 | 7.53E−03 | 1.31E−09 |
| 142 | 1.19E+06 | 2.69E−03 | 2.27E−09 |
| 78 | 8.73E+05 | 2.40E−03 | 2.75E−09 |
| 187 | 1.00E+07 | 1.63E−02 | 1.59E−09 |
| 72 | 1.02E+06 | 3.82E−03 | 3.74E−09 |
| 62 | 3.52E+06 | 6.21E−03 | 1.76E−09 |
| 137 | 7.51E+06 | 9.50E−03 | 1.27E−09 |
| 172 | 1.12E+06 | 2.50E−03 | 2.24E−09 |
| 127 | 6.92E+06 | 8.99E−03 | 1.30E−09 |
| 132 | 1.13E+06 | 2.44E−03 | 2.16E−09 |
| 182 | 5.62E+06 | 6.92E−03 | 1.23E−09 |
| 177 | 3.70E+06 | 5.49E−03 | 1.49E−09 |
| 186 | 8.48E+05 | 2.43E−03 | 2.86E−09 |
| 171 | 6.57E+06 | 1.09E−02 | 1.65E−09 |
| 181 | 1.00E+07 | 2.09E−02 | 1.58E−09 |
| 77 | 3.13E+06 | 1.75E−02 | 5.61E−09 |
| 76 | 3.16E+06 | 1.55E−02 | 4.90E−09 |
| 81 | 7.46E+05 | 4.94E−03 | 6.62E−09 |
| 82 | 7.62E+05 | 4.75E−03 | 6.23E−09 |
| 136 | 7.56E+05 | 5.07E−03 | 6.70E−09 |
| 135 | 9.91E+05 | 5.12E−03 | 5.17E−09 |
| 140 | 7.63E+05 | 5.41E−03 | 7.09E−09 |
| 141 | 4.18E+06 | 1.87E−02 | 4.47E−09 |
| 125 | 3.23E+06 | 1.54E−02 | 4.76E−09 |
| 130 | 1.08E+06 | 5.09E−03 | 4.71E−09 |
| 131 | 9.96E+05 | 4.81E−03 | 4.83E−09 |
| 176 | 9.94E+05 | 2.78E−03 | 2.80E−09 |
| 59 | 9.45E+05 | 1.36E−03 | 1.44E−09 |
| 188 | 7.32E+05 | 1.21E−03 | 1.65E−09 |
| 73 | 7.18E+06 | 5.46E−03 | 7.60E−10 |
| 71 | 4.63E+06 | 1.07E−02 | 2.31E−09 |
| 67 | 3.05E+06 | 1.02E−02 | 3.34E−09 |
| 80 | 3.89E+06 | 2.48E−03 | 6.38E−10 |
| 139 | 9.87E+05 | 9.53E−04 | 9.66E−10 |
| 185 | 5.32E+06 | 9.11E−04 | 1.71E−10 |
| 57 | 6.12E+06 | 3.90E−03 | 6.37E−10 |
| 60 | 1.44E+06 | 1.66E−03 | 1.15E−09 |
| 126 | 1.29E+06 | 1.53E−03 | 1.19E−09 |
| 129 | 9.94E+06 | 5.27E−03 | 5.30E−10 |
| 175 | 4.53E+06 | 6.31E−03 | 1.39E−09 |

ND, not determined.

Experiment 3: The amino acid sequence of recombinant MBP-mutCALR Type 2 fusion protein used in this Experiment 3 is:

(SEQ ID NO: 331)
HHHHHHMKIEEGKLVIWINGDKGYNGLAEVGKKFEKDTGIKVTVEHPDKLEEKFPQVAATGDGP
DIIFWAHDRFGGYAQSGLLAEITPDKAFQDKLYPFTWDAVRYNGKLIAYPIAVEALSLIYNKDL
LPNPPKTWEEIPALDKELKAKGKSALMENLQEPYFTWPLIAADGGYAFKYENGKYDIKDVGVDN
AGAKAGLTFLVDLIKNKHMNADTDYSIAEAAFNKGETAMTINGPWAWSNIDTSKVNYGVTVLPT
FKGQPSKPFVGVLSAGINAASPNKELAKEFLENYLLTDEGLEAVNKDKPLGAVALKSYEEELVK
DPRIAATMENAQKGEIMPNIPQMSAFWYAVRTAVINAASGRQTVDEALKDAQTRSSSLEVLFQG
PGLNDIFEAQKIEWHEENLYFQGEPAVYFKEQFLDGDGWTSRWIESKHKSDFGKFVLSSGKFYG
DEEKDKGLQTSQDARFYALSASFEPFSNKGQTLVVQFTVKHEQNIDCGGGYVKLFPNSLDQTDM
HGDSEYNIMFGPDICGPGTKKVHVIFNYKGKNVLINKDIRCKDDEFTHLYTLIVRPDNTYEVKI
DNSQVESGSLEDDWDFLPPKKIKDPDASKPEDWDERAKIDDPTDSKPEDWDKPEHIPDPDAKKP
EDWDEEMDGEWEPPVIQNPEYKGEWKPRQIDNPDYKGTWIHPEIDNPEYSPDPSIYAYDNFGVL
GLDLWQVKSGTIFDNFLITNDEAYAEEFGNETWGVTKAAEKQMKDKQDEEQRLKEEEEDKKRKE
EEEAEDNCTRRMMRTKMRMRRMRRTRRKMRRKMSPARPRTSCREACLQGWTEA

Recombinant MBP-mutCALR Type 2 fusion protein was custom made at Cepter (cepterbiopartners.com). Data for the interaction of antibodies with recombinant MBP-mutCALR Type 2 fusion protein were collected as described in Experiment 2. The chip used was a Biacore Series S Sensor Chip CM4 with anti-huFc immobilized onto the chip surface. Recombinant MBP-mutCALR Type 2 fusion protein was prepared at nominal concentrations of 1.1, 3.3, 10, 30, and 90 nM. Typical capture levels were in the range of 16-37RU. The results are shown in Table 8.

TABLE 8
Binding affinities and kinetic association and dissociation
rate constants to mutCALR for the indicated antibodies.

| Clone No. | kₐ (1/Ms) | k_{d} (1/s) | KD (M) |
|---|---|---|---|
| 162 | 6.07E+05 | 6.82E−03 | 1.12E−08 |
| 74 | 8.54E+05 | 5.79E−03 | 6.78E−09 |
| 65 | 6.68E+05 | 4.24E−03 | 6.35E−09 |

### Example 3: Binding of Anti-mutCALR Antibodies to Cell Surface MPL and mutCALR

To assess binding of CALR antibodies to cell surface CALR, parental BaF3 (DSMZ) cells or BaF3 cells stably expressing only human MPL or both human MPL (Uniprot Number: P40238-1) and human mutCALR Type 1 and Type 2 were used. On the day of the assay, cells were washed and resuspended in assay buffer. Approximately 200,000 cells/well were added to 96-well plates and stained with the indicated concentration of antibodies for 30 minutes on ice. Cells were then washed and stained with goat anti-human secondary conjugated to R-phycoerythrin (R-PE) (Jackson Immuno Research Laboratories) for 30 minutes on ice. The cells were then washed and analyzed by flow cytometry. Geometric Mean Fluorescence Intensity (GMFI) of cell binding was graphed and EC50, hill slope, and area under the curve (AUC) were determined after four parameter curve fitting using GraphPad Prism Software (version 7.04).

TABLE 9
EC50 values determined by in vitro binding
studies using MPL-mutCALR cells.

| | MPL-mutCALR | MPL-mutCALR | |
|---|---|---|---|
| Clone No. | EC50 (μg/ml) | Hill Slope | MPL-mutCALR AUC |
| 38 | 1.44 | 0.45 | 36975 |
| 1 | 0.01 | 0.47 | 52843 |
| 2 | 0.01 | 0.38 | 60393 |
| 4 | ND | ND | 74025 |
| 5 | 0.09 | 0.31 | 57069 |
| 31 | 0.01 | 0.85 | 45746 |
| 32 | 0.01 | 0.57 | 52533 |
| 6 | 0.30 | 0.22 | 58250 |
| 53 | 0.02 | 0.68 | 64567 |
| 33 | 0.15 | 0.89 | 38688 |
| 39 | ND | ND | 51467 |
| 52 | ND | ND | 65829 |
| 7 | 0.19 | 0.24 | 66803 |
| 8 | 4.12 | 0.27 | 67416 |
| 40 | 0.07 | 0.50 | 52855 |
| 9 | ND | ND | 75254 |
| 10 | ND | ND | 72082 |
| 11 | 0.05 | 0.31 | 69724 |
| 34 | 0.02 | 0.53 | 71898 |
| 12 | 0.02 | 0.46 | 61044 |
| 13 | 0.04 | 0.52 | 57748 |
| 35 | 0.02 | 0.43 | 62105 |
| 36 | 0.01 | 0.67 | 73813 |
| 37 | 9.64 | 0.18 | 80925 |
| 14 | ND | ND | ND |
| 41 | ND | 0.17 | 60610 |
| 15 | ND | ND | 87585 |
| 16 | 0.04 | 0.35 | 63157 |
| 17 | ND | ND | 72716 |
| 18 | ND | ND | 91880 |
| 3 | ND | ND | 101685 |
| 19 | ND | ND | 91584 |
| 20 | ND | ND | 92745 |
| 21 | 0.70 | 0.22 | 70095 |
| 22 | ND | ND | 124443 |
| 23 | ND | ND | 65885 |
| 42 | ND | ND | 61297 |
| 24 | 29.30 | 0.41 | 67826 |
| 25 | 77.33 | 0.36 | 65151 |
| 26 | 0.42 | 0.46 | 41515 |
| 27 | ND | 0.28 | 55234 |
| 28 | 4.15 | 0.37 | 60413 |
| 29 | 3.19 | 0.39 | 77885 |
| 30 | ND | 0.28 | 63793 |
| 43 | Low Binding | Low Binding | 27869 |
| 44 | No Binding | No Binding | 22711 |
| 45 | Low Binding | Low Binding | 29834 |
| 46 | No binding | No Binding | 28469 |
| 47 | No Binding | No Binding | 26698 |
| 48 | Low Binding | Low Binding | 30304 |
| 49 | 0.89 | 1.72 | 36401 |
| 50 | 0.80 | 1.73 | 36656 |
| 51 | 1.15 | 1.31 | 37302 |

ND, not determined.

TABLE 10
EC50 values for variant clones determined by in
vitro binding studies using MPL-mutCALR cells.

| | MPL-mutCALR | MPL-mutCALR | |
|---|---|---|---|
| Clone No. | EC50 (μg/mL) | Hill Slope | MPL-mutCALR AUC |
| 64 | 0.0072 | 0.73 | 73166 |
| 70 | 0.0019 | 0.50 | 84756 |
| 55 | 0.0101 | 0.59 | 73079 |
| 75 | ND | ND | 84873 |
| 68 | 0.0033 | 0.49 | 68742 |
| 74 | 0.0022 | 0.60 | 81764 |
| 69 | 0.0021 | 0.49 | 71783 |
| 79 | 0.0033 | 0.28 | 71948 |
| 58 | 0.0086 | 0.70 | 69796 |
| 84 | 0.0029 | 0.34 | 82395 |
| 170 | 0.0232 | 0.56 | 76385 |
| 63 | 0.0055 | 0.89 | 69069 |
| 65 | 0.0059 | 0.48 | 81192 |
| 124 | ND | ND | 93695 |
| 179 | 0.0058 | 0.65 | 60232 |
| 138 | 0.0026 | 0.44 | 85777 |
| 134 | ND | ND | 96714 |
| 180 | 0.0109 | 0.24 | 74272 |
| 189 | 0.0100 | 0.60 | 57238 |
| 174 | 0.0040 | 0.64 | 63472 |
| 128 | ND | ND | 88882 |
| 184 | 0.0063 | 0.64 | 61557 |
| 178 | 0.0041 | 0.89 | 63621 |
| 173 | 0.0043 | 0.91 | 67293 |
| 61 | 0.0063 | 0.68 | 62366 |
| 183 | 0.0050 | 0.87 | 70657 |
| 56 | 0.0069 | 0.74 | 64360 |
| 66 | 0.0058 | 0.76 | 64963 |
| 83 | 0.0034 | 0.54 | 62800 |
| 133 | 0.0017 | 0.39 | 85219 |
| 143 | 0.0079 | 1.79 | 69938 |
| 142 | 0.0023 | 0.80 | 71120 |
| 78 | 0.0055 | 0.50 | 64267 |
| 187 | 0.0035 | 0.71 | 49545 |
| 72 | 0.0033 | 0.88 | 58004 |
| 62 | 0.0054 | 1.00 | 58771 |
| 137 | 0.0017 | 0.56 | 69749 |
| 172 | 0.0124 | 1.33 | 53603 |
| 127 | 0.0014 | 0.73 | 73519 |
| 132 | 0.0004 | 0.45 | 73424 |
| 182 | 0.0052 | 0.62 | 52069 |
| 177 | 0.0034 | 0.70 | 52926 |
| 186 | 0.0039 | 0.73 | 52984 |
| 171 | 0.0217 | 0.83 | 50342 |
| 181 | 0.0035 | 0.75 | 55586 |
| 77 | 0.0067 | 0.74 | 54229 |
| 76 | 0.0082 | 0.95 | 55058 |
| 81 | 0.0082 | 0.78 | 52888 |
| 82 | 0.0048 | 0.67 | 50241 |
| 136 | 0.0020 | 0.66 | 64988 |
| 135 | 0.0032 | 0.85 | 56343 |
| 140 | 0.0043 | 0.86 | 54721 |
| 141 | 0.0063 | 0.69 | 60764 |
| 125 | 0.0021 | 0.76 | 62652 |
| 130 | 0.0017 | 0.77 | 58276 |
| 131 | 0.0011 | 0.47 | 58328 |
| 176 | 0.0031 | 0.53 | 48363 |
| 59 | 0.0077 | 0.54 | 75686 |
| 188 | 0.0044 | 0.82 | 67953 |
| 73 | 0.0020 | 0.50 | 67561 |
| 71 | 0.0097 | 0.92 | 55899 |
| 67 | 0.0081 | 0.72 | 63887 |

ND, not determined.

### Example 4: Anti-mutCALR Antibody Mediated Inhibition of pSTAT5

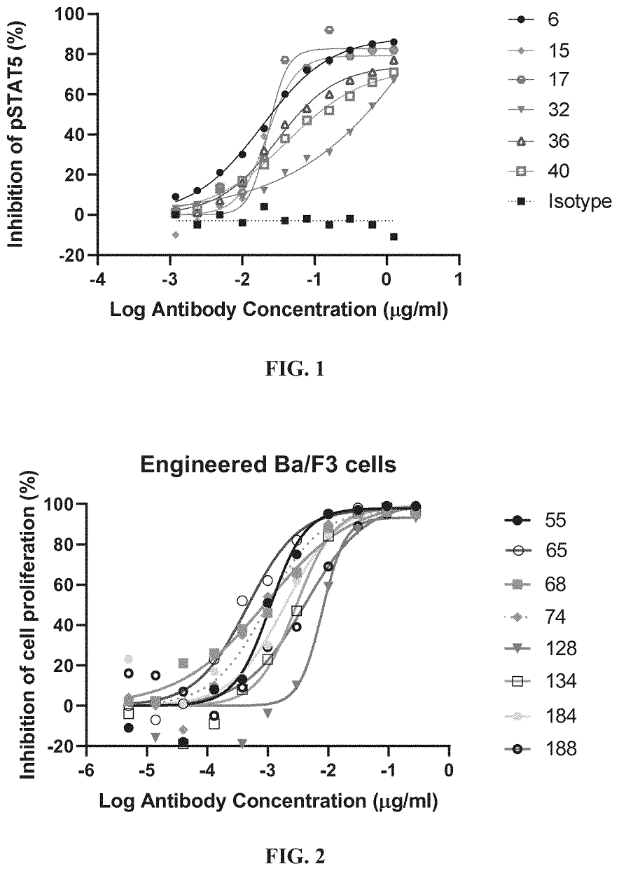

To test the ability of anti-mutCALR antibodies to inhibit phosphorylation of STAT5, Ba/F3 cells (DSMZ) expressing MPL and Type 1 mutCALR variants were generated by nucleofection (Amaxa Cell Line Nucleofection Kit V, Lonza, Basel, Switzerland) and cultured in RPMI 1640+10% FBS+selection antibiotics. Prior (24 hours) to pSTAT5 assessment, cells were cultured in selection-free media and then plated at 200,000 cells per well (96 well plate) in RPMI 1640, 10% FBS. Antibodies were added to the cells and incubated for 2 hours followed by cell lysing and quantification of pSTAT-5 levels by MSD (Phospho-STAT5a,b Whole Cell Lysate Kit, MSD, Kenilworth, N.J.). Anti-mutCALR antibodies inhibit phosphorylation of STAT5 in a dose-dependent manner (FIG. 1).

TABLE 11
IC₅₀ values for anti-mutCALR antibody
mediated inhibition of pSTAT5.

| Clone No. | IC50 (μg/mL) |
|---|---|
| 54 | 12.7 |
| 38 | 3.55 |
| 1 | 0.47 |
| 2 | 0.12 |
| 4 | 0.04 |
| 5 | 0.03 |
| 31 | 26.8 |
| 32 | 0.47 |
| 6 | 0.03 |
| 53 | 0.402 |
| 33 | 80.4 |
| 39 | 3.97 |
| 52 | 0.346 |
| 7 | 0.049 |
| 8 | 0.043 |
| 40 | 0.742 |
| 9 | 0.056 |
| 10 | 0.055 |
| 11 | 0.052 |
| 34 | 0.072 |
| 12 | 0.061 |
| 13 | 0.091 |
| 35 | 0.444 |
| 36 | 0.092 |
| 37 | 0.039 |
| 14 | 0.06 |
| 41 | 0.364 |
| 15 | 0.041 |
| 16 | 0.028 |
| 17 | 0.031 |
| 18 | 0.036 |
| 3 | 0.021 |
| 19 | 0.062 |
| 20 | 0.018 |
| 21 | 0.032 |
| 22 | 0.035 |
| 23 | 0.03 |
| 42 | 0.196 |
| 24 | 0.031 |
| 25 | 0.024 |
| 26 | 0.125 |
| 27 | 0.037 |
| 28 | 0.193 |
| 29 | 0.037 |
| 30 | 0.054 |
| 43 | 6.9 |
| 44 | 7 |
| 45 | 13 |
| 46 | 3.9 |
| 47 | 10 |
| 48 | 2.6 |
| 49 | 9.3 |
| 50 | 28 |
| 51 | 14 |

### Example 5: Anti-mutCALR Antibody Mediated Inhibition of Cell Proliferation

To test the ability of anti-mutCALR antibodies to inhibit cell proliferation, engineered Ba/F3 cells transfected with MPL and mutCALR Type 1 were plated at 5,000 cells per well in RPMI 1640+2% FBS, antibodies added, incubated for 72 hours, and followed by assessment of cell viability using the CellTiter-Glo Luminescent Cell Viability Assay (Promega, Madison, Wis.) and Top Count (Perkin Elmer, Boston, Mass.) or Pherastar (BMG Labtech, Ortenberg, Germany) for luminescence quantification. Anti-mutCALR antibodies inhibit mutCALR-induced oncogenic cell proliferation in a dose-dependent manner in both the Ba/F3 engineered cells (FIG. 2).

TABLE 12
IC₅₀ values for anti-mutCALR antibody mediated inhibition
of cell proliferation (Ba/F3 engineered cells) obtained
after high-throughput primary screening.

| Clone No. | IC50 (μg/ml) |
|---|---|
| 54 | ND |
| 38 | 1.9070 |
| 1 | 0.8345 |
| 2 | 0.0714 |
| 4 | 0.0022 |
| 5 | 0.0049 |
| 31 | 13.0100 |
| 32 | 0.2252 |
| 6 | 0.0032 |
| 53 | 0.3992 |
| 33 | 16.8800 |
| 39 | 0.1042 |
| 52 | 0.0337 |
| 7 | 0.0113 |
| 8 | 0.0067 |
| 40 | 0.0384 |
| 9 | 0.0046 |
| 10 | 0.0072 |
| 11 | 0.0104 |
| 34 | 0.0593 |
| 12 | 0.0043 |
| 13 | 0.0303 |
| 35 | 0.0880 |
| 36 | 0.0905 |
| 37 | ND |
| 14 | ND |
| 41 | 0.3420 |
| 15 | 0.0037 |
| 16 | 0.0315 |
| 17 | 0.0129 |
| 18 | 0.0090 |
| 3 | 0.0095 |
| 19 | 0.0152 |
| 20 | 0.0136 |
| 21 | ~0.009274 |
| 22 | 0.0077 |
| 23 | 0.0065 |
| 42 | ND |
| 24 | 0.0045 |
| 25 | 0.0035 |
| 26 | 0.1094 |
| 27 | 0.0040 |
| 28 | 0.0068 |
| 29 | ~0.002898 |
| 30 | 0.0113 |
| 43 | 1.0420 |
| 44 | 2.8910 |
| 45 | 0.6996 |
| 46 | 0.2779 |
| 47 | 0.5726 |
| 48 | 0.1908 |
| 49 | 0.2133 |
| 50 | 1.2120 |
| 51 | 0.9771 |
| 55 | 0.0011 |
| 68 | 0.0009 |
| 74 | 0.0010 |
| 170 | 0.0019 |
| 65 | 0.0003 |
| 124 | 0.0040 |
| 134 | 0.0036 |
| 128 | 0.0098 |
| 184 | 0.0019 |
| 188 | 0.0046 |

ND, not determined.

### Example 6: Anti-mutCALR Antibody Inhibits Oncogenic Cell Proliferation Induced by Both Type 1 and Type 2 CALR Mutations

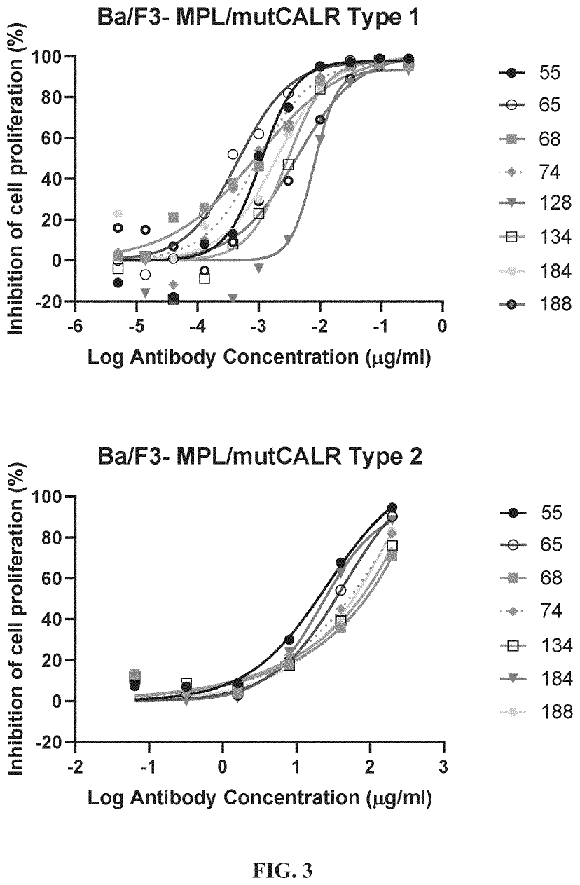

To test the ability of anti-mutCALR antibodies to inhibit oncogenic cell proliferation triggered by the Type 1 and Type 2 CALR mutations, Ba/F3 cells (DSMZ) were engineered to express MPL+mutCALR Type 1 (52 bp deletion, SEQ ID NO:320) or MPL+mutCALR Type 2 (5 bp insertion; SEQ ID NO:321). Cells were plated at 5,000 cells per well in RPMI 1640+2% FBS, antibodies added, incubated for 72 hours, and followed by assessment of cell viability using the CellTiter-Glo Luminescent Cell Viability Assay (Promega, Madison, Wis.) and Top Count (Perkin Elmer, Boston, Mass.) or Pherastar (BMG Labtech, Ortenberg, Germany) for luminescence quantification. Anti-mutant CALR antibodies show potency to inhibit mutCALR-induced oncogenic cell proliferation associated with both Type 1 and Type 2 mutations (FIG. 3).

### Example 7: Anti-mutCALR Antibody Mediated Inhibition of MPL Dimerization

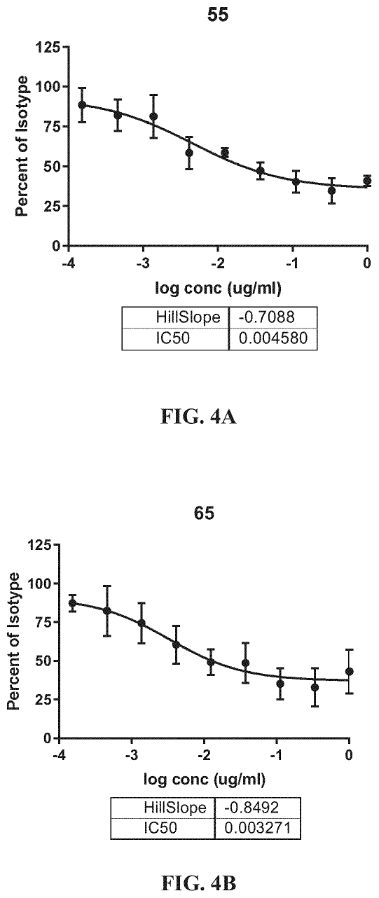

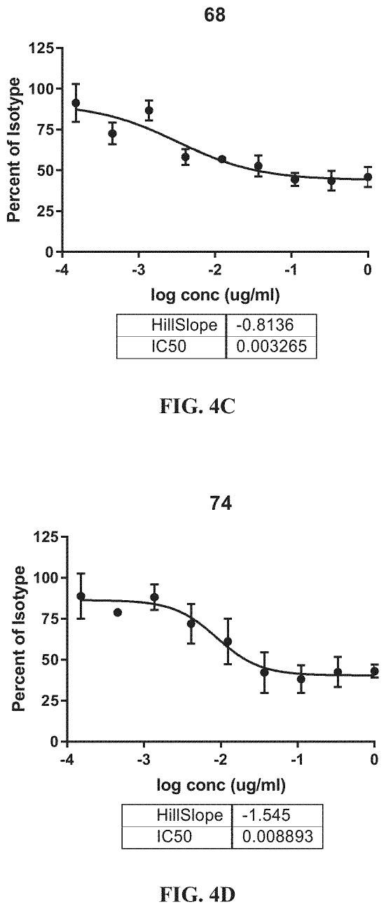

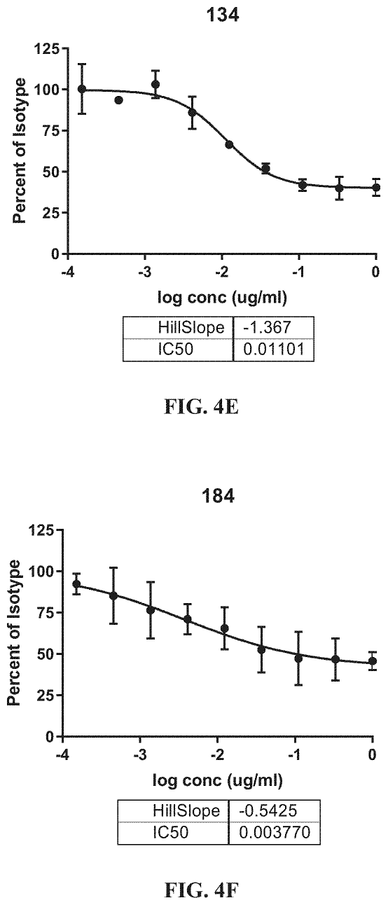

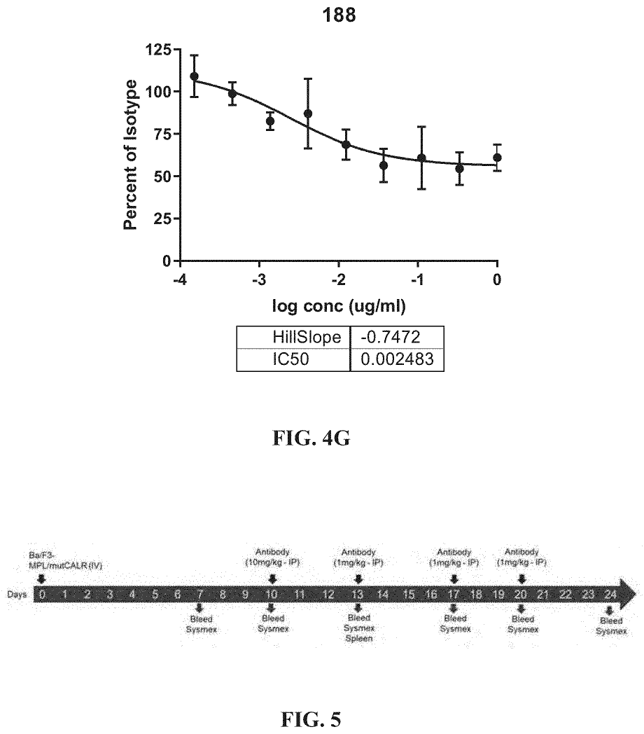

Inhibition of MPL dimerization was tested as a potential mechanism of action of the anti-mutCALR antibodies. HAP1 cells knocked out for human JAK2 (Horizon Discovery, Ltd.) were transiently transfected with vectors encoding the MPL-LgBiT and MPL-smBiT fusion proteins (Promega Corp.). Also included in the transfection were vectors encoding the full-length human JAK2 and the full-length WT or mutant CALR Type 1 protein (full-length cds cloned into the pD2529 vector, ATUM Bio). The cells were transfected in 96 well plates using Trans-IT 2020 reagent (Mirus Bio LLC) with equivalent amounts of each plasmid. Cells were then incubated at 37° C., 5% CO₂. 6 hours post-transfection, antibodies were diluted into growth medium (IMDM, 10% FBS) and added to the cells at the indicated concentrations. The plates were incubated overnight and the growth medium was replaced with 100 μl OPTI-MEM I, no phenol red, containing the same concentrations of antibodies. After 1 hour at 37° C., 5% CO₂, 25 μl NanoGlo Live Cell Reagent (Promega) was added to each well. Plates were returned to the incubator for 30 minutes before luminescence was read on a PHERAstar FSX (BMG Labtech). Data was analyzed using GraphPad PRISM Software (version 7.04) and expressed as percent inhibition compared to isotype control. As shown in FIGS. 4A-4G, anti-mutCALR antibodies (clones 55, 65, 68, 74, 134, 184, and 188) can inhibit MPL dimerization.

The amino acid sequences for the MPL-LgBiT and MPL-smBiT fusion proteins used in this Example are:

MPL-smBIT
(SEQ ID NO: 336)
MPSWALFMVTSCLLLAPQNLAQVSSQDVSLLASDSEPLKCFSRTFEDLTCFWDEEEAAPS
GTYQLLYAYPREKPRACPLSSQSMPHFGTRYVCQFPDQEEVRLFFPLHLWVKNVELNOTR
TORVLFVDSVGLPAPPSIIKAMGGSQPGELQISWEEPAPEISDFLRYELRYGPRDPKNST
GPTVIQLIATETCCPALQRPHSASALDOSPCAQPTMPWQDGPKQTSPSREASALTAEGGS
CLISGLQPGNSYWLQLRSEPDGISLGGSWGSWSLPVTVDLPGDAVALGLQCFTLDLKNVT
COWQQQDHASSQGFFYHSRARCCPRDRYPIWENCEEEEKTNPGLQTPQFSRCHFKSRNDS
ITHILVEVTTAPGTVHSYLGSPFWIHQAVRLPTPNLHWREISSGHLELEWQHPSSWAAQE
TCYQLRYTGEGHQDWKVLEPPLGARGGTLELRPRSRYRLQLRARLNGPTYQGPWSSWSDP
TRVETATETAWISLVTALHLVLGLSAVLGLLLLRWQFPAHYRRLRHALWPSLPDLHRVLG
QYLRDTAALSPPKATVSDTCEEVEPSLLEILPKSSERTPLPLCSSQAQMDYRRLQPSCLG
TMPLSVCPPMAESGSCCTTHIANHSYLPLSYWQQPVSQGSSGGGGSGGGGSSGVTGYRLF
EEIL
MPL-LgBiT
(SEQ ID NO: 337)
MPSWALFMVTSCLLLAPQNLAQVSSQDVSLLASDSEPLKCFSRTFEDLTCFWDEEEAAPS
GTYQLLYAYPREKPRACPLSSQSMPHFGTRYVCQFPDQEEVRLFFPLHLWVKNVELNQTR
TQRVLFVDSVGLPAPPSIIKAMGGSQPGELQISWEEPAPEISDFLRYELRYGPRDPKNST
GPTVIQLIATETCCPALQRPHSASALDOSPCAQPTMPWQDGPKQTSPSREASALTAEGGS
CLISGLQPGNSYWLQLRSEPDGISLGGSWGSWSLPVTVDLPGDAVALGLQCFTLDLKNVT
COWQQQDHASSQGFFYHSRARCCPRDRYPIWENCEEEEKTNPGLQTPQFSRCHFKSRNDS
IIHILVEVTTAPGTVHSYLGSPEWIHQAVRLPTPNLHWREISSGHLELEWQHPSSWAAQE
TCYQLRYTGEGHQDWKVLEPPLGARGGTLELRPRSRYRLQLRARLNGPTYQGPWSSWSDP
TRVETATETAWISLVTALHLVLGLSAVLGLLLLRWQFPAHYRRLRHALWPSLPDLHRVLG
QYLRDTAALSPPKATVSDTCEEVEPSLLEILPKSSERTPLPLCSSQAQMDYRRLQPSCLG
TMPLSVCPPMAESGSCCTTHIANHSYLPLSYWQQPVSQGSSGGGGSGGGGSSGVFTLEDE
VGDWEQTAAYNLDQVLEQGGVSSLLQNLAVSVTPIQRIVRSGENALKIDIHVIIPYEGLS
ADQMAQIEEVFKVVYPVDDHHFKVILPYGTLVIDGVTPNMLNYFGRPYEGIAVFDGKKIT
VTGTLWNGNKIIDERLITPDGSMLERVTINS

### Example 8: Effect of Anti-mutCALR Antibody in Mice Injected with Tumor Cells Expressing MPL-mutCALR

To test the functional ability of anti-mutCALR antibodies in vivo, antibodies were evaluated in a mouse model of tumor growth. Engineered Ba/F3 tumor cells expressing MPL/mutCALR Type 1 were inoculated intravenously in NSG immunodeficient mice (NOD-scid IL2Rgamma^{null}, The Jackson Laboratories, Bar Harbor, Me.). Tumors were allowed to grow for 10 days when mice were randomized into antibody or isotype control treatment groups. Different doses of antibodies were administered intraperitoneally and tumor growth was followed over time by assessing the presence and number of tumor cells in the blood (Sysmex, Kobe, Japan). Further, full hematology and tumor infiltration in the spleen and bone marrow were evaluated for assessment of antibody potencies. A representative in vivo study is shown in FIG. 5.

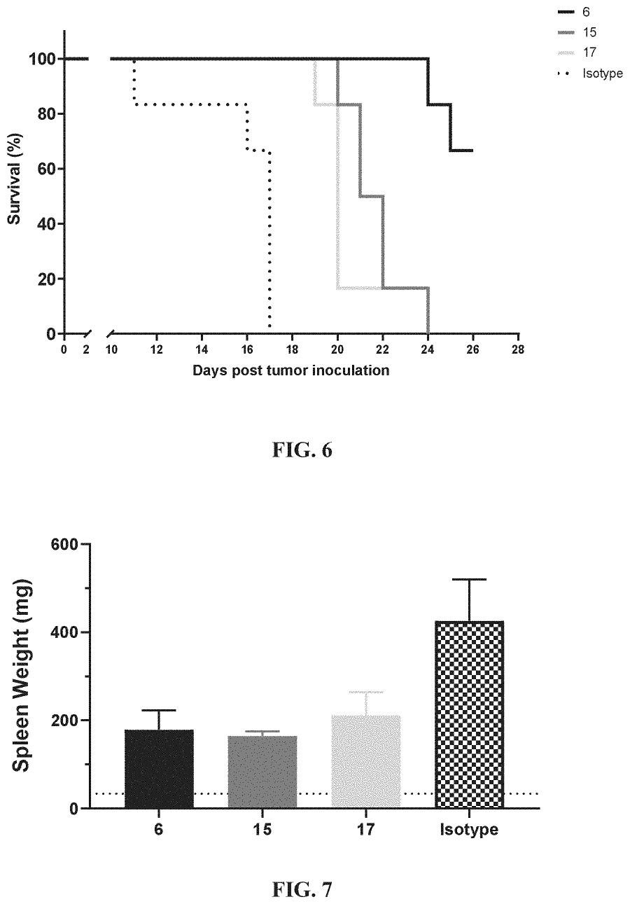

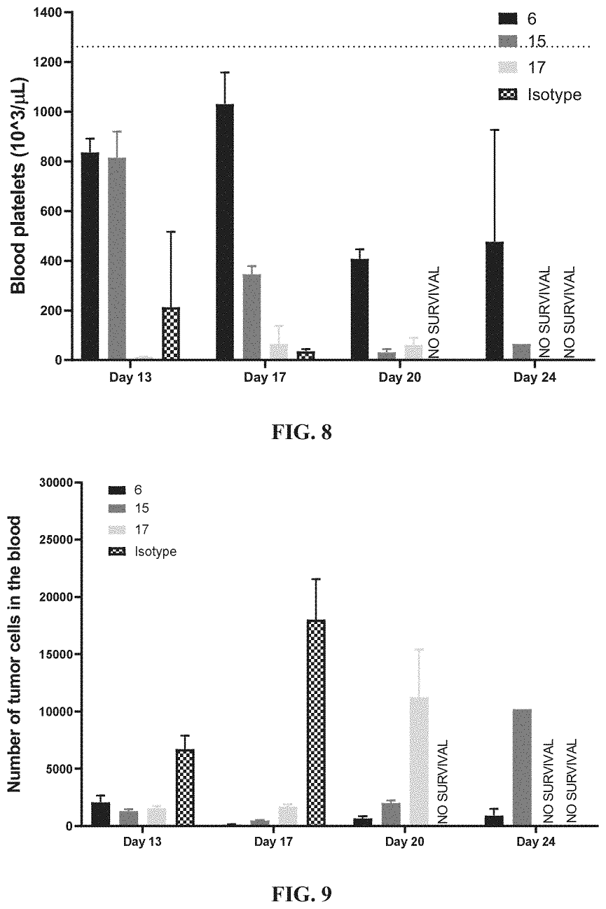

The anti-mutCALR antibodies prolonged mouse survival (FIG. 6) and prevented splenomegaly (FIG. 7), thrombocytopenia (FIG. 8), and proliferation of tumor cells in the blood (FIG. 9).

### Example 9: Anti-mutCALR Antibodies Potentiate the Therapeutic Response of Ruxolitinib in the Inhibition of Oncogenic Cell Proliferation Triggered by mutCALR Type 1 or Type 2

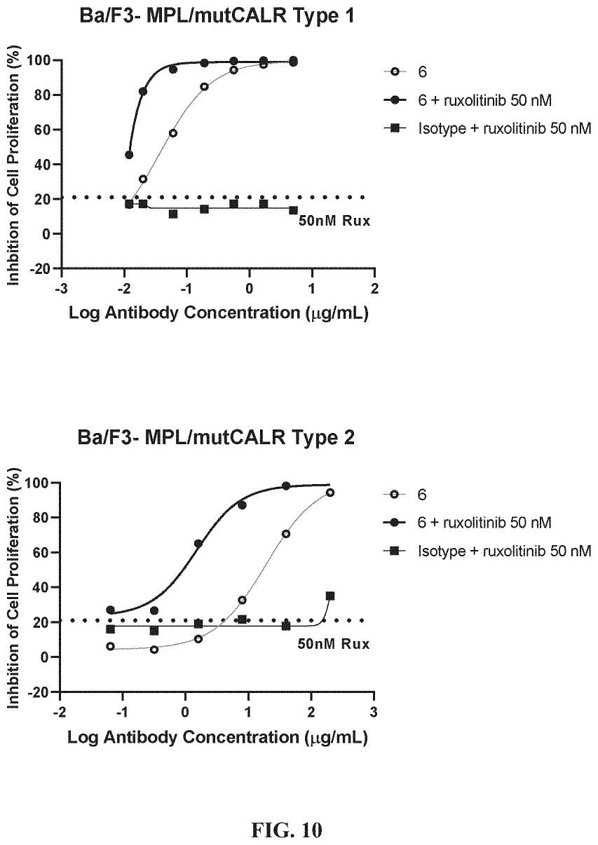

To test the ability of anti-mutCALR antibodies to potentiate the therapeutic response of the JAK1/2 inhibitor ruxolitinib, Ba/F3 cells were engineered to express MPL+mutCALR Type 1 (52 bp deletion) or MPL+mutCALR Type 2 (5 bp insertion) as described above. Cells were plated at 5,000 cells per well in RPMI 1640+2% FBS, treated with 50 nM of ruxolitinib and/or anti-mutCALR antibodies. Cells were incubated for 72 hours followed by assessment of cell viability using the CellTiter-Glo Luminescent Cell Viability Assay (Promega, Madison, Wis.) and Top Count (Perkin Elmer, Boston, Mass.) or Pherastar (BMG Labtech, Ortenberg, Germany) for luminescence quantification. Ruxolitinib used at a concentration of 50 nM inhibited the level of oncogenic cell proliferation by approximately 20% (dotted line). Anti-mutant CALR antibody clone 6 potentiated the ability of ruxolitinib to inhibit cell proliferation in cells carrying the CALR Type 1 (FIG. 10; top) or Type 2 (FIG. 10; bottom) mutation. IC₅₀ values for various clones are shown in Table 13.

TABLE 13
IC₅₀ values for anti-mutCALR antibody or antibody plus
ruxolitinib mediated inhibition of cell proliferation
(Ba/F3-TPOR/mutCALR type 1 engineered cells).

Log IC50 (μg/mL)

| Clone No. | Antibody alone | Antibody + ruxolitinib |
|---|---|---|
| 6 | −1.354 | −4.270* |
| 15 | −1.558* | −6.186* |
| 17 | −1.585* | −4.962* |
| 32 | 0.180* | −0.586* |
| 36 | −8.511 | −3.880* |
| 38 | −0.035 | 0.542 |
| 40 | −36.7 | −1.595* |
| 51 | −1.585* | −4.962* |
| 52 | 6.906 | −0.709 |
| 53 | −0.173 | −0.424 |
| 74 | −0.438 | −1.458 |

*extrapolated from fitted curve

### Example 10: Anti-mutCALR Antibodies Compete with MPL In Vitro for the Binding to mutCALR

The amino acid sequence of recombinant His tag_hMPL used in this example is:

(SEQ ID NO: 338)
SQDVSLLASDSEPLKCFSRTFEDLTCFWDEEEAAPSGTYQLLYAYPREKPRACPLSSQSMPHFG
TRYVCQFPDQEEVRLFFPLHLWVKNVFLNQTRTQRVLFVDSVGLPAPPSIIKAMGGSQPGELQI
SWEEPAPEISDFLRYELRYGPRDPKNSTGPTVIQLIATETCCPALQRPHSASALDOSPCAQPTM
PWQDGPKQTSPSREASALTAEGGSCLISGLQPGNSYWLQLRSEPDGISLGGSWGSWSLPVTVDL
PGDAVALGLQCFTLDLKNVTCQWQQQDHASSQGFFYHSRARCCPRDRYPIWENCEEEEKTNPGL
QTPQFSRCHFKSRNDSIIHILVEVTTAPGTVHSYLGSPEWIHQAVRLPTPNLHWREISSGHLEL
EWQHPSSWAAQETCYQLRYTGEGHQDWKVLEPPLGARGGTLELRPRSRYRLQLRARLNGPTYQG
PWSSWSDPTRVETATETAWHHHHHH

The amino acid sequence of recombinant Flag tag_GFP_tev_hmutCALR Type 1 used in this example is:

(SEQ ID NO: 326)
MDYKDDDDKGGVSKGEELFTGVVPILVELDGDVNGHKFSVSGEGEGDATYGKLTLKFICTTGKL
PVPWPTLVTTLTYGVQCFSRYPDHMKQHDFFKSAMPEGYVQERTIFFKDDGNYKTRAEVKFEGD
TLVNRIELKGIDFKEDGNILGHKLEYNYNSHNVYIMADKQKNGIKVNFKIRHNIEDGSVQLADH
YQQNTPIGDGPVLLPDNHYLSTQSKLSKDPNEKRDHMVLLEFVTAAGITLGMDELYKENLYFQG
NKGSIEGREPAVYFKEQFLDGDGWTSRWIESKHKSDFGKFVLSSGKFYGDEEKDKGLQTSQDAR
FYALSASFEPFSNKGQTLVVQFTVKHEQNIDCGGGYVKLFPNSLDQTDMHGDSEYNIMFGPDIC
GPGTKKVHVIFNYKGKNVLINKDIRCKDDEFTHLYTLIVRPDNTYEVKIDNSQVESGSLEDDWD
FLPPKKIKDPDASKPEDWDERAKIDDPTDSKPEDWDKPEHIPDPDAKKPEDWDEEMDGEWEPPV
IQNPEYKGEWKPRQIDNPDYKGTWIHPEIDNPEYSPDPSIYAYDNFGVLGLDLWQVKSGTIFDN
FLITNDEAYAEEFGQETWGVTKAAEKQMKDKQDEEQRTRRMMRTKMRMRRMRRTRRKMRRKMSP
ARPRTSCREACLQGWTEALEVLFQGPGSGAKDEL

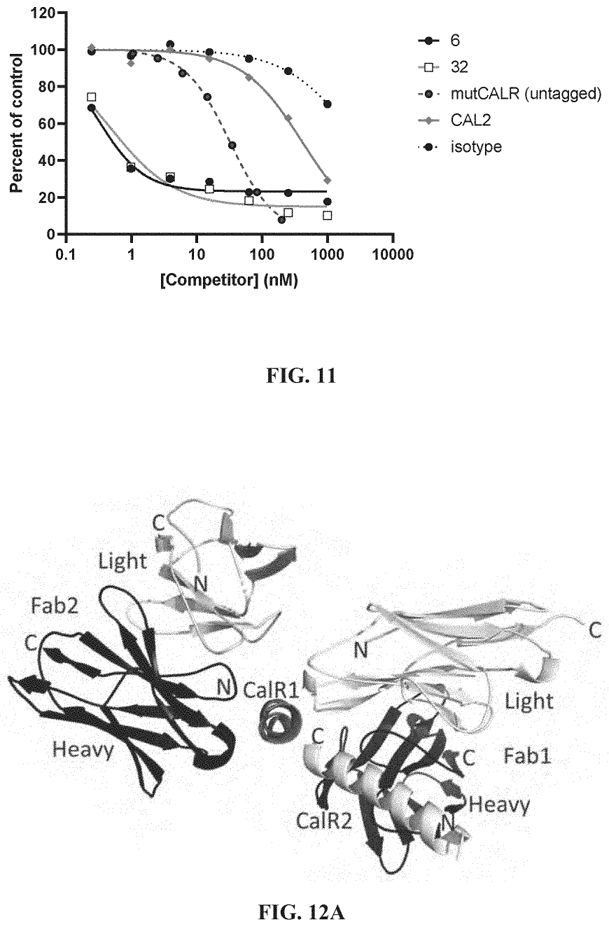

Recombinant His tag_hMPL and recombinant Flag tag_GFP_tev_hmutCALR Type 1 proteins (80 nM MPL+5 nM mutCALR) were incubated in assay buffer (HEPES, pH 7.5 50 nM; Prionex 0.05%; NaCl 100 nM; Pluoronic F-127 0.01%; CaCl₂ 1 mM; MgCl2 1 mM; DTT) for 1 h at room temperature to allow for the formation of the mutCALR/MPL complex. In a 384-well plate, 5 μl of anti-mutCALR antibodies 6 and 32, non-functional CAL2 antibody (Dianova), or untagged mutCALR (competitor) was incubated with 5 μl of the mutCALR/MPL protein mix for 1 h at room temperature. HTRF detection solution (2 nM anti-FLAG-Europium, and 100 nM anti-6×His-SureLight® APC) was then added and the plate was read on BMG PHERAstar FSX using kinetic HTRF protocol, obtaining readings every 15 minutes for 120 minutes total. Data showed that binding equilibrium was reached by 90 min and the 90 min time point HTRF Ratio values were used for analysis. HTRF Ratio from each test well containing FLAG-GFP-tev-mutCALR+MPL-6×His solution was background-subtracted using control well (MPL_His tag not included). Percent-of-control values were obtained by dividing the background-subtracted HTRF Ratios for each test well by the buffer control well in the same plate row. The data shows that untagged mutCALR competes with FLAG tag_mutCALR for binding to MPL (positive control) while the isotype does not interfere with the mutCALR/MPL interaction (negative control). Whereas the non-functional CAL2 antibody showed some signal inhibition at high concentration (>100 nM), the anti-mutCALR antibodies 6 and 32 fully inhibited the interaction of MPL and mutCALR, indicating that the anti-mutCALR antibodies compete with MPL for binding to mutCALR (FIG. 11).

### Example 11: Structural Determination of Fab Fragment from Anti-mutCALR Antibody Bound to mutCALR 21-Mer Peptide

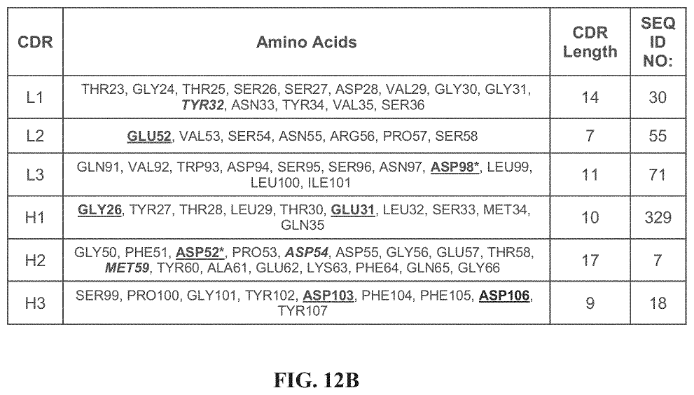

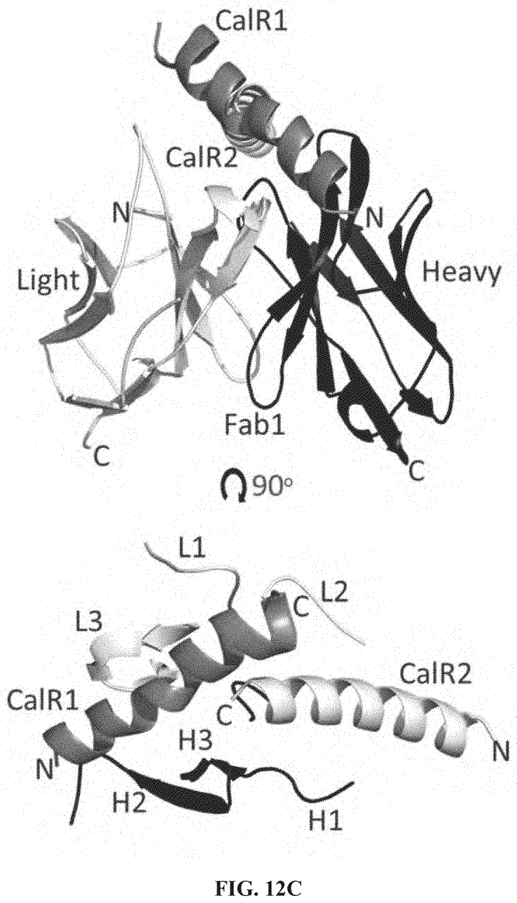

For Examples 11 and 12, and FIGS. 12, 13, 14, 15, 16 and 17, the CCG numbering scheme from MOE (Molecular Operating Environment, 2019.01; Chemical Computing Group ULC, 1010 Sherbooke St. West, Suite #910, Montreal, QC, Canada, H3A 2R7, 2021) was used for CDR definitions and antibody sequence numbering.

The Fab portion of anti-mutCALR antibody (clone 55; also referred to as antibody 55) was expressed and purified as follows:

The Fab portion of antibody 55 was expressed in Expi293F cells (Thermo Fisher, cat. #A14635) by transient transfection for 5 days. The Fab was purified from clarified supernatants by binding to CaptureSelect CHI-XL Affinity Matrix (ThermoFisher), washing with PBS buffer, and eluting with 50 mM sodium acetate, pH 4.0. After elution, Fab was buffer exchanged to PBS and concentrated using Ultra-15 centrifugal filter unit, 10 kDa MWCO (Amicon) and then stored at −80° C.

Sequences:
Fab Heavy Chain:
(SEQ ID NO: 327)
EVOLVQSGAEVKKPGASVKVSCKVSGYTLTELSMQWVRQAPGKGLEWMGGFDPDDGETMYAEKF
QGRLTVTEDTSTDTVYMELRSLTSEDTALYFCATSPGYDFFDYWGQGTLVTVSSASTKGPSVFP
LAPSSKSTSGGTAALGCLVKDYFPEPVTVSWNSGALTSGVHTFPAVLQSSGLYSLSSVVTVPSS
SLGTQTYICNVNHKPSNTKVDKRVEPKSCDKTHT
Fab Light Chain:
(SEQ ID NO: 213)
QSALTQPASVSGSPGQSITISCTGTSSDVGGYNYVSWYQQHPGKAPKLMIYEVSNRPSGVSNRF
SGSNSGNTATLTINRVEAGDEADYYCQVWDSSNDLLIFGGGTKLTVLGQPKAAPSVTLFPPSSE
ELQANKATLVCLISDFYPGAVTVAWKADSSPVKAGVETTTPSKQSNNKYAASSYLSLTPEQWKS
HRSYSCQVTHEGSTVEKTVAPTECS

Crystallization was performed as follows:

Fab: The Fab portion of antibody 55 was concentrated to 10 mg/mL in TBS buffer. Sparse matrix crystallization screens were set up using an NT8 crystallization robot (Formulatrix, Bedford, Mass.). Drops containing 200 nl (Fab fragment)+200 nl (reservoir) were used for the setup and the plates were incubated at 4° C., 13° C. and 20° C. Crystals of the Fab portion of antibody 55 appeared in condition C10 of the JCSG Top96 Screen (0.1 M HEPES pH 7.5, 20% (w/v) PEG 8,000 (Rigaku, Bainbridge Island, Wash.) after five days incubation at 4° C.

Fab-mutCALR Peptide: The Fab portion of antibody 55 was mixed with mutCALR peptide (Acetyl-DEEQRTRRMMRTKMRMRRMRR-NH₂; SEQ ID NO:339) at a ratio of 1:1.5 molar excess, and then concentrated to a final concentration of 35 mg/mL. Sparse matrix crystallization screens were set up using an NT8 crystallization robot (Formulatrix, Bedford, Mass.). Drops containing 200 nl (Fab plus mutCALR peptide)+200 nl (reservoir) were used for the setup and the plates were incubated at 4° C., 13° C. and 20° C. Initial crystal hits grew from condition C7 of the JCSG Top96 Screen (Rigaku, Bainbridge Island, Wash.) after 3 days incubation at 4° C. This initial hit condition was further refined, leading to the mutCALR peptide bound crystals. The final condition for Fab-mutCALR peptide was 0.1 M Bicine pH 7.94, 19% w/v PEG 6000. These crystals grew in approximately five days.

All crystals were flash cooled in liquid nitrogen for X-ray data collection.

Data Collection, Processing and Refinement was performed as follows:

Diffraction data was collected at 100K using synchrotron radiation at the Advanced Photon Source (IMCA-CAT beamline 17-ID). Diffraction data indexing, integration and scaling were performed with the AutoPROC package. Data collection statistics, phasing and refinement are given in Table 14.

For the Fab-Apo data, MoRda (Keegan and Winn. (2007) Acta Cryst. D63, 447-57; Keegan et al. (2018) Acta Cryst. D74, 167-82; and Winn et al. (2011) Acta. Cryst. D67, 235-42) was used to place two copies of a Fab fragment as a template (PDB ID: 5AZE). For the Fab-mutCALR peptide data Phaser was used to place fragments of the Fab-Apo model and subsequent rounds of manual model building were performed resulting in a model containing two Fabs and 3 peptides. For both datasets, subsequent cycles of density modifications, model building and refinement were carried out using Refmac (Murshudov et al. (1997) Acta Cryst. D53, 240-255), Coot (Emsley et al. (2010) Acta Crystallographica Section D—Biological Crystallography, 66, 486-501) and Phenix (Liebschner et al. (2019) Acta Cryst. (2019). D75, 861-77) until the structures converged at reasonable R-work and R-free (Table 14). The final models were analyzed for good stereochemistry, geometry and clash scores using MolProbity (Table 14).

Results:

The structure of Fab-Apo was determined to 2.9 Å and consists of residues Light chains 2-213, and Heavy chains 2-219 (2 Fabs) and has good electron density throughout, with the exception of residues Chain L: Asp28-Tyr34, Chain H: Ser99-Gly101, Lys134-Gly139, Chain M: Gly24-Tyr34, Chain I: Ser133-Gly138 which are not modelled due to weak electron density. The structure was refined to an R-work/R-free of 23% and 30%, respectively, and has good stereochemistry throughout with 4 Ramachandran outliers which is acceptable for this resolution (48^{th} percentile for this residue range, Table 14).

The structure of Fab-mutCALR peptide was determined to 3.2 Å. The two Light chains consist of residues Chain L: 2-215, Chain M:2-216; and two Heavy chains H:2-219 and I:2-185. There are two Fabs and two peptides in the asymmetric unit. The model has good electron density throughout, with the exception of the following residues: Chain H, Ser132-Gly139; Chain M, Thr25-Gly31; and Chain I, Ala130-Ala142, which are not modelled due to weak electron density. The structure was refined to an R-work/R-free of 23% and 32%, respectively, and has good stereochemistry throughout with 7 Ramachandran outliers which is acceptable for this resolution (56th percentile for this residue range, Table 14).

TABLE 14
Data collection statistics, phasing and refinement.

| | | | Fab fragment:CalR | |
|---|---|---|---|---|
| | Fab fragment | | peptide | |
| Resolution range | 54.52-2.9 | (3.004-2.9) | 58.3-3.2 | (3.314-3.2) |
| Space group | P 1 21 1 | | P 21 21 21 | |
| Unit cell | 79.921 51.61 109.702 | | 40.646 121.228 | |
| | 90 96.306 90 | | 199.482 90 90 90 | |
| Total reflections | 38451 | (3220) | 32029 | (2718) |
| Unique reflections | 19565 | (1638) | 16045 | (1359) |
| Multiplicity | 2.0 | (2.0) | 2.0 | (2.0) |
| Completeness (%) | 97.16 | (82.11) | 93.80 | (82.11) |
| Mean I/sigma(I) | 7.97 | (2.29) | 13.31 | (6.61) |
| Wilson B-factor | 36.39 | | 39.75 | |
| R-merge | 0.08796 | (0.3616) | 0.05226 | (0.1321) |
| R-meas | 0.1244 | (0.5113) | 0.0739 | (0.1868) |
| R-pim | 0.08796 | (0.3616) | 0.05226 | (0.1321) |
| CC½ | 0.983 | (0.677) | 0.992 | (0.929) |
| CC* | 0.996 | (0.899) | 0.998 | (0.981) |
| Reflections used | 19559 | (1638) | 16040 | (1359) |
| in refinement | | | | |
| Reflections used | 1652 | (138) | 786 | (61) |
| for R-free | | | | |
| R-work | 0.2301 | (0.2879) | 0.2288 | (0.2838) |
| R-free | 0.3032 | (0.3904) | 0.3174 | (0.3856) |
| CC(work) | 0.905 | (0.757) | 0.899 | (0.803) |
| CC(free) | 0.776 | (0.542) | 0.771 | (0.623) |

| | | |
|---|---|---|
| Number of non- | 5916 | 6101 |
| hydrogen atoms | | |
| macromolecules | 5916 | 6101 |
| Protein residues | 825 | 824 |
| RMS(bonds) | 0.010 | 0.012 |
| RMS(angles) | 1.38 | 1.41 |
| Ramachandran | 95.79 | 91.17 |
| favored (%) | | |
| Ramachandran | 3.72 | 7.96 |
| allowed (%) | | |
| Ramachandran | 0.50 | 0.87 |
| outliers (%) | | |
| Rotamer outliers (%) | 0 | 0 |
| Clashscore | 9.83 | 14.96 |
| Average B-factor | 29.20 | 30.27 |
| macromolecules | 29.20 | 30.27 |

As shown in FIG. 12A, the asymmetric unit of the crystal structure includes one Fab molecule bound to two mutCALR peptides (referred to as CalR1 and CalR2). The structure includes 2 light chains (shown in white), 2 heavy chains (shown in black) and 2 identical peptides representing a portion of the mutant CALR C-terminal domain (CalR1, gray and CalR2, white). The Fab binds to the two mutCALR peptides in two distinct binding confirmations (FIG. 12A). Only Fv regions of the Fab are displayed for clarity.

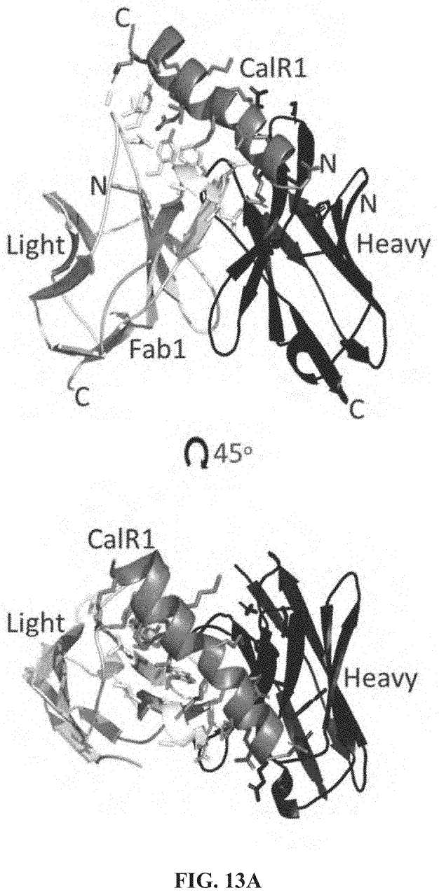

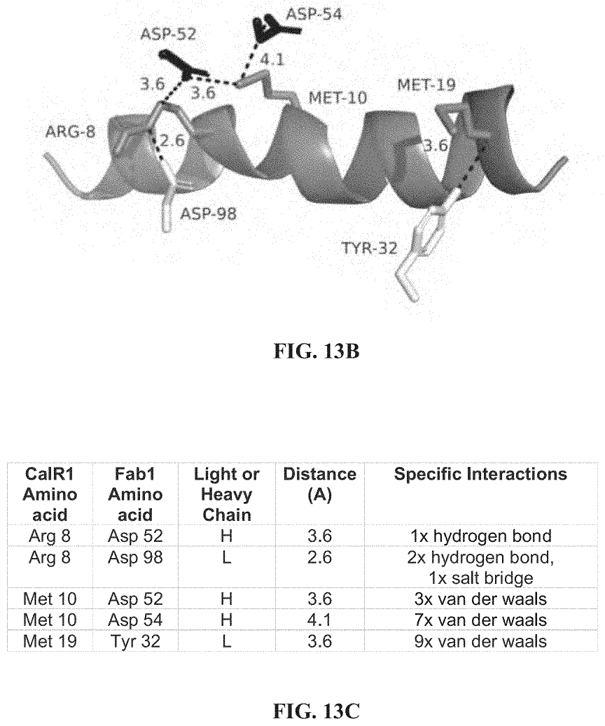

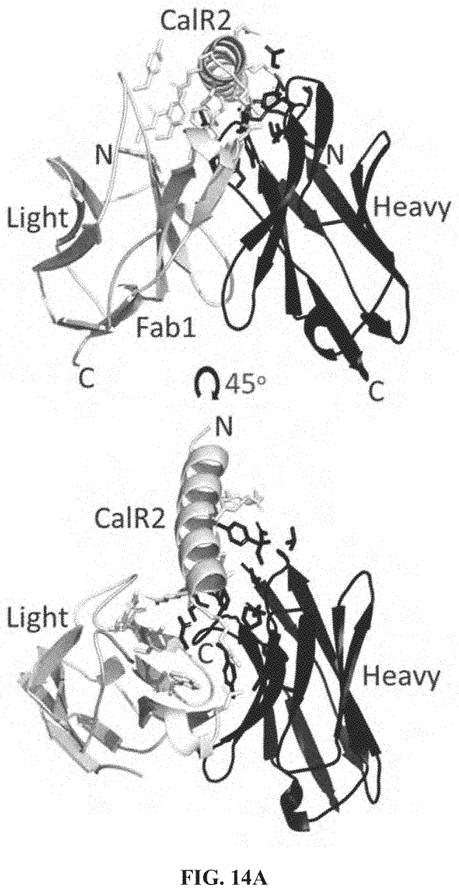

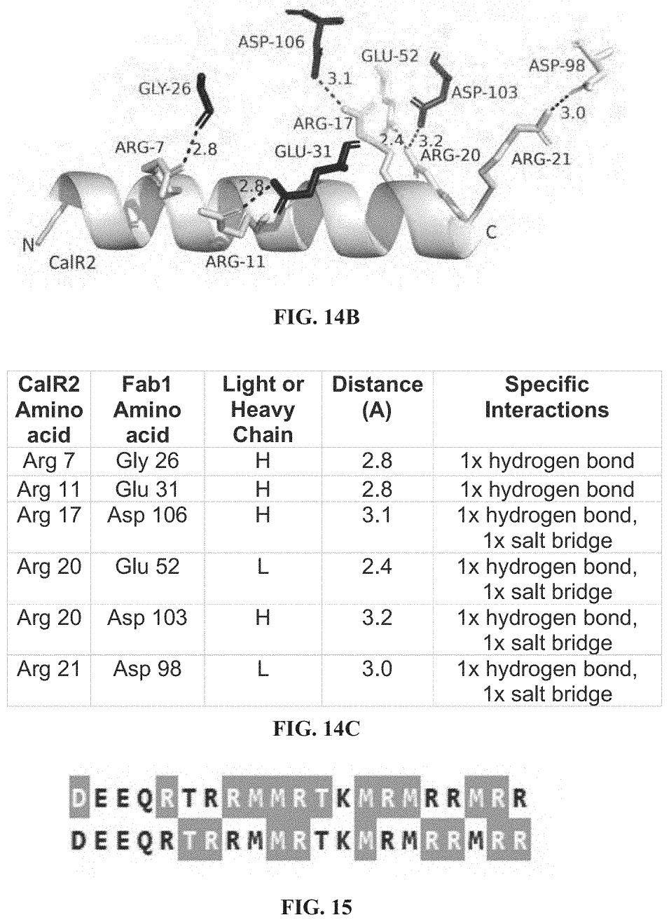

FIG. 12B shows the composition of the CDR loops (L1, L2, L3, H1, H2, and H3), the amino acid composition, and the length of the Fab. CDR predictions performed using the CCG scheme in MOE (Molecular Operating Environment). Amino acids highlighted in bold/italics are noted for significant contributions to CalR1 peptide binding as detailed in FIGS. 13B-13C. Amino acids highlighted in bold/underlined are noted for significant contributions to CalR2 peptide binding as detailed in FIGS. 14B-14C. Amino acids denoted by an asterix contribute to both CalR1 and CalR2 binding. FIG. 12C shows the Arrangement of CalR1 (gray) and CalR2 (white) in the CDR region of Fab1 (top) and the arrangement of the CDR loops (L1, L2, L3, H1, H2, and H3) and orientation of CalR1 (gray) and CalR2 (white) in the CDR region of Fab1 (bottom).

FIGS. 13A-13B show residues involved in binding of the Fab to CalR1. A magnified image of selected CalR1-Fab1 interacting residues is shown in FIG. 13B. FIG. 13C provides details for the selected CalR1-Fab1 interacting residues shown in FIG. 13B. FIGS. 14A-14B show residues involved in binding of the Fab to CalR2. A magnified image of selected CalR2-Fab1 interacting residues is shown in FIG. 14B. FIG. 14C provides details for the selected CalR2-Fab1 interacting residues shown in FIG. 14B. In FIGS. 13A-13C and FIGS. 14A-14C, Residues were selected based on distance and interaction type determined by MOE (Molecular Operating Environment). Heavy chain residues are shown in black and light chain residues are shown white. Distances in Angstroms are shown by dotted lines. Interacting residues are shown as sticks and were calculated using InterfaceResidues.py script and Pymol (protein.osaka-u.ac.jp/rcsfp/supracryst/suzuki/jpxtal/Katsutani/en/interface.php).

FIG. 15 shows the sequence of mutant CALR peptide with the CalR1 conformation binding residues (top) and CalR2 conformation binding residues (bottom) in grey shading.

### Example 12: Structural Determination of Fab Fragment from Anti-mutCALR Antibody Bound with Type 1 Mutant CalR 31-Mer Peptide

The Fab fragment having the sequences shown below was purified as described above for Example 11.

Sequences:
Fab Heavy Chain
(SEQ ID NO: 330)
EVQLVQSGAEVKKPGASVKVSCKVSGYTLTELSMQWVROAPGKGLEWMGGFDPDDAETMYAEKF
QGRLTVTEDTSTDTVYMELSSLRSEDTAVYFCATSPGYDFFDYWGQGTLVTVSSASTKGPSVFP
LAPSSKSTSGGTAALGCLVKDYFPEPVTVSWNSGALTSGVHTFPAVLQSSGLYSLSSVVTVPSS
SLGTQTYICNVNHKPSNTKVDKRVEPKSCDKTHT
Fab Light Chain
(SEQ ID NO: 260)
SYVLTQPPSVSVAPGKTARITCTGTSSDVGGYNYVSWYQQKPGQAPVLVVYEVSNRPSGIPERF
SGSNSGNTATLTISRVEAGDEADYYCQVWDSSNDLLIFGGGTKLTVLGQPKAAPSVTLFPPSSE
ELQANKATLVCLISDFYPGAVTVAWKADSSPVKAGVETTTPSKQSNNKYAASSYLSLTPEQWKS
HRSYSCQVTHEGSTVEKTVAPTECS
31-mer mutCALR peptide:
(SEQ ID NO: 340)
H-DEEQRTRRMMRTKMRMRRMRRTRRKMRRKMS-OH

Crystallization was performed as follows: Recombinant Fab fragment was mixed with the 31-mer peptide at a ratio of 1:3 molar excess, and then concentrated to a final concentration of 21 mg/mL. Sparse matrix crystallization screens were set up using an NT8 crystallization robot (Formulatrix, Bedford, Mass.). Drops containing 200 nl (Fab fragment plus CalR peptide)+200 nl (reservoir) were used for the setup and the plates were incubated at 4° C., 13° C. and 20° C. Crystals grew from condition E7 of the JCSG Top96 Screen (Rigaku, Bainbridge Island, Wash.) after one day of incubation at 20° C. The final crystal condition for antibody X:31-mer peptide complex was TRIS 0.1M, pH8.5, Polyethylene glycol 400 40% v/v, and 0.2 M Lithium sulfate.

All crystals were flash cooled in liquid nitrogen for X-ray data collection.

Data Collection, Processing and Refinement was performed as follows: Diffraction data was collected at 100K at (λ=0.9201 Å) with an Eiger 9M detector using synchrotron radiation at beamline 17-ID-1 at the Center for BioMolecular Structure at the National Synchrotron Light Source II, Brookhaven, N.Y., USA. 1800 images were collected using a rotation of 0.1V per image. Diffraction data indexing, integration and scaling were performed using Fast_dp. Data collection statistics, phasing and refinement are given in Table 15.

For the Fab fragment-31-mer peptide data, Phaser was used to place fragments of the Fab fragment model and subsequent rounds of manual model building were performed resulting in a model containing 1 Fab and I peptide. Subsequent cycles of density modifications, model building and refinement were carried out using Refmac, Coot and Phenix until the structure converged at reasonable R-work and R-free (Table 15). The final model were analyzed for good stereochemistry, geometry and clash scores using MolProbity (Table 15).

TABLE 15
Data collection statistics, phasing and refinement.

| | Fab fragment:CalR peptide (31-mer) | |
|---|---|---|
| Resolution range | 29.62-2.04 | (2.113-2.04) |
| Space group | C 1 2 1 | |
| Unit cell | 105.37 71.477 92.347 90 105.807 90 | |
| Total reflections | 82513 | (8206) |
| Unique reflections | 42070 | (4190) |
| Multiplicity | 2.0 | (2.0) |
| Completeness (%) | 99.76 | (99.90) |
| Mean I/sigma(I) | 10.09 | (1.61) |
| Wilson B-factor | 38.61 | |
| R-merge | 0.04238 | (0.4129) |
| R-meas | 0.05993 | (0.584) |
| R-pim | 0.04238 | (0.4129) |
| CC½ | 0.997 | (0.679) |
| CC* | 0.999 | (0.899) |
| Reflections used in refinement | 42064 | (4190) |
| Reflections used for R-free | 1995 | (198) |
| R-work | 0.2017 | (0.2759) |
| R-free | 0.2262 | (0.3079) |
| CC(work) | 0.957 | (0.789) |
| CC(free) | 0.949 | (0.760) |

| | |
|---|---|
| Number of non-hydrogen atoms | 3690 |
| macromolecules | 3440 |
| ligands | 38 |
| solvent | 212 |
| Protein residues | 464 |
| RMS(bonds) | 0.008 |
| RMS(angles) | 1.13 |
| Ramachandran favored (%) | 95.18 |
| Ramachandran allowed (%) | 4.39 |
| Ramachandran outliers (%) | 0.44 |
| Rotamer outliers (%) | 0 |
| Clashscore | 5.02 |
| Average B-factor | 47.84 |
| macromolecules | 47.77 |

Results:

The structure of Fab fragment-31-mer mutCalR peptide was determined at 2.0 Å with space group C 1 2 1. Each asymmetric unit contains one Fab molecule and one 31-mer peptide molecule. The Fab molecule consists of a light chain, Chain L: 2-215, and a heavy chain, Chain H:1-221. The model has well defined electron density throughout, with the exception of the following residues: Chain H, Gly42-Lys43, which are not modelled due to weak electron density. The structure was refined to an R-work/R-free of 20.1% and 22.6%, respectively, and has good stereochemistry throughout with 2 Ramachandran outliers.

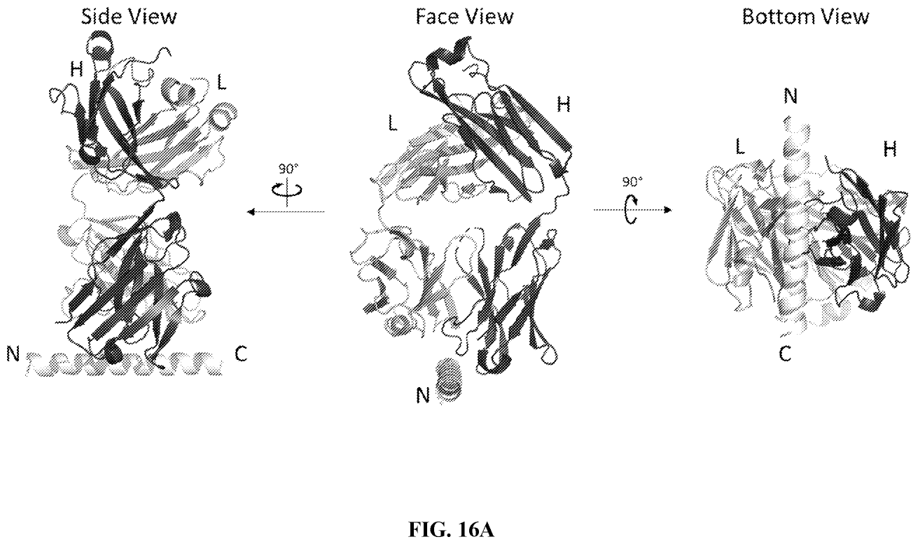

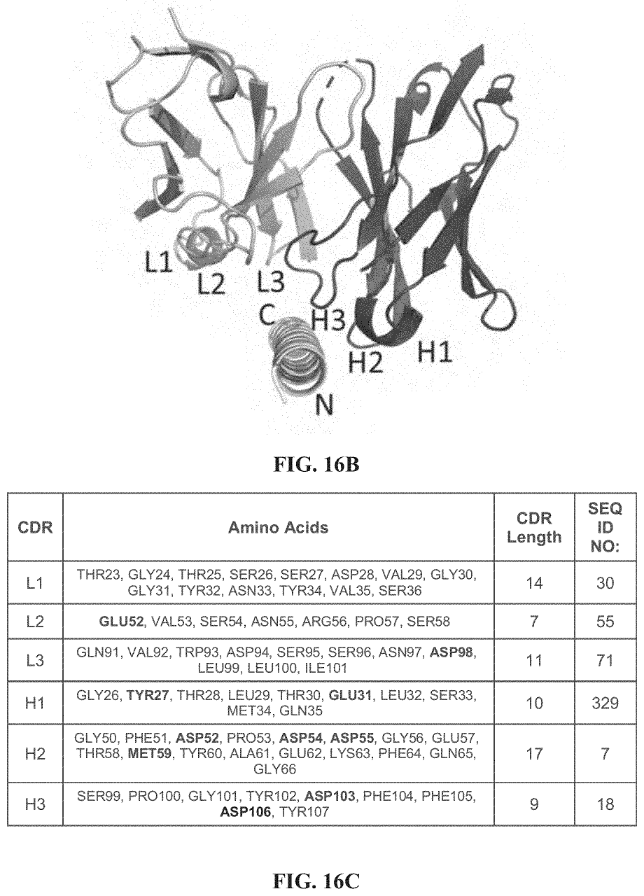

FIG. 16A shows views of the protein structure in an asymmetric unit. The asymmetric unit consists of 1 light chain, 1 heavy chain from the Fab fragment and a 31-amino acid peptide from Type 1 mutant CalR. FIG. 16B shows a zoom-in view for the region of CDR and mutant CalR peptide. Constant regions of the Fab are excluded for clarity. FIG. 16C shows the CDR composition of the Fab fragment. Bold font denotes amino acids that contribute to the epitope recognition in mutant CalR. CCG numbering scheme was used for defining the CDR in MOE (Molecular Operating Environment, 2019.01; Chemical Computing Group ULC, 1010 Sherbooke St. West, Suite #910, Montreal, QC, Canada, H3A 2R7, 2021).

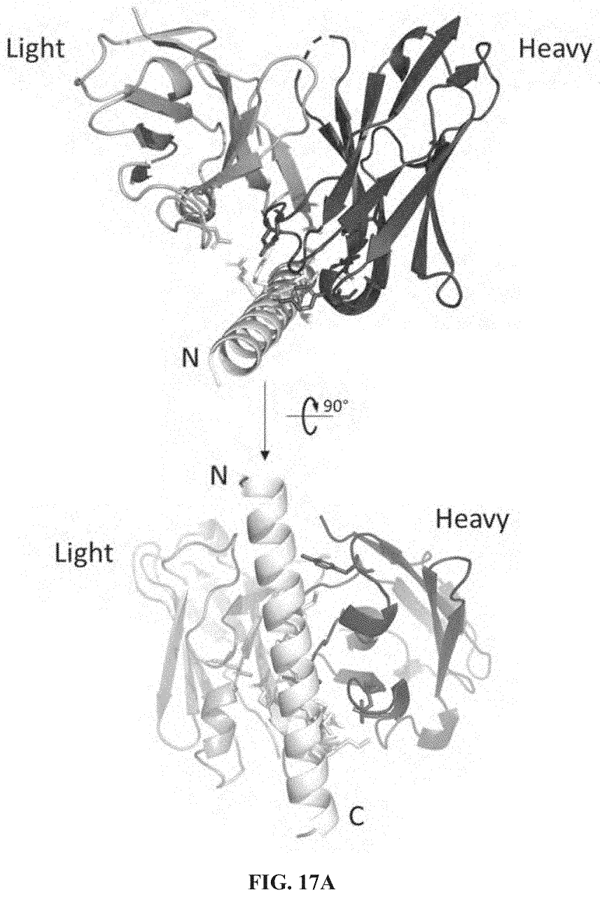

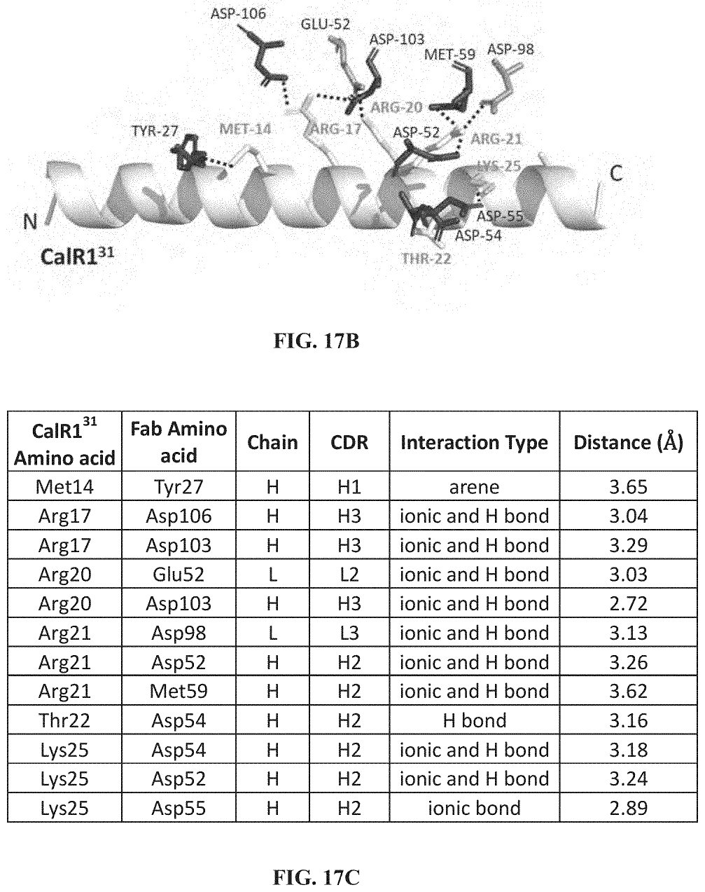

FIG. 17A shows an illustration of interacting residues located across CDR regions from the heavy chain and the light chain. FIG. 17B shows selected residues for interaction analysis between the antibody and the antigen CalR31 (31-mer mutant CalR peptide). Residues selected based on distance and interaction type determined by MOE (Molecular Operating Environment, 2019.01; Chemical Computing Group ULC, 1010 Sherbooke St. West, Suite #910, Montreal, QC, Canada, H3A 2R7, 2021). The side chain of residues are shown in stick representation with numbering adopted from the CCG scheme in MOE. FIG. 17C shows results from a detailed interaction analysis between the CalR31 and the CDR of antibody. The distance between interacting pairs were calculated in MOE. Distances in Angstroms are shown by dotted lines.

### Example 13: Efficacy of Anti-mutCALR Antibody in a Mouse Model of Essential Thrombocythemia

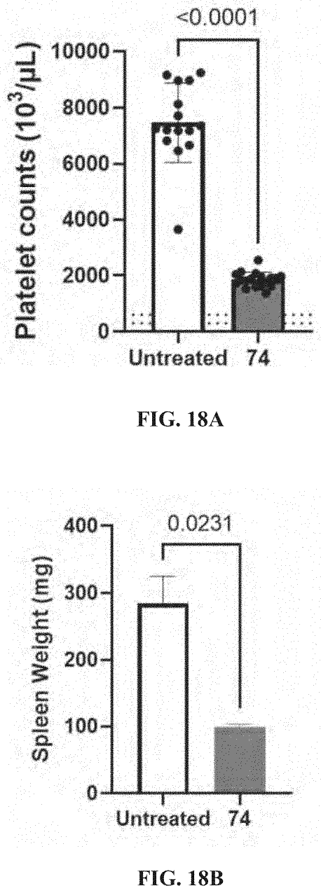

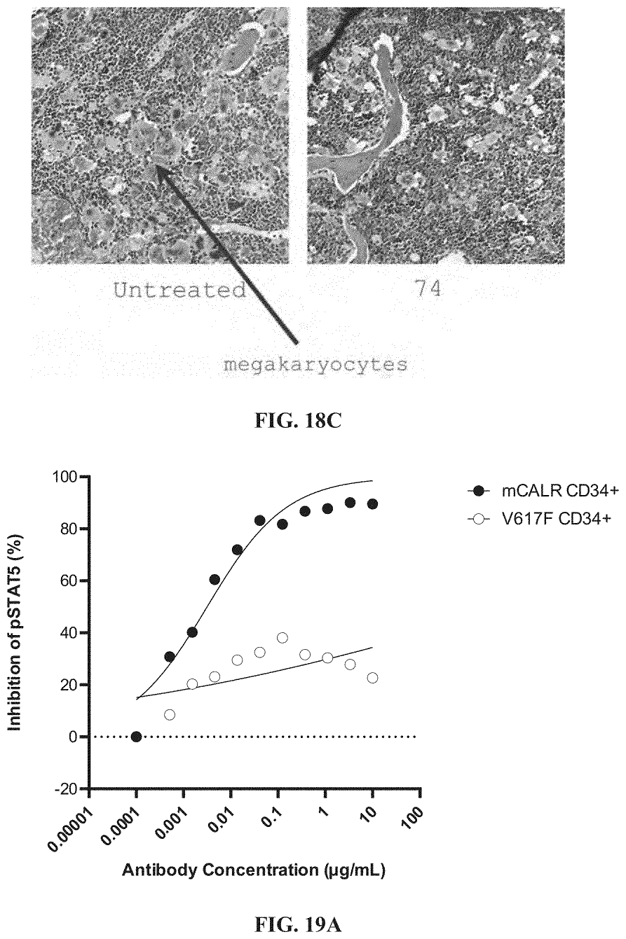

To test the therapeutic potential of anti-mutCALR antibodies in vivo, an antibody was evaluated in a mouse model of essential thrombocythemia (ET). In this mouse model, a conditional allele that expresses a mutCALR protein with a C-terminal sequence (DEL52) identical to what found in MPN patients was knocked in to the mouse CALR sequence. The engineered mice (CALRdel/del) develop an ET-like disease with marked thrombocytosis, splenomegaly, and abnormal megakaryocytosis (Li et. al. Blood 2018; 131:649). The expression of mutCALR in the engineered mice was induced with intraperitoneal injections of polyinosinic:polycytidylic acid (poly I:C) (250 μg/dose; every other day for a total of 3 injections). Treatment initiated 19 weeks post-poly I:C induction and consisted of intravenous injections of anti-mutCALR antibody (clone 74) at 50 mg/kg QW for a total of 4 weeks. ET phenotype was confirmed by assessing the platelets counts in the blood (Sysmex, Kobe, Japan), spleen size and bone marrow histology. A representative study is shown in FIGS. 18A-18C.

The anti-mutCALR antibody restored normal platelet counts (FIG. 18A), spleen volume (FIG. 18B), and the bone marrow cell environment (FIG. 18C).

Taken together, these results demonstrate the efficacy of the anti-mutCALR antibodies in treating ET.

### Example 14: Efficacy of Anti-mutCALR Antibody in Primary Patient Cells

CD34⁺ cells isolated from MPN patients carrying the CALR mutation were used to characterize the ability of an anti-mutCALR antibody to inhibit the mutCALR-derived oncogenic function. Peripheral blood mononuclear cells (PBMCs) were isolated from non-identified blood samples from MPN patients by Ficoll gradient extraction (Fisher Scientific) and CD34⁺ cells were enriched using magnetic enrichment (Miltenyi Biotec). CD34⁺ cells were cultured for seven days in SFEM-II media (STEMCELL Technologies) containing hSCF, hFLT3L, TPO, LDL2698, SR1, and UM171.

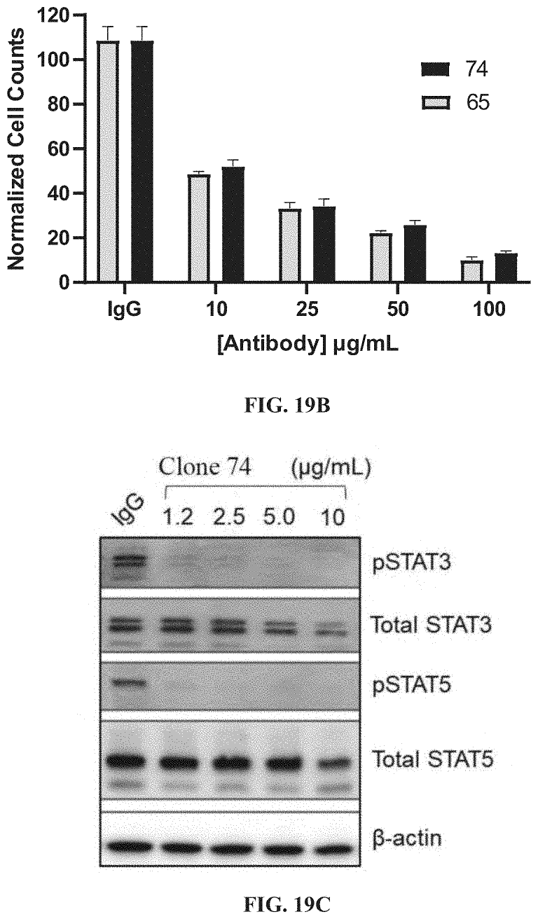

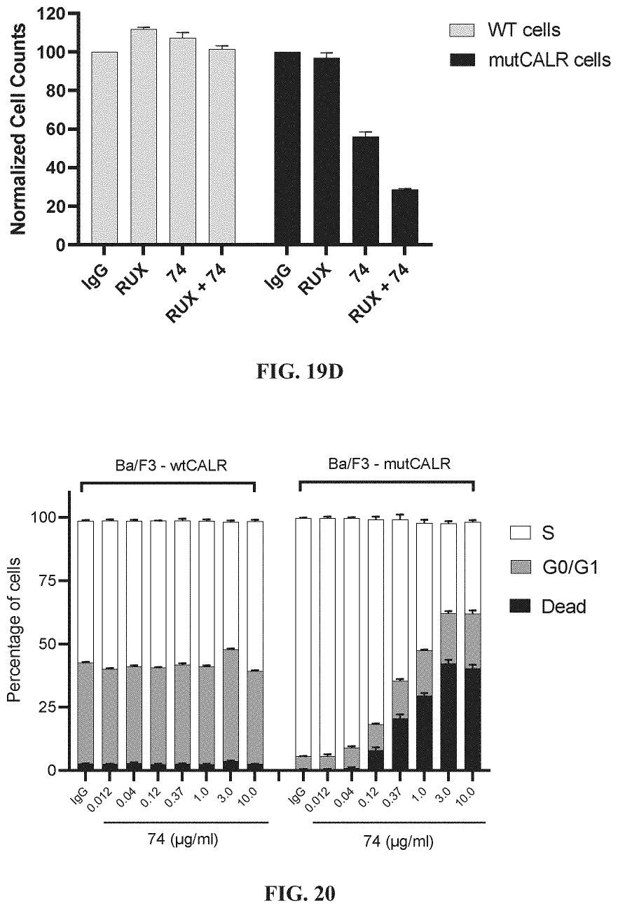

CD34+ cells (50,000 cells/well) were then plated into 96-well plates and treated with a mutCALR or isotype control antibody for 2 hours. Following treatment, plates were centrifuged, the supernatant was aspirated and subsequently washed with PBS. After centrifugation the cell pellets were lysed using lysing buffer (Cell Signaling Technologies) supplemented with 1× Halt™ Protease and Phosphatase Inhibitor Cocktail (Thermo Fisher Scientific). Lysates were added to the Phospho (Tyr694)/Total STAT5a,b whole cell lysate kit (Meso Scale Diagnostics) and phospho-STAT5 levels were then quantitated using Meso Sector S 600 (Meso Scale Diagnostics). Clone 74 selectively inhibited pSTAT5 in CD34+ cells harboring mutCALR in a dose-dependent manner, while the isotype control (IgG) at 10 μg/mL had no impact on CD34⁺ cells. Moreover, pSTAT5 was not inhibited in CD34+ cells harboring the V617F JAK2 mutation (FIG. 19A).

To evaluate the ability of antibodies to inhibit mutCALR oncogenic function and imbalanced proliferation of megakaryocytes, CD34⁺ cells (50,000 cells/well) were added to a 12-well plate with SFEM-II supplemented with hSCF, hGCSF, hIL3, and hIL6 and treated with a mutCALR antibody or isotype control for 6 days. Cells were stained and analyzed by flow cytometry (LSRFortessam X-20 analyzer, BD Biosciences). Antibodies used were: APC anti-human CD38 antibody (BioLegend), FITC anti-human lineage cocktail (BioLegend), PE/Cyanine7 anti-human CD34 antibody (BioLegend), PE anti-human CD41 antibody (BioLegend), APC mouse anti-human CD42b antibody (BD Pharmingen). Megakaryocytes were identified as the CD41⁺CD42b⁺ cells. The anti-mutCALR antibodies (clones 74 and 65) selectively prevented the differentiation of mutCALR CD34⁺ cells into mature megakaryocytes in a dose-dependent manner, while isotype control (IgG) had no impact on this population. A representative experiment is shown in FIG. 19B.

In another experiment, the CD34⁺ cells described above were added at 50,000 cells/well to a 12-well plate containing 2.0 mL culture media with specified concentrations of clone 74 or isotype control. The treatment period was 12 hours. After 12 hours, cells were collected, washed and lysed using lysing buffer (Cell Signaling Technologies) supplemented with 1× Halt™ Protease and Phosphatase Inhibitor Cocktail (Thermo Fisher Scientific). Protein samples (6 μg) were separated in pre-casted 4-12% TrisGglycine gels (Thermo Fisher Scientific) and transferred to nitrocellulose membranes using iBlot 2 Dry Blotting System and iBlot™ 2 Transfer Stacks (Thermo Fisher Scientific). The nitrocellulose membranes were blocked with StartingBlock (Thermo Fisher Scientific) for one hour and probed with antibodies to detect pSTAT5 (Cell Signaling), STAT5 (Cell Signaling), pSTAT3 (Cell Signaling), STAT3 (Cell Signaling), and β-actin (Cell Signaling). Detection was performed using horseradish peroxidase (HRP)-conjugated secondary rabbit antibody (Cell Signaling) and chemiluminescence HRP substrate (Thermo Scientific). Clone 74 selectively inhibited pSTAT3 and pSTAT5 in CD34+ cells harboring mutCALR in a dose-dependent manner, while the isotype control (IgG) at 10 μg/mL had no impact on CD34⁺ cells (FIG. 19C).

In another experiment, Clone 74 and ruxolitinib were combined to determine the impact of co-treatment on megakaryopoiesis produced by CD34⁺ cells from human cord blood (WT cells) or an MPN patient with mutCALR (mutCALR cells). After six days in culture, 25 nM ruxolitinib alone did not impact the frequency of megakaryocytes produced by either WT or mutCALR-expressing CD34 cells. In contrast, 25 μg/mL of clone 74 alone selectively reduced the production of megakaryocytes from mutCALR⁺ CD34⁺ cells but not from WT CD34⁺ cells. Further, the combination of 25 nM ruxolitinib and 25 μg/mL of clone 74 led to further depletion of pathogenic megakaryocytes produced by mutCALR⁺ CD34⁺ cells (FIG. 19D).

Taken together, these results demonstrate the ability of the anti-mutCALR antibodies to inhibit mutCALR-derived oncogenic functions in MPN patient cells.

### Example 15: Effect of Anti-mutCALR Antibody in the Cell Cycle

Engineered BaF3 cells (10,000 cells/well) were added to a 12-well plate containing 2.0 mL culture media with serially diluted antibodies. The treatment period was for 24 hours. After 22 hours of incubation, Ba/F3 cells were pulse-labeled with BrdU for 2 hours. After 24 hours, Ba/F3 cells were collected, washed with PBS and incubated with BD Cytofix/Cytoperm buffer for 20 minutes on ice. Cells were then washed with the BD Perm/Wash buffer and the cell pellets were resuspended in 100 μL of BD Cytoperm and Permeabilization buffer and incubated on ice for 10 minutes. Cells were then washed and resuspended in 100 μl of BD Cytofix/Cytoperm buffer and incubated at room temperature for 5 minutes. Cells were washed and treated with 100 μL of 300 μg/mL solution of DNase, and incubated for 1 hour at room temperature, and were then washed and resuspended in 50 μl of BD Perm/Wash buffer containing anti-BrdU-APC antibody for 20 minutes in the dark at room temperature. After additional washing, cells were resuspended in PBS containing 2% FBS and 7-AAD and analyzed for cell cycle profiles on the LSRFortessa™ X-20 analyzer (BD Biosciences). Clone 74 was screened to determine its effect on the cell cycle using WT BaF3 cells or Ba/F3-TPOR/mutCALR type 1. Clone 74 selectively induced apoptosis in BaF3 cells carrying mutCALR in a dose-dependent manner, whereas isotype control (IgG) had no effect on these cells. In contrast, clone 74 did not impact cell cycle profiles of WT BaF3 cells (FIG. 20).

### Example 16: Activity of Anti-mutCALR Antibody

Cell binding of various anti-mutCALR antibodies as compared to clone 4 was assessed using engineered BaF3 cells stably expressing human MPL and human mutCALR Type 1. The antibodies used in this experiment were obtained commercially (AB1: CAL2 antibody, Dianova) or synthesized from sequences found in the literature (AB2: anti-mutCALR antibody including VH and VL regions of antibody B3 from WO2020175689 fused to human constant regions; AB3: anti-mutCALR antibody including the BJ095 VH region and the BJ097 VL region from WO2019178362 fused to human constant regions; and AB4: anti-mutCALR antibody including VH and VL regions of antibody 8B2-H6 from WO2016087514 fused to human constant regions).

Sequences:
AB2 heavy chain:
(SEQ ID NO: 341)
QVQLQQSGAELVKPGSSVKISCKASGYTFTRNFIHWIKQQPGNGLEWIGWIFPGDGDTEYNQKF
NGKATLTADKSSSTAYMQLSSLTSEDSAVYFCARGNYNYEYFDYWGQGVMVTVSSASTKGPSVE
PLAPSSKSTSGGTAALGCLVKDYFPEPVTVSWNSGALTSGVHTFPAVLQSSGLYSLSSVVTVPS
SSLGTQTYICNVNHKPSNTKVDKRVEPKSCDKTHTCPPCPAPELLGGPSVFLFPPKPKDTLMIS
RTPEVTCVVVDVSHEDPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEALHNHYTQKSLSLSPG
AB2 light chain:
(SEQ ID NO: 342)
DIQMTQSPASLSASLGETVSIECLASEDIYSYLAWYQQKPGKSPQLLIFAANRLQDGVPSRFSG
SGSGTQFSLKISGMQPEDEGDYFCLQGSKFPYTFGPGTKLELNRTVAAPSVFIFPPSDEQLKSG
TASVVCLLNNFYPREAKVOWKVDNALQSGNSQESVTEQDSKDSTYSLSSTLTLSKADYEKHKVY
ACEVTHQGLSSPVTKSENRGEC
AB3 heavy chain:
(SEQ ID NO: 343)
QVQLVQSGAEVKKPGASVKVSCKASGYSFTGYYIHWVRQAPGQGLEWIGYISAYNGASSYNOKF
KGRATFTVDTSISTAYMELSRLRSDDTAVYYCASSMDYWGQGTLVTVSSASTKGPSVFPLAPSS
KSTSGGTAALGCLVKDYFPEPVTVSWNSGALTSGVHTFPAVLQSSGLYSLSSVVTVPSSSLGTQ
TYICNVNHKPSNTKVDKRVEPKSCDKTHTCPPCPAPELLGGPSVFLFPPKPKDTLMISRTPEVT
CVVVDVSHEDPEVKENWYVDGVEVHNAKTKPREEQYNSTYRVVSVLTVLHQDWLNGKEYKCKVS
NKALPAPIEKTISKAKGQPREPQVYTLPPSREEMTKNQVSLTCLVKGFYPSDIAVEWESNGQPE
NNYKTTPPVLDSDGSFFLYSKLTVDKSRWQQGNVFSCSVMHEALHNHYTQKSLSLSPG
*AB3 light chain:
(SEQ ID NO: 344)
DVVMTQSPLSLPVTLGQPASISCKSSQSLLDSDGKTYLNWLQQRPGQSPKRLIYLVSKLDSGVP
QLKSGTASVVCLLNNFYPREAKVQWKVDNALQSGNSQESVTEQDSKDSTYSLSSTLTLSKADYE
KHKVYACEVTHQGLSSPVTKSFNRGEC
*There was an EI→IE transposition when synthesizing the VL
sequence at the end of the variable region; otherwise, the VL
sequence is identical to the BJ097 VL region.
AB4 Heavy Chain:
(SEQ ID NO: 345)
EVQLKQSGPELVKTGASVKISCKASGYSFTGYYIHWVKQSHGKSLEWIGYISCYNGASSYNQKF
KGKATFTVDTSSSTAYMQFNSLTSGDSAVYYCASSMDYWGQGTSVTVSSASTKGPSVFPLAPSK
STSGGTAALGCLVKDYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKPKDTLMISRTPEVCV
VVDVSHEDPEVKENWYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSDIAVEWESNGQPNNY
KTTPPVLDSDGSFFLYSKLTVDKSRWQQGNVFSCSVMHEALHNHYTQKSLSLSPG
AB4 Light Chain:
(SEQ ID NO: 346)
DVVMTQTPLTLSVTIGQPASISCKSSQSLLDSDGKTYLNWLLQRPGQSPKRLIYLVSKLDSGVP
DRFTGSGSGTDFTLKISRVEAEDLGVYHCWQGTHEPYTFGGGTKLEIKRTVAAPSVFIFPPSDQ
LKSGTASVVCLLNNFYPREAKVQWKVDNALQSGNSQESVTEQDSKDSTYSLSSTLTLSKADYEK
HKVYACEVTHQGLSSPVTKSENRGEC

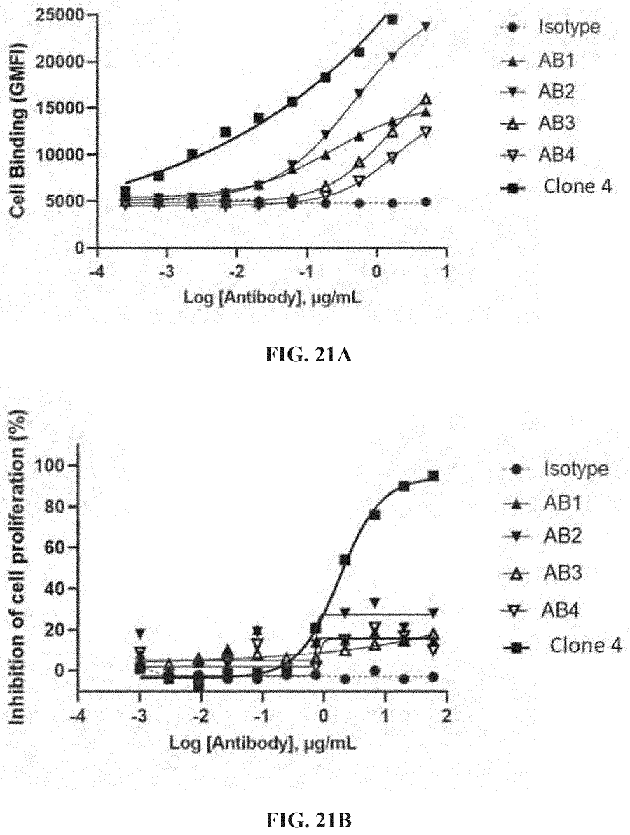

Approximately 200,000 cells/well were added to 96-well plates and stained with the indicated concentration of antibodies for 30 minutes on ice. Cells were then washed and stained with goat anti-human secondary conjugated to R-phycoerythrin (R-PE) (Jackson Immuno Research Laboratories) for 30 minutes on ice. The cells were then washed again and analyzed by flow cytometry. Geometric Mean Fluorescence Intensity (GMFI) of cell binding was graphed using GraphPad Prism Software (version 7.04). As shown in FIG. 21A, the anti-mutant CALR antibodies show differential levels of binding to cells expressing mutant CALR.

The ability of the five antibodies shown here to inhibit cell proliferation was tested. Inhibition of cell proliferation was assessed using engineered BaF3 cells expressing MPL/mutCALR Type 1. Cells were plated at 5,000 cells per well in RPMI 1640+2% FBS, antibodies were added and incubated for 72 hours, followed by assessment of cell viability using the CellTiter-Glo Luminescent Cell Viability Assay (Promega, Madison, Wis.) and Top Count (Perkin Elmer, Boston, Mass.) or Pherastar (BMG Labtech, Ortenberg, Germany) for luminescence quantification. As shown in FIG. 21B, among the antibodies shown to bind mutant CALR, only clone 4 is able to substantially inhibit cell proliferation. The data shown in FIGS. 21A and 21B were compiled from different experiments.

The ability of other antibodies of the present disclosure to inhibit cell proliferation as compared to AB1, AB2, AB3 and AB4 was tested in the same manner as described above. The identified anti-mutCALR antibody clones listed below inhibited mutCALR-induced oncogenic cell proliferation in a dose-dependent manner, while AB1, AB2, AB3 and AB4 showed no functional activity. The IC₅₀ values are shown in Table 16.

TABLE 16
IC₅₀ values for anti-mutCALR antibody mediated
inhibition of cell proliferation

| Clone No. | IC50 (μg/mL) |
|---|---|
| 53 | 9.5 |
| 39 | 21 |
| 35 | 2.18 |
| 36 | 0.98 |
| 41 | 3.98 |
| 4 | 0.10 |
| 51 | 10 |
| 37 | 0.201 |
| AB1 | >60 |
| AB2 | >60 |
| AB3 | >60 |
| AB4 | >60 |

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody that binds to human mutant calreticulin (CALR), wherein:
the antibody comprises a heavy chain variable region (VH) comprising a VH CDR1, a VH CDR2, and a VH CDR3, wherein:
the VH CDR1 comprises the amino acid sequence X₁X₂X₃X₄X₅, wherein X₁ is S, E, or D; wherein X₂ is Y, L, or S; wherein X₃ is A, S, or F; wherein X₄ is I or M; and wherein X₅ is S, Q, or H;
the VH CDR2 comprises the amino acid sequence X₆X₇X₈PX₉X₁₀X₁₁X₁₂X₁₃X₁₄YAX₁₅X₁₆X₁₇X₁₈G (SEQ ID NO:97), wherein X₆ is L or G; wherein X₇ is V, F, or I; wherein X₈ is D or I; wherein X₉ is E, D, or I; wherein X₁₀ is D, G, F, A, S, or E; wherein X₁₁ is G or A; wherein X₁₂ is E or T; wherein X₁₃ is T or A; wherein X₁₄ is I, M, or N; wherein X₁₅ is E or Q; wherein X₁₆ is K or R; wherein X₁₇ is F or L; and wherein X₁₈ is R or Q;
the VH CDR3 comprises the amino acid sequence X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇X₃₈ (SEQ ID NO:98), wherein X₁₉ is P, E, or absent; wherein X₂₀ is G, E, or absent; wherein X₂₁ is G, W, S, or absent; wherein X₂₂ is I, D, S, P, or absent; wherein X₂₃ is S, L, I, T, G, or absent; wherein X₂₄ is P, T, Q, I, D, R, or absent; wherein X₂₅ is G, D, or absent; wherein X₂₆ is E, Y, P, L, D, or S; wherein X₂₇ is E, D, A, or G; wherein X₂₈ is S, F, A, E, Y, or W; wherein X₂₉ is Y or F; wherein X₃₀ is G, D, or W; wherein X₃₁ is P, Y, I, or H; wherein X₃₂ is Y or absent; wherein X₃₃ is Y or absent; wherein X₃₄ is Y or absent; wherein X₃₅ is G or absent; wherein X₃₆ is M or absent; wherein X₃₇ is D or absent; wherein X₃₈ is V or absent;
wherein the antibody comprises a light chain variable region (VL) comprising a VL CDR1, a VL CDR2, and a VL CDR3, wherein:
the VL CDR1 comprises the amino acid sequence X₃₉X₄₀X₄₁X₄₂X₄₃X₄₄X₄₅X₄₆X₄₇X₄₈X₄₉X₅₀X₅₁X₅₂X₅₃X₅₄ (SEQ ID NO:99), wherein X₃₉ is T, A, or absent; wherein X₄₀ is G or absent; wherein X₄₁ is Q, G, V, T, or S; wherein X₄₂ is A, G, S, or N; wherein X₄₃ is S, N, D, T, or Y; wherein X₄₄ is Q, Y, N, D, S, or K; wherein X₄₅ is D, I, F, V, S, or T; wherein X₄₆ is N or absent; wherein X₄₇ is I or absent; wherein X₄₈ is I, G, or R wherein X₄₉ is S, G, A, D, I, R, or T; wherein X₅₀ is Y or absent; wherein X₅₁ is N, K, I, or E; wherein X₅₂ is Y, S, N, D, H, F, R, or G; wherein X₅₃ is L or V; and wherein X₅₄ is N, H, S, D, or F;
the VL CDR2 comprises the amino acid sequence X₅₅X₅₆X₅₇X₅₈X₅₉X₆₀X₆₁, wherein X₅₅ is T, D, E, Q, or R; wherein X₅₆ is A, D, V, or N; wherein X₅₇ is S, G, N, or R; wherein X₅₈ is I, N, D, or K; wherein X₅₉ is L, R, or W; wherein X₆₀ is E or P; and wherein X₆₁ is S, T, or L;
the VL CDR3 comprises the amino acid sequence X₆₂X₆₃X₆₄X₆₅X₆₆X₆₇X₆₈X₆₉X₇₀X₇₁X₇₂, wherein X₆₂ is Q, S, C, or G; wherein X₆₃ is Q, V, S, T, or A; wherein X₆₄ is Q, L, W, or Y; wherein X₆₅ is Q, N, D, I, T, A, or G; wherein X₆₆ is S, P, G, N, or A; wherein X₆₇ is N, Y, I, S, N, L, or D; wherein X₆₈ is E, P, S, I, N, H, L, or T; wherein X₆₉ is D, T, S, or absent; wherein X₇₀ is P, H, L, R, F, A, Q, or absent; wherein X₇₁ is W, L, V, Y, S, A, or E; and wherein X₇₂ is T, V, or I.

2. The antibody of claim 1, wherein:
the VH CDR1 comprises the amino acid sequence of any one of SEQ ID NOs:1-6;
the VH CDR2 comprises the amino acid sequence of any one of SEQ ID NOs:7-17 and 92-95;
the VH CDR3 comprises the amino acid sequence of any one of SEQ ID NOs:18-25;
the VL CDR1 comprises the amino acid sequence of any one of SEQ ID NOs:26-52 or 118;
the VL CDR2 comprises the amino acid sequence of any one of SEQ ID NOs:53-68; and
the VL CDR3 comprises the amino acid sequence of any one of SEQ ID NOs:69-91.

3. The antibody of claim 1, wherein the VH CDR1, the VH CDR2, and the VH CDR3 each correspond to the VH CDRs set forth in Tables 1-2 for a single clone selected from the group consisting of clones 1-53 and 55-215, and wherein the VL CDR1, the VL CDR2, and the VL CDR3 each correspond to the VL CDRs set forth in Tables 1-2 for a single clone selected from the group consisting of clones 1-53 and 55-215.

4. The antibody of claim 1, wherein:
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:26; the VL CDR2 comprises the amino acid sequence of SEQ ID NO:53; and the VL CDR3 comprises the amino acid sequence of SEQ ID NO:69;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:27; the VL CDR2 comprises the amino acid sequence of SEQ ID NO:54; and the VL CDR3 comprises the amino acid sequence of SEQ ID NO:70;
the VH CDR1 comprises the amino acid sequence of SEQ ID NO:1; the VH CDR2 comprises the amino acid sequence of SEQ ID NO:8; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:29; the VL CDR2 comprises the amino acid sequence of SEQ ID NO:54; and the VL CDR3 comprises the amino acid sequence of SEQ ID NO:72;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:29; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:73;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:56; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:31; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:30; the VL CDR2 comprises the amino acid sequence of SEQ ID NO:57; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:9; the VH CDR3 comprises the amino acid sequence of SEQ ID NO:18; the VL CDR1 comprises the amino acid sequence of SEQ ID NO:32; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:33; the VL CDR2 comprises the amino acid sequence of SEQ ID NO:54; and the VL CDR3 comprises the amino acid sequence of SEQ ID NO:73;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:34; the VL CDR2 comprises the amino acid sequence of SEQ ID NO:56; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:27; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:35; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:36; the VL CDR2 comprises the amino acid sequence of SEQ ID NO:54; and the VL CDR3 comprises the amino acid sequence of SEQ ID NO:72;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:58; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:37; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:38; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:32; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:39; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:40; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:10; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:41; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:70;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:40; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:74;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:75;
the VH CDR1 comprises the amino acid sequence of SEQ ID NO:1; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:118; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:2; the VH CDR2 comprises the amino acid sequence of SEQ ID NO:11; the VH CDR3 comprises the amino acid sequence of SEQ ID NO:19; the VL CDR1 comprises the amino acid sequence of SEQ ID NO:30; the VL CDR2 comprises the amino acid sequence of SEQ ID NO:59; and the VL CDR3 comprises the amino acid sequence of SEQ ID NO:76;
the VH CDR1 comprises the amino acid sequence of SEQ ID NO:2; the VH CDR2 comprises the amino acid sequence of SEQ ID NO:11; the VH CDR3 comprises the amino acid sequence of SEQ ID NO:19; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:77;
the VH CDR1 comprises the amino acid sequence of SEQ ID NO:2; the VH CDR2 comprises the amino acid sequence of SEQ ID NO:11; the VH CDR3 comprises the amino acid sequence of SEQ ID NO:19; the VL CDR1 comprises the amino acid sequence of SEQ ID NO:42; the VL CDR2 comprises the amino acid sequence of SEQ ID NO:59; and the VL CDR3 comprises the amino acid sequence of SEQ ID NO:76;
the VH CDR1 comprises the amino acid sequence of SEQ ID NO:2; the VH CDR2 comprises the amino acid sequence of SEQ ID NO:11; the VH CDR3 comprises the amino acid sequence of SEQ ID NO:19; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:78;
the VH CDR1 comprises the amino acid sequence of SEQ ID NO:2; the VH CDR2 comprises the amino acid sequence of SEQ ID NO:12; the VH CDR3 comprises the amino acid sequence of SEQ ID NO:19; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:77;
the VH CDR1 comprises the amino acid sequence of SEQ ID NO:2; the VH CDR2 comprises the amino acid sequence of SEQ ID NO:11; the VH CDR3 comprises the amino acid sequence of SEQ ID NO:19; the VL CDR1 comprises the amino acid sequence of SEQ ID NO:43; the VL CDR2 comprises the amino acid sequence of SEQ ID NO:59; and the VL CDR3 comprises the amino acid sequence of SEQ ID NO:79;
the VH CDR1 comprises the amino acid sequence of SEQ ID NO:2; the VH CDR2 comprises the amino acid sequence of SEQ ID NO:11; the VH CDR3 comprises the amino acid sequence of SEQ ID NO:19; the VL CDR1 comprises the amino acid sequence of SEQ ID NO:30; the VL CDR2 comprises the amino acid sequence of SEQ ID NO:60; and the VL CDR3 comprises the amino acid sequence of SEQ ID NO:79;
the VH CDR1 comprises the amino acid sequence of SEQ ID NO:3; the VH CDR2 comprises the amino acid sequence of SEQ ID NO:13; the VH CDR3 comprises the amino acid sequence of SEQ ID NO:20; the VL CDR1 comprises the amino acid sequence of SEQ ID NO:44; the VL CDR2 comprises the amino acid sequence of SEQ ID NO:61; and the VL CDR3 comprises the amino acid sequence of SEQ ID NO:80;
the VH CDR1 comprises the amino acid sequence of SEQ ID NO:3; the VH CDR2 comprises the amino acid sequence of SEQ ID NO:11; the VH CDR3 comprises the amino acid sequence of SEQ ID NO:20; the VL CDR1 comprises the amino acid sequence of SEQ ID NO:45; the VL CDR2 comprises the amino acid sequence of SEQ ID NO:61; and the VL CDR3 comprises the amino acid sequence of SEQ ID NO:80;
the VH CDR1 comprises the amino acid sequence of SEQ ID NO:4; the VH CDR2 comprises the amino acid sequence of SEQ ID NO:14; the VH CDR3 comprises the amino acid sequence of SEQ ID NO:21; the VL CDR1 comprises the amino acid sequence of SEQ ID NO:46; the VL CDR2 comprises the amino acid sequence of SEQ ID NO:62; and the VL CDR3 comprises the amino acid sequence of SEQ ID NO:81;
the VH CDR1 comprises the amino acid sequence of SEQ ID NO:5; the VH CDR2 comprises the amino acid sequence of SEQ ID NO:14; the VH CDR3 comprises the amino acid sequence of SEQ ID NO:21; the VL CDR1 comprises the amino acid sequence of SEQ ID NO:46; the VL CDR2 comprises the amino acid sequence of SEQ ID NO:62; and the VL CDR3 comprises the amino acid sequence of SEQ ID NO:81;
the VH CDR1 comprises the amino acid sequence of SEQ ID NO:5; the VH CDR2 comprises the amino acid sequence of SEQ ID NO:14; the VH CDR3 comprises the amino acid sequence of SEQ ID NO:21; the VL CDR1 comprises the amino acid sequence of SEQ ID NO:46; the VL CDR2 comprises the amino acid sequence of SEQ ID NO:63; and the VL CDR3 comprises the amino acid sequence of SEQ ID NO:81;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:47; the VL CDR2 comprises the amino acid sequence of SEQ ID NO:64; and the VL CDR3 comprises the amino acid sequence of SEQ ID NO:82;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:48; the VL CDR2 comprises the amino acid sequence of SEQ ID NO:64; and the VL CDR3 comprises the amino acid sequence of SEQ ID NO:83;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:49; the VL CDR2 comprises the amino acid sequence of SEQ ID NO:65; and the VL CDR3 comprises the amino acid sequence of SEQ ID NO:84;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:48; the VL CDR2 comprises the amino acid sequence of SEQ ID NO:64; and the VL CDR3 comprises the amino acid sequence of SEQ ID NO:85;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:50; the VL CDR2 comprises the amino acid sequence of SEQ ID NO:65; and the VL CDR3 comprises the amino acid sequence of SEQ ID NO:84;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:28; the VL CDR2 comprises the amino acid sequence of SEQ ID NO:66; and the VL CDR3 comprises the amino acid sequence of SEQ ID NO:86;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:47; the VL CDR2 comprises the amino acid sequence of SEQ ID NO:64; and the VL CDR3 comprises the amino acid sequence of SEQ ID NO:84;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:118; the VL CDR2 comprises the amino acid sequence of SEQ ID NO:66; and the VL CDR3 comprises the amino acid sequence of SEQ ID NO:87;
the VH CDR1 comprises the amino acid sequence of SEQ ID NO:3; the VH CDR2 comprises the amino acid sequence of SEQ ID NO:15; the VH CDR3 comprises the amino acid sequence of SEQ ID NO:22; the VL CDR1 comprises the amino acid sequence of SEQ ID NO:50; the VL CDR2 comprises the amino acid sequence of SEQ ID NO:64; and the VL CDR3 comprises the amino acid sequence of SEQ ID NO:88;
the VH CDR1 comprises the amino acid sequence of SEQ ID NO:5; the VH CDR2 comprises the amino acid sequence of SEQ ID NO:11; the VH CDR3 comprises the amino acid sequence of SEQ ID NO:23; the VL CDR1 comprises the amino acid sequence of SEQ ID NO:44; the VL CDR2 comprises the amino acid sequence of SEQ ID NO:61; and the VL CDR3 comprises the amino acid sequence of SEQ ID NO:89;
the VH CDR1 comprises the amino acid sequence of SEQ ID NO:5; the VH CDR2 comprises the amino acid sequence of SEQ ID NO:16; the VH CDR3 comprises the amino acid sequence of SEQ ID NO:24; the VL CDR1 comprises the amino acid sequence of SEQ ID NO:51; the VL CDR2 comprises the amino acid sequence of SEQ ID NO:67; and the VL CDR3 comprises the amino acid sequence of SEQ ID NO:90;
the VH CDR1 comprises the amino acid sequence of SEQ ID NO:1; the VH CDR2 comprises the amino acid sequence of SEQ ID NO:92; the VH CDR3 comprises the amino acid sequence of SEQ ID NO:18; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:93; the VH CDR3 comprises the amino acid sequence of SEQ ID NO:18; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:94; the VH CDR3 comprises the amino acid sequence of SEQ ID NO:18; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:95; the VH CDR3 comprises the amino acid sequence of SEQ ID NO:18; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:92; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:93; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:94; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:95; the VH CDR3 comprises the amino acid sequence of SEQ ID NO:18; the VL CDR1 comprises the amino acid sequence of SEQ ID NO:28; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:92; the VH CDR3 comprises the amino acid sequence of SEQ ID NO:18; the VL CDR1 comprises the amino acid sequence of SEQ ID NO:27; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:93; the VH CDR3 comprises the amino acid sequence of SEQ ID NO:18; the VL CDR1 comprises the amino acid sequence of SEQ ID NO:27; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:94; the VH CDR3 comprises the amino acid sequence of SEQ ID NO:18; the VL CDR1 comprises the amino acid sequence of SEQ ID NO:27; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:95; the VH CDR3 comprises the amino acid sequence of SEQ ID NO:18; the VL CDR1 comprises the amino acid sequence of SEQ ID NO:27; the VL CDR2 comprises the amino acid sequence of SEQ ID NO:54; and the VL CDR3 comprises the amino acid sequence of SEQ ID NO:71;
the VH CDR1 comprises the amino acid sequence of SEQ ID NO:1; the VH CDR2 comprises the amino acid sequence of SEQ ID NO:92; the VH CDR3 comprises the amino acid sequence of SEQ ID NO:18; the VL CDR1 comprises the amino acid sequence of SEQ ID NO:36; the VL CDR2 comprises the amino acid sequence of SEQ ID NO:54; and the VL CDR3 comprises the amino acid sequence of SEQ ID NO:72;
the VH CDR1 comprises the amino acid sequence of SEQ ID NO:1; the VH CDR2 comprises the amino acid sequence of SEQ ID NO:93; the VH CDR3 comprises the amino acid sequence of SEQ ID NO:18; the VL CDR1 comprises the amino acid sequence of SEQ ID NO:36; the VL CDR2 comprises the amino acid sequence of SEQ ID NO:54; and the VL CDR3 comprises the amino acid sequence of SEQ ID NO:72;
the VH CDR1 comprises the amino acid sequence of SEQ ID NO:1; the VH CDR2 comprises the amino acid sequence of SEQ ID NO:94; the VH CDR3 comprises the amino acid sequence of SEQ ID NO:18; the VL CDR1 comprises the amino acid sequence of SEQ ID NO:36; the VL CDR2 comprises the amino acid sequence of SEQ ID NO:54; and the VL CDR3 comprises the amino acid sequence of SEQ ID NO:72;
the VH CDR1 comprises the amino acid sequence of SEQ ID NO:1; the VH CDR2 comprises the amino acid sequence of SEQ ID NO:95; the VH CDR3 comprises the amino acid sequence of SEQ ID NO:18; the VL CDR1 comprises the amino acid sequence of SEQ ID NO:36; the VL CDR2 comprises the amino acid sequence of SEQ ID NO:54; and the VL CDR3 comprises the amino acid sequence of SEQ ID NO:72; or
the VH CDR1 comprises the amino acid sequence of SEQ ID NO:329; the VH CDR2 comprises the amino acid sequence of SEQ ID NO:7; the VH CDR3 comprises the amino acid sequence of SEQ ID NO:18; the VL CDR1 comprises the amino acid sequence of SEQ ID NO:30; the VL CDR2 comprises the amino acid sequence of SEQ ID NO:55; and the VL CDR3 comprises the amino acid sequence of SEQ ID NO:71.

5. The antibody of claim 1, wherein:
the VH is at least 80% identical to the amino acid sequence of any one of SEQ ID NOs:165-181 and 183-208; and
the VL is at least 80% identical to the amino acid sequence of any one of SEQ ID NOs:264-304 and 306-318.

6. The antibody of claim 1, wherein
the VH comprises the amino acid sequence of any one of SEQ ID NOs:165-181 and 183-208; and
the VL comprises the amino acid sequence of any one of SEQ ID NOs:264-304 and 306-318.

7. The antibody of claim 1, wherein
the VH comprises the amino acid sequence of SEQ ID NO:165 and the VL comprises the amino acid sequence of SEQ ID NO:264;
the VH comprises the amino acid sequence of SEQ ID NO:165 and the VL comprises the amino acid sequence of SEQ ID NO:265;
the VH comprises the amino acid sequence of SEQ ID NO:166 and the VL comprises the amino acid sequence of SEQ ID NO:266;
the VH comprises the amino acid sequence of SEQ ID NO:165 and the VL comprises the amino acid sequence of SEQ ID NO:266;
the VH comprises the amino acid sequence of SEQ ID NO:165 and the VL comprises the amino acid sequence of SEQ ID NO:267;
the VH comprises the amino acid sequence of SEQ ID NO:165 and the VL comprises the amino acid sequence of SEQ ID NO:268;
the VH comprises the amino acid sequence of SEQ ID NO:165 and the VL comprises the amino acid sequence of SEQ ID NO:269;
the VH comprises the amino acid sequence of SEQ ID NO:167 and the VL comprises the amino acid sequence of SEQ ID NO:270;
the VH comprises the amino acid sequence of SEQ ID NO:165 and the VL comprises the amino acid sequence of SEQ ID NO:271;
the VH comprises the amino acid sequence of SEQ ID NO:165 and the VL comprises the amino acid sequence of SEQ ID NO:272;
the VH comprises the amino acid sequence of SEQ ID NO:168 and the VL comprises the amino acid sequence of SEQ ID NO:273;
the VH comprises the amino acid sequence of SEQ ID NO:169 and the VL comprises the amino acid sequence of SEQ ID NO:274;
the VH comprises the amino acid sequence of SEQ ID NO:170 and the VL comprises the amino acid sequence of SEQ ID NO:275;
the VH comprises the amino acid sequence of SEQ ID NO:171 and the VL comprises the amino acid sequence of SEQ ID NO:276;
the VH comprises the amino acid sequence of SEQ ID NO:165 and the VL comprises the amino acid sequence of SEQ ID NO:277;
the VH comprises the amino acid sequence of SEQ ID NO:165 and the VL comprises the amino acid sequence of SEQ ID NO:278;
the VH comprises the amino acid sequence of SEQ ID NO:165 and the VL comprises the amino acid sequence of SEQ ID NO:279;
the VH comprises the amino acid sequence of SEQ ID NO:165 and the VL comprises the amino acid sequence of SEQ ID NO:280;
the VH comprises the amino acid sequence of SEQ ID NO:172 and the VL comprises the amino acid sequence of SEQ ID NO:281;
the VH comprises the amino acid sequence of SEQ ID NO:165 and the VL comprises the amino acid sequence of SEQ ID NO:282;
the VH comprises the amino acid sequence of SEQ ID NO:173 and the VL comprises the amino acid sequence of SEQ ID NO:283;
the VH comprises the amino acid sequence of SEQ ID NO:165 and the VL comprises the amino acid sequence of SEQ ID NO:284;
the VH comprises the amino acid sequence of SEQ ID NO:165 and the VL comprises the amino acid sequence of SEQ ID NO:285;
the VH comprises the amino acid sequence of SEQ ID NO:174 and the VL comprises the amino acid sequence of SEQ ID NO:286;
the VH comprises the amino acid sequence of SEQ ID NO:165 and the VL comprises the amino acid sequence of SEQ ID NO:287;
the VH comprises the amino acid sequence of SEQ ID NO:165 and the VL comprises the amino acid sequence of SEQ ID NO:288;
the VH comprises the amino acid sequence of SEQ ID NO:165 and the VL comprises the amino acid sequence of SEQ ID NO:289;
the VH comprises the amino acid sequence of SEQ ID NO:165 and the VL comprises the amino acid sequence of SEQ ID NO:290;
the VH comprises the amino acid sequence of SEQ ID NO:165 and the VL comprises the amino acid sequence of SEQ ID NO:291;
the VH comprises the amino acid sequence of SEQ ID NO:165 and the VL comprises the amino acid sequence of SEQ ID NO:292;
the VH comprises the amino acid sequence of SEQ ID NO:175 and the VL comprises the amino acid sequence of SEQ ID NO:293;
the VH comprises the amino acid sequence of SEQ ID NO:175 and the VL comprises the amino acid sequence of SEQ ID NO:294;
the VH comprises the amino acid sequence of SEQ ID NO:175 and the VL comprises the amino acid sequence of SEQ ID NO:295;
the VH comprises the amino acid sequence of SEQ ID NO:175 and the VL comprises the amino acid sequence of SEQ ID NO:296;
the VH comprises the amino acid sequence of SEQ ID NO:176 and the VL comprises the amino acid sequence of SEQ ID NO:294;
the VH comprises the amino acid sequence of SEQ ID NO:175 and the VL comprises the amino acid sequence of SEQ ID NO:297;
the VH comprises the amino acid sequence of SEQ ID NO:175 and the VL comprises the amino acid sequence of SEQ ID NO:298;
the VH comprises the amino acid sequence of SEQ ID NO:177 and the VL comprises the amino acid sequence of SEQ ID NO:299;
the VH comprises the amino acid sequence of SEQ ID NO:178 and the VL comprises the amino acid sequence of SEQ ID NO:300;
the VH comprises the amino acid sequence of SEQ ID NO:179 and the VL comprises the amino acid sequence of SEQ ID NO:301;
the VH comprises the amino acid sequence of SEQ ID NO:180 and the VL comprises the amino acid sequence of SEQ ID NO:301;
the VH comprises the amino acid sequence of SEQ ID NO:180 and the VL comprises the amino acid sequence of SEQ ID NO:302;
the VH comprises the amino acid sequence of SEQ ID NO:181 and the VL comprises the amino acid sequence of SEQ ID NO:303;
the VH comprises the amino acid sequence of SEQ ID NO:182 and the VL comprises the amino acid sequence of SEQ ID NO:304;
the VH comprises the amino acid sequence of SEQ ID NO:183 and the VL comprises the amino acid sequence of SEQ ID NO:306;
the VH comprises the amino acid sequence of SEQ ID NO:182 and the VL comprises the amino acid sequence of SEQ ID NO:307;
the VH comprises the amino acid sequence of SEQ ID NO:182 and the VL comprises the amino acid sequence of SEQ ID NO:308;
the VH comprises the amino acid sequence of SEQ ID NO:182 and the VL comprises the amino acid sequence of SEQ ID NO:309;
the VH comprises the amino acid sequence of SEQ ID NO:184 and the VL comprises the amino acid sequence of SEQ ID NO:310;
the VH comprises the amino acid sequence of SEQ ID NO:182 and the VL comprises the amino acid sequence of SEQ ID NO:311;
the VH comprises the amino acid sequence of SEQ ID NO:185 and the VL comprises the amino acid sequence of SEQ ID NO:312;
the VH comprises the amino acid sequence of SEQ ID NO:186 and the VL comprises the amino acid sequence of SEQ ID NO:313;
the VH comprises the amino acid sequence of SEQ ID NO:187 and the VL comprises the amino acid sequence of SEQ ID NO:314;
the VH comprises the amino acid sequence of SEQ ID NO:188 and the VL comprises the amino acid sequence of SEQ ID NO:268;
the VH comprises the amino acid sequence of SEQ ID NO:189 and the VL comprises the amino acid sequence of SEQ ID NO:268;
the VH comprises the amino acid sequence of SEQ ID NO:190 and the VL comprises the amino acid sequence of SEQ ID NO:268;
the VH comprises the amino acid sequence of SEQ ID NO:191 and the VL comprises the amino acid sequence of SEQ ID NO:268;
the VH comprises the amino acid sequence of SEQ ID NO:192 and the VL comprises the amino acid sequence of SEQ ID NO:268;
the VH comprises the amino acid sequence of SEQ ID NO:193 and the VL comprises the amino acid sequence of SEQ ID NO:268;
the VH comprises the amino acid sequence of SEQ ID NO:194 and the VL comprises the amino acid sequence of SEQ ID NO:268;
the VH comprises the amino acid sequence of SEQ ID NO:195 and the VL comprises the amino acid sequence of SEQ ID NO:268;
the VH comprises the amino acid sequence of SEQ ID NO:196 and the VL comprises the amino acid sequence of SEQ ID NO:268;
the VH comprises the amino acid sequence of SEQ ID NO:165 and the VL comprises the amino acid sequence of SEQ ID NO:315;
the VH comprises the amino acid sequence of SEQ ID NO:188 and the VL comprises the amino acid sequence of SEQ ID NO:315;
the VH comprises the amino acid sequence of SEQ ID NO:189 and the VL comprises the amino acid sequence of SEQ ID NO:315;
the VH comprises the amino acid sequence of SEQ ID NO:190 and the VL comprises the amino acid sequence of SEQ ID NO:315;
the VH comprises the amino acid sequence of SEQ ID NO:191 and the VL comprises the amino acid sequence of SEQ ID NO:315;
the VH comprises the amino acid sequence of SEQ ID NO:192 and the VL comprises the amino acid sequence of SEQ ID NO:315;
the VH comprises the amino acid sequence of SEQ ID NO:193 and the VL comprises the amino acid sequence of SEQ ID NO:315;
the VH comprises the amino acid sequence of SEQ ID NO:194 and the VL comprises the amino acid sequence of SEQ ID NO:315;
the VH comprises the amino acid sequence of SEQ ID NO:195 and the VL comprises the amino acid sequence of SEQ ID NO:315;
the VH comprises the amino acid sequence of SEQ ID NO:196 and the VL comprises the amino acid sequence of SEQ ID NO:315;
the VH comprises the amino acid sequence of SEQ ID NO:165 and the VL comprises the amino acid sequence of SEQ ID NO:316;
the VH comprises the amino acid sequence of SEQ ID NO:188 and the VL comprises the amino acid sequence of SEQ ID NO:316;
the VH comprises the amino acid sequence of SEQ ID NO:189 and the VL comprises the amino acid sequence of SEQ ID NO:316;
the VH comprises the amino acid sequence of SEQ ID NO:190 and the VL comprises the amino acid sequence of SEQ ID NO:316;
the VH comprises the amino acid sequence of SEQ ID NO:191 and the VL comprises the amino acid sequence of SEQ ID NO:316;
the VH comprises the amino acid sequence of SEQ ID NO:192 and the VL comprises the amino acid sequence of SEQ ID NO:316;
the VH comprises the amino acid sequence of SEQ ID NO:193 and the VL comprises the amino acid sequence of SEQ ID NO:316;
the VH comprises the amino acid sequence of SEQ ID NO:194 and the VL comprises the amino acid sequence of SEQ ID NO:316;
the VH comprises the amino acid sequence of SEQ ID NO:195 and the VL comprises the amino acid sequence of SEQ ID NO:316;
the VH comprises the amino acid sequence of SEQ ID NO:196 and the VL comprises the amino acid sequence of SEQ ID NO:316;
the VH comprises the amino acid sequence of SEQ ID NO:197 and the VL comprises the amino acid sequence of SEQ ID NO:268;
the VH comprises the amino acid sequence of SEQ ID NO:198 and the VL comprises the amino acid sequence of SEQ ID NO:268;
the VH comprises the amino acid sequence of SEQ ID NO:199 and the VL comprises the amino acid sequence of SEQ ID NO:268;
the VH comprises the amino acid sequence of SEQ ID NO:200 and the VL comprises the amino acid sequence of SEQ ID NO:268;
the VH comprises the amino acid sequence of SEQ ID NO:201 and the VL comprises the amino acid sequence of SEQ ID NO:268;
the VH comprises the amino acid sequence of SEQ ID NO:197 and the VL comprises the amino acid sequence of SEQ ID NO:315;
the VH comprises the amino acid sequence of SEQ ID NO:198 and the VL comprises the amino acid sequence of SEQ ID NO:315;
the VH comprises the amino acid sequence of SEQ ID NO:199 and the VL comprises the amino acid sequence of SEQ ID NO:315;
the VH comprises the amino acid sequence of SEQ ID NO:200 and the VL comprises the amino acid sequence of SEQ ID NO:315;
the VH comprises the amino acid sequence of SEQ ID NO:201 and the VL comprises the amino acid sequence of SEQ ID NO:315;
the VH comprises the amino acid sequence of SEQ ID NO:197 and the VL comprises the amino acid sequence of SEQ ID NO:316;
the VH comprises the amino acid sequence of SEQ ID NO:198 and the VL comprises the amino acid sequence of SEQ ID NO:316;
the VH comprises the amino acid sequence of SEQ ID NO:199 and the VL comprises the amino acid sequence of SEQ ID NO:316;
the VH comprises the amino acid sequence of SEQ ID NO:200 and the VL comprises the amino acid sequence of SEQ ID NO:316;
the VH comprises the amino acid sequence of SEQ ID NO:201 and the VL comprises the amino acid sequence of SEQ ID NO:316;
the VH comprises the amino acid sequence of SEQ ID NO:170 and the VL comprises the amino acid sequence of SEQ ID NO:268;
the VH comprises the amino acid sequence of SEQ ID NO:202 and the VL comprises the amino acid sequence of SEQ ID NO:268;
the VH comprises the amino acid sequence of SEQ ID NO:203 and the VL comprises the amino acid sequence of SEQ ID NO:268;
the VH comprises the amino acid sequence of SEQ ID NO:204 and the VL comprises the amino acid sequence of SEQ ID NO:268;
the VH comprises the amino acid sequence of SEQ ID NO:205 and the VL comprises the amino acid sequence of SEQ ID NO:268;
the VH comprises the amino acid sequence of SEQ ID NO:206 and the VL comprises the amino acid sequence of SEQ ID NO:268;
the VH comprises the amino acid sequence of SEQ ID NO:207 and the VL comprises the amino acid sequence of SEQ ID NO:268;
the VH comprises the amino acid sequence of SEQ ID NO:208 and the VL comprises the amino acid sequence of SEQ ID NO:268;
the VH comprises the amino acid sequence of SEQ ID NO:170 and the VL comprises the amino acid sequence of SEQ ID NO:315;
the VH comprises the amino acid sequence of SEQ ID NO:202 and the VL comprises the amino acid sequence of SEQ ID NO:315;
the VH comprises the amino acid sequence of SEQ ID NO:203 and the VL comprises the amino acid sequence of SEQ ID NO:315;
the VH comprises the amino acid sequence of SEQ ID NO:204 and the VL comprises the amino acid sequence of SEQ ID NO:315;
the VH comprises the amino acid sequence of SEQ ID NO:205 and the VL comprises the amino acid sequence of SEQ ID NO:315;
the VH comprises the amino acid sequence of SEQ ID NO:206 and the VL comprises the amino acid sequence of SEQ ID NO:315;
the VH comprises the amino acid sequence of SEQ ID NO:207 and the VL comprises the amino acid sequence of SEQ ID NO:315;
the VH comprises the amino acid sequence of SEQ ID NO:208 and the VL comprises the amino acid sequence of SEQ ID NO:315;
the VH comprises the amino acid sequence of SEQ ID NO:170 and the VL comprises the amino acid sequence of SEQ ID NO:316;
the VH comprises the amino acid sequence of SEQ ID NO:202 and the VL comprises the amino acid sequence of SEQ ID NO:316;
the VH comprises the amino acid sequence of SEQ ID NO:203 and the VL comprises the amino acid sequence of SEQ ID NO:316;
the VH comprises the amino acid sequence of SEQ ID NO:204 and the VL comprises the amino acid sequence of SEQ ID NO:316;
the VH comprises the amino acid sequence of SEQ ID NO:205 and the VL comprises the amino acid sequence of SEQ ID NO:316;
the VH comprises the amino acid sequence of SEQ ID NO:206 and the VL comprises the amino acid sequence of SEQ ID NO:316;
the VH comprises the amino acid sequence of SEQ ID NO:207 and the VL comprises the amino acid sequence of SEQ ID NO:316;
the VH comprises the amino acid sequence of SEQ ID NO:208 and the VL comprises the amino acid sequence of SEQ ID NO:316;
the VH comprises the amino acid sequence of SEQ ID NO:188 and the VL comprises the amino acid sequence of SEQ ID NO:277;
the VH comprises the amino acid sequence of SEQ ID NO:189 and the VL comprises the amino acid sequence of SEQ ID NO:277;
the VH comprises the amino acid sequence of SEQ ID NO:190 and the VL comprises the amino acid sequence of SEQ ID NO:277;
the VH comprises the amino acid sequence of SEQ ID NO:191 and the VL comprises the amino acid sequence of SEQ ID NO:277;
the VH comprises the amino acid sequence of SEQ ID NO:192 and the VL comprises the amino acid sequence of SEQ ID NO:277;
the VH comprises the amino acid sequence of SEQ ID NO:193 and the VL comprises the amino acid sequence of SEQ ID NO:277;
the VH comprises the amino acid sequence of SEQ ID NO:194 and the VL comprises the amino acid sequence of SEQ ID NO:277;
the VH comprises the amino acid sequence of SEQ ID NO:195 and the VL comprises the amino acid sequence of SEQ ID NO:277;
the VH comprises the amino acid sequence of SEQ ID NO:196 and the VL comprises the amino acid sequence of SEQ ID NO:277;
the VH comprises the amino acid sequence of SEQ ID NO:165 and the VL comprises the amino acid sequence of SEQ ID NO:317;
the VH comprises the amino acid sequence of SEQ ID NO:188 and the VL comprises the amino acid sequence of SEQ ID NO:317;
the VH comprises the amino acid sequence of SEQ ID NO:189 and the VL comprises the amino acid sequence of SEQ ID NO:317;
the VH comprises the amino acid sequence of SEQ ID NO:190 and the VL comprises the amino acid sequence of SEQ ID NO:317;
the VH comprises the amino acid sequence of SEQ ID NO:191 and the VL comprises the amino acid sequence of SEQ ID NO:317;
the VH comprises the amino acid sequence of SEQ ID NO:192 and the VL comprises the amino acid sequence of SEQ ID NO:317;
the VH comprises the amino acid sequence of SEQ ID NO:193 and the VL comprises the amino acid sequence of SEQ ID NO:317;
the VH comprises the amino acid sequence of SEQ ID NO:194 and the VL comprises the amino acid sequence of SEQ ID NO:317;
the VH comprises the amino acid sequence of SEQ ID NO:195 and the VL comprises the amino acid sequence of SEQ ID NO:317;
the VH comprises the amino acid sequence of SEQ ID NO:196 and the VL comprises the amino acid sequence of SEQ ID NO:317;
the VH comprises the amino acid sequence of SEQ ID NO:197 and the VL comprises the amino acid sequence of SEQ ID NO:277;
the VH comprises the amino acid sequence of SEQ ID NO:198 and the VL comprises the amino acid sequence of SEQ ID NO:277;
the VH comprises the amino acid sequence of SEQ ID NO:199 and the VL comprises the amino acid sequence of SEQ ID NO:277;
the VH comprises the amino acid sequence of SEQ ID NO:200 and the VL comprises the amino acid sequence of SEQ ID NO:277;
the VH comprises the amino acid sequence of SEQ ID NO:201 and the VL comprises the amino acid sequence of SEQ ID NO:277;
the VH comprises the amino acid sequence of SEQ ID NO:197 and the VL comprises the amino acid sequence of SEQ ID NO:317;
the VH comprises the amino acid sequence of SEQ ID NO:198 and the VL comprises the amino acid sequence of SEQ ID NO:317;
the VH comprises the amino acid sequence of SEQ ID NO:199 and the VL comprises the amino acid sequence of SEQ ID NO:317;
the VH comprises the amino acid sequence of SEQ ID NO:200 and the VL comprises the amino acid sequence of SEQ ID NO:317;
the VH comprises the amino acid sequence of SEQ ID NO:201 and the VL comprises the amino acid sequence of SEQ ID NO:317;
the VH comprises the amino acid sequence of SEQ ID NO:170 and the VL comprises the amino acid sequence of SEQ ID NO:277;
the VH comprises the amino acid sequence of SEQ ID NO:202 and the VL comprises the amino acid sequence of SEQ ID NO:277;
the VH comprises the amino acid sequence of SEQ ID NO:203 and the VL comprises the amino acid sequence of SEQ ID NO:277;
the VH comprises the amino acid sequence of SEQ ID NO:204 and the VL comprises the amino acid sequence of SEQ ID NO:277;
the VH comprises the amino acid sequence of SEQ ID NO:205 and the VL comprises the amino acid sequence of SEQ ID NO:277;
the VH comprises the amino acid sequence of SEQ ID NO:206 and the VL comprises the amino acid sequence of SEQ ID NO:277;
the VH comprises the amino acid sequence of SEQ ID NO:207 and the VL comprises the amino acid sequence of SEQ ID NO:277;
the VH comprises the amino acid sequence of SEQ ID NO:208 and the VL comprises the amino acid sequence of SEQ ID NO:277;
the VH comprises the amino acid sequence of SEQ ID NO:170 and the VL comprises the amino acid sequence of SEQ ID NO:317;
the VH comprises the amino acid sequence of SEQ ID NO:202 and the VL comprises the amino acid sequence of SEQ ID NO:317;
the VH comprises the amino acid sequence of SEQ ID NO:203 and the VL comprises the amino acid sequence of SEQ ID NO:317;
the VH comprises the amino acid sequence of SEQ ID NO:204 and the VL comprises the amino acid sequence of SEQ ID NO:317;
the VH comprises the amino acid sequence of SEQ ID NO:205 and the VL comprises the amino acid sequence of SEQ ID NO:317;
the VH comprises the amino acid sequence of SEQ ID NO:206 and the VL comprises the amino acid sequence of SEQ ID NO:317;
the VH comprises the amino acid sequence of SEQ ID NO:207 and the VL comprises the amino acid sequence of SEQ ID NO:317;
the VH comprises the amino acid sequence of SEQ ID NO:208 and the VL comprises the amino acid sequence of SEQ ID NO:317;
the VH comprises the amino acid sequence of SEQ ID NO:188 and the VL comprises the amino acid sequence of SEQ ID NO:279;
the VH comprises the amino acid sequence of SEQ ID NO:189 and the VL comprises the amino acid sequence of SEQ ID NO:279;
the VH comprises the amino acid sequence of SEQ ID NO:190 and the VL comprises the amino acid sequence of SEQ ID NO:279;
the VH comprises the amino acid sequence of SEQ ID NO:191 and the VL comprises the amino acid sequence of SEQ ID NO:279;
the VH comprises the amino acid sequence of SEQ ID NO:192 and the VL comprises the amino acid sequence of SEQ ID NO:279;
the VH comprises the amino acid sequence of SEQ ID NO:193 and the VL comprises the amino acid sequence of SEQ ID NO:279;
the VH comprises the amino acid sequence of SEQ ID NO:194 and the VL comprises the amino acid sequence of SEQ ID NO:279;
the VH comprises the amino acid sequence of SEQ ID NO:195 and the VL comprises the amino acid sequence of SEQ ID NO:279;
the VH comprises the amino acid sequence of SEQ ID NO:196 and the VL comprises the amino acid sequence of SEQ ID NO:279;
the VH comprises the amino acid sequence of SEQ ID NO:165 and the VL comprises the amino acid sequence of SEQ ID NO:318;
the VH comprises the amino acid sequence of SEQ ID NO:188 and the VL comprises the amino acid sequence of SEQ ID NO:318;
the VH comprises the amino acid sequence of SEQ ID NO:189 and the VL comprises the amino acid sequence of SEQ ID NO:318;
the VH comprises the amino acid sequence of SEQ ID NO:190 and the VL comprises the amino acid sequence of SEQ ID NO:318;
the VH comprises the amino acid sequence of SEQ ID NO:191 and the VL comprises the amino acid sequence of SEQ ID NO:318;
the VH comprises the amino acid sequence of SEQ ID NO:192 and the VL comprises the amino acid sequence of SEQ ID NO:318;
the VH comprises the amino acid sequence of SEQ ID NO:193 and the VL comprises the amino acid sequence of SEQ ID NO:318;
the VH comprises the amino acid sequence of SEQ ID NO:194 and the VL comprises the amino acid sequence of SEQ ID NO:318;
the VH comprises the amino acid sequence of SEQ ID NO:195 and the VL comprises the amino acid sequence of SEQ ID NO:318;
the VH comprises the amino acid sequence of SEQ ID NO:196 and the VL comprises the amino acid sequence of SEQ ID NO:318;
the VH comprises the amino acid sequence of SEQ ID NO:197 and the VL comprises the amino acid sequence of SEQ ID NO:279;
the VH comprises the amino acid sequence of SEQ ID NO:198 and the VL comprises the amino acid sequence of SEQ ID NO:279;
the VH comprises the amino acid sequence of SEQ ID NO:199 and the VL comprises the amino acid sequence of SEQ ID NO:279;
the VH comprises the amino acid sequence of SEQ ID NO:200 and the VL comprises the amino acid sequence of SEQ ID NO:279;
the VH comprises the amino acid sequence of SEQ ID NO:201 and the VL comprises the amino acid sequence of SEQ ID NO:279;
the VH comprises the amino acid sequence of SEQ ID NO:197 and the VL comprises the amino acid sequence of SEQ ID NO:318;
the VH comprises the amino acid sequence of SEQ ID NO:198 and the VL comprises the amino acid sequence of SEQ ID NO:318;
the VH comprises the amino acid sequence of SEQ ID NO:199 and the VL comprises the amino acid sequence of SEQ ID NO:318;
the VH comprises the amino acid sequence of SEQ ID NO:200 and the VL comprises the amino acid sequence of SEQ ID NO:318;
the VH comprises the amino acid sequence of SEQ ID NO:201 and the VL comprises the amino acid sequence of SEQ ID NO:318;
the VH comprises the amino acid sequence of SEQ ID NO:170 and the VL comprises the amino acid sequence of SEQ ID NO:279;
the VH comprises the amino acid sequence of SEQ ID NO:202 and the VL comprises the amino acid sequence of SEQ ID NO:279;
the VH comprises the amino acid sequence of SEQ ID NO:203 and the VL comprises the amino acid sequence of SEQ ID NO:279;
the VH comprises the amino acid sequence of SEQ ID NO:204 and the VL comprises the amino acid sequence of SEQ ID NO:279;
the VH comprises the amino acid sequence of SEQ ID NO:205 and the VL comprises the amino acid sequence of SEQ ID NO:279;
the VH comprises the amino acid sequence of SEQ ID NO:206 and the VL comprises the amino acid sequence of SEQ ID NO:279;
the VH comprises the amino acid sequence of SEQ ID NO:207 and the VL comprises the amino acid sequence of SEQ ID NO:279;
the VH comprises the amino acid sequence of SEQ ID NO:208 and the VL comprises the amino acid sequence of SEQ ID NO:279;
the VH comprises the amino acid sequence of SEQ ID NO:170 and the VL comprises the amino acid sequence of SEQ ID NO:318;
the VH comprises the amino acid sequence of SEQ ID NO:202 and the VL comprises the amino acid sequence of SEQ ID NO:318;
the VH comprises the amino acid sequence of SEQ ID NO:203 and the VL comprises the amino acid sequence of SEQ ID NO:318;
the VH comprises the amino acid sequence of SEQ ID NO:204 and the VL comprises the amino acid sequence of SEQ ID NO:318;
the VH comprises the amino acid sequence of SEQ ID NO:205 and the VL comprises the amino acid sequence of SEQ ID NO:318;
the VH comprises the amino acid sequence of SEQ ID NO:206 and the VL comprises the amino acid sequence of SEQ ID NO:318;
the VH comprises the amino acid sequence of SEQ ID NO:207 and the VL comprises the amino acid sequence of SEQ ID NO:318; or
the VH comprises the amino acid sequence of SEQ ID NO:208 and the VL comprises the amino acid sequence of SEQ ID NO:318.

8.-18. (canceled)

19. The antibody of claim 1, wherein the antibody comprises:
a heavy chain comprising the amino acid sequence of any one of SEQ ID NOs:119-135 and 137-164; and
a light chain comprising the amino acid sequence of any one of SEQ ID NOs:209-249 and 251-263.

20. The antibody of claim 1, wherein the antibody comprises:
a heavy chain and a light chain each corresponding to the heavy chain and the light chain set forth in Tables 4-5 for a single clone selected from the group consisting of clones 1-53 and 55-215.

21.-31. (canceled)

32. An antibody that binds to human mutant calreticulin (CALR), wherein:
the antibody comprises a heavy chain variable region (VH) comprising a VH CDR1; a VH CDR2; and a VH CDR3; wherein:
the VH CDR1 comprises the amino acid sequence ELSMQ (SEQ ID NO:1);
the VH CDR2 comprises the amino acid sequence GFDPDDX₁₀₁ETMYAEX₁₀₂X₁₀₃QG (SEQ ID NO:102); wherein X₁₀₁ is D or G; wherein X₁₀₂ is K or R; and wherein X₁₀₃ is F or L;
the VH CDR3 is SPGYDFFDY (SEQ ID NO:18);
wherein the antibody comprises a light chain variable region (VL) comprising a VL CDR1, a VL CDR2, and a VL CDR3, wherein:
the VL CDR1 comprises the amino acid sequence GGX₁₀₄X₁₀₅X₁₀₆GX₁₀₇X₁₀₈X₁₀₉VX₁₁₀ (SEQ ID NO:103), wherein X₁₀₄ is N, D, or S; wherein X₁₀₅ is Y, N, or D; wherein X₁₀₆ is I or T; wherein X₁₀₇ is S, D, I, R, or T; wherein X₁₀₈ is K, E, or I; wherein X₁₀₉ is S, I, R, G, N, or A; and wherein X₁₁₀ is H, F, or N;
the VL CDR2 comprises the amino acid sequence DDX₁₁₁DRPX₁₁₂ (SEQ ID NO:104), wherein X₁₁₁ is G, S, or R; and wherein X₁₁₂ is S or L; and
the VL CDR3 comprises the amino acid sequence QVWDX₁₁₃X₁₁₄X₁₁₅DX₁₁₆X₁₁₇X₁₁₈ (SEQ ID NO:105), wherein X₁₁₃ is S or A; wherein X₁₁₄ is I or S; wherein X₁₁₅ is S, I, or N; wherein X₁₁₆ is H, L, or Q; wherein X₁₁₇ is V or L; and wherein X₁₁₈ is V or I.

33. (canceled)

34. An antibody that binds to human mutant calreticulin (CALR), wherein:
the antibody comprises a heavy chain variable region (VH) comprising a VH CDR1; a VH CDR2; and a VH CDR3; wherein:
the VH CDR1 comprises the amino acid sequence ELSMQ (SEQ ID NO:1);
the VH CDR2 comprises the amino acid sequence GFDPDDX₁₀₁ETMYAEX₁₀₂X₁₀₃QG (SEQ ID NO:102); wherein X₁₀₁ is D or G; wherein X₁₀₂ is K or R; and wherein X₁₀₃ is F or L;
the VH CDR3 is SPGYDFFDY (SEQ ID NO:18);
wherein the antibody comprises a light chain variable region (VL) comprising a VL CDR1, a VL CDR2, and a VL CDR3, wherein:
the VL CDR1 comprises the amino acid sequence TGTSSDVGGYNYVS (SEQ ID NO:30);
the VL CDR2 comprises the amino acid sequence X₁₁₉VSX₁₂₀RPS (SEQ ID NO:106); wherein X₁₁₉ is E or D; and wherein X₁₂₀ is N or K; and
the VL CDR3 comprises the amino acid sequence QVWDSSNDLLI (SEQ ID NO:71).

35. (canceled)

36. An antibody that binds to human mutant calreticulin (CALR), wherein:
the antibody comprises a heavy chain variable region (VH) comprising a VH CDR1; a VH CDR2; and a VH CDR3; wherein:
the VH CDR1 comprises the amino acid sequence DYFIH (SEQ ID NO:2);
the VH CDR2 comprises the amino acid sequence LVDPEDGETIYAEX₁₂₁FQG (SEQ ID NO:107), wherein X₁₂₁ is K or R;
the VH CDR3 comprises the amino acid sequence PGGILTDPDAFDI (SEQ ID NO:19);
wherein the antibody comprises a light chain variable region (VL) comprising a VL CDR1, a VL CDR2, and a VL CDR3, wherein:
the VL CDR1 comprises the amino acid sequence X₁₂₂GTX₁₂₃SDVGGYNX₁₂₄VS (SEQ ID NO:108), wherein X₁₂₂ is T or A; wherein X₁₂₃ is S or G; and wherein X₁₂₄ is Y or H;
the VL CDR2 comprises the amino acid sequence X₁₂₅VX₁₂₆X₁₂₇RPS (SEQ ID NO:109), wherein X₁₂₅ is D or E; wherein X₁₂₆ is N or S; and wherein X₁₂₇ is K or N; and
the VL CDR3 comprises the amino acid sequence SSYX₁₂₈X₁₂₉SSTX₁₃₀X₁₃₁V (SEQ ID NO:110), wherein X₁₂₈ is I or T; wherein X₁₂₉ is P or S; wherein X₁₃₀ is R, P, F, or absent; and wherein X₁₃₁ is W or Y.

37. An antibody that binds to human mutant calreticulin (CALR), wherein:
the antibody comprises a heavy chain variable region (VH) comprising a VH CDR1; a VH CDR2; and a VH CDR3; wherein:
the VH CDR1 comprises the amino acid sequence SYAIS (SEQ ID NO:3);
the VH CDR2 comprises the amino acid sequence LVDPEDGETIYAEKFX₁₃₂G (SEQ ID NO:111), wherein X₁₃₂ is R or Q;
the VH CDR3 comprises the amino acid sequence EESYGP (SEQ ID NO:20);
wherein the antibody comprises a light chain variable region (VL) comprising a VL CDR1, a VL CDR2, and a VL CDR3, wherein:
the VL CDR1 comprises the amino acid sequence QASQDISNYLX₁₃₃ (SEQ ID NO:112), X₁₃₃ is N or D;
the VL CDR2 comprises the amino acid sequence DASNLET (SEQ ID NO:61); and
the VL CDR3 comprises the amino acid sequence QQLNSYPLT (SEQ ID NO:80).

38. An antibody that binds to human mutant calreticulin (CALR), wherein:
the antibody comprises a heavy chain variable region (VH) comprising a VH CDR1; a VH CDR2; and a VH CDR3; wherein:
the VH CDR1 comprises the amino acid sequence EX₁₃₄SMH (SEQ ID NO:113), wherein X₁₃₄ is S or L;
the VH CDR2 comprises the amino acid sequence LVDPEDGETIYAQKFQG (SEQ ID NO:14);
the VH CDR3 comprises the amino acid sequence EEWSGDGDDAFDI (SEQ ID NO:21);
wherein the antibody comprises a light chain variable region (VL) comprising a VL CDR1, a VL CDR2, and a VL CDR3, wherein:
the VL CDR1 comprises the amino acid sequence SGSSSNIGSYSVS (SEQ ID NO:46);
the VL CDR2 comprises the amino acid sequence DX₁₃₅NKRPS (SEQ ID NO:114), wherein X₁₃₅ is N or D; and
the VL CDR3 comprises the amino acid sequence GTWDSSLSAWV (SEQ ID NO:81).

39. An antibody that binds to human mutant calreticulin (CALR), wherein:
the antibody comprises a heavy chain variable region (VH) comprising a VH CDR1; a VH CDR2; and a VH CDR3; wherein:
the VH CDR1 comprises the amino acid sequence SYAIS (SEQ ID NO:3);
the VH CDR2 comprises the amino acid sequence GIIPIFGTANYAQKFQG (SEQ ID NO:15);
the VH CDR3 comprises the amino acid sequence SPLRGSGWYWHYYYGMDV (SEQ ID NO:22);
wherein the antibody comprises a light chain variable region (VL) comprising a VL CDR1, a VL CDR2, and a VL CDR3, wherein:
the VL CDR1 comprises the amino acid sequence GGNX₁₃₆IX₁₃₇X₁₃₈KX₁₃₉VH (SEQ ID NO:115), wherein X₁₃₆ is N or K; wherein X₁₃₇ is R or G; wherein X₁₃₈ is A, S, R, or T; and wherein X₁₃₉ is H or S;
the VL CDR2 comprises the amino acid sequence X₁₄₀DX₁₄₁X₁₄₂RPS (SEQ ID NO: 116), wherein X₁₄₀ is Q or R; wherein X₁₄₁ is S or R; and wherein X₁₄₂ is N or K; and
the VL CDR3 comprises the amino acid sequence QX₁₄₃WX₁₄₄SX₁₄₅TX₁₄₆V (SEQ ID NO:117), wherein X₁₄₃ is A or V; wherein X₁₄₄ is D or G; wherein X₁₄₅ is S or N; and wherein X₁₄₆ is V, A, or E.

40. An antibody that binds to human mutant calreticulin (CALR), wherein:
the antibody comprises a heavy chain variable region (VH) comprising a VH CDR1; a VH CDR2; and a VH CDR3; wherein:
the VH CDR1 comprises the amino acid sequence TSGMGVG (SEQ ID NO:6);
the VH CDR2 comprises the amino acid sequence HIWWDDDKYYNPYLKN (SEQ ID NO:17);
the VH CDR3 comprises the amino acid sequence SAYGSTYGY (SEQ ID NO:25);
wherein the antibody comprises a light chain variable region (VL) comprising a VL CDR1, a VL CDR2, and a VL CDR3, wherein:
the VL CDR1 comprises the amino acid sequence KASQSVDYDGDSFMN (SEQ ID NO:52);
the VL CDR2 comprises the amino acid sequence TASILES (SEQ ID NO:68); and
the VL CDR3 comprises the amino acid sequence QQSNEDPWT (SEQ ID NO:91).

41.-43. (canceled)

44. An antibody that binds to human mutant calreticulin (CALR), wherein the antibody:
inhibits one or more signaling pathways downstream of thrombopoietin receptor (MPL) in a cell expressing human mutant CALR;
inhibits oncogenic cell proliferation in a cell expressing human mutant CALR;
inhibits dimerization of MPL in a cell expressing human mutant CALR; and/or
inhibits binding of MPL to human mutant CALR.

45.-48. (canceled)

49. An antibody that binds to human mutant calreticulin (CALR), wherein the antibody has modulated Fc effector function.

50.-54. (canceled)

55. An antibody that binds to human mutant calreticulin (CALR), wherein the antibody binds to human mutant CALR at an epitope within DEEQRTRRMMRTKMRMRRMRR (SEQ ID NO:328).

56.-65. (canceled)

66. An antibody that binds to human mutant calreticulin (CALR), wherein the antibody binds two non-identical epitopes within DEEQRTRRMMRTKMRMRRMRR (SEQ ID NO:328).

67. An antibody that binds to human mutant calreticulin (CALR), wherein the antibody binds to human mutant CALR at a first epitope within DEEQRTRRMMRTKMRMRRMRR (SEQ ID NO:328) and at a second epitope within of DEEQRTRRMMRTKMRMRRMRR (SEQ ID NO:328), wherein the first epitope and the second epitope are non-identical.

68.-71. (canceled)

72. An antibody that binds to human mutant CALR and competes for binding to human mutant CALR with an antibody that has a heavy chain comprising the amino acid sequence of SEQ ID NO:327 and a light chain comprising the amino acid sequence of SEQ ID NO:213.

73. An antibody that binds to human mutant CALR and inhibits or reduces binding of human mutant CALR to thrombopoietin receptor (MPL).

74.-85. (canceled)

86. A nucleic acid or a set of nucleic acids, which collectively encodes the antibody of claim 1.

87. An expression vector or a set of expression vectors comprising the nucleic acid or the set of nucleic acids of claim 86 operably linked to a promoter.

88. An isolated cell comprising a nucleic acid or a set of nucleic acids, which collectively encodes the antibody of claim 1, or an expression vector or a set of expression vectors comprising the nucleic acid or the set of nucleic acids, which collectively encodes the antibody of claim 1, operably linked to a promoter.

89. A method of making an antibody, comprising culturing the cell of claim 88 and isolating the antibody.

90. A pharmaceutical composition comprising the antibody of claim 1, and a pharmaceutically acceptable carrier.

91. A method of treating a myeloproliferative neoplasm in a human subject in need thereof, the method comprising administering to the human subject an effective amount of the antibody of claim 1.

92. (canceled)

93. The method of claim 91, further comprising administering to the human subject an additional therapy selected from the group consisting of a Janus tyrosine kinase (JAK) inhibitor, a phosphoinositide 3-kinase (PI3K) inhibitor, a standard of care therapy, or a combination thereof.

94.-96. (canceled)

97. The method of claim 93, wherein the administration of the antibody of claim 1 in combination with the JAK inhibitor produces a synergistic effect.

98. The method of claim 97, wherein the JAK inhibitor is ruxolitinib.

99. A kit comprising the antibody of claim 1, and instructions for use in treating a myeloproliferative neoplasm in a human subject in need thereof, optionally with instructions for use in combination with an additional therapy.

100. A method of detecting a CALR exon 9 mutation in a biological sample, the method comprising obtaining a biological sample from a human subject who has or is suspected of having a myeloproliferative neoplasm and contacting the sample with the antibody of claim 1 such that the antibody binds to a mutCALR protein if the mutCALR protein is present in the biological sample.

101. A method of diagnosing a human subject with a myeloproliferative neoplasm, the method comprising obtaining a biological sample from a human subject who has or is suspected of having a myeloproliferative neoplasm and contacting the sample with the antibody of claim 1 such that the antibody binds to a mutCALR protein if the mutCALR protein is present in the biological sample.
